(19) United States
(12) Patent Application Publication
(10) Pub. No.: US 2023/0357134 A1
(43) Pub. Date: Nov. 9, 2023

(54) AGENTS FOR USE IN THE TREATMENT OF AMYLOIDOSIS

(71) Applicant: UCL BUSINESS LTD, London (GB)

(72) Inventors: Vittorio BELLOTTI, London (GB); Alessandra CORAZZA, London (GB); Palma Patrizia MANGIONE, London (GB); Mark Brian PEPYS, London (GB); Christopher SWAIN, London (GB); Graham Walter TAYLOR, London (GB)

(21) Appl. No.: 18/245,235

(22) PCT Filed: Sep. 16, 2021
(86) PCT No.: PCT/GB2021/052404

§ 371 Date: Mar. 14, 2023

(30) Foreign Application Priority Data
Sep. 16, 2020 (GB) 2014589.2

(51) Int. Cl. C07C 229/58; C07D 237/24; C07D 213/80; C07D 263/57; C07D 231/12; C07D 263/34; A61P 43/00

(52) U.S. Cl. CPC C07C 229/58; C07D 237/24; C07D 213/80; C07D 263/57; C07D 231/12; C07D 263/34; A61P 43/00

(57) ABSTRACT

The present invention relates to compounds for stabilising the native tetrameric form of transthyretin and protecting it from proteolytic cleavage; compounds for use in the prevention and treatment of transthyretin amyloidosis; and agents and medicaments comprising such compounds. The compounds are based on a general structure A-L-B, wherein A is a group of formula (II) or of formula (III): or of formula (IV) or of formula (V) B is a group of formula (III), (IV), or (V), or a group of formula (VI) or a group of formula —R10Z, wherein: Z is selected from —CO2R′, —CONR′R″, —SO2R′ wherein R′ and R″ are independently H or C1-C4 alkyl; and R10 is a C1-C4 alkylene or alkenylene group; and L represents a linker group which is a saturated or unsaturated chain of 5 to 13 carbon atoms.

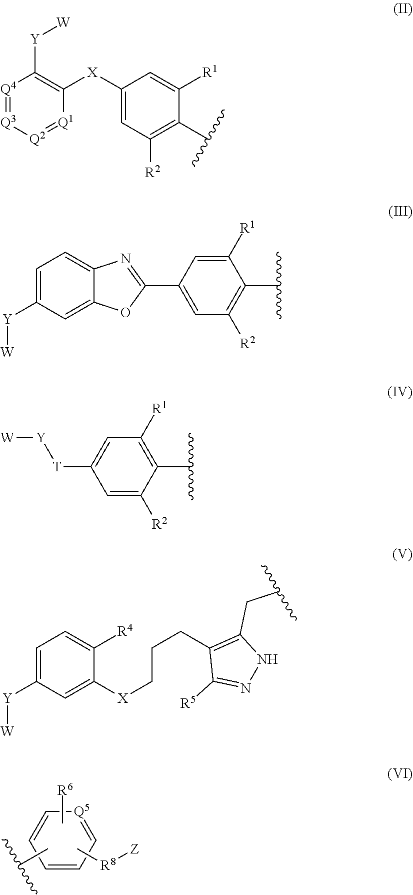

### FIELD OF THE INVENTION

The present invention relates to compounds for stabilising the native tetrameric form of transthyretin and protecting it from proteolytic cleavage; compounds for use in the prevention and treatment of transthyretin amyloidosis; and agents and medicaments comprising such compounds.

### BACKGROUND OF THE INVENTION

Amyloidosis is a serious disease caused by extracellular deposition of insoluble abnormal fibrils composed of one or other of the body's own proteins, including, importantly, normal wild type or genetic variants of the plasma protein, transthyretin (TTR) (Pepys, 2006).

Systemic amyloidosis, with deposits in the viscera, blood vessels and connective tissue, is usually fatal, causing about one per thousand deaths in developed countries. About 25 different unrelated human proteins form amyloid fibrils in vivo. Amyloid is deposited when there is: (i) sustained exposure to either normal or increased concentrations of a normal, potentially amyloidogenic, protein; (ii) when an abnormal amyloidogenic protein is produced as a consequence of an acquired disease; or (iii) when a gene mutation encodes an amyloidogenic variant protein. Fibrillogenesis results from reduced stability of the native fold of the fibril precursor protein, so that under physiological conditions it populates partly unfolded intermediate states which aggregate as stable amyloid fibrils with a pathognomonic cross-β sheet core structure (Sunde et al. 1997).

Wild type TTR, the normal plasma protein which transports thyroid hormone and retinol binding protein, is inherently amyloidogenic and forms microscopic amyloid deposits of uncertain clinical significance in all individuals aged over 80 years. Massive deposits in the heart can also occur, causing fatal wild type cardiac ATTR amyloidosis, previously called senile cardiac transthyretin amyloidosis. The inherent amyloidogenicity of wild type TTR is markedly enhanced by most of the reported >120 different point mutations which encode single residue substitutions in the TTR sequence (http://amyloidosismutations.com). These mutations cause autosomal dominant adult onset hereditary ATTR amyloidosis, a universally fatal condition affecting about 10,000 patients worldwide. The usual clinical presentation is familial amyloid polyneuropathy, with predominant peripheral and autonomic neuropathy, but there is commonly also serious involvement of the heart, kidneys and eyes. The condition typically presents after the causative gene has been transmitted to the proband's offspring, ensuring persistence of this devastating disease. Amyloidogenic mutations occur in all ethnic groups, but by far the most common, V³⁰M, clusters in three geographical foci: Northern Portugal, Northern Sweden and parts of Japan. A common amyloidogenic variant in the UK and Eire is T60A. TTR amyloidosis predominantly affecting the heart is particularly associated with the V122I variant, which is very rare in Caucasians but is carried by 4% of African Americans: 1.3 million people, including 13,000 individuals homozygous for the mutation (Jacobson, 1997). It is the second most common pathogenic mutation in that population after sickle cell haemoglobin. Cardiac ATTR amyloidosis presents as progressive, ultimately fatal, heart failure with preserved ejection fraction, is rarely suspected and is often misdiagnosed as coronary heart disease.

Liver transplantation provides an effective treatment for some patients with V30M ATTR amyloidosis, provided the procedure is done early enough. TTR is synthesized by hepatocytes and by the choroid plexus. Liver transplantation removes the source of the amyloidogenic variant TTR in the plasma and replaces it with wild type TTR. Liver transplantation does not affect variant TTR production by the choroid plexus and thus does not protect against amyloid deposition in the eye and leptomeninges. Furthermore, the procedure is available for only a minority of patients. There is a severe shortage of donor livers and the diagnosis of ATTR amyloidosis is often too late for optimal results to be obtained. In addition, patients with mutations other than V30M usually develop rapidly progressive cardiac amyloidosis after transplantation. In patients with predominant cardiac amyloid, heart transplantation is a possible option, but most are too old and are not acceptable recipients for extremely scarce donor organs.

In view of the limitations of transplantation therapy, therapeutic drug approaches have been investigated and there are currently two different approaches, each with licensed drugs available for use. The first approach uses small molecule ligands that are bound by transthyretin to stabilise the protein in the circulation and prevent it from misfolding to form amyloid fibrils. The licensed drug intended to achieve this objective is tafamidis (Pfizer's Vyndagel). The very old generic NSAID drug, diflunisal, has similar properties and an experimental compound, AG10, is currently being trialled. The second approach uses gene expression knock down drugs, Alnylam's siRNA drug, patisiran (Onpattro), and Akcea's ASO drug, inotersen (Tegsedi), to suppress TTR production by the liver. The clinical evidence for efficacy of the existing TTR stabilisers is limited, especially via their purported mode of action. The gene knockdown approaches markedly lower plasma transthyretin concentrations and have demonstrated therapeutic efficacy. However, gene expression knock down drugs are extremely costly and are not feasible for prophylactic use. Vyndagel is also extremely expensive and neither it nor diflunisal have shown sufficient efficacy to encourage belief in potential prophylactic use.

We have lately characterized for the first time the precise molecular process by which transthyretin actually forms amyloid in vivo (Mangione et al., 2014; Marcoux et al., 2015; Mangione e al., 2018; Raimondi et al., 2020) and have invented a novel family of compounds that potently and almost completely inhibits it. These compounds are suitable for medicinal use to both prevent and treat ATTR amyloidosis of all types. They will have the same role in ATTR amyloidosis that statins have in atherosclerotic cardiovascular disease. They are more potent and much more effective inhibitors of TTR amyloid fibrillogenesis than all the existing TTR stabilisers. Furthermore, our elucidation of the actual pathophysiological mechanism of TTR amyloidogenesis shows that even the lowered plasma TTR concentration produced by the gene expression knock down drugs will still allow TTR amyloid fibril formation. Thus, in addition to uniquely being prophylactic, our compounds will also act synergistically with TTR knockdown.

The native TTR molecule is a homotetramer of molecular weight 55,044 Da, and the non-covalently associated protomers, of mass 13,761, each contain 127 residues with a β-sandwich fold. The native tetramer binds a single retinol binding protein molecule and contains two identical negatively cooperative L-thyroxine (T4) binding pockets. Amyloid fibril formation by TTR was, until recently, generally considered to involve dissociation of the tetramer, partial unfolding of the protomers and then aggregation into the amyloid cross-β core structure. One therapeutic drug approach is taught in WO03/013508 which describes agents comprising ligands capable of being bound by transthyretin which are covalently co-linked by a linker. The purpose of these agents is to form complexes between separate transthyretin tetramers in the subject to be treated. This approach relies on the complexes being recognised by the body as abnormal and rapidly cleared from the circulation. In this way, the amyloidogenic protein is no longer available as a source for amyloid deposition. This is the same goal as is achieved by the TTR gene knockdown treatments, Onpattro and Tegsedi. However, the exemplified bivalent compounds proved to be ineffective in vivo and were not developable as drugs.

The alternative TTR stabilisation approach, using small molecule ligands which are specifically bound in the thyroid hormone binding pocket to prevent dissociation of the native homotetrameric TTR into dimers and protomers leading to fibrillogenic aggregation, is described, for example, in WO2004/05635. This shows an array of biphenyl and benzoxazole compounds, including 2-(3,5-dichlorophenyl)benzo[d]oxazole-6-carboxylic acid (tafamidis), which is marketed by Pfizer as Vyndaqel and has the following structure.

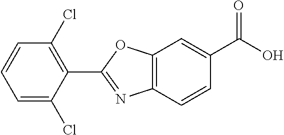 [SMILES: O=C(O)c1ccc2nc(-c3c(Cl)cccc3Cl)oc2c1]

In the academic literature, in vitro studies have shown that bis-arylamine compounds are bound with reasonably high affinity in the binding pocket in TTR and that this binding can stabilise the tetrameric form. Oza et al (2002) describe the structures of various compounds of this type. They distinguish between a “forward mode” of binding and a “reverse mode”, depending on which aryl ring locates to the interior and the exterior of the TTR tetramer. Oza et al is particularly concerned with analogues of diclofenac, which has a ring bearing a chlorine substituent and a second ring bearing a carboxylate group. When the ligands are soaked into TTR crystals, the X-ray structures of the complex with diclofenac analogue, 2-(3,5-dichlorophenyl amino)benzoic acid and that with diclofenac itself, show binding in the “reverse mode” with the carboxylate bearing ring occupying the inner binding cavity of the TTR binding pockets. Wiseman et al (2005) also shows 2-(3,5-dichlorophenylamino)benzoic acid, compound (2), binding in this reverse mode. Green et al (2003) describe various bivalent compounds comprising two head group ligands for the thyroxine binding site of TTR, joined together by a linking chain. Based on the reported X-ray structure of monovalent ligand soaked crystals, they synthesised their compounds with the linker covalently attached to the aryl group intended for binding by the inner binding cavity of the binding pockets. However, these molecules were not bound by native TTR. A complex with these bivalent ligands bound by TTR was only produced ifTTR was first completely denatured by harsh chaotropic agents, then mixed with the compounds and the TTR tetramer allowed to reassemble around them. The complex thus formed contained the head groups within each binding pocket with the linker situated in the central channel that traverses the tetramer core. Although TTR that had been refolded around the Green bivalent compounds was extremely stable, and could not be denatured like native TTR, the compounds were ineffective in the standard TTR fibrillogenesis assay because they were not bound by native TTR. These results suggest that bivalent compounds would not be effective for TTR stabilisation and thus would not be successful in the treatment or prevention of TTR amyloidosis.

WO2009/040405 describes compounds for stabilising the tetrameric form of transthyretin. The compounds have a palindromic structure consisting of two bis-arylamine groups that mimic the binding effect of thyroxine, joined through a linker L which is a linear or branched chain of 7 to 13 carbon atoms. A preferred compound according to this prior art is designated mds84 and has the following structure:

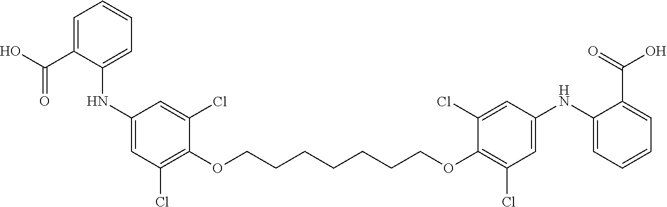 [SMILES: O=C(O)c1ccccc1Nc1cc(Cl)c(OCCCCCCCOc2c(Cl)cc(Nc3ccccc3C(=O)O)cc2Cl)c(Cl)c1]

Comparative studies showed that, surprisingly, and in contrast to the monovalent ligands such as 2-(3,5-dichlorophenylamino)benzoic acid and tafamidis, or the bivalent ligands reported by Green and colleagues (Green, 200³), which lack the dichloroaryl structure, mds84 is bound by TTR with much higher affinity and avidity, indeed pseudoirreversibly. The unique palindromic design of mds84, with the specific orientation of the two differently substituted aryl rings at each end of the molecule, enables this compound to enter the native TTR tetramer, traverse the molecule, simultaneously occupying both binding pockets with the two head groups and the core channel with the linker. These properties make mds84 a ‘superstabiliser’ of TTR. However, the properties of mds84 are not suitable for its development as a drug. In particular, mds84 is extremely hydrophobic, with a log P of 12.6. It has a short half-life in vivo, and very poor bioavailability for an oral drug.

A need remains for further compounds for the prevention and treatment of systemic transthyretin, ATTR, amyloidosis.

In pursuing the quest for such compounds, we elucidated, for the first time, the pathophysiological mechanism by which wild type and variant transthyretin actually form amyloid fibrils in vivo. Contrary to the generally adopted previous model, which involved simple denaturation of transthyretin by prolonged exposure to pH 4, the true mechanism is more complex. In fact, there is no in vivo compartment, relevant to systemic amyloidosis, in which transthyretin could possibly be exposed to prolonged acid conditions. Furthermore, such exposure simply denatures and aggregated transthyretin with production of very few genuine amyloid fibrils, if any. In contrast, we have shown that transthyretin amyloid fibrillogenesis requires a crucial specific tryptic proteolytic cleavage at residue 48 in a single protomer. This destabilises the whole tetramer and, in the presence of physiological magnitude physical forces, leads to high yield transformation of the cleaved native transthyretin into prolific authentic amyloid fibrils (Marcoux et al. 2015). The enzyme responsible for the cleavage in vivo is plasmin (Mangione et al. 2018). The contrast between this novel mechano-enzymatic mechanism and the simple low pH denaturation model previously used universally is very stark. The mechano-enzymatic mechanism is uniquely consistent with in vivo biological conditions. It generates fibrils with thermodynamic stability very similar to natural ex vivo TTR amyloid fibrils, and has nucleation dependent kinetics. None of these features exist in the low pH model. The nucleation dependent kinetics of fibrillogenesis are particularly consistent with the clinical pathophysiology of the disease in which amyloid nuclei created by amyloidogenic TTR variants accelerate the disease by enabling recruitment of wild type TTR.

Design of effective ‘superstabilisers’ oftransthyretin, to prevent amyloid fibrillogenesis, thus absolutely requires testing in the mechano-enzymatic mechanism. Surprisingly, the compounds of the present invention are markedly more potent and effective than tafamidis, AG10 and any of the other single head group monovalent ligands that have been reported or developed, and are correspondingly superior for medicinal use to prevent and treat transthyretin amyloidosis (Verona et al. 2017).

### SUMMARY OF THE INVENTION

The present invention aims to provide agents or compounds with improved properties over those described in the prior art and which are suitable for use particularly in the prevention and/or treatment of all forms of transthyretin, ATTR, amyloidosis.

Accordingly, in a first aspect, there is provided an agent for stabilising the tetrameric form of transthyretin, specifically to inhibit the mechano-enzymatic mechanism of transthyretin amyloid fibrillogenesis, which comprises a compound of the general formula (I) or a pharmaceutically acceptable salt, ester or prodrug thereof:

A-L-B (I)
wherein:
A is a group of formula (II):

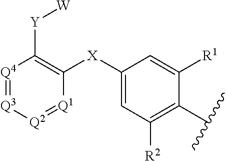

or of formula (III):

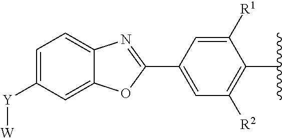

or of formula (IV):

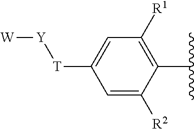

or of formula (V):

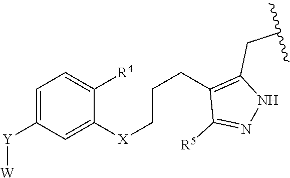

wherein: Y is independently a direct bond or a C1-C4 alkylene group which may be linear, branched, or may include a cyclopropyl group; W is —COOH or a tetrazole group;
   Q¹, Q², Q and Q⁴ are independently CH or N, provided that no more than two of Q¹, Q², Q³ and Q⁴ are N;
   X is independently —NH—, —O—, —S—, —CH₂—, —NR—, —CO—, —CONH—, —CONR—, —C═N—O—, —NHCO—, —NRCO—, —O—N═C—, —SO—, —SO₂— or a direct bond, wherein R is C1-C3 alkyl optionally substituted by one or more halogen atoms;
   each of R¹, R² and R⁴ is independently selected from H, F, Cl, Br, I, CN, CF₃, OCF₃, R′, OR′, NR′R′, SOR′ or SO₂R′, wherein R′ are each independently C1-C3 alkyl optionally substituted by one or more halogen atoms; and
   R⁵ is selected from C1-C3 alkyl or C1-C3 alkoxy optionally substituted by one or more halogen atoms or —OH groups; and
   T is selected from groups of the following formulas (IVa) to (IVf):

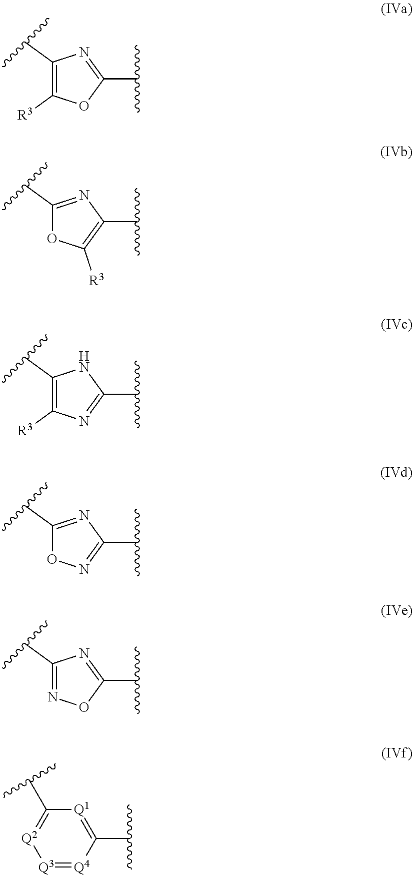

wherein R³ is selected from C1-C3 alkyl or C1-C3 alkoxy optionally substituted by one or more halogen atoms or —OH groups;
B is a group of formula (III), (IV), or (V), or a group of formula (VI):

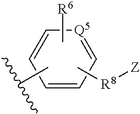

or a group of formula —R¹⁰Z,
wherein: Q⁵ is N or CR⁷;
   each of R⁶ and R⁷ is independently selected from H, F, Cl, Br, I, CF₃, CN, OCF₃, R′, OR′, NR′R′, SOR′ or SO₂R′, wherein R and R′ are each independently C1-C3 alkyl optionally substituted by one or more halogen atoms, and provided that R⁶ and R⁷ are not both H;
   R⁸ is -Alk-, —CONH(Alk)-, or —COO(Alk)-, where Alk is a C1-C4 alkylene or alkenylene group which may be linear or branched or may include a cyclopropyl group, or R⁸ is a group of formula (VII):

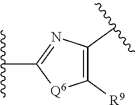

wherein Q⁶ is selected from O or S and R⁹ is C1-C4 alkyl or alkoxy;
Z is selected from —CO₂R′, —CONR′R″, —SO₂R′ wherein R′ and R″ are independently H or C1-C4 alkyl; and
R¹⁰ is a C1-C4 alkylene or alkenylene group;
and
L represents a linker group which is a saturated or unsaturated chain of 5 to 13 carbon atoms in which optionally from one to three of the carbon atoms are replaced by O, S, NR′, SO, SO₂, or CONR′, wherein R′ is H or C1-C3 alkyl, and wherein the said chain is unsubstituted or substituted by one or more groups comprising halogen, OH, C1-C3 alkyl, C2-C3 alkenyl, C2-C3 alkynyl or C1-C3 alkoxy.

Suitable and/or preferred agents according to the invention as defined above are described in detail below, and in the accompanying claims.

The invention also encompasses any stereoisomer, enantiomer or geometric isomer of the agents disclosed therein, and mixtures thereof.

In a second aspect, the present invention provides an agent according to the first aspect of the invention for use in the prevention and/or treatment of all forms of systemic transthyretin, ATTR, amyloidosis.

In a further aspect, the present invention provides the use of an agent according to the first aspect of the invention, for the manufacture of a medicament for prevention and/or treatment of all forms of acquired, wild type, and hereditary variant, systemic transthyretin, ATTR amyloidosis, including those presentations formerly called senile systemic amyloidosis, senile cardiac amyloidosis, familial amyloid polyneuropathy and familial amyloid cardiomyopathy.

In a further aspect, the present invention provides a pharmaceutical composition comprising an agent according to the first aspect of the invention in admixture with one or more pharmaceutically acceptable excipients, diluents or carriers.

In a further aspect, the present invention provides a method for stabilising the tetrameric form of transthyretin in a patient in need thereof, thereby inhibiting the mechano-enzymatic mechanism of transthyretin amyloid fibrillogenesis, comprising administering to the patient a therapeutic amount of an agent according to the first aspect of the invention or a pharmaceutical composition according to the invention. Suitably, the patient is a patient exhibiting adult onset acquired, wild type, wtATTR, transthyretin systemic amyloidosis, hereditary, variant systemic, ATTR, transthyretin amyloidosis, familial amyloid polyneuropathy or familial cardiac transthyretin amyloidosis, and/or a patient exhibiting an amyloidogenic gene mutation such as V30M, T60A or V122I, or any of the more than 120 other amyloid transthyretin gene mutations.

In a further aspect, the present invention provides a method of treatment of systemic transthyretin amyloidosis, comprising administering to the patient a therapeutic amount of an agent according to the first aspect of the invention or a pharmaceutical composition according to the invention. In embodiments, the systemic transthyretin amyloidosis may be any of the different form of wild type or hereditary systemic, ATTR, transthyretin amyloidosis.

### DETAILED DESCRIPTION OF THE INVENTION

In the present patent application, including the accompanying claims, the aforementioned substituents have the following meanings: Halogen atom means fluorine, chlorine, bromine or iodine.

Alkyl groups and portions thereof (unless otherwise defined) maybe a straight or branched chain.

The term “C1-Cn alkyl” as used here refers to a straight or branched chain or cyclic carbon chain consisting of 1 to n carbon atoms, which can be optionally substituted by one or more halogens.

The term “C2-Cn alkenyl” as used here refers to a chain consisting of 2 to n carbon atoms, which contains one double bond which can be located in any position of the respective unsaturated radical.

The term “C2-Cn alkynyl” as used here refers to a chain consisting of 2 to n carbon atoms, which contains one triple bond which can be located in any position of the respective unsaturated radical.

The term “C1-Cn alkoxy” as used here refers to a straight or branched or cyclic carbon chain consisting of 1 to n carbon atoms, which is connected via an oxygen atom to another group.

Pharmaceutically-acceptable salts of the agents disclosed herein include salts with a base or acid, which may be organic or inorganic. Salts of inorganic bases include those of alkali metals, alkaline earth metals and ammonium salts. Organic bases include pyridine, trimethylamine, triethylamine, and ethanolamine. Inorganic acids include hydrochloric acid, sulphuric acid, nitric acid and phosphoric acid. Organic acids include amino acids which may be basic or acidic, formic acid, acetic acid, citric acid, tartaric acid, fumaric acid and oxalic acid.

It is noted that in this disclosure, terms such as “comprises”, “comprised”, “comprising”, “contains”, “containing” and the like can have the meaning attributed to them; e.g., they can mean “includes”, “included”, “including” and the like. Terms such as “consisting essentially of and “consists essentially of” have the meaning attributed to them, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of and “consisting of have the meaning ascribed to them; namely, that these terms are closed ended.

In a first aspect, the present invention provides an agent for stabilising the tetrameric form of transthyretin, which comprises a compound of the general formula (T) or a pharmaceutically acceptable salt, ester or prodrug thereof:

A-L-B (I)
wherein:
A is a group of formula (II):

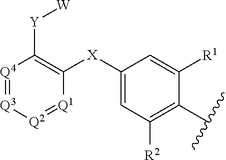

or of formula (III):

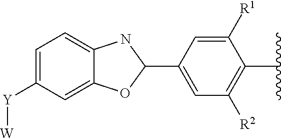

or of formula (IV):

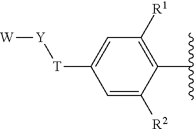

or of formula (V):

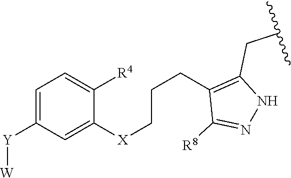

wherein: Y is independently a direct bond or a C1-C4 alkylene group which may be linear, branched, or may include a cyclopropyl group;
   W is —COOH or a tetrazole group; Q¹, Q², Q³ and Q⁴ are independently CH or N, provided that no more than two of Q¹, Q², Q³ and Q⁴ are N;
   X is independently —NH—, —O—, —S—, —CH₂—, —NR—, —CO—, —CONH—, —CONR—, —C═N—O—, —NHCO—, —NRCO—, —O—N═C—, —SO—, —SO₂— or a direct bond, wherein R is C1-C3 alkyl optionally substituted by one or more halogen atoms;
   each of R¹, R² and R⁴ is independently selected from H, F, Cl, Br, I, CN, CF₃, OCF₃, R′, OR′, NR′R′, SOR′ or SO₂R′, wherein R′ are each independently C1-C3 alkyl optionally substituted by one or more halogen atoms; and
   R⁵ is selected from C1-C3 alkyl or C1-C3 alkoxy optionally substituted by one or more halogen atoms or —OH groups; and
   T is selected from groups of the following formulas (IVa) to (IVf):

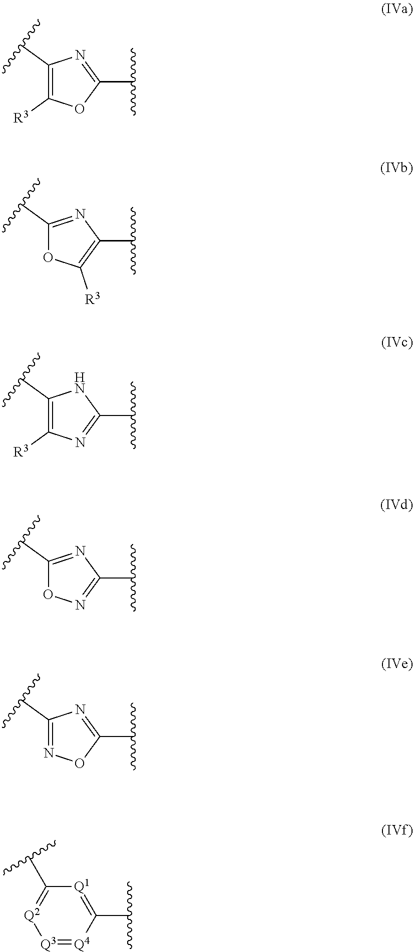

wherein R³ is selected from C1-C3 alkyl or C1-C3 alkoxy optionally substituted by one or more halogen atoms or —OH groups;
B is a group of formula (III), (IV) or (V), or a group of formula (VI):

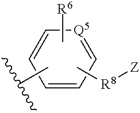

or a group of formula —R¹⁰Z,
wherein: Q⁵ is N or CR⁷;
   each of R⁶ and R⁷ is independently selected from H, F, Cl, Br, I, CF₃, CN, OCF₃, R′, OR′, NR′R′, SOR′ or SO₂R′ wherein R and R′ are each independently C1-C3 alkyl optionally substituted by one or more halogen atoms, and provided that R⁶ and R⁷ are not both H;
   R⁸ is -Alk-, —CONH(Alk)-, or —COO(Alk)-, where Alk is a C1-C4 alkylene or alkenylene group which may be linear or branched or may include a cyclopropyl group, or R⁸ is a group of formula (VII):

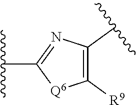

wherein Q⁶ is selected from O or S and R⁹ is C1-C4 alkyl or alkoxy;
Z is selected from —CO₂R′, —CONR′R″, —SO₂R′ wherein R′ and R″ are independently H or C1-C4 alkyl; and
R¹⁰ is a C1-C4 alkylene or alkenylene group;
and L represents a linker group which is a saturated or unsaturated chain of 5 to 13 carbon atoms in which optionally from one to three of the carbon atoms are replaced by O, S, NR′, SO, SO₂, or CONR′, wherein R′ is H or C1-C3 alkyl, and wherein the said chain is unsubstituted or substituted by one or more groups comprising halogen, OH, C1-C3 alkyl, C2-C3 alkenyl, C2-C3 alkynyl or C1-C3 alkoxy.

It has been found that compounds according to the present invention potently stabilise the tetrameric transthyretin molecule, protecting it from the crucial proteolytic cleavage that is essential for triggering transthyretin amyloid fibrillogenesis by the pathophysiological mechano-enzymatic mechanism. The compounds are rapidly and specifically bound with high affinity and avidity by native tetrameric transthyretin and occupy simultaneously both of the ligand binding pockets. Under physiological conditions of solvent pH, ionic strength and composition, the compounds are bound both by isolated pure transthyretin and by transthyretin in whole plasma, and displace thyroid hormone from its binding pocket in transthyretin. The compounds are bound such that each ligand simultaneously occupies both the two binding pockets with the linker running through the core of the transthyretin molecule (Verona et al. 2017). Such binding markedly stabilises the tetrameric assembly and native fold of transthyretin, and prevents the pathogenic proteolytic cleavage that leads, with mechanical forces, to dissociation, misfolding and amyloid fibril formation. Furthermore, binding of the compounds in this way by native transthyretin in whole plasma means that they are greatly superior as drugs compared with monofunctional compounds of the prior art such as tafamidis, diflunisal and AG10. The compounds have lower log P than mds84, and better bioavailability. Suitably, they have lower molecular weight than mds84.

In the groups of formula (II) or (III), suitably: Y is a direct bond or —CH₂; and/or W is —COOH or tetrazole; and/or X is —NH—; and/or R″ is halogen or H; and/or R′ is halogen; and and/or either (a) all of Q¹-Q⁴ are CH, or (b) one of Q¹, Q² or Q³ is N and/or the remainder of Q¹-Q⁴ are CH or (c) Q² and Q³ are N, and Q¹ and Q⁴ are CH or (d) Q³ and Q⁴ are N, and Q¹ and Q² are H. More suitably: Y is a direct bond; and/or W is —COOH; and/or X is —NH—, and/or R¹ is Cl or H; and/or R² is Cl; and/or either (a) all of Q¹-Q⁴ are CH, or (b) one of Q¹, Q² or Q³ is N and the remainder of Q¹-Q⁴ are CH.

In embodiments, the groups of formula (II) are selected from:

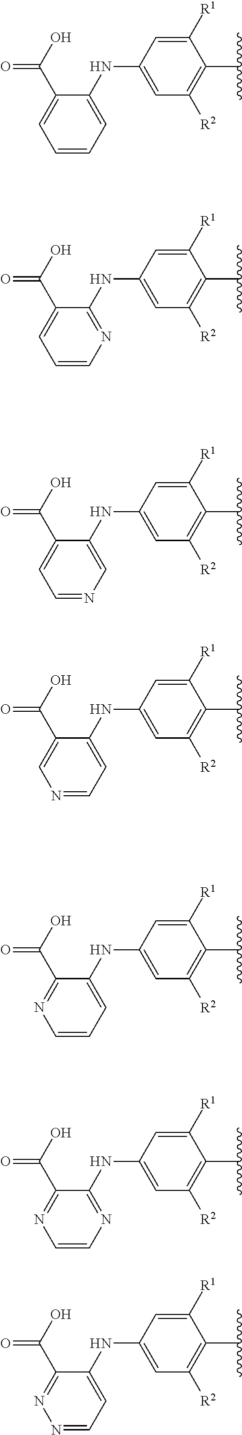

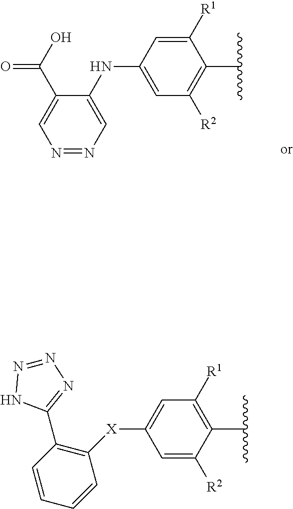

wherein X, R¹ and R² are as defined above, suitably wherein R¹ and R² are independently selected from II and Cl.

In embodiments, the groups of formula (III) are selected from:

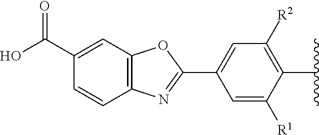

wherein R¹ and R² are as defined above, suitably wherein R″ and R² are independently selected from H and Cl.

In the groups of formula (IV), suitably: Y is a direct bond or —CH₂—; and/or W is —COOH or tetrazole; and/or R¹ is halogen or H; and/or R² is halogen; and/or R³ is —CH₃ or —C₂H₅. More suitably: Y is a direct bond; and/or W is —COOH; and/or R¹ is Cl or H; and/or R² is Cl; and/or R³ is —C₂H₅; and/or T is selected from groups (IVa) to (IVe), in particular group (IVa).

In embodiments, the groups of formula (IV) are selected from:

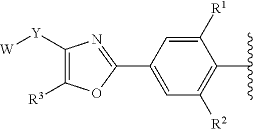

wherein the substituents and more suitable substituents are as defined above, for example groups of the following formula:

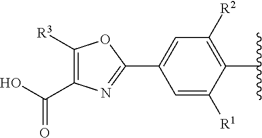

wherein R¹ and R² are as defined above, for example they may be independently selected from H and C1, and R³ is H, methyl or ethyl.

In the groups of formula (V), suitably: Y is a direct bond or —CH₂—; and/or W is —COOH or tetrazole; and/or X is —O— or —NH—; and/or R⁴ is F, and/or R′ is —CH₃ or —C₂H₅. More suitably: Y is a direct bond; and/or W is —COOH; and/or X is —O—; and/or R⁴ is F, and/or R⁵ is —CH₃.

In embodiments, B is a group of formula (VI) having the following structure:

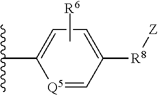

for example wherein B has the following structure:

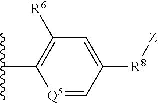

In embodiments, B is a group of formula (VI) wherein Q⁵ is CH, Z is COOH, and R⁸ is a direct bond, CH₂ (methylene), C₂H₄ (ethylene) or C₃H₆ (n-propylene).

Suitably, B is a group of formula (VI). In these embodiments, suitably: Q⁵ is CR⁷ wherein R⁷ is Cl; and/or R′ is Cl or H; and/or Z is —COOH, and/or R⁸ is selected from —CH₂—, —C₂H₄—, or —CONHCH(CH₃)—; or the group R⁸Z is selected from:

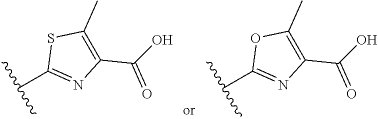

More suitably, Q⁵ is CR⁷ wherein R⁷ is Cl; and/or R⁶ is Cl or H; and/or Z is —COOH, and/or R⁸ is —C₂H₄—.

In alternative embodiments, Group B has formula R¹⁰Z, wherein R¹⁰ is —CH₂— or —C₂H₄— and/or Z is —COOH.

It can be seen that group A is a bis aryl group, which may be more hydrophilic than the bis aryl end groups of the prior art. Suitably, B is a mono-aryl group. In particular, B is suitably a mono-aryl group where A is a group of Formula (II). This helps to reduce the overall molecular weight and lipophilicity of the compound.

L represents a linker group which is a saturated or unsaturated chain of 5 to 13 carbon atoms with optional carbon replacements and/or substituents as defined above. Suitably, the linker group is a saturated or unsaturated chain of from 6 to 10 carbon atoms with optional carbon replacements and/or substituents as defined above. In embodiments, the chain is unsubstituted, or substituted only with one or two —OH groups.

The linker group L is suitably linked to the groups A and B by carbon-carbon single bonds, or by ether, thioether, amino (—NH—), keto (—CO—), ester or amide linkages at the terminal ends of the linker group. More suitably, the linker group L is suitably linked to the groups A and B by ether (—O—) linkages.

In embodiments, the linker group L is a dialkylene oxide group or a trialkylene oxide group of formula (VII):

—O—R¹¹—X—R¹²—O—(R¹³—O)ₘ

wherein: m is 0 or 1; X is O, NH, CH(OH), C(═O)NH, SO, or SO₂; and R¹¹, R¹² and R¹³ are independently methylene, ethylene, n-propylene or n-butylene groups optionally substituted by one or more groups selected from the group consisting of halogen, OH, C1-C3 alkyl, C2-C3 alkenyl, C2-C3 alkynyl or C1-C3 alkoxy. Suitably, X is O, SO or SO₂, most suitably X is O.

In embodiments of formula (VII), m is 0, R″ and R¹² are independently ethylene or n-propylene groups, and X is O, NIH, CH(OH), C(O)NH, SO, or SO₂.

In embodiments, the linker group is suitably one of the following linker groups:

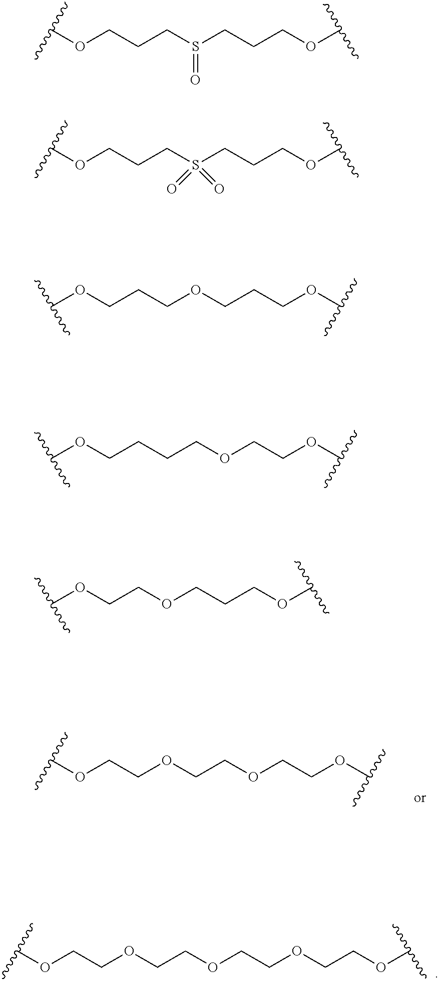

In other embodiments, the linker group is a linear or branched chain of 5 to 13 carbon atoms substituted with one, two or three —OH groups. In these embodiments, the linker group is suitably one of the following linker groups:

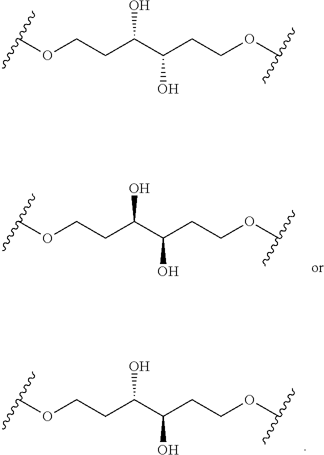 [SMILES: *OCC[C@H](O)[C@@H](O)CCO* ; *OCC[C@@H](O)[C@H](O)CCO* ; *OCC[C@@H](O)[C@@H](O)CCO*]

The above linkers are suitably more hydrophilic than the alkylene linker of mds84, and may thereby lower the log P and otherwise improve the pharmacological properties of the compounds in comparison to mds84.

In yet other embodiments, the linker group is a group of formula (VII):

—O—R¹⁰—O—

wherein R¹⁰ is an alkylene or alkenylene group comprising from 5 to 10 carbon atoms in the chain, optionally substituted by one or more groups selected from the group consisting of halogen, OH, C1-C3 alkyl, C2-C3 alkenyl, C2-C3 alkynyl or C1-C3 alkoxy. As noted above, the group R¹⁰ in these embodiments is suitably not branched and/or not substituted.

Suitably, in these embodiments, the linker group is as follows:

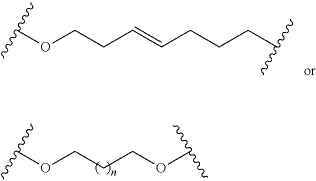

wherein n=2, 3, 4, 5, 6, 7 or 8.

In the foregoing, lists of suitable and more suitable chemical definitions are given, separated by the words “and/or”. It is to be understood, that sub-groups of compounds having all individual definitions, and any combination of suitable or more suitable definitions, are specifically envisaged and disclosed thereby.

The agents of general formula (I) may suitably have one of the following specific formulae:

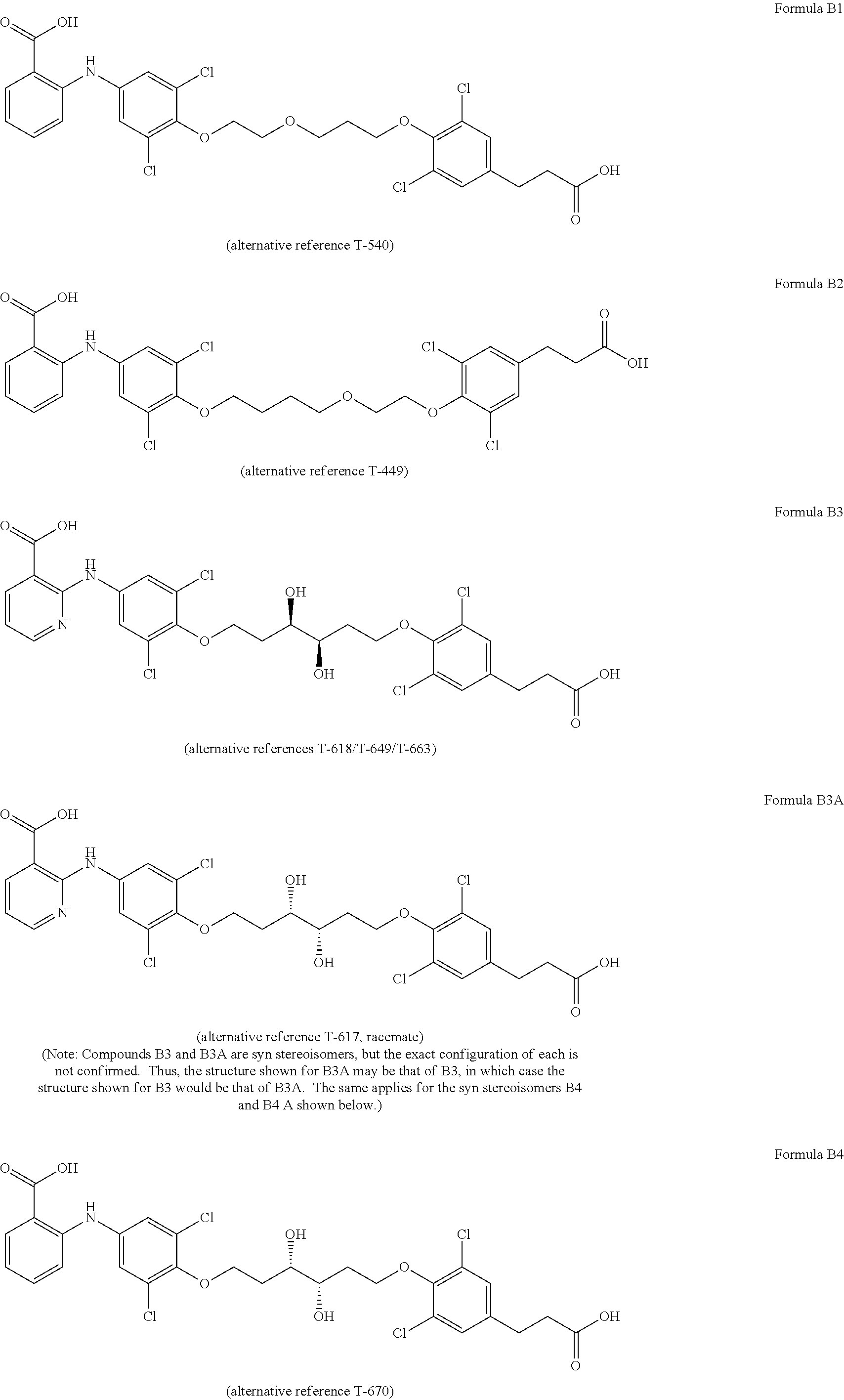

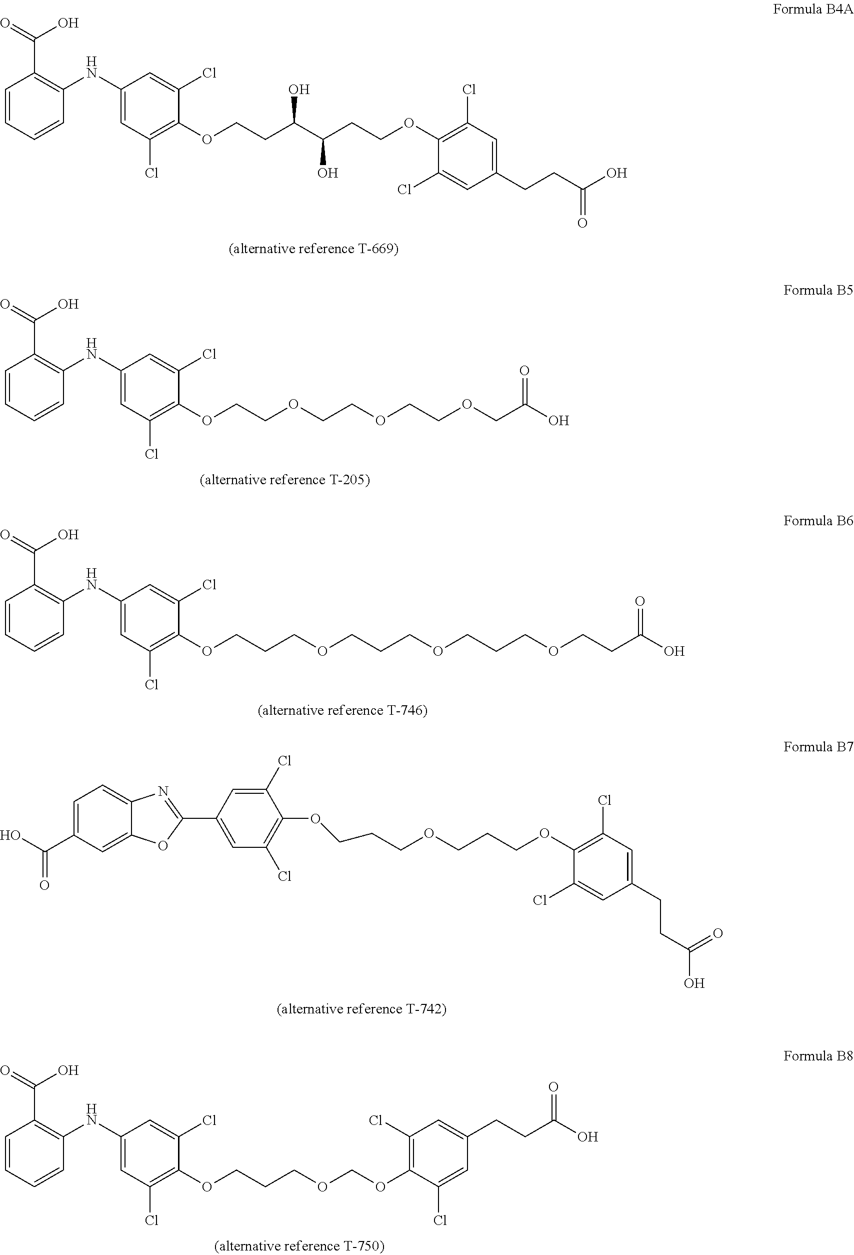

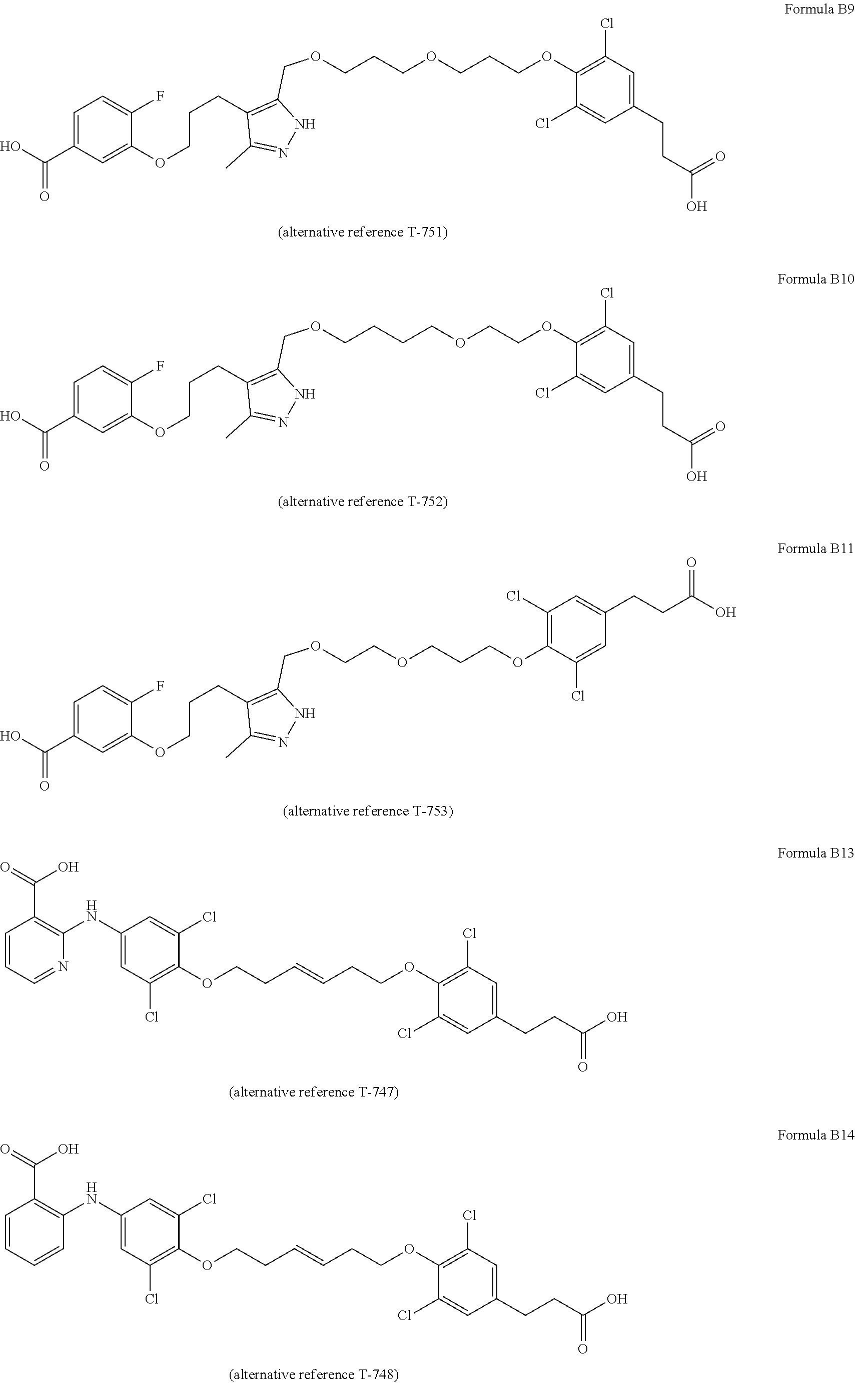

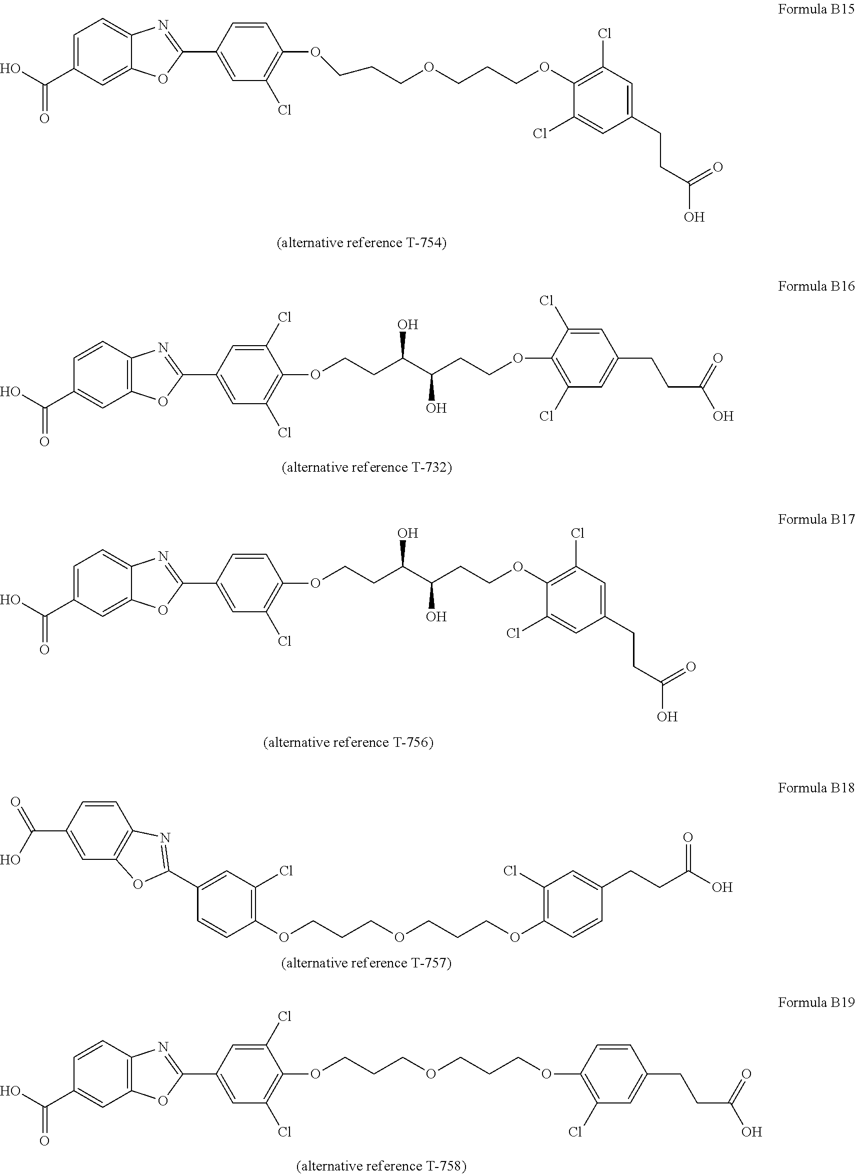

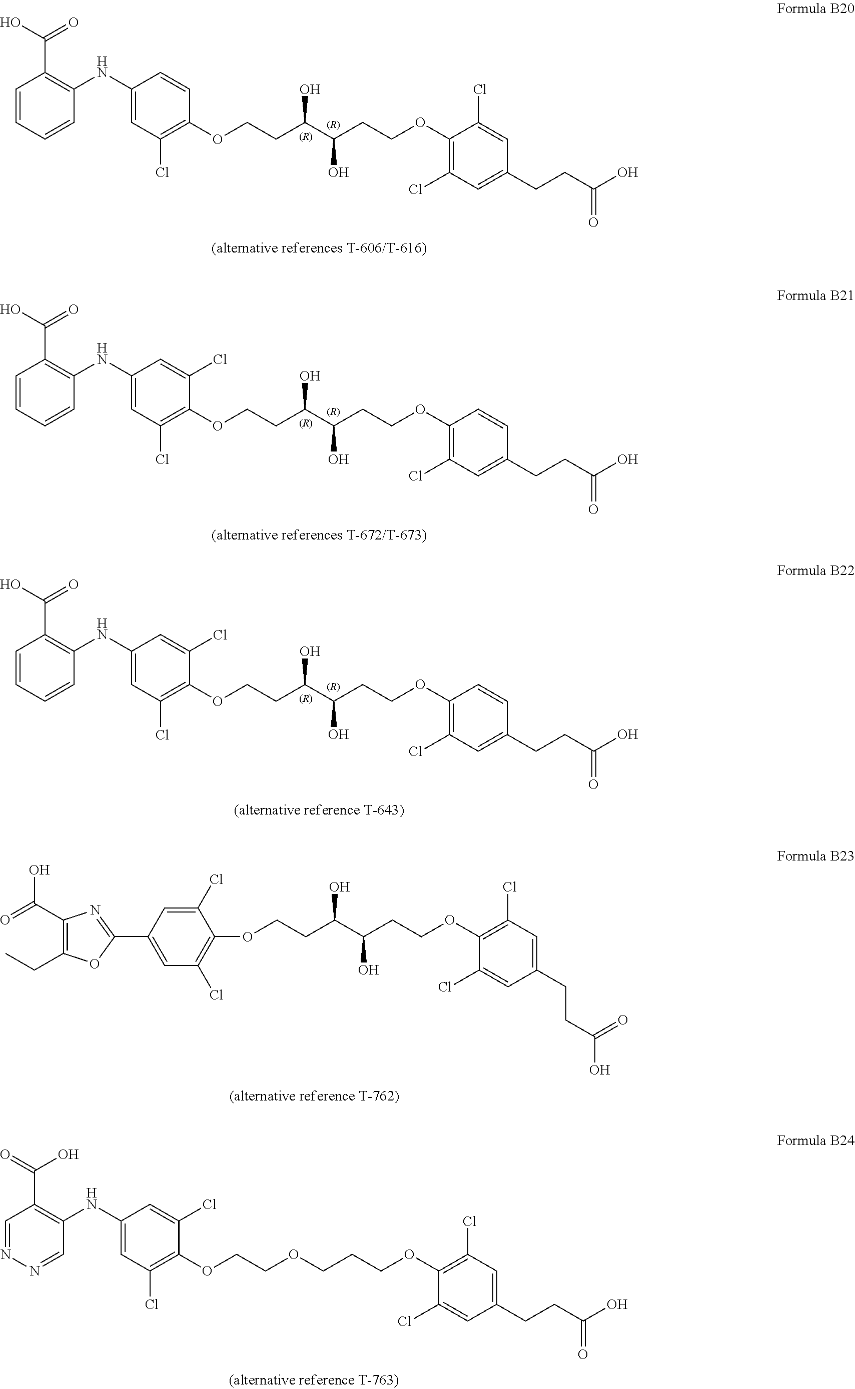

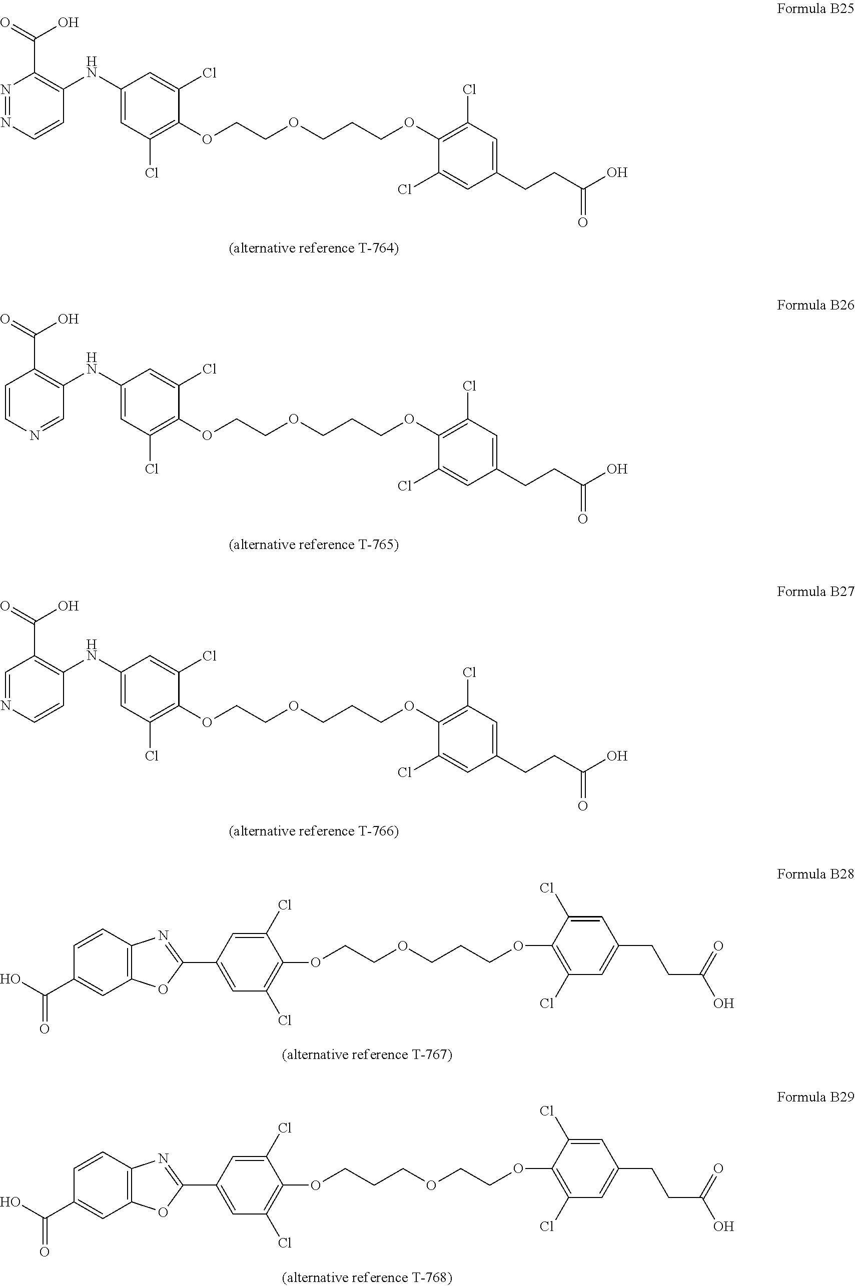

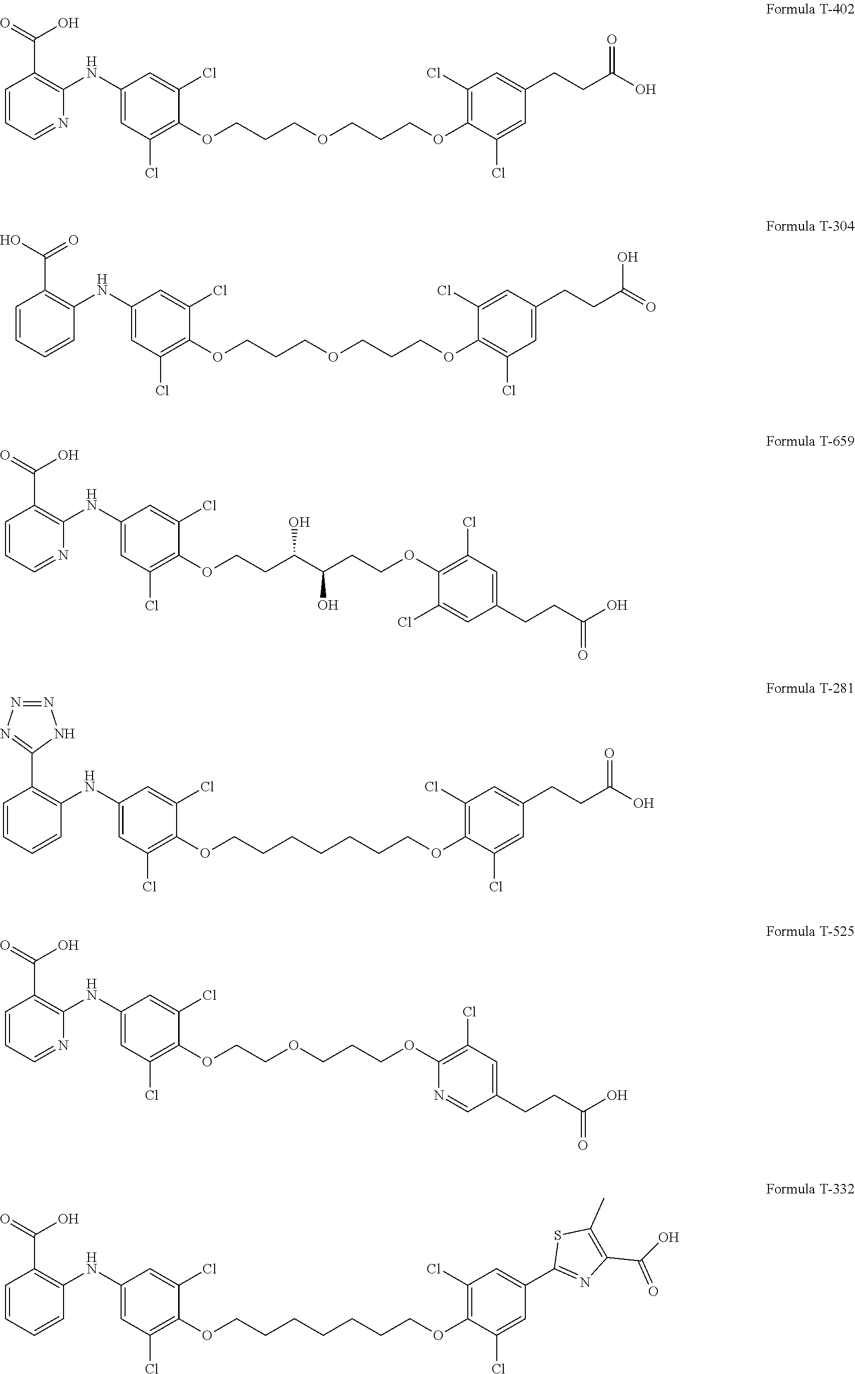

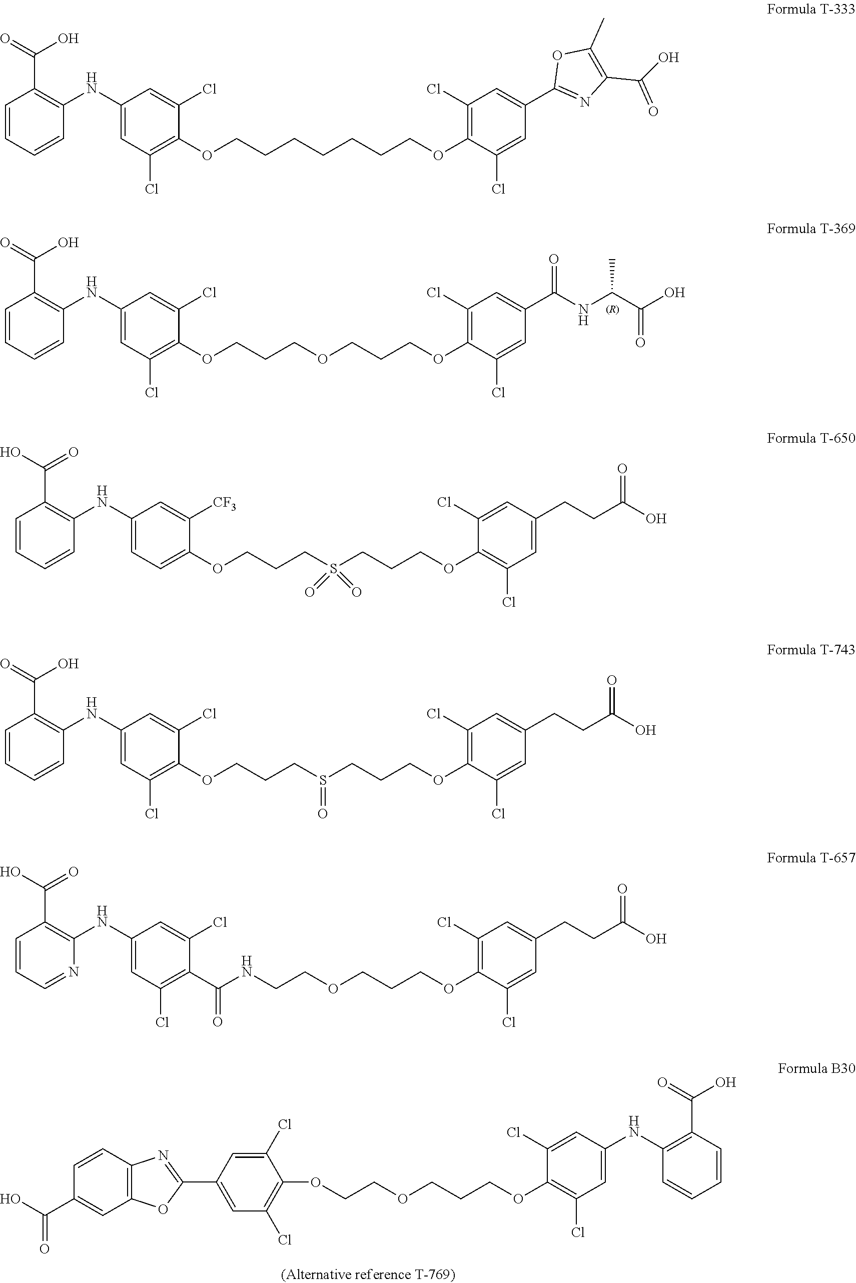

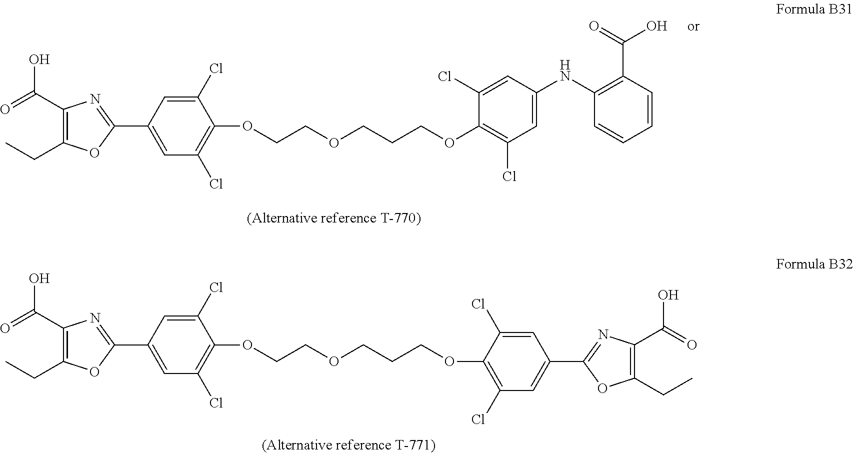

or a pharmaceutically acceptable salt or ester thereof.

Suitably, the compound of Formula (I) has a [D₅₀] for displacement of ¹²⁵I-T4 from isolated TTR as measured by the method disclosed herein of less than about 1 μM, preferably less than about 0.25 μM, more preferably less than about 0.15 μM.

Suitably, the compound of Formula (I) has a [D₅₀] for displacement of ¹²⁵I-T4 from TTR in whole human plasma as measured by the method disclosed herein of less than about 15 μM, preferably less than about 10 μM, more preferably less than about 5 μM.

Suitably, the compound of Formula (I) inhibits mechano-enzymatic fibrillogenesis of TTR-122Ile as determined by turbidity measurement and thioflavin T fluorescence measurement according to the methods disclosed herein such that the % aggregation at 96 h is less than about 25%, preferably less than about 15%.

Suitably, the compound of Formula (I) has a hydrophilic/lipophilic partition coefficient (log P) less than about 10, suitably less than about 8, more suitably less than about 6. The log P is suitably defined for the water/n-octanol system, and may be determined by calculation, for example using the ACD/log P software available from Advanced Chemistry Developments Inc., Toronto, CA. In alternative embodiments, the log P may be determined by chromatography, for example with ASTM E1147-92 (2005).

In a further aspect, the present invention provides an agent according to the first aspect of the invention for use in the treatment or prevention of transthyretin amyloidosis. In another aspect, the present invention provides the use of an agent according to the first aspect of the invention for the manufacture of a medicament for treatment or prevention of transthyretin amyloidosis. Suitably, the transthyretin amyloidosis is a systemic amyloidosis.

The present invention further provides a pharmaceutical composition comprising an agent according to the first aspect of the invention in admixture with one or more pharmaceutically acceptable excipients, diluents or carriers.

The present invention further provides a method for stabilising the tetrameric form of transthyretin, in order to inhibit the proteolytic cleavage that is essential for amyloid fibril formation, in a patient in need thereof, comprising administering to the patient a therapeutic amount of an agent according to the first aspect of the invention, or a pharmaceutical composition according to the invention.

The types of amyloidosis treatable with the agents of the present invention include senile cardiac transthyretin amyloidosis, autosomal dominant adult onset hereditary transthyretin amyloidosis, familial amyloid polyneuropathy of transthyretin type, and all other forms of transthyretin amyloidosis. The transthyretin by which the agents may be bound is wild type transthyretin or a variant form, including transthyretin having the single residue substitutions V30M, T60A, V122I or any of the other >120 different transthyretin variants which have been reported to cause transthyretin amyloidosis.

Pharmaceutical compositions may be formulated comprising an agent or a pharmaceutically acceptable salt, ester or prodrug thereof according to the present invention optionally incorporating a pharmaceutically acceptable carrier, diluent or excipient (including combinations thereof). By the term “pharmaceutically acceptable salt” is meant salts the anions or cations of which are known and accepted in the art for the formation of salts for pharmaceutical use. Acid addition salts, for example, may be formed by mixing a solution of the agent with a solution of a pharmaceutically acceptable, non-toxic acids, which include but are not limited to hydrochloric acid, oxalic acid, fumaric acid, maleic acid, succinic acid, acetic acid, citric acid, tartaric acid, carbonic acid or phosphoric acid. Where the agent carries a carboxylic acid group, the invention also contemplates salts thereof, preferably non-toxic, pharmaceutically acceptable salts thereof, which include, but are not limited to the sodium, potassium, calcium and quaternary ammonium salts thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Antioxidants and suspending agents may be also used.

The pharmaceutical compositions may be in the form of a prodrug comprising the agent or a derivative thereof which becomes active only when metabolised by the recipient. The exact nature and quantities of the components of such pharmaceutical compositions may be determined empirically and will depend in part upon the route of administration of the composition. Where appropriate, the pharmaceutical compositions of the present invention can be administered by inhalation, in the form of a suppository or pessary, topically (including ophthalmically) in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly, subcutaneously or intra-arterially.

For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of an agent, or a nontoxic, pharmaceutically acceptable salt thereof. The liquid forms in which the compositions of the present invention may be incorporated for administration orally or by injection include aqueous emulsions with edible oils such as cottonseed oil, sesame oil, coconut oil and peanut oil, as well as elixirs and similar pharmaceutical vehicles. Suitable dispersing or suspending agents for aqueous suspension include synthetic and natural gums such as tragacanth, acacia, alginate, dextran, sodium carboxymethylcellulose, methylcellulose, polyvinyl-pyrrolidone and gelatin.

For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner. For convenience of use, dosages according to the present invention are preferably administered orally but this will depend on the actual drug and its bioavailability.

Use of the compounds of the present invention aims to saturate with the ligand drug all circulating and, possibly, other soluble transthyretin molecules in the body. The dose of drug required is therefore that which provides at least 1 mol of drug per mol of transthyretin produced each day. The daily production of transthyretin in normal healthy individuals is between 9.5 and 13 μmol/day in a 70 kg subject (Robbins J., 2002). There is no situation in which transthyretin production is upregulated and synthesis is reduced in all inflammatory, infective and tissue damaging diseases associated with an acute phase response and in malnutrition. For a compound of molecular mass 700 Da molar, equivalence with daily transthyretin production corresponds to 6.65-9.1 mg/day. If the drug were to be 100% bioavailable either orally or after parenteral administration, then that dose range itself would be the minimum necessary. If the drug were given orally and was then, for example, just 10% bioavailable, the minimum daily dose would be ˜70-100 mg. Depending on the exact affinity, pharmacokinetics and pharmacodynamics of the drug, the dose might need to be up to 1 g or more per day.

The precise form of pharmaceutical composition and dosage thereof may also be dependent on the subject to be treated, including body weight, route of administration and disease conditions. These would be determined as a matter of routine by the skilled addressee.

### EXAMPLES/EXPERIMENTAL

### 1. Transthyretin

All the TTR isoforms used (mainly wild type and Val122Ile TTR) were produced by recombinant technology using a peTM11 plasmid coding for an N-terminal His₆-tag and a TEV cleavage site unless otherwise stated. The plasmid was transformed into E. coli BL21 (DE3) cells. To generate unlabelled TTR (for ligand screening) cells were grown in Luria Bertani medium in the presence of 30 μg/ml kanamycin. For NMR studies triple labelled TTR was produced using a deuterated background and Ross medium containing ¹⁵N ammonium sulphate and ¹³C-glucose as the only sources of nitrogen and carbon respectively. Both unlabelled and labelled TTR were expressed and purified as described in Corazza et al (2019).

### 2. Synthesis of Ligands

### Synthesis of B1 (T-540)

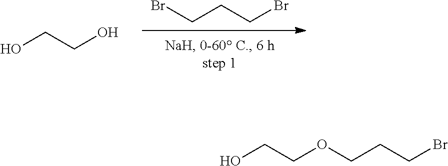

### 2-(3-bromopropoxy)ethanol

NaH (8.79 g, 219.76 mmol, 60% purity, 1.1 eq) was added to ethylene glycol (124 g, 2.00 mol, 111.71 mL, 10 eq) at 0° C. in portions during for 2 hr. After addition, 1,3-dibromopropane (40.33 g, 199.78 mmol, 20.37 mL, 1 eq) was added to the above mixture, it was stirred at 60° C. for 4 hr. The reaction mixture was diluted with H₂O (60 mL) and extracted with EtOAc (50 mL*3). The combined organic layers were washed with brine (70 mL), dried over Na₂SO₄, filtered and concentrated under reduced pressure to give a residue. The residue was purified by column chromatography (SiO₂, Petroleum ether/Ethyl acetate=10/1 to 2/1) to afford 2-(3-bromopropoxy)ethanol (8.6 g, 46.98 mmol, 23.52% yield) as colorless oil.

¹H NMR: ET20197-86-P1AA (METHANOL-d4, 400 MHz)

δ 6.01-5.86 (m, 1H), 5.36-5.11 (m, 1H), 4.02 (d, J=5.6 Hz, 1H), 3.72-3.64 (m, 2H), 3.60 (t, J=5.9 Hz, 1H), 3.57-3.50 (m, 3H), 2.10 (quin, J=6.2 Hz, 1H).

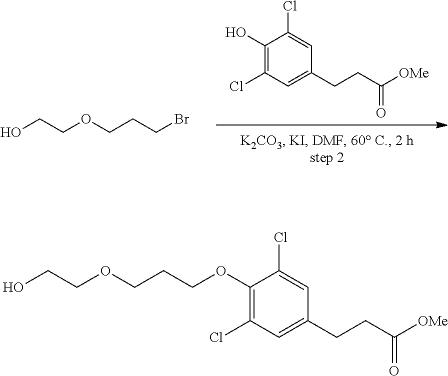 [SMILES: COC(=O)CCc1cc(Cl)c(O)c(Cl)c1 ; OCCOCCCBr ; COC(=O)CCc1cc(Cl)c(OCCCOCCO)c(Cl)c1]

### Methyl 3-[3,5-dichloro-4-[3-(2-hydroxyethoxy)propoxy]phenyl]propanoate

To a solution of methyl 3-(3,5-dichloro-4-hydroxy-phenyl)propanoate (1.5 g, 6.02 mmol, 1 eq) and 2-(3-bromopropoxy)ethanol (1.32 g, 7.23 mmol, 1.2 eq) in DMF (120 mL) was added K₂CO₃ (2.50 g, 18.07 mmol, 3 eq) and KI (999.64 mg, 6.02 mmol, 1 eq). The mixture was stirred at 60° C. for 2 hr. The reaction mixture was diluted with H₂O (100 mL) and extracted with EtOAc (80 mL*3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=10/1 to 2/1) to afford methyl 3-[3,5-dichloro-4-[3-(2-hydroxyethoxy)propoxy]phenyl]propanoate (1.4 g, 3.99 mmol, 66.19% yield) as yellow oil.

¹H NMR: ET20197-106-P1AA (METHANOL-d4, 400 MHz)

δ 7.23 (s, 2H), 4.07 (m, 2H), 3.73 (t, J=6.3 Hz, 2H), 3.70-3.65 (m, 2H), 3.64 (s, 3H), 3.57-3.53 (m, 2H), 2.86-2.82 (m, 2H), 2.65-2.60 (m, 2H), 2.08 (quin, J=6.3 Hz, 2H).

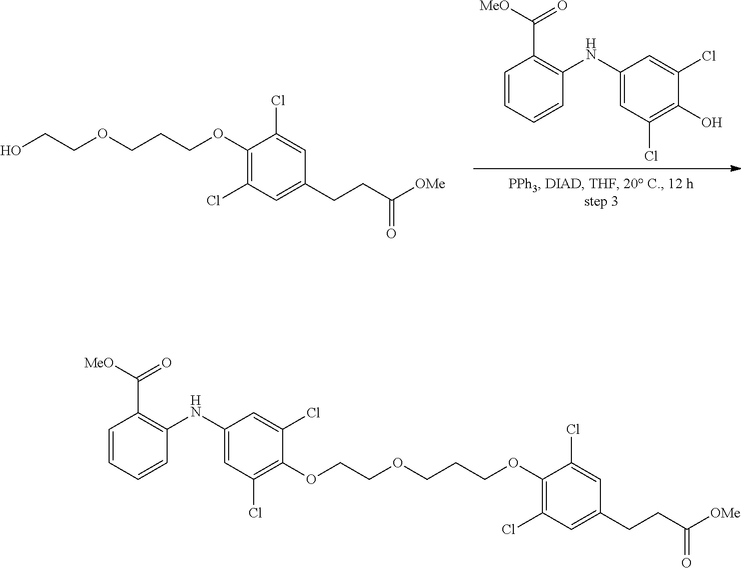 [SMILES: COC(=O)CCc1cc(Cl)c(OCCCOCCO)c(Cl)c1 ; COC(=O)c1ccccc1Nc1cc(Cl)c(O)c(Cl)c1 ; COC(=O)CCc1cc(Cl)c(OCCCOCCOc2c(Cl)cc(Nc3ccccc3C(=O)OC)cc2Cl)c(Cl)c1]

### Methyl 2-[3,5-dichloro-4-[2-[3-[2,6-dichloro-4-(3-methoxy-3-oxo-propyl)phenoxy]propoxy]ethoxy]anilino]benzoate

To a solution of methyl 3-[3,5-dichloro-4-[3-(2-hydroxyethoxy)propoxy]phenyl]propanoate (1.4 g, 3.99 mmol, 1 eq) and methyl 2-(3,5-dichloro-4-hydroxy-anilino)benzoate (1.24 g, 3.99 mmol, 1 eq) in THF (15 mL) was added PPh₃ (1.57 g, 5.98 mmol, 1.5 eq) and DIAD (1.21 g, 5.98 mmol, 1.16 mL, 1.5 eq). The mixture was stirred at 20° C. for 12 hr. The reaction mixture was concentrated under reduced pressure to remove solvent. The residue was diluted with H₂O (40 mL) and extracted with EtOAc (25 mL*3). The combined organic layers were washed with brine (30 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜10%. Ethylacetate/Petroleum ethergradient @ 75 mL/min) to afford methyl 2-[3,5-dichloro-4-[2-[3-[2,6-dichloro-4-(3-methoxy-3-oxo-propyl)phenoxy]propoxy]ethoxy]anilino]benzoate (780 mg, 90% purity) as yellow oil.

(Note: The reaction was combined with another reaction ET20197-111 in 150 mg scale for work up and purification.)

¹H NMR: ET20197-120-P1AB (CHLOROFORM-d, 400 MHz)

δ 9.42 (s, 1H), 7.98 (dd, J=1.5, 8.1 Hz, 1H), 7.38 (ddd, J=1.5, 7.2, 8.5 Hz, 1H), 7.24-7.20 (m, 1H), 7.18 (s, 2H), 7.12 (s, 2H), 6.81 (ddd, J=1.1, 7.1, 8.0 Hz, 1H), 4.22-4.18 (m, 2H), 4.14-4.11 (m, 2H), 3.91 (s, 3H), 3.90-3.86 (m, 2H), 3.83 (t, J=6.3 Hz, 2H), 3.68 (s, 3H), 2.89-2.82 (m, 2H), 2.63-2.57 (m, 2H), 2.15 (quin, J=6.3 Hz, 2H).

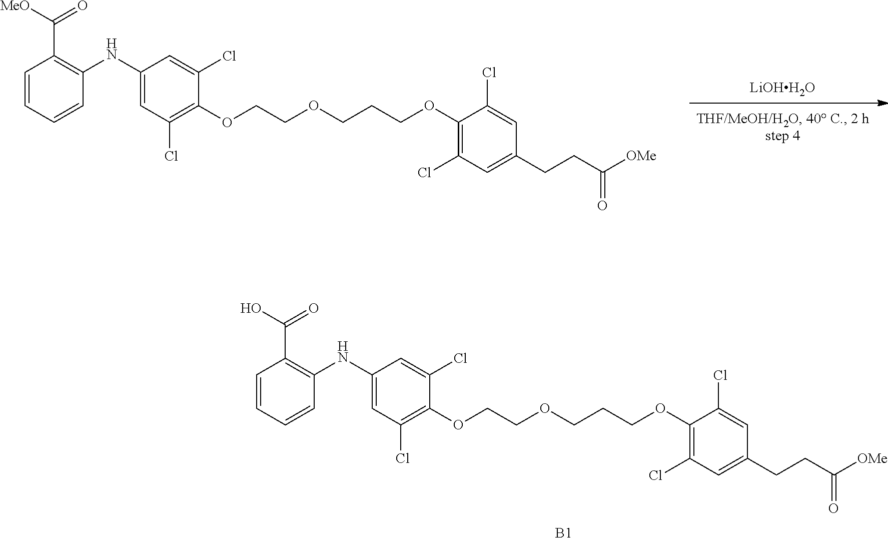 [SMILES: COC(=O)CCc1cc(Cl)c(OCCCOCCOc2c(Cl)cc(Nc3ccccc3C(=O)OC)cc2Cl)c(Cl)c1 ; "LiOH·H2O" ; COC(=O)CCc1cc(Cl)c(OCCCOCCOc2c(Cl)cc(Nc3ccccc3C(=O)O)cc2Cl)c(Cl)c1 ; "B1"]

### 2-[4-[2-[3-[4-(2-carboxyethyl)-2,6-dichloro-phenoxy]propoxy]ethoxy]-3,5-dichloro-anilino]benzoic acid

To a solution of methyl 2-[3,5-dichloro-4-[2-[3-[2,6-dichloro-4-(3-methoxy-3-oxopropyl) phenoxy]propoxy]ethoxy]anilino]benzoate (580 mg, 898.73 umol, 1 eq) in THF (28 mL) and MeOH (14 mL) was added LiOH·H₂O (226.28 mg, 5.39 mmol, 6 eq) in H₂O (14 mL). The mixture was stirred at 40° C. for 2 hr. The mixture was concentrated to give the residue. The residue was diluted with H₂O (50 mL) and extracted with DCM (30 mL*2). The organic layer was discarded. The aqueous layer was adjusted with HCl (1N) to pH˜2 and extracted with DCM (70 mL*4). The organic layer was washed with brine (30 mL*3), dried over Na₂SO₄, filtered and concentrated to give the residue. The residue was purified by prep-HPLC (HCl condition) to afford 2-[4-[2-[3-[4-(2-carboxyethyl)-2,6-dichloro-phenoxy]propoxy]ethoxy]-3,5-dichloro-anilino]benzoic acid (301.9 mg, 99.57% purity) as a white solid.

The reaction was combined with other reactions ET20197-124 in 100 mg and ET201960-1 in 100 mg scale for work up and purification.

LCMS: ET20197-126-P1A (M+H⁺): 618.0 @ 2.193 min (10-80% ACN in H₂O, 3.0 min)

¹H NMR: ET20197-126-P1BB (DMSO-d₆, 400 MHz)

δ 9.48 (s, 1H), 7.91 (dd, J=1.6, 7.9 Hz, 1H), 7.44 (dt, J=1.6, 7.8 Hz, 1H), 7.33 (d, J=10.0 Hz, 4H), 7.23 (d, J=8.1 Hz, 1H), 6.92-6.84 (m, 1H), 4.14-4.07 (m, 2H), 4.02 (t, J=6.5 Hz, 2H), 3.78-3.73 (m, 2H), 3.68 (t, J=6.2 Hz, 2H), 2.79-2.73 (m, 2H), 2.56-2.52 (m, 2H), 2.00 (quin, J=6.3 Hz, 2H)

DSC: ET20197-126-P1D (Peak: 170.3° C., onset 168.8° C., end 171.3° C.)

Melting point: ET20197-126-P1M (167.9-169.5° C.)

### Synthesis of B2 (T-499)

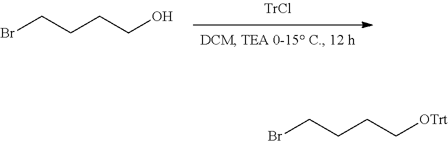

### [4-bromobutoxy(diphenyl)methyl]benzene

To a mixture of 4-bromobutan-1-ol (5 g, 32.68 mmol, 1 eq) and [chloro(diphenyl)methyl]benzene (13.66 g, 49.01 mmol, 1.5 eq) in DCM (100 mL) was added TEA (6.61 g, 65.35 mmol, 9.10 mL, 2 eq) at 0° C. The reaction mixture was slowly warmed to 15° C. and stirred for 12 h. The reaction was concentrated to give the residue. The residue was purified by column (SiO₂, PE˜PE:EtOAc=20:1) to afford [4-bromobutoxy(diphenyl)methyl]benzene (5.7 g, 14.42 mmol, 44.12% yield) as a colorless oil.

¹H NMR: ET20197-64-P1 AA (CHLOROFORM-d, 400 MHz)

δ 7.38-7.31 (m, 5H), 7.25-7.10 (m, 10H), 3.29 (t, J=6.8 Hz, 2H), 3.01 (t, J=6.2 Hz, 2H), 1.96-1.82 (m, 2H), 1.71-1.61 (m, 2H)

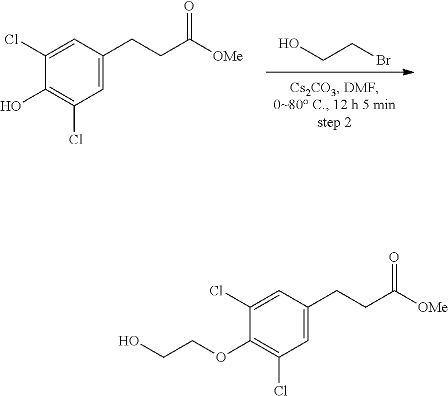

### Methyl 3-[3,5-dichloro-4-(2-hydroxyethoxy)phenyl]propanoate

To a solution of methyl 3-(3,5-dichloro-4-hydroxy-phenyl)propanoate (10 g, 40.15 mmol, 1 eq) in DMF (100 mL) was added Cs₂CO₃ (26.16 g, 80.29 mmol, 2 eq) at 0° C. After 5 min 2-bromoethanol (7.53 g, 60.22 mmol, 4.28 mL, 1.5 eq) and the mixture was stirred at 80° C. for 12 h. The reaction was diluted with H₂O (50 mL) and extracted with EtOAc (70 mL*3). The organic layer was washed with brine (20 mL*2), dried over Na₂SO₄, filtered and concentrated to give the residue. The residue was purified by column (SiO₂, PE/EtOAc=10/1-3/1) to afford methyl 3-[3,5-dichloro-4-(2-hydroxyethoxy)phenyl] propanoate (3.5 g, 11.94 mmol, 29.74% yield) as a colorless oil.

¹H NMR: ET20197-62-P1BB (CHLOROFORM-d, 400 MHz)

δ 7.20-7.09 (m, 2H), 4.20-4.13 (m, 2H), 3.98-3.90 (m, 2H), 3.75-3.62 (m, 3H), 2.90-2.83 (m, 2H), 2.64-2.56 (m, 2H)

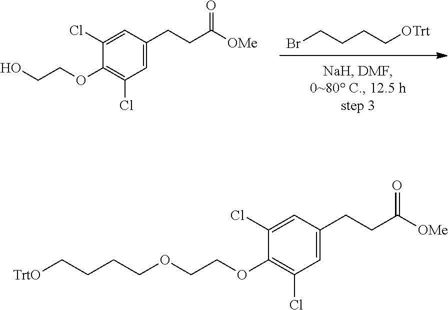

### Methyl 3-[3,5-dichloro-4-[2-(4-trityloxybutoxy)ethoxy]phenyl]propanoate

To a solution of methyl 3-[3,5-dichloro-4-(2-hydroxyethoxy)phenyl]propanoate (2.5 g, 8.53 mmol, 1 eq) in DMF (30 mL) was added NaH (1.02 g, 25.58 mmol, 60% purity, 3 eq) at 0° C. The reaction was stirred at 0° C. for 0.5 h. And [4-bromobutoxy(diphenyl)methyl]benzene (4.05 g, 10.23 mmol, 1.2 eq) was added to the mixture at 0° C. The reaction was stirred at 80° C. for 12 h. The black mixture was quenched with NH₄Cl aq. (100 mL) and extracted with EtOAc (100 mL*5). The organic was washed with brine (100 mL), dried over Na₂SO₄, filtered and concentrated to give a residue. The residue was purified by column (SiO₂, PE˜PE/EtOAc=20/1) to afford methyl 3-[3,5-dichloro-4-[2-(4-trityloxybutoxy)ethoxy]phenyl]propanoate (1.4 g, 2.30 mmol, 27.02% yield) as a light brown oil.

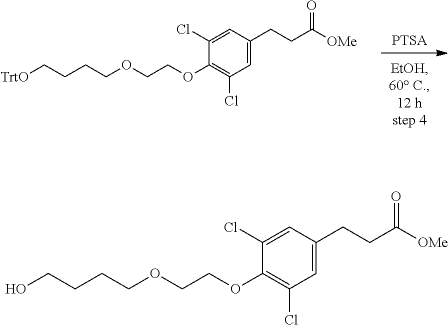

### Methyl 3-[3,5-dichloro-4-[2-(4-hydroxybutoxy)ethoxy]phenyl]propanoate

To a solution of methyl 3-[3,5-dichloro-4-[2-(4-trityloxybutoxy)ethoxy]phenyl]propanoate (1.4 g, 2.30 mmol, 1 eq) in EtOH (10 mL) was added PTSA (3.97 g, 23.04 mmol, 10 eq). The solution was stirred at 60° C. for 12 h. The reaction was concentrated to remove the solvent and the residue was diluted with EtOAc (80 mL) and washed with H₂O (20 mL*3), brine (20 mL*2). The organic layer was dried over Na₂SO₄, filtered and concentrated to give the residue. The residue was purified by column (SiO₂, PE/EtOAc=20/1-3/1) to afford methyl 3-[3,5-dichloro-4-[2-(4-hydroxybutoxy)ethoxy]phenyl]propanoate (500 mg, 1.10 mmol, 47.53% yield, 80% purity) as a light brown oil.

LCMS: ET20197-105-P1D (M+H⁺): 365.0 @ 1.225 min (5-95% ACN in H₂O, 2.0 min)

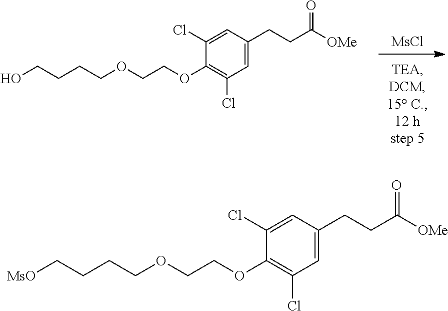 [SMILES: "MsCl" ; COC(=O)CCc1cc(Cl)c(OCCOCCCCO)c(Cl)c1 ; COC(=O)CCc1cc(Cl)c(OCCOCCCCOS(C)(=O)=O)c(Cl)c1]

### Methyl 3-[3,5-dichloro-4-[2-(4-methylsulfonyloxybutoxy)ethoxy]phenyl]propanoate

To a solution of methyl 3-[3,5-dichloro-4-[2-(4-hydroxybutoxy)ethoxy]phenyl]propanoate (500 mg, 1.37 mmol, 1 eq) and TEA (692.61 mg, 6.84 mmol, 952.70 uL, 5 eq) in DCM (20 mL) was added MsCl (0.6 g, 5.24 mmol, 405.41 uL, 3.83 eq). The reaction was stirred at 15° C. for 12 h. The mixture was washed with H₂O (20 mL*2) and brine (20 mL), dried over Na₂SO₄, filtered and concentrated to give methyl 3-[3,5-dichloro-4-[2-(4-methylsulfonyloxy butoxy)ethoxy]phenyl]propanoate (500 mg, 1.11 mmol, 80.74% yield, 98% purity) as a yellow solid.

LCMS: ET20197-125-PIP (M+H⁺): 464.9 @ 1.312 min (5-95% ACN in H₂O, 3.0 min)

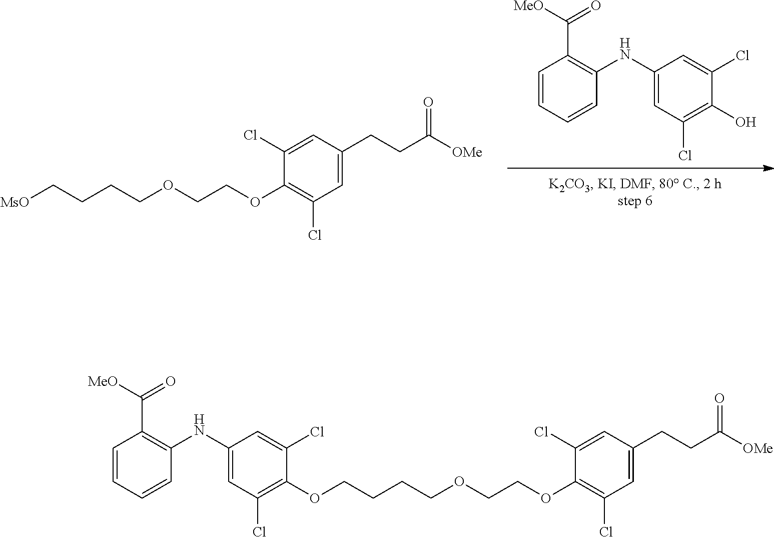 [SMILES: COC(=O)CCc1cc(Cl)c(OCCOCCCCOS(C)(=O)=O)c(Cl)c1 ; COC(=O)c1ccccc1Nc1cc(Cl)c(O)c(Cl)c1 ; COC(=O)CCc1cc(Cl)c(OCCOCCCCOc2c(Cl)cc(Nc3ccccc3C(=O)OC)cc2Cl)c(Cl)c1]

### Methyl 2-[3,5-dichloro-4-[4-[2-[2,6-dichloro-4-(3-methoxy-3-oxo-propyl)phenoxy]ethoxy]butoxy]anilino]benzoate

A mixture of methyl 3-[3,5-dichloro-4-[2-(4-methylsulfonyloxybutoxy)ethoxy]phenyl]propanoate (0.5 g, 1.13 mmol, 1 eq), methyl 2-(3,5-dichloro-4-hydroxy-anilino)benzoate (352.04 mg, 1.13 mmol, 1 eq), K₂CO₃ (467.62 mg, 3.38 mmol, 3 eq), KI (187.22 mg, 1.13 mmol, 1 eq) in DMF (10 mL) was degassed and purged with N₂ for 3 times, and then the mixture was stirred at 80° C. for 2 hr under N₂ atmosphere. The reaction was diluted with H₂O (30 mL) and extracted with DCM (50 mL*3). The organic layer was washed with brine (30 mL), dried over Na₂SO₄, filtered and concentrated to give methyl 2-[3,5-dichloro-4-[4-[2-[2,6-dichloro-4-(3-methoxy-3-oxo-propyl)phenoxy]ethoxy]butoxy]anilino]benzoate (0.6 g, 309.38 umol, 27.43% yield, 34% purity) as a brown solid.

LCMS: ET20197-129-P1D (M+H⁺): 660.2 @ 1.631 min (5-95% ACN in H₂O, 2.0 min)

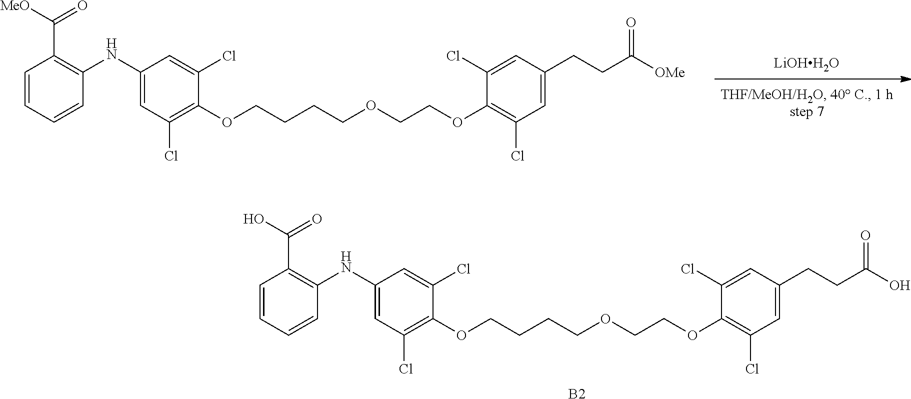 [SMILES: COC(=O)CCc1cc(Cl)c(OCCOCCCCOc2c(Cl)cc(Nc3ccccc3C(=O)OC)cc2Cl)c(Cl)c1 ; "LiOH·H2O" ; O=C(O)CCc1cc(Cl)c(OCCOCCCCOc2c(Cl)cc(Nc3ccccc3C(=O)O)cc2Cl)c(Cl)c1 ; "B2"]

### 2-[4-[4-[2-[4-(2-carboxyethyl)-2,6-dichloro-phenoxy]ethoxy]butoxy]-3,5-dichloro-anilino]benzoic acid

To a solution of methyl 2-[3,5-dichloro-4-[4-[2-[2,6-dichloro-4-(3-methoxy-3-oxopropyl) phenoxy]ethoxy]butoxy]anilino]benzoate (0.6 g, 909.95 umol, 1 eq) in THF (20 mL) and MeOH (10 mL), H₂O (10 mL) was added LiOH·H₂O (229.11 mg, 5.46 mmol, 6 eq). The mixture was stirred at 40° C. for 1 h. The reaction was concentrated to give the residue. The residue was diluted with H₂O (30 mL) and adjusted with 1N HCl to pH-2. The mixture was extracted with DCM (50 mL*3). The organic layer was concentrated to give the residue. The residue was purified by prep-HPLC (HCl condition) to afford 2-[4-[4-[2-[4-(2-carboxyethyl)-2,6-dichloro-phenoxy]ethoxy]butoxy]-3,5-dichloro-anilino]benzoic acid (294.7 mg, 466.79 umol, 51.30% yield, 100% purity) as a off white solid.

m.p: 131.2-132.6° C. by melting points apparatus (ET20197-131-PIM).

LCMS: ET20197-131-P1A (M+H⁺): 632.0@ 2.225 min (10-80% ACN in H₂O, 3.0 min)

¹H NMR: ET20197-131-P1AA (DMSO-d6, 400 MHz)

δ 9.50 (s, 1H), 7.91 (dd, J=1.5, 7.9 Hz, 1H), 7.44 (ddd, J=1.5, 7.2, 8.5 Hz, 1H), 7.34 (d, J=7.5 Hz, 4H), 7.24 (d, J=7.9 Hz, 1H), 6.91-6.84 (m, 1H), 4.14-4.08 (m, 2H), 3.95 (t, J=6.2 Hz, 2H), 3.78-3.70 (m, 2H), 3.54 (t, J=6.1 Hz, 2H), 2.81-2.73 (m, 2H), 2.55 (t, J=7.5 Hz, 2H), 1.86-1.67 (m, 4H)

Melting point: ET20197-131-P1M (131.2-132.6° C.)

### Synthesis of B3&B3A (T-618, T-663, T-664 and T-649)

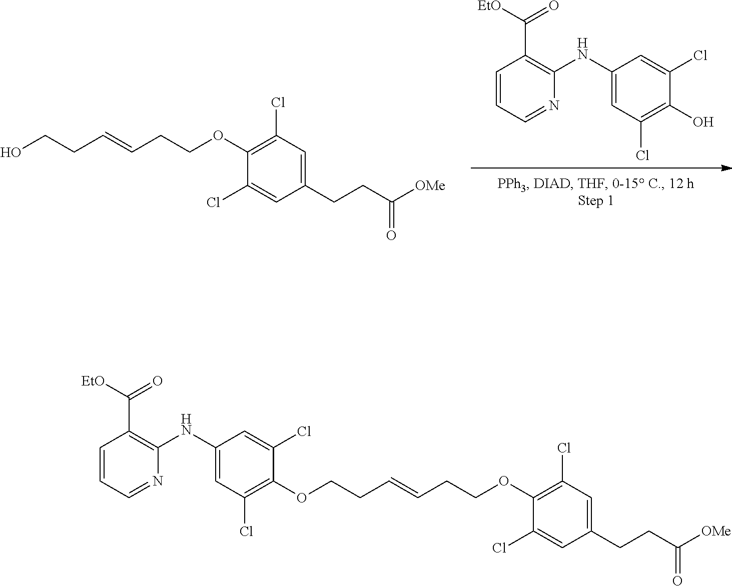 [SMILES: COC(=O)CCc1cc(Cl)c(OCC/C=C/CCO)c(Cl)c1 ; CCOC(=O)c1cccnc1Nc1cc(Cl)c(O)c(Cl)c1 ; CCOC(=O)c1cccnc1Nc1cc(Cl)c(OCC/C=C/CCOc2c(Cl)cc(CCC(=O)OC)cc2Cl)c(Cl)c1]

### Ethyl 2-[3,5-dichloro-4-[(E)-6-[2,6-dichloro-4-(3-methoxy-3-oxo-propyl)phenoxy]hex-3-enoxy]anilino]pyridine-3-carboxylate

To a mixture of methyl 3-[3,5-dichloro-4-[(E)-6-hydroxyhex-3-enoxy]phenyl]propanoate (8 g, 23.04 mmol, 1 eq), ethyl 2-(3,5-dichloro-4-hydroxy-anilino)pyridine-3-carboxylate (8.29 g, 25.34 mmol, 1.1 eq) and PPh₃ (12.09 g, 46.08 mmol, 2 eq) in THF (160 mL) was added DIAD (9.32 g, 46.08 mmol, 8.96 mL, 2 eq) in THF (40 mL) at 0° C. The reaction mixture was stirred at 15° C. for 12 h. The reaction mixture was concentrated to give a residue. The residue was first purified by flash silica gel chromatography (ISCO®; 80 g SepaFlash® Silica Flash Column, Eluent of 0˜3% Ethylacetate/Petroleum ethergradient @ 80 mL/min) and second purified by reversed MPLC(TFA condition) to afford ethyl 2-[3,5-dichloro-4-[(E)-6-[2,6-dichloro-4-(3-methoxy-3-oxo-propyl)phenoxy]hex-3-enoxy]anilino]pyridine-3-carboxylate (6 g, 9.05 mmol, 39.28% yield, 99% purity) as a brown oil.

¹H NMR: ET20960-6-P1AB (CHLOROFORM-d, 400 MHz)

δ 10.24 (s, 1H), 8.40 (dd, J=2.0, 4.6 Hz, 1H), 8.26 (dd, J=2.0, 7.9 Hz, 1H), 7.75 (s, 2H), 7.12 (s, 2H), 6.79 (dd, J=4.9, 7.7 Hz, 1H), 5.78-5.66 (m, 2H), 4.40 (q, J=7.2 Hz, 2H), 4.03 (dt, J=2.0, 6.9 Hz, 4H), 3.69 (s, 3H), 2.90-2.82 (m, 2H), 2.64-2.51 (m, 6H), 1.42 (t, J=7.1 Hz, 3H).

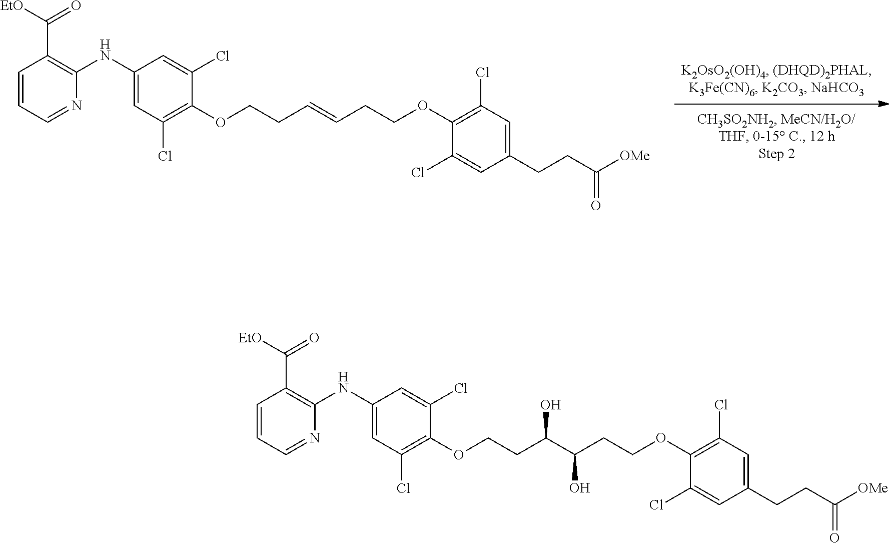 [SMILES: CCOC(=O)c1cccnc1Nc1cc(Cl)c(OCC/C=C/CCOc2c(Cl)cc(CCC(=O)OC)cc2Cl)c(Cl)c1 ; CCOC(=O)c1cccnc1Nc1cc(Cl)c(OCC[C@@H](O)[C@H](O)CCOc2c(Cl)cc(CCC(=O)OC)cc2Cl)c(Cl)c1]

### Ethyl 2-[3,5-dichloro-4-[(3R,4R)-6-[2,6-dichloro-4-(3-methoxy-3-oxo-propyl)phenoxy]-3,4-dihydroxy-hexoxy]anilino]pyridine-3-carboxylate

A mixture of methanesulfonamide (144.92 mg, 1.52 mmol, 1 eq), NaHCO₃ (383.95 mg, 4.57 mmol, 177.76 uL, 3 eq), K₃[Fe(CN)₆] (1.50 g, 4.57 mmol, 1.25 mL, 3 eq), K₂OsO₄·2H₂O (56.13 mg, 152.35 umol, 0.1 eq), (DHQD)₂PHAL (296.70 mg, 380.88 umol, 0.25 eq) and K₂CO₃ (631.67 mg, 4.57 mmol, 3 eq) in H₂O (20 mL), THF (40 mL) and MeCN (20 mL) was stirred at 20° C. for 30 min, then ethyl 2-[3,5-dichloro-4-[(E)-6-[2,6-dichloro-4-(3-methoxy-3-oxopropyl)phenoxy]hex-3-enoxy]anilino]pyridine-3-carboxylate (1 g, 1.52 mmol, 1 eq) was added at 0° C. The final reaction mixture was stirred at 20° C. for 11.5 h. The reaction mixture was filtered and partitioned between H₂O (50 mL) and EtOAc (50 mL). The organic layer was dried over Na₂SO₄ and concentrated to give a residue. The residue was purified by silica gel column (PE:EtOAc=5:1 to 3:1) to afford ethyl 2-[3,5-dichloro-4-[(3R,4R)-6-[2,6-dichloro-4-(3-methoxy-3-oxo-propyl)phenoxy]-3,4-dihydroxy-hexoxy]anilino]pyridine-3-carboxylate (0.9 g, 1.29 mmol, 84.71% yield, 99% purity) as a white gum.

¹H NMR: ET20960-7-P1AA (CHLOROFORM-d, 400 MHz)

δ 10.27 (s, 1H), 8.40 (dd, J=2.1, 4.8 Hz, 1H), 8.27 (dd, J=2.0, 7.8 Hz, 1H), 7.77 (s, 2H), 7.15 (s, 2H), 6.80 (dd, J=4.8, 7.8 Hz, 1H), 4.40 (q, J=7.1 Hz, 2H), 4.30-4.18 (m, 4H), 4.09-4.00 (m, 2H), 3.69 (s, 3H), 2.96-2.80 (m, 4H), 2.66-2.57 (m, 2H), 2.12 (q, J=5.9 Hz, 4H), 1.43 (t, J=7.1 Hz, 3H).

SFC: ET20960-7-P1S (Retention time: P1=1.82 min; P2=1.91 min, 87.44% ee value)

The major peak (P2) is the desired product.

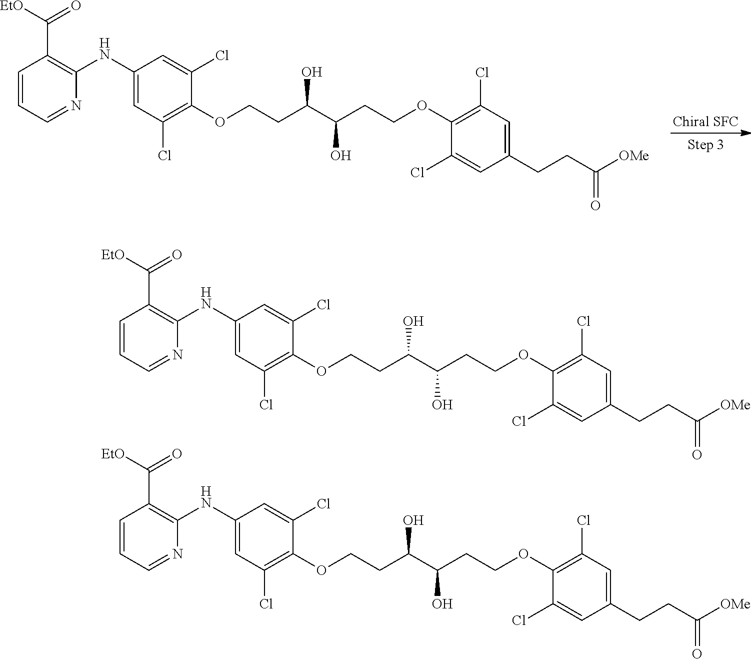 [SMILES: CCOC(=O)c1cccnc1Nc1cc(Cl)c(OCC[C@@H](O)[C@H](O)CCOc2c(Cl)cc(CCC(=O)OC)cc2Cl)c(Cl)c1 ; CCOC(=O)c1cccnc1Nc1cc(Cl)c(OCC[C@@H](O)[C@H](O)CCOc2c(Cl)cc(CCC(=O)OC)cc2Cl)c(Cl)c1 ; CCOC(=O)c1cccnc1Nc1cc(Cl)c(OCC[C@H](O)[C@@H](O)CCOc2c(Cl)cc(CCC(=O)OC)cc2Cl)c(Cl)c1]

### Ethyl 2-[3,5-dichloro-4-[(3S,4S)-6-[2,6-dichloro-4-(3-methoxy-3-oxo-propyl)phenoxy]-3,4-dihydroxy-hexoxy]anilino]pyridine-3-carboxylate & Ethyl 2-[3,5-dichloro-4-[(3R,4R)-6-[2,6-dichloro-4-(3-methoxy-3-oxo-propyl)phenoxy]-3,4-dihydroxy-hexoxy]anilino]pyridine-3-carboxylate

Ethyl 2-[3,5-dichloro-4-[(3R,4R)-6-[2,6-dichloro-4-(3-methoxy-3-oxo-propyl)phenoxy]-3,4-dihydroxy-hexoxy]anilino]pyridine-3-carboxylate (945 mg, 87.44% ee value) was separated by Chiral SFC separation (Instrument: Waters prep-SFC 80Q; Column: Chiralpak OJ-H, 250*25 mm i.d. 10 u; Mobile phase: A for CO₂ and B for MEOH (0.1% NH3·H₂O); Gradient: B %=40%; Flow rate: 70 g/min; Column temperature: 40° C.; System back pressure:100 bar) to afford the product 2 (pure, 700 mg) and the mixture of product 1 and product 2 (racemic, 120 mg). The racemic product was twice separated by Chiral SFC (Instrument: Waters prep-SFC 80Q; Column: Chiralpak AD-H, 250*25 mm i.d. 5 u; Mobile phase: A for CO₂ and B for MeOH (0.1% NH₃·H₂O); Gradient: B %=40%; Flow rate: 70 g/min; Column temperature: 40° C.; System back pressure:100 bar) to afford the pure product 1 (40 mg).

P1: Ethyl 2-[3,5-dichloro-4-[(3S,4S)-6-[2,6-dichloro-4-(3-methoxy-3-oxo-propyl)phenoxy]-3,4-dihydroxy-hexoxy]anilino]pyridine-3-carboxylate (40 mg, 55.56 umol, 4.06% yield, 95.9% purity) was obtained as a light-yellow gum.

SFC: ET20960-8-P1S (Retention time: 1.83 min; 100% ee velue)

P2: Ethyl 2-[3,5-dichloro-4-[(3R,4R)-6-[2,6-dichloro-4-(3-methoxy-3-oxo-propyl)phenoxy]
3,4-dihydroxy-hexoxy]anilino]pyridine-3-carboxylate (700 mg, 1.01 mmol, 73.70% yield, 99.5% purity) was obtained as a light-yellow gum.

SFC: ET20960-8-P2S (Retention time: 1.91 min; 100% ee velue)

¹H NMR: ET20960-8-P2AA (CHLOROFORM-d, 400 MHz)

δ 10.27 (s, 1H), 8.41 (dd, J=2.0, 4.8 Hz, 1H), 8.27 (dd, J=2.0, 7.8 Hz, 1H), 7.77 (s, 2H), 7.15 (s, 2H), 6.80 (dd, J=4.8, 7.8 Hz, 1H), 4.40 (q, J=7.2 Hz, 2H), 4.30-4.17 (m, 4H), 4.09-4.01 (m, 2H), 3.69 (s, 3H), 2.87 (t, J=7.6 Hz, 4H), 2.65-2.57 (m, 2H), 2.18-2.07 (m, 4H), 1.43 (t, J=7.2 Hz, 3H).

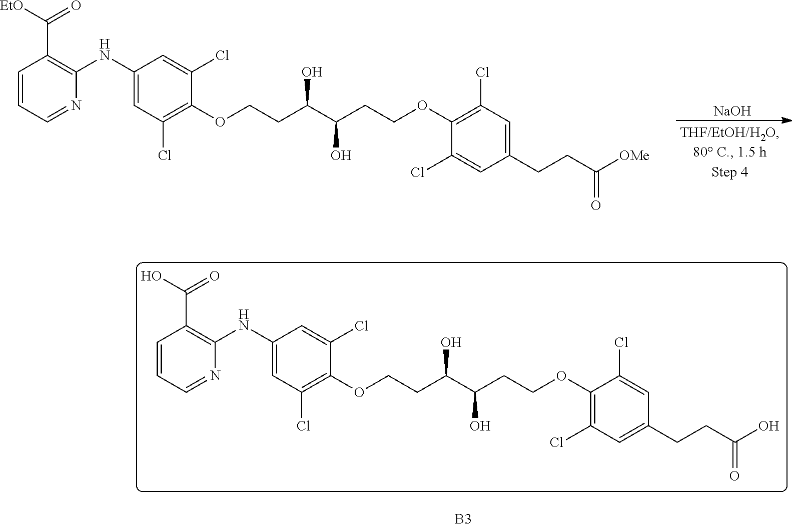 [SMILES: CCOC(=O)c1cccnc1Nc1cc(Cl)c(OCC[C@@H](O)[C@H](O)CCOc2c(Cl)cc(CCC(=O)OC)cc2Cl)c(Cl)c1 ; "NaOH" ; O=C(O)CCc1cc(Cl)c(OCC[C@@H](O)[C@H](O)CCOc2c(Cl)cc(Nc3ncccc3C(=O)O)cc2Cl)c(Cl)c1 ; "B3"]

### 2-[4-[(3R,4R)-6-[4-(2-carboxyethyl)-2,6-dichloro-phenoxy]-3,4-dihydroxy-hexoxy]-3,5-dichloro-anilino]pyridine-3-carboxylic acid

To a solution of ethyl 2-[3,5-dichloro-4-[(3R,4R)-6-[2,6-dichloro-4-(3-methoxy-3-oxo-propyl) phenoxy]-3,4-dihydroxyhexoxy]anilino]pyridine-3-carboxylate (0.6 g, 869.07 umol, 1 eq) in EtOH (9 mL), THF (4 mL) and H₂O (5 mL) was added NaOH (180.75 mg, 4.52 mmol, 5.2 eq), the reaction mixture was stirred at 80° C. for 1.5 h. The reaction mixture was concentrated to give a residue. The residue was dissolved with H₂O (5 mL), basified to pH=3 with H₂SO₄ (2 N) and extracted with EtOAc (10 mL*2). The combined organic layer was dried over Na₂SO₄, concentrated and followed lyophilized to give the crude product. The product was purified by FA prep-HPLC, HCl prep-HPLC and finally neutral prep-HPLC to afford 2-[4-[(3R,4R)-6-[4-(2-carboxyethyl)-2,6-dichloro-phenoxy]-3,4-dihydroxy-hexoxy]-3,5-dichloro-anilino]pyridine-3-carboxylic acid (257.6 mg, 99.85% purity) as a white solid. (Note: The reaction was combined with another reaction (ET20960-10) in 100 mg scale for purification and work up.)

LCMS: ET20960-11-P1P1 (M+H*): 649.0@ 1.916 min (10-80% ACN in H₂O, 4.5 min)

¹H NMR: ET20960-11-P1N (METHANOL-d4, 400 MHz)

δ 8.35-8.22 (m, 2H), 7.80 (s, 2H), 7.26 (s, 2H), 6.81 (dd, J=4.9, 7.7 Hz, 1H), 4.28-4.09 (m, 4H), 3.99-3.88 (m, 2H), 2.85 (t, J=7.5 Hz, 2H), 2.63-2.52 (m, 2H), 2.21-2.08 (m, 2H), 2.06-1.93 (m, 2H).

SFC: ET20960-11-P1P (Retention time: 4.50 min; 100% ee value)

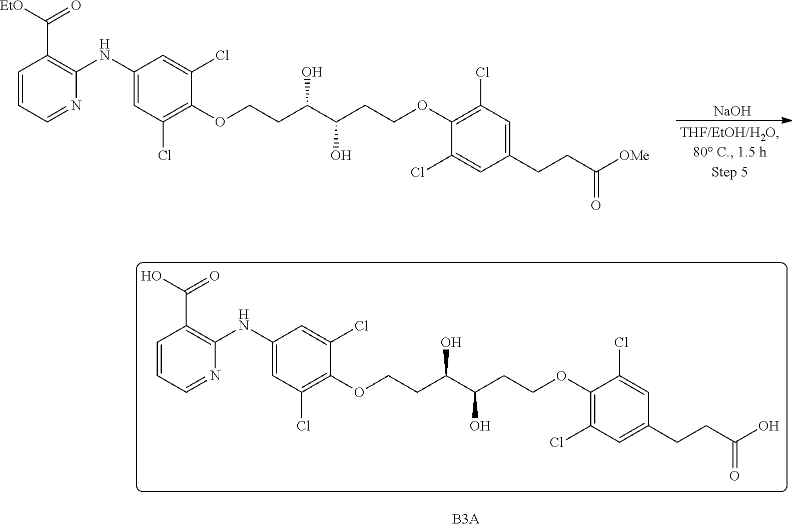 [SMILES: CCOC(=O)c1cccnc1Nc1cc(Cl)c(OCC[C@H](O)[C@@H](O)CCOc2c(Cl)cc(CCC(=O)OC)cc2Cl)c(Cl)c1 ; "NaOH" ; O=C(O)CCc1cc(Cl)c(OCC[C@@H](O)[C@H](O)CCOc2c(Cl)cc(Nc3ncccc3C(=O)O)cc2Cl)c(Cl)c1 ; "B3A"]

### 2-[4-[(3S,4S)-6-[4-(2-carboxyethyl)-2,6-dichloro-phenoxy]-3,4-dihydroxy-hexoxy]-3,5-dichloro-anilino]pyridine-3-carboxylicacid

To a solution of ethyl 2-[3,5-dichloro-4-[(3S,4S)-6-[2,6-dichloro-4-(3-methoxy-3-oxo-propyl) phenoxy]-3,4-dihydroxyhexoxy]anilino]pyridine-3-carboxylate (0.04 g, 57.94 umol, 1 eq) in EtOH (1 mL), THF (0.4 mL) and H₂O (0.5 mL) was added NaOH (12.05 mg, 301.28 umol, 5.2 eq), the reaction mixture was stirred at 80° C. for 1.5 h. The reaction mixture was concentrated to give a residue. The residue was dissolved with H₂O (2 mL), basified to pH=3 with H₂SO₄ (2 N) and extracted with EtOAc (5 mL*2). The combined organic layer was dried over Na₂SO₄, concentrated to give a residue. The residue was purified by FA prep-HPLC, HCl-prep-HPLC (column: Agela Durashell C18 150*25 5 u; mobile phase: [water (10 Mm NH₄HCO₃)-ACN]; B %: 15%-45%, 10 min) to afford the pure product. 2-[4-[(3S,4S)-6-[4-(2-carboxyethyl)-2,6-dichloro-phenoxy]-3,4-dihydroxy-hexoxy]-3,5-dichloro-anilino]pyridine-3-carboxylic acid (2.2 mg, 3.34 umol, 5.76% yield, 98.4% purity) as a white solid.

LCMS: ET20960-13-P1B1 (M+H⁺): 649.0@ 1.917 min (10-80% ACN in H₂O, 4.5 min)

¹H NMR: ET20197-13-P1N (METHANOL-d4, 400 MHz)

δ 8.36-8.23 (m, 2H), 7.80 (s, 2H), 7.25 (s, 2H), 6.89-6.79 (m, 1H), 4.26-4.09 (m, 4H), 3.97-3.88 (m, 2H), 2.89-2.81 (m, 2H), 2.64-2.53 (m, 2H), 2.21-2.07 (m, 2H), 2.05-1.91 (m, 2H).

SFC: ET20960-13-P1S1 (Retention time: 5.599 min; 95.74% ee value)

### Synthesis of B4&B4A (T-670 and T-669)

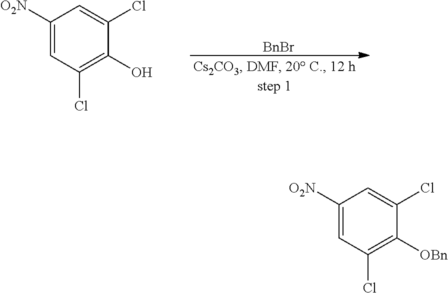 [SMILES: O=[N+]([O-])c1cc(Cl)c(O)c(Cl)c1 ; "BnBr" ; O=[N+]([O-])c1cc(Cl)c(OCc2ccccc2)c(Cl)c1]

### 2-benzyloxy-1,3-dichloro-5-nitro-benzene

To a mixture of 2,6-dichloro-4-nitro-phenol (50 g, 240.39 mmol, 1 eq) and Cs₂CO₃ (195.81 g, 600.97 mmol, 2.5 eq) in NMP (800 mL) was added bromomethylbenzene (61.67 g, 360.58 mmol, 42.83 mL, 1.5 eq) drop-wise at 20° C., the reaction mixture was stirred at 20° C. for 12 h. The reaction mixture was filtered and diluted with EtOAc (1200 mL), washed with sub-saturated brine (600 mL*3). The organic layer was dried over Na₂SO₄ and concentrated to give a residue. The residue was purified by silica gel column (PE:EtOAc=100:1 to 100:4) and then washed by MeOH (50 mL) to afford 2-benzyloxy-1,3-dichloro-5-nitro-benzene (30 g) as a white solid.

The reaction was combined with another reaction (ET20960-4) in 2 g scale for purification and work up.

¹H NMR: ET20960-6-P1AB (CHLOROFORM-d, 400 MHz)

δ 8.24 (s, 2H), 7.54 (dd, J=1.7, 7.5 Hz, 2H), 7.46-7.36 (m, 3H), 5.18 (s, 2H).

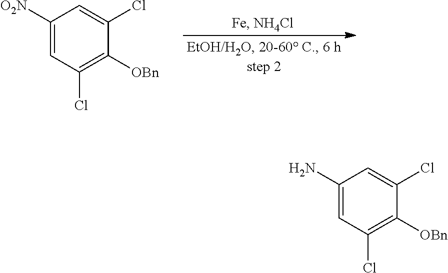

### 4-benzyloxy-3,5-dichloro-aniline

To a solution of 2-benzyloxy-1,3-dichloro-5-nitro-benzene (30 g, 100.63 mmol, 1 eq) in EtOH (600 mL) and H₂O (120 mL) was added Fe (28.10 g, 503.15 mmol, 5 eq) and NH₄Cl (26.91 g, 503.15 mmol, 5 eq) at 20° C. in portions, the reaction mixture was stirred at 60° C. for 6 hr. The reaction mixture was filtered and concentrated to give a residue. The residue was purified by silica gel column (PE:EtOAc=5:1 to 1:1) to afford 4-benzyloxy-3,5-dichloro-aniline (25 g, 93.24 mmol, 92.65% yield) as a white solid.

¹H NMR: ET20960-9-P1AA (CHLOROFORM-d, 400 MHz)

δ 7.57 (d, J=6.8 Hz, 2H), 7.46-7.30 (m, 3H), 6.64 (s, 2H), 4.96 (s, 2H), 3.64 (br s, 2H)

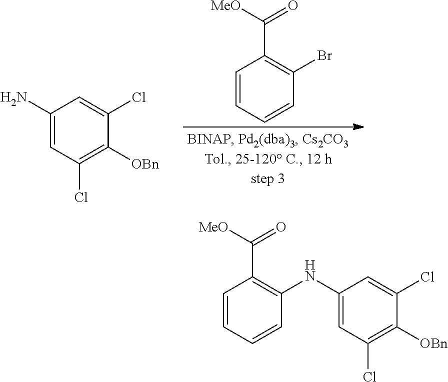 [SMILES: Nc1cc(Cl)c(OCc2ccccc2)c(Cl)c1 ; COC(=O)c1ccccc1Br ; COC(=O)c1ccccc1Nc1cc(Cl)c(OCc2ccccc2)c(Cl)c1]

### Methyl 2-(4-benzyloxy-3,5-dichloro-anilino)benzoate

To a mixture of 4-benzyloxy-3,5-dichloro-aniline (25 g, 93.24 mmol, 1 eq), methyl 2-bromobenzoate (30.07 g, 139.85 mmol, 19.66 mL, 1.5 eq) and Cs₂CO₃ (75.94 g, 233.09 mmol, 2.5 eq) in Tol. (300 mL) was added BINAP (4.35 g, 6.99 mmol, 0.075 eq) and Pd₂(dba)₃ (4.27 g, 4.66 mmol, 0.05 eq) at 25° C., the reaction mixture was degassed and purged with N₂ for 3 times and then stirred at 120° C. for 12 h. The reaction mixture was filtered and the filter cake was washed with MeOH (300 mL) and hexane (200 mL) to give a residue. The residue was purified by silica gel column (PE:EtOAc=20:1) to afford methyl 2-(4-benzyloxy-3,5-dichloro-anilino)benzoate (32 g, 79.55 mmol, 85.32% yield) as a light-yellow solid.

¹H NMR: ET20960-12-P1AA (CHLOROFORM-d, 400 MHz)

δ 9.50 (s, 1H), 8.04 (dd, J=1.4, 8.0 Hz, 1H), 7.63 (d, J=7.0 Hz, 2H), 7.51-7.39 (m, 4H), 7.31 (d, J=6.2 Hz, 1H), 6.88 (t, J=7.5 Hz, 1H), 5.09 (s, 2H), 3.96 (s, 3H).

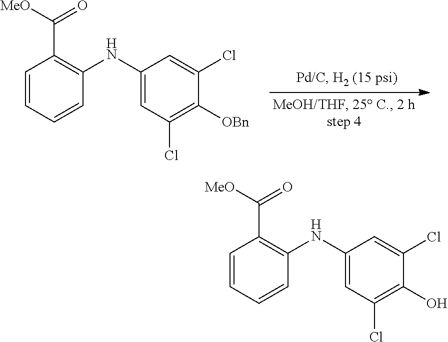 [SMILES: COC(=O)c1ccccc1Nc1cc(Cl)c(OCc2ccccc2)c(Cl)c1 ; COC(=O)c1ccccc1Nc1cc(Cl)c(O)c(Cl)c1]

### Methyl 2-(3,5-dichloro-4-hydroxy-anilino)benzoate

To a solution of methyl 2-(4-benzyloxy-3,5-dichloro-anilino)benzoate (15 g, 37.29 mmol, 1 eq) in MeOH (300 mL) and THF (300 mL) was added Pd/C (4 g, 10% purity), the reaction mixture degassed and purged with H₂ for 3 times, the reaction mixture was stirred under H₂ (15 psi) at 25° C. for 2 hr. The reaction mixture was filtered and the filtrate was concentrated to give a residue. The residue was purified by silica gel column (PE:EtOAc=35:1) to afford methyl 2-(3,5-dichloro-4-hydroxy-anilino)benzoate (99.4% purity) (22 g) as a light yellow solid.

The reaction was combined with another reaction (ET20960-15) in 1 g scale for work up and purification.

¹H NMR: ET20960-16-P1AA (CHLOROFORM-d, 400 MHz)

δ 9.32 (br s, 1H), 7.97 (dd, J=1.4, 8.0 Hz, 1H), 7.39-7.31 (m, 1H), 7.20 (s, 2H), 7.05 (d, J=8.4 Hz, 1H), 6.77 (t, J=7.5 Hz, 1H), 5.72 (s, 1H), 3.91 (s, 3H).

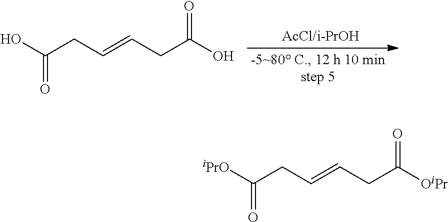 [SMILES: O=C(O)C/C=C/CC(=O)O ; CC(C)OC(=O)C/C=C/CC(=O)OC(C)C]

### Diisopropyl (E)-hex-3-enedioate

Acetyl chloride (26.40 g, 336.32 mmol, 24 mL, 4.04 eq) was slowly added to i-PrOH (180 mL) at −5° C. After stirred at −5° C. for 10 min, (E)-hex-3-enedioic acid (12 g, 83.26 mmol, 1 eq) was added, and the reaction was heated to 80° C. and stirred for 12 h. The reaction was concentrated to give a residue. The residue was diluted with EtOAc (500 mL) and washed with aq.NaHCO₃ (100 mL*4). The organic layer was washed with brine (200 mL), dried over Na₂SO₄, filtered and concentrated under vacuum to give a residue. The residue was purified by column (SiO₂, PE/EtOAc=100/1-50/1) to afford diisopropyl (E)-hex-3-enedioate (28 g, 122.65 mmol, 73.66% yield) as a yellow solid.

¹H NMR: ET20197-40-P1AA (CHLOROFORM-d, 400 MHz)

δ 5.68 (tt, J=1.7, 3.7 Hz, 2H), 5.00 (spt, J=6.3 Hz, 2H), 3.07-3.03 (m, 4H), 1.23 (d, J=6.4 Hz, 12H)

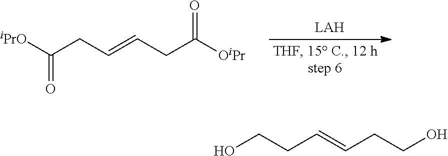 [SMILES: CC(C)OC(=O)C/C=C/CC(=O)OC(C)C ; OCC/C=C/CCO]

### (E)-hex-3-ene-1,6-diol

To a suspension of LAH (16.63 g, 438.05 mmol, 4 eq) in THF (100 mL) was added diisopropyl (E)-hex-3-enedioate (25 g, 109.51 mmol, 1 eq) in THF (100 mL) dropwise, the reaction mixture was stirred at 15° C. for 12 h. The reaction mixture was quenched with H₂O (16.7 mL) at 0° C. dropwise, and then NaOH (aq, 15% w/w, 16.7 mL) was added, followed by H₂O (50 mL) was added at 0° C. The suspension was filtered and the filtrate was concentrated to afford (E)-hex-3-ene-1,6-diol (12 g, 103.31 mmol, 94.33% yield) as a light yellow oil.

¹H NMR: ET20197-66-P1 AA (CHLOROFORM-d, 400 MHz)

δ 5.55-5.47 (m, 2H), 3.62 (q, J=5.1 Hz, 4H), 2.85-2.72 (m, 2H), 2.37-2.19 (m, 4H)

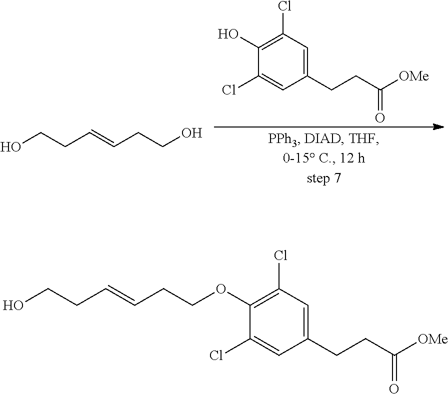 [SMILES: COC(=O)CCc1cc(Cl)c(O)c(Cl)c1 ; OCC/C=C/CCO ; COC(=O)CCc1cc(Cl)c(OCC/C=C/CCO)c(Cl)c1]

### Methyl 3-[3,5-dichloro-4-[(E)-6-hydroxyhex-3-enoxy]phenyl] propanoate

To a solution of (E)-hex-3-ene-1,6-diol (12 g, 103.31 mmol, 2.57 eq), methyl 3-(3,5-dichloro-4-hydroxy-phenyl)propanoate (10 g, 40.15 mmol, 1 eq) and PPh₃ (21.06 g, 80.29 mmol, 2 eq) in THF (300 mL) was added a solution of DIAD (16.24 g, 80.29 mmol, 15.61 mL, 2 eq) in THF (50 mL) at 0° C., the reaction mixture was stirred at 15° C. for 12 h. The reaction was concentrated to give the residue. The residue was purified by column (SiO₂, PE/EtOAc=50/1 ˜5/1) to afford methyl 3-[3,5-dichloro-4-[(E)-6-hydroxyhex-3-enoxy]phenyl]propanoate (20 g, 83% LCMS purity) as a light yellow oil.

LCMS: ET20197-91-P1 A (M+H⁺): 347.1 @ 1.252 min (5-95% ACN in H₂O, 2.0 min)

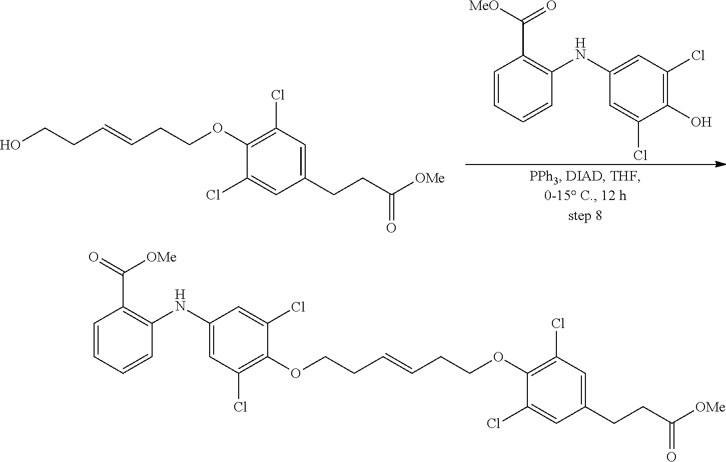 [SMILES: COC(=O)CCc1cc(Cl)c(OCC/C=C/CCO)c(Cl)c1 ; COC(=O)c1ccccc1Nc1cc(Cl)c(O)c(Cl)c1 ; COC(=O)CCc1cc(Cl)c(OCC/C=C/CCOc2c(Cl)cc(Nc3ccccc3C(=O)OC)cc2Cl)c(Cl)c1]

### Methyl 2-[3,5-dichloro-4-[4-[2-[2,6-dichloro-4-(3-methoxy-3-oxo-propyl)phenoxy]ethoxy]butoxy]anilino]benzoate

To a mixture of methyl 3-[3,5-dichloro-4-[(E)-6-hydroxyhex-3-enoxy]phenyl]propanoate (6 g, 17.28 mmol, 1.2 eq), methyl 2-(3,5-dichloro-4-hydroxy-anilino)benzoate (4.49 g, 14.40 mmol, 1 eq) and PPh₃ (7.55 g, 28.80 mmol, 2 eq) in THF (25 mL) was added DIAD (5.82 g, 28.80 mmol, 5.60 mL, 2 eq) at 0° C., the reaction mixture was stirred at 15° C. for 12 h. The reaction was concentrated to give the residue. The residue was purified by flash silica gel chromatography (ISCO®; 120 g SepaFlash® Silica Flash Column, Eluent of 0 ˜2% Ethylacetate/Petroleum ethergradient @ 100 mL/min) to afford methyl 2-[3,5-dichloro-4-[(E)-6-[2,6-dichloro-4-(3-methoxy-3-oxo-propyl)phenoxy]hex-3-enoxy]anilino]benzoate (4.6 g, 6.67 mmol, 46.32% yield, 93% purity) as a yellow solid.

¹H NMR: ET20197-118-P1AA (CHLOROFORM-d, 400 MHz)

δ 9.41 (s, 1H), 7.98 (dd, J=1.6, 8.1 Hz, 1H), 7.38 (ddd, J=1.6, 7.2, 8.5 Hz, 1H), 7.23-7.17 (m, 3H), 7.13 (s, 2H), 6.81 (ddd, J=1.0, 7.2, 8.1 Hz, 1H), 5.79-5.70 (m, 2H), 4.04 (dt, J=3.2, 6.9 Hz, 4H), 3.91 (s, 3H), 3.69 (s, 3H), 2.90-2.80 (m, 2H), 2.61 (br t, J=7.6 Hz, 6H).

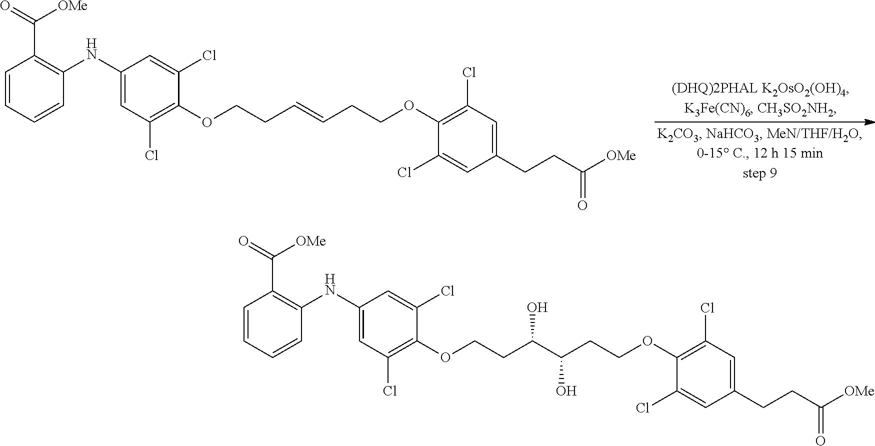 [SMILES: COC(=O)CCc1cc(Cl)c(OCC/C=C/CCOc2c(Cl)cc(Nc3ccccc3C(=O)OC)cc2Cl)c(Cl)c1 ; COC(=O)CCc1cc(Cl)c(OCC[C@H](O)[C@@H](O)CCOc2c(Cl)cc(Nc3ccccc3C(=O)OC)cc2Cl)c(Cl)c1]

### Methyl 2-[3,5-dichloro-4-[(3S,4S)-6-[2,6-dichloro-4-(3-methoxy-3-oxo-propyl)phenoxy]-3,4-dihydroxyhexoxy]anilino]benzoate

A solution of NaHCO₃ (130.98 mg, 1.56 mmol, 60.64 uL, 1 eq), (DHQ)₂PHAL (60.73 mg, 77.96 umol, 0.05 eq), K₂OsO₄·2H₂O (11.49 mg, 31.18 umol, 0.02 eq), K₂CO₃ (538.72 mg, 3.90 mmol, 2.5 eq), K₃[Fe(CN)₆] (1.28 g, 3.90 mmol, 1.07 mL, 2.5 eq) and MeSO₂NH₂ (148.31 mg, 1.56 mmol, 1 eq) in H₂O (20 mL) and MeCN (20 mL) was stirred for 15 min at 15° C. The mixture was cooled to 0° C. and methyl 2-[3,5-dichloro-4-[(E)-6-[2,6-dichloro-4-(3-methoxy-3-oxo-propyl)phenoxy]hex-3-enoxy]anilino]benzoate (1 g, 1.56 mmol, 1 eq) in THF (20 mL) was added. The reaction was stirred at 15° C. for 12 h. Na₂SO; (10 g) in H₂O (50 mL) was added, and the mixture was stirred at 15° C. for 10 min. The mixture was extracted with EtOAc (4×100 mL). The combined organic layers were washed with brine (50 mL*2), dried over Na₂SO₄ and concentrated under vacuum to give a residue. The residue was purified by column (SiO₂, PE/EtOAc=10/1 ˜1/1) to give methyl 2-[3,5-dichloro-4-[(3S,4S)-6-[2,6-dichloro-4-(3-methoxy-3-oxo-propyl)phenoxy]-3,4-dihydroxyhexoxy]anilino]benzoate (900 mg, 99.8% purity, 86.2% de value) as a colorless oil.

SFC: ET12197-144-P1S (Retention time: P1: 2.85 min; P2: 3.57 min; 86.2% ee value)

The major peak (P2) is the desired product.

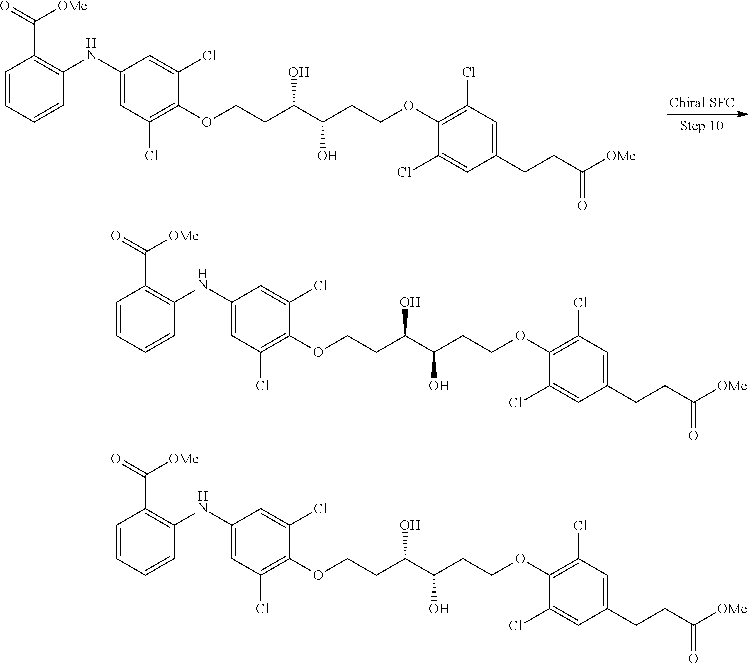 [SMILES: COC(=O)CCc1cc(Cl)c(OCC[C@H](O)[C@@H](O)CCOc2c(Cl)cc(Nc3ccccc3C(=O)OC)cc2Cl)c(Cl)c1 ; COC(=O)CCc1cc(Cl)c(OCC[C@@H](O)[C@H](O)CCOc2c(Cl)cc(Nc3ccccc3C(=O)OC)cc2Cl)c(Cl)c1 ; COC(=O)CCc1cc(Cl)c(OCC[C@H](O)[C@@H](O)CCOc2c(Cl)cc(Nc3ccccc3C(=O)OC)cc2Cl)c(Cl)c1]

### Methyl 2-[3,5-dichloro-4-[(3S,4S)-6-[2,6-dichloro-4-(3-methoxy-3-oxo-propyl)phenoxy]-3,4-dihydroxyhexoxy]anilino]benzoate

Methyl 2-[3,5-dichloro-4-[(3S,4S)-6-[2,6-dichloro-4-(3-methoxy-3-oxo-propyl)phenoxy]-3,4-dihydroxyhexoxy]anilino]benzoate (900 mg, 86.2% ee value), ET20197-141 (90 mg, 80.84% ee value) and ET20197-142 (50 mg, 81.82% ee value) were separated by SFC (Instrument: Waters prep-SFC 80Q; Column: Chiralpak AD-H, 250*25 mm i.d. 5 u; Mobile phase: A for CO₂ and B for MEOH (0.1% NH₃·H₂O); Gradient: B %=40%; Flow rate: 70 g/min; Column temperature: 40° C.; System back pressure:100 bar) to give:

P1: methyl 2-[3,5-dichloro-4-[(3R,4R)-6-[2,6-dichloro-4-(3-methoxy-3-oxo-propyl) phenoxy]-3,4-dihydroxyhexoxy]anilino]benzoate (50 mg, 81% purity, 100% ee value) was obtained as a colorless oil.

SFC: ET20197-144-P1S (Retention time: 2.80 min; 100% ee value)

P2: methyl 2-[3,5-dichloro-4-[(3S,4S)-6-[2,6-dichloro-4-(3-methoxy-3-oxo-propyl) phenoxy]-3,4-dihydroxyhexoxy]anilino]benzoate (700 mg, 99.7% purity, 100% ee value) was obtained as a colorless oil.

SFC: ET20197-144-P2S (Retention time: 3.60 min; 100% ee value)

¹H NMR: ET20197-144-P2AA (CHLOROFORM-d, 400 MHz)

δ 9.43 (s, 1H), 7.98 (dd, J=1.4, 8.0 Hz, 1H), 7.42-7.35 (m, 1H), 7.24-7.18 (m, 3H), 7.15 (s, 2H), 6.82 (t, J=7.5 Hz, 1H), 4.30-4.18 (m, 4H), 4.09-4.01 (m, 2H), 3.91 (s, 3H), 3.69 (s, 3H), 2.87 (t, J=7.6 Hz, 2H), 2.65-2.58 (m, 2H), 2.16-2.09 (m, 4H)

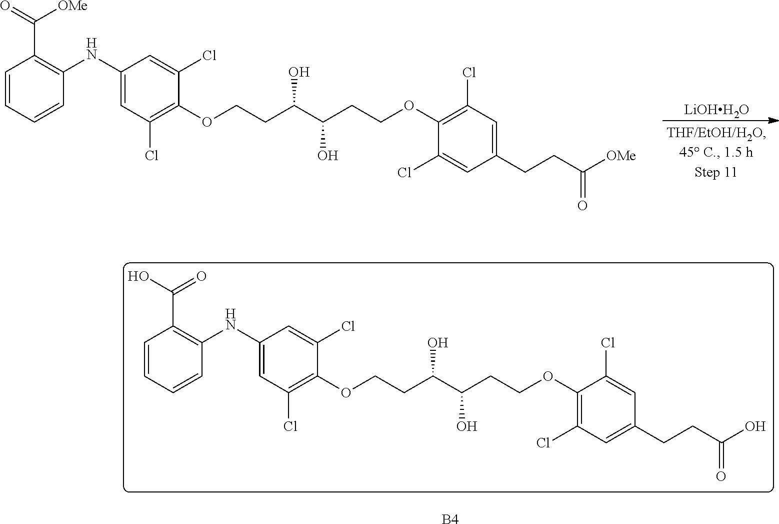 [SMILES: COC(=O)CCc1cc(Cl)c(OCC[C@H](O)[C@@H](O)CCOc2c(Cl)cc(Nc3ccccc3C(=O)OC)cc2Cl)c(Cl)c1 ; "LiOH·H2O" ; O=C(O)CCc1cc(Cl)c(OCC[C@H](O)[C@@H](O)CCOc2c(Cl)cc(Nc3ccccc3C(=O)O)cc2Cl)c(Cl)c1 ; "B4"]

### 2-[4-[(3S,4S)-6-[4-(2-carboxyethyl)-2,6-dichloro-phenoxy]-3,4-dihydroxy-hexoxy]-3,5-dichloro-anilino]benzoic acid

To a solution of methyl 2-[3,5-dichloro-4-[(3S,4S)-6-[2,6-dichloro-4-(3-methoxy-3-oxo-propyl)phenoxy]-3,4-dihydroxyhexoxy]anilino]benzoate (600 mg, 888.39 umol, 1 eq) in THF (20 mL), MeOH (10 mL) and H₂O (10 mL) was added LiOH·H₂O (223.68 mg, 5.33 mmol, 6 eq). The reaction was stirred at 45° C. for 1.5 hr. The reaction was concentrated to remove the solvent. The residue was adjusted with 1N HCl to pH 3-4 and extracted with EtOAc (80 mL*3). The combined organic layers were washed with brine (50 mL*3), dried over Na₂SO₄, filtered and concentrated under reduced pressure to give a residue. The residue was purified by prep-HPLC (HCl condition) to afford 2-[4-[(3S,4S)-6-[4-(2-carboxyethyl)-2,6-dichloro-phenoxy]-3,4-dihydroxy-hexoxy]-3,5-dichloro-anilino]benzoic acid (300 mg, 100% purity) as a white solid

The reaction was combined with another reaction ET20197-148 in 0.1 g scale for work up and purification.

LCMS: ET20197-149-P1A1 (M+H⁺): 648.0@ 3.000 min (10-80% ACN in H₂O, 4.5 min)

¹H NMR: ET20197-149-P1BB (METHANOL-d4, 400 MHz)

δ 8.00 (dd, J=1.5, 7.9 Hz, 1H), 7.44-7.37 (m, 1H), 7.28-7.21 (m, 5H), 6.84 (t, J=7.6 Hz, 1H), 4.27-4.10 (m, 4H), 3.97-3.89 (m, 2H), 2.85 (t, J=7.5 Hz, 2H), 2.63-2.55 (m, 2H), 2.20-2.09 (m, 2H), 2.00 (td, J=6.0, 8.2 Hz, 2H)

SFC: ET20197-149-P1S1 (Retention time: 7.777 min; 99.4% ee value)

DSC: ET20197-149-P1D (Peak: 114.3° C., onset: 103.6° C., end: 117.4° C.)

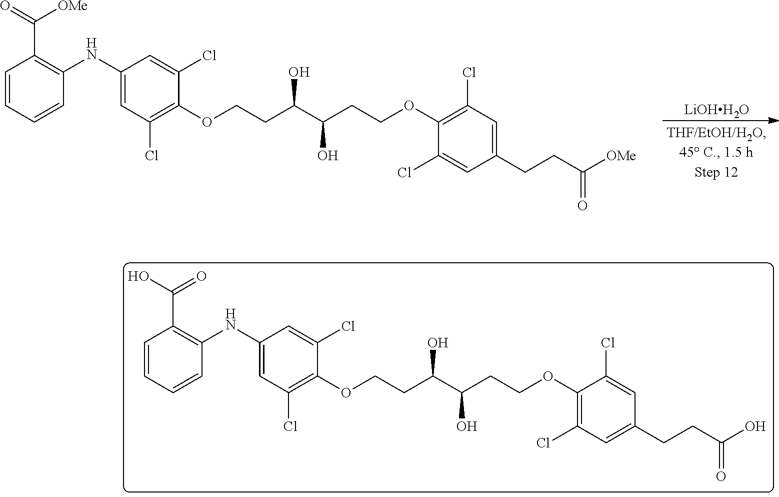 [SMILES: COC(=O)CCc1cc(Cl)c(OCC[C@@H](O)[C@H](O)CCOc2c(Cl)cc(Nc3ccccc3C(=O)OC)cc2Cl)c(Cl)c1 ; "LiOH·H2O" ; O=C(O)CCc1cc(Cl)c(OCC[C@@H](O)[C@H](O)CCOc2c(Cl)cc(Nc3ccccc3C(=O)O)cc2Cl)c(Cl)c1]

### 2-[4-[(3R,4R)-6-[4-(2-carboxyethyl)-2,6-dichloro-phenoxy]-3,4-dihydroxy-hexoxy]-3,5-dichloro-anilino]benzoicacid

To a solution of methyl 2-[3,5-dichloro-4-[(3R,4R)-6-[2,6-dichloro-4-(3-methoxy-3-oxo-propyl) phenoxy]-3,4-dihydroxyhexoxy]anilino]benzoate (50 mg, 74.03 umol, 1 eq) in THF (2 mL), MeOH (1 mL) and H₂O (1 mL) was added LiOH·H₂O (18.64 mg, 444.19 umol, 6 eq). The reaction was stirred at 45° C. for 1.5 hr. The reaction was concentrated to remove the solvent. The residue was adjusted with 1N HCl to pH 3-4 and extracted with EtOAc (50 mL*3). The combined organic layers were washed with brine (30 mL*2), dried over Na₂SO₄, filtered and concentrated under reduced pressure to give a residue. The residue was purified by prep-HPLC (HCl condition) to afford 2-[4-[(3R,4R)-6-[4-(2-carboxyethyl)-2,6-dichloro-phenoxy]-3,4-dihydroxy-hexoxy]-3,5-dichloro-anilino]benzoic acid (15.4 mg, 23.79 umol, 32.13% yield, 100%/6 purity) as a white solid.

LCMS: ET20197-150-P1A1 (M+H⁺): 648.0@ 3.000 min (10-80% ACN in H₂O, 4.5 min)

¹H NMR: ET20197-150-P1BB (METHANOL-d₄, 400 MHz)

δ 8.00 (dd, J=1.5, 8.0 Hz, 1H), 7.45-7.38 (m, 1H), 7.28-7.20 (m, 5H), 6.84 (t, J=7.5 Hz, 1H), 4.27-4.10 (m, 4H), 3.98-3.89 (m, 2H), 2.85 (t, J=7.4 Hz, 2H), 2.65-2.54 (m, 2H), 2.20-2.08 (m, 2H), 2.06-1.93 (m, 2H)

SFC: ET20197-149-P1S1 (Retention time: 6.656 min; 92.16% ee value)

Melting point: ET20197-131-P1M (109.5-110.3° C.)

### Synthesis of B5 (T-205)

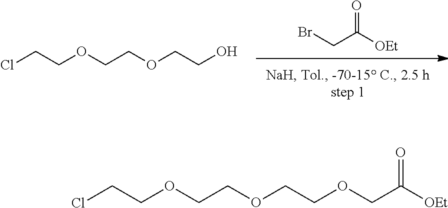 [SMILES: CCOC(=O)CBr ; OCCOCCOCCCl ; CCOC(=O)COCCOCCOCCCl]

### Ethyl 2-[2-[2-(2-chloroethoxy)ethoxy]ethoxy]acetate

NaH (711.67 mg, 17.79 mmol, 60% purity, 1 eq) was suspended in Tol. (20 mL) and 2-[2-(2-chloroethoxy)ethoxy]ethanol (3 g, 17.79 mmol, 1 eq) was gradually added thereto at −70° C. Ethyl 2-bromoacetate (2.97 g, 17.79 mmol, 1.97 mL, 1 eq) was added into the above mixture at −70° C. dropwise. And after 30 min, the reaction mixture was allowed to 15° C. and stirred for 2 hours. Acetic acid was added to the solution at 0° C. and the solution was neutralized to pH 6-7, then the solvent was evaporated under reduced pressure to give a residue. The residue was purified by a silica gel column chromatography (SiO₂, Petroleum ether/EtOAc=10/1 ˜3/1) to afford ethyl 2-[2-[2-(2-chloroethoxy)ethoxy]ethoxy]acetate (3.5 g, 7.70 mmol, 43.25% yield, 56% purity) as a colorless oil.

¹H NMR: ET20197-2-P1AA (CHLOROFORM-d, 400 MHz)

δ 4.22-4.07 (m, 4H), 3.78-3.60 (m, 12H), 1.31-1.25 (m, 3H)

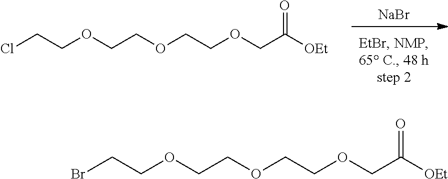 [SMILES: CCOC(=O)COCCOCCOCCCl ; "NaBr" ; CCOC(=O)COCCOCCOCCBr]

### Ethyl 2-[2-[2-(2-bromoethoxy)ethoxy]ethoxy]acetate

Ethyl 2-[2-[2-(2-chloroethoxy)ethoxy]ethoxy]acetate (1 g, 3.93 mmol, 1 eq) was dissolved in NMP (5 mL), then bromoethane (4.28 g, 39.26 mmol, 2.93 mL, 10 eq) and NaBr (80.79 mg, 785.22 umol, 25.25 uL, 0.2 eq) were added thereto and the mixture was heated at 65° C. for 48 hours. The reaction was diluted with H₂O (20 mL) and extracted with EtOAc (50 mL*3). The organic layer was washed with brine (30 mL), dried over Na₂SO₄, filtered and concentrated under reduce pressure to ethyl 2-[2-[2-(2-bromoethoxy)ethoxy]ethoxy]acetate (500 mg, crude) as a brown oil.

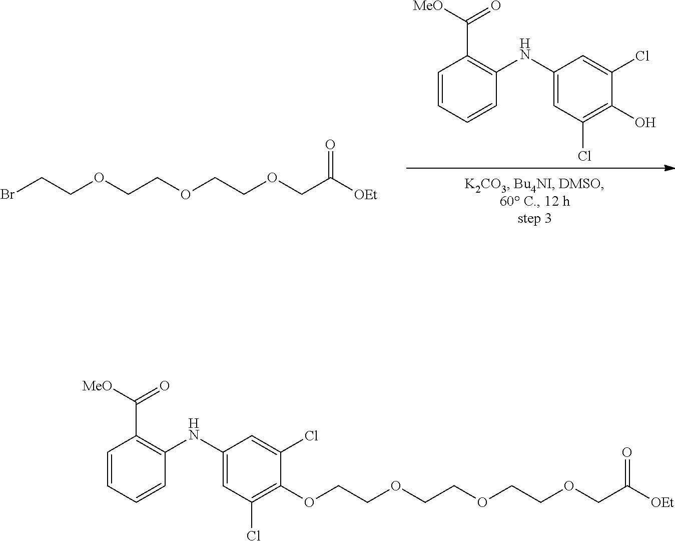 [SMILES: CCOC(=O)COCCOCCOCCBr ; COC(=O)c1ccccc1Nc1cc(Cl)c(O)c(Cl)c1 ; CCOC(=O)COCCOCCOCCOc1c(Cl)cc(Nc2ccccc2C(=O)OC)cc1Cl]

### Methyl 2-[3,5-dichloro-4-[2-[2-[2-(2-ethoxy-2-oxo-ethoxy)ethoxy]ethoxy]ethoxy]anilino]benzoate

A mixture of ethyl 2-[2-[2-(2-bromoethoxy)ethoxy]ethoxy]acetate (766.71 mg, 2.56 mmol, 2 eq), methyl 2-(3,5-dichloro-4-hydroxy-anilino)benzoate (400 mg, 1.28 mmol, 1 eq), K₂CO₃ (177.10 mg, 1.28 mmol, 1 eq), Bu₄NI (473.32 mg, 1.28 mmol, 1 eq) in DMSO (10 mL) was degassed and purged with N₂ for 3 times, and then the mixture was stirred at 60° C. for 12 hr under N₂ atmosphere. The mixture was diluted with H₂O (10 mL) and extracted with ethyl acetate (30 mL*3). The organic layer was washed with H₂O (10 mL), 0.1 N HCl (10 mL) and H₂O (20 mL) successively and dried over Na₂SO₄, filtered, and concentrated to give the residue. The residue was purified by a silica gel column chromatography (SiO₂, PE/EtOAc=20/1 ˜3/1) to afford methyl 2-[3,5-dichloro-4-[2-[2-[2-(2-ethoxy-2-oxo-ethoxy)ethoxy]ethoxy]ethoxy] anilino] benzoate (430 mg, 88% purity) as a light yellow oil.

The reaction was combined with another reaction ET20197-41 in 0.1 g scale for work up and purification.

LCMS: ET20197-48-P1C (M+H⁺): 530.0 @ 1.436 min (5-95% ACN in H₂O, 2.0 min)

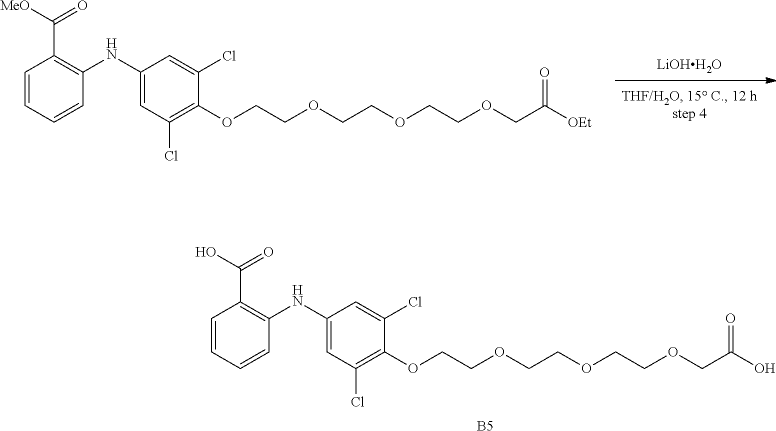

### 2-[4-[2-[2-[2-(carboxymethoxy)ethoxy]ethoxy]ethoxy]-3,5-dichloro-anilino]benzoic acid

To a solution of methyl 2-[3,5-dichloro-4-[2-[2-[2-(2-ethoxy-2-oxo-ethoxy)ethoxy]ethoxy]ethoxy]anilino]benzoate (380 mg, 716.45 umol, 1 eq) in H₂O (4.2 mL) and THF (5.6 mL) was added LiOH·H₂O (180.39 mg, 4.30 mmol, 6 eq) at 15° C. The mixture was stirred at 15° C. for 12 hr. The mixture was concentrated to give the residue. The residue was diluted with H₂O (20 mL) and extracted with DCM (50 mL*2, discarded). The aqueous layer was adjusted to pH 3-4 with HCl (1N) and extracted with EtOAc (20 mL*3). The organic layer was concentrated to give the residue. The residue was purified by prep-HPLC (HCl condition) to afford 2-[4-[2-[2-[2-(carboxy methoxy)ethoxy]ethoxy] ethoxy]-3,5-dichloro-anilino]benzoic acid (160.1 mg, 99.20% purity) as a light yellow solid.

The reaction was combined with another reaction ET20197-52 in 50 mg scale for work up and purification.

LCMS: ET20197-54-PIA (M+H⁺): 486.0 @ 2.435 min (10-80% ACN in H₂O, 4.5 min)

¹H NMR: ET20197-54-P1AA (DMSO-d6, 400 MHz)

δ 12.84 (br s, 2H), 9.51 (br s, 1H), 7.91 (dd, J=1.8, 7.9 Hz, 1H), 7.46 (dt, J=1.8, 7.9 Hz, 1H), 7.35 (s, 2H), 7.25 (d, J=8.3 Hz, 1H), 6.88 (t, J=7.5 Hz, 1H), 4.11-4.06 (m, 2H), 4.02 (s, 2H), 3.81-3.74 (m, 2H), 3.63-3.52 (m, 8H)

DSC: ET20197-54-P1D (Peak: 80.5° C., onset 73.8° C., end 83.2° C.)

### Synthesis of B6 (T-746)

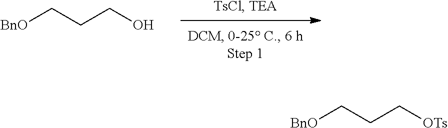 [SMILES: "TsCl, TEA" ; OCCCOCc1ccccc1 ; Cc1ccc(S(=O)(=O)OCCCOCc2ccccc2)cc1]

### 3-benzyloxypropyl 4-methylbenzenesulfonate

To a solution of 3-benzyloxypropan-1-ol (50 g, 300.81 mmol, 47.62 mL, 1 eq) and TEA (60.88 g, 601.63 mmol, 83.74 mL, 2 eq) in DCM (700 mL) was added TosCl (68.82 g, 360.98 mmol, 1.2 eq) in portions at 0° C., the reaction mixture was stirred at 25° C. for 6 hr. The reaction mixture was concentrated to give a residue. The residue was purified by silica gel column (PE:EtOAc=PE:EtOAc=25:1) to afford 3-benzyloxypropyl 4-methylbenzene sulfonate (81 g, 252.81 mmol, 84.04% yield) as colorless oil.

¹H NMR: ET20960-38-P1AA (CHLOROFORM-d, 400 MHz)

δ 7.81 (d, J=8.3 Hz, 2H), 7.40-7.24 (m, 7H), 4.43 (s, 2H), 4.19 (t, J=6.2 Hz, 2H), 3.53 (t, J 5.9 Hz, 2H), 2.53-2.38 (m, 3H), 1.97 (quin, J=6.1 Hz, 2H)

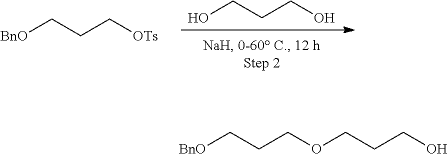 [SMILES: OCCCO ; Cc1ccc(S(=O)(=O)OCCCOCc2ccccc2)cc1 ; OCCCOCCCOCc1ccccc1]

### 3-(3-benzyloxypropoxy)propan-1-ol

To propane-1,3-diol (192.37 g, 2.53 mol, 183.21 mL, 10 eq) was added NaH (11.12 g, 278.09 mmol, 60% purity, 1.1 eq) at 0° C. The mixture was stirred at 0° C. for 0.5 hr. Then 3-benzyloxypropyl 4-methylbenzenesulfonate (81 g, 252.81 mmol, 1 eq) was added at 0° C., The mixture was stirred at 25° C. for 11.5 hr. The reaction mixture was diluted with saturated NH₄Cl solution 600 mL and extracted with EtOAc 300 mL×2. The combined organic layers were washed with brine 300 mL, dried over Na₂SO₄, filtered and concentrated under reduced pressure to give a residue. The residue was purified by column chromatography (SiO₂, Petroleum ether/Ethyl acetate=8/1 to 1/1) to afford 3-(3-benzyloxypropoxy)propan-1-ol (47 g, 209.55 mmol, 82.89% yield) as colorless oil.

¹H NMR: ET21585-1-P1AA (CHLOROFORM-d, 400 MHz)

δ 7.39-7.27 (m, 5H), 4.51 (s, 2H), 3.76 (t, J=5.6 Hz, 2H), 3.62 (t, J=5.7 Hz, 2H), 3.56 (t, J=6.4 Hz, 4H), 2.47-2.23 (m, 1H), 1.89 (quin, J=6.3 Hz, 2H), 1.83 (quin, J=5.7 Hz, 2H)

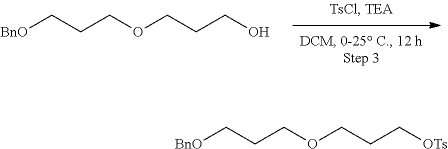 [SMILES: OCCCOCCCOCc1ccccc1 ; Cc1ccc(S(=O)(=O)OCCCOCCCOCc2ccccc2)cc1]

### 3-(3-benzyloxypropoxy)propyl 4-methylbenzenesulfonate

To a solution of 3-(3-benzyloxypropoxy)propan-1-ol (37 g, 164.96 mmol, 1 eq) and TEA (33.38 g, 329.92 mmol, 45.92 mL, 2 eq) in DCM (400 mL) was added 4-methylbenzene sulfonyl chloride (37.74 g, 197.95 mmol, 1.2 eq) at 0° C. The mixture was stirred at 25° C. for 12 hr. The reaction mixture was quenched with H₂O (500 mL), extracted with EtOAc 150 mL×2. The combined organic layers were washed with brine 300 mL, dried over Na₂SO₄, filtered and concentrated under reduced pressure to give a residue. The residue was purified by column chromatography (SiO₂, Petroleum ether/Ethyl acetate=20/1 to 10/1) to afford 3-(3-benzyloxypropoxy)propyl 4-methylbenzenesulfonate (57 g, 150.60 mmol, 91.30% yield) as colorless oil.

¹H NMR: ET21585-1-P1AA (CHLOROFORM-d, 400 MHz)

δ 7.79 (d, J=8.4 Hz, 2H), 7.39-7.28 (m, 7H), 4.49 (s, 2H), 4.13 (t, J=6.2 Hz, 2H), 3.51 (t, J=6.3 Hz, 2H), 3.47-3.39 (m, 4H), 2.44 (s, 3H), 1.89 (quin, J=6.1 Hz, 2H), 1.80 (quin, J=6.3 Hz, 2H)

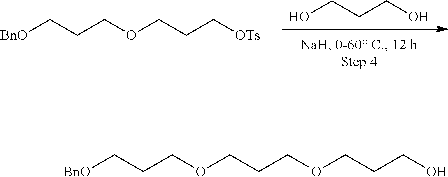 [SMILES: OCCCO ; Cc1ccc(S(=O)(=O)OCCCOCCCOCc2ccccc2)cc1 ; OCCCOCCCOCCCOCc1ccccc1]

### 3-[3-(3-benzyloxypropoxy)propoxy]propan-1-ol

To propane-1,3-diol (114.60 g, 1.51 mol, 109.14 mL, 10 eq) was added NaH (6.63 g, 165.66 mmol, 60% purity, 1.1 eq) at 0° C. The mixture was stirred at 0° C. for 0.5 hr. Then 3-(3-benzyloxypropoxy)propyl 4-methylbenzenesulfonate (57 g, 150.60 mmol, 1 eq) was added at 0° C., The mixture was stirred at 60° C. for 11.5 hr. The reaction mixture was quenched with saturated NH₄Cl solution 500 mL and extracted with EtOAc 300 mL×2. The combined organic layers were washed with brine 300 mL, dried over Na₂SO₄, filtered and concentrated under reduced pressure to give a residue. The residue was purified by column chromatography (SiO₂, Petroleum ether/Ethyl acetate=10/1 to 1/1) to afford 3-[3-(3-benzyloxypropoxy)propoxy]propan-1-ol (32 g, 113.32 mmol, 75.25% yield) as colorless oil.

¹H NMR: ET21585-3-P1AA (CHLOROFORM-d, 400 MHz)

δ 7.38-7.25 (m, 5H), 4.51 (s, 2H), 3.76 (t, J=5.7 Hz, 2H), 3.65-3.46 (m, 10H), 2.45 (br s, 1H), 1.93-1.77 (m, 6H).

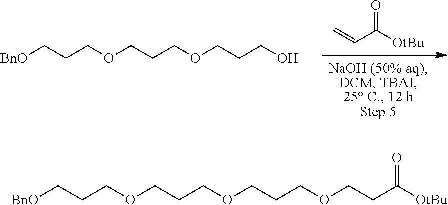

### Tert-butyl 3-[3-[3-(3-benzyloxypropoxy)propoxy]propoxy]propanoate

To a solution of 3-[3-(3-benzyloxypropoxy)propoxy]propan-1-ol (2 g, 7.08 mmol, 1 eq) and tert-butyl prop-2-enoate (2.72 g, 21.25 mmol, 3.08 mL, 3 eq) in DCM (40 mL) was added NaOH (16 mL, 50% purity) and TBAI (261.62 mg, 708.28 umol, 0.1 eq). The mixture was stirred at 25° C. for 12 hr. The reaction mixture was diluted with H₂O 80 mL, extracted with EtOAc 40 mL×2. The combined organic layers were washed with brine 50 mL, dried over Na₂SO₄, filtered and concentrated under reduced pressure to give a residue. The residue was purified by column chromatography (SiO₂, Petroleum ether/Ethyl acetate=20/1 to 10/1) to afford tert-butyl 3-[3-[3-(3-benzyloxypropoxy) propoxy]propoxy]propanoate (2.3 g, 5.60 mmol, 79.10% yield) as colorless oil.

¹H NMR: ET21585-4-P1AA (CHLOROFORM-d, 400 MHz)

δ 7.31-7.19 (m, 5H), 4.44 (s, 2H), 3.59 (t, J=6.5 Hz, 2H), 3.52-3.38 (m, 12H), 2.41 (t, J=6.5 Hz, 2H), 1.86-1.71 (m, 6H), 1.39 (s, 9H)

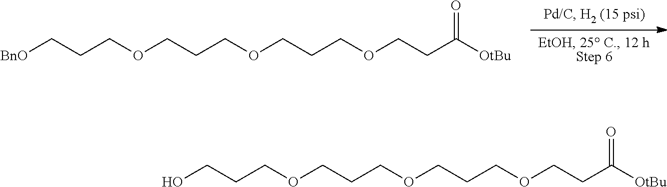 [SMILES: CC(C)(C)OC(=O)CCOCCCOCCCOCCCOCc1ccccc1 ; "EtOH, 25° C., 12 hStep 6" ; "Pd/C, H2 (15 psi)" ; CC(C)(C)OC(=O)CCOCCCOCCCOCCCO]

### Tert-butyl 3-[3-[3-(3-hydroxypropoxy)propoxy]propoxy]propanoate

To a solution of tert-butyl 3-[3-[3-(3-benzyloxypropoxy)propoxy]propoxy]propanoate (2.3 g, 5.60 mmol, 1 eq) in EtOH (20 mL) was added Pd/C (2 g, 10% purity) under Ar. The suspension was degassed under vacuum and purged with H₂ several times. The mixture was stirred under H₂ (15 psi) at 25° C. for 12 hours. The reaction mixture was filtered and concentrated under reduced pressure to give a residue. The residue was purified by column chromatography (SiO₂, Petroleum ether/Ethyl acetate=10/1 to 3/1) to afford tert-butyl 3-[3-[3-(3-hydroxypropoxy)propoxy]propoxy]propanoate (1.7 g, 5.31 mmol, 94.70% yield) as colorless oil.

¹H NMR: ET21585-6-P1AA (CHLOROFORM-d, 400 MHz)

δ 3.76 (t, J=5.6 Hz, 2H), 3.62 (td, J=6.1, 15.5 Hz, 4H), 3.55-3.43 (m, 8H), 2.47 (t, J=6.5 Hz, 2H), 2.40-2.24 (m, 1H), 1.87-1.77 (m, 6H), 1.44 (s, 9H)

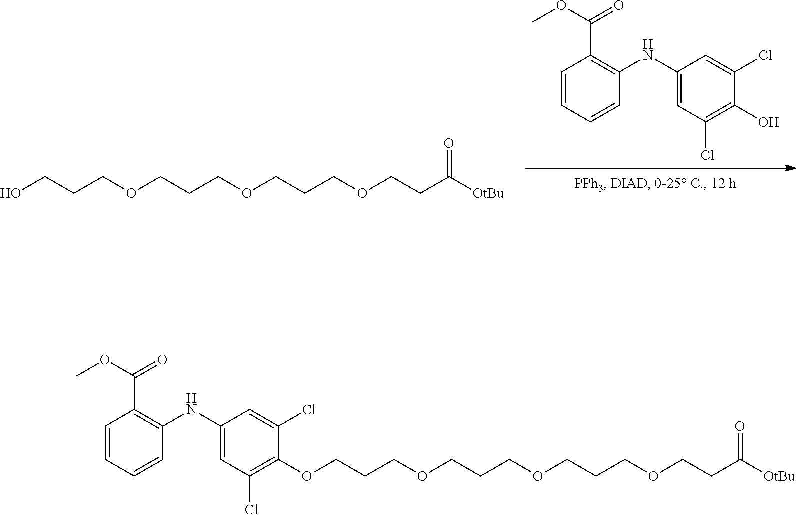 [SMILES: CC(C)(C)OC(=O)CCOCCCOCCCOCCCO ; COC(=O)c1ccccc1Nc1cc(Cl)c(O)c(Cl)c1 ; COC(=O)c1ccccc1Nc1cc(Cl)c(OCCCOCCCOCCCOCCC(=O)OC(C)(C)C)c(Cl)c1]

### Methyl 2-[4-[3-[3-[3-(3-tert-butoxy-3-oxo-propoxy)propoxy]propoxy]propoxy]-3,5-dichloro-anilino]benzoate

To a solution of tert-butyl 3-[3-[3-(3-hydroxypropoxy)propoxy]propoxy]propanoate (500 mg, 1.56 mmol, 1 eq) and methyl 2-(3,5-dichloro-4-hydroxy-anilino)benzoate (487.09 mg, 1.56 mmol, 1 eq) in THF (7.5 mL) was added PPh₃ (613.93 mg, 2.34 mmol, 1.5 eq) and DIAD (473.30 mg, 2.34 mmol, 455.10 uL, 1.5 eq) at 0° C. The reaction mixture was warmed to 25° C. and stirred for 12 h. The reaction mixture was diluted with H₂O 30 mL and extracted with EtOAc 20 mL×3. The combined organic layers were washed with brine 30 mL, dried over Na₂SO₄, filtered and concentrated under reduced pressure to give a residue. The residue was purified by flash silica gel chromatography (ISCO®; 12 g SepaFlash® Silica Flash Column, Eluent of 0˜20% Ethyl acetate/Petroleum ethergradient @ 50 mL/min) to afford methyl 2-[4-[3-[3-[3-(3-tert-butoxy-3-oxo-propoxy)propoxy]propoxy]propoxy]-3,5-dichloro-anilino]benzoate (1 g) as yellow oil.

The reaction was combined with another reaction ET21585-11 in 0.1 g scale for work up and purification.

¹H NMR: ET21585-12-P1AA (CHLOROFORM-d, 400 MHz)

δ 9.42 (s, 1H), 7.98 (dd, J=1.8, 7.9 Hz, 1H), 7.41-7.36 (m, 1H), 7.19 (s, 2H), 6.82 (t, J=7.7 Hz, 1H), 6.33 (br s, 1H), 4.10 (t, J=6.4 Hz, 2H), 3.91 (s, 3H), 3.73-3.63 (m, 4H), 3.52 (tt, J=6.5, 12.8 Hz, 8H), 2.48 (t, J=6.6 Hz, 2H), 2.11 (quin, J=6.2 Hz, 2H), 1.90-1.78 (m, 4H), 1.46 (s, 9H)

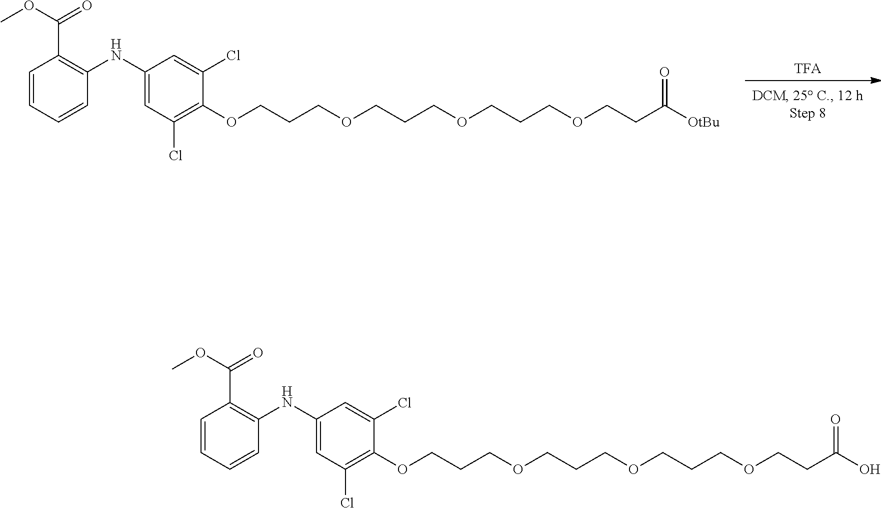 [SMILES: COC(=O)c1ccccc1Nc1cc(Cl)c(OCCCOCCCOCCCOCCC(=O)OC(C)(C)C)c(Cl)c1 ; "TFA" ; COC(=O)c1ccccc1Nc1cc(Cl)c(OCCCOCCCOCCCOCCC(=O)O)c(Cl)c1]

### 3-[3-[3-[3-[2,6-dichloro-4-(2-methoxycarbonylanilino)phenoxy]propoxy]propoxy]propoxy]propanoic acid

To a solution of methyl 2-[4-[3-[3-[3-(3-tert-butoxy-3-oxo-propoxy)propoxy]propoxy]propoxy]-3,5-dichloro-anilino]benzoate (900 mg, 1.46 mmol, 1 eq) in DCM (10 mL) was added TFA (2 mL). The mixture was stirred at 25° C. for 12 hr. The reaction mixture was diluted with H₂O 20 mL and extracted with DCM 20 mL×3. The combined organic layers were washed with brine 30 mL, dried over Na₂SO₄, filtered and concentrated under reduced pressure to afford 3-[3-[3-[3-[2,6-dichloro-4-(2-methoxycarbonylanilino)phenoxy]propoxy]propoxy]propoxy] propanoic acid (1 g) as dark green oil.

The reaction was combined with another reaction ET21585-14 in 0.1 g scale for work up.

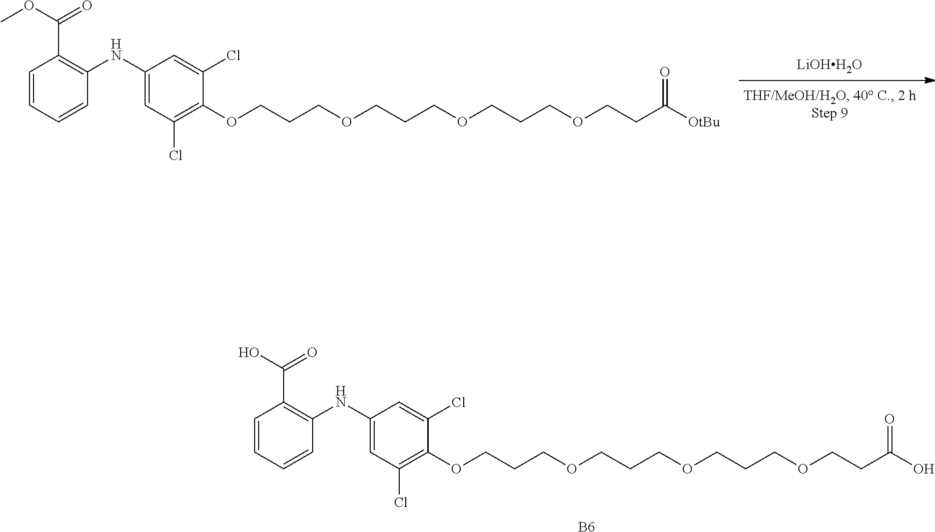

### 2-[4-[3-[3-[3-(2-carboxyethoxy)propoxy]propoxy]propoxy]-3,5-dichloro-anilino]benzoic acid

To a solution of 3-[3-[3-[3-[2,6-dichloro-4-(2-methoxycarbonylanilino)phenoxy]propoxy]propoxy]propoxy]propanoic acid (900 mg, 1.61 mmol, 1 eq) in THF (35 mL) and MeOH (35 mL) was added LiOH·H₂O (270.49 mg, 6.45 mmol, 4 eq) in H₂O (70 mL). The mixture was stirred at 40° C. for 2 hr. The reaction mixture was diluted with H₂O 20 mL, acidified to pH=4 with HCl (1 N) and extracted with EtOAc 20 mL×2. The combined organic layers were washed with brine 20 mL, dried over Na₂SO₄, filtered and concentrated under reduced pressure to give a residue. The residue was purified by prep-HPLC (HCl condition) to afford 2-[4-[3-[3-[3-(2-carboxyethoxy)propoxy]propoxy]propoxy]-3,5-dichloro-anilino]benzoic acid (326 mg) as a yellow solid.

The reaction was combined with another reaction ET21585-21 in 0.1 g scale for work up and purification.

LCMS: ET20197-54-P1A (M+H): 544.1 @ 2.000 min (10-80% ACN in H₂O, 3 min)

¹H NMR: ET21585-23-P1AA (CHLOROFORM-d, 400 MHz)

δ 9.21 (s, 1H), 8.04 (dd, J=1.5, 8.0 Hz, 1H), 7.41 (ddd, J=1.7, 7.1, 8.6 Hz, 1H), 7.21-7.15 (m, 3H), 6.87-6.79 (m, 1H), 4.12 (t, J=6.1 Hz, 2H), 3.78-3.66 (m, 4H), 3.57 (dt, J=4.3, 6.2 Hz, 4H), 3.51 (dt, J=2.8, 6.3 Hz, 4H), 2.64 (t, J=6.1 Hz, 2H), 2.11 (quin, J=6.2 Hz, 2H), 1.86 (sxt, J=6.3 Hz, 4H)

### Synthesis of B7 (T-742)

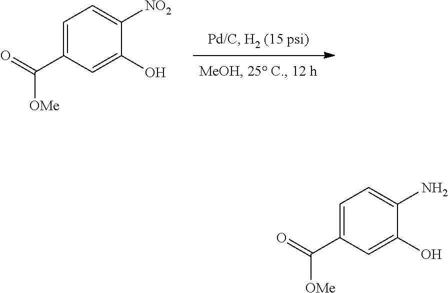 [SMILES: COC(=O)c1ccc([N+](=O)[O-])c(O)c1 ; "Pd/C, H2 (15 psi)" ; COC(=O)c1ccc(N)c(O)c1]

### Methyl 4-amino-3-hydroxy-benzoate

To a suspension of Pd/C (3 g, 10% purity) in MeOH (160 mL) was added methyl 3-hydroxy-4-nitro-benzoate (10 g, 50.72 mmol, 1 eq), the reaction mixture was degassed and purged with H₂ for 3 times, and stirred under H₂ (15 psi) at 25° C. for 12 h. The reaction mixture was filtered and the filtrate was concentrated to give a residue. The residue was purified by silica gel column (PE:EtOAc=3:1) to afford methyl 4-amino-3-hydroxy-benzoate (6.3 g, 37.69 mmol, 74.30% yield) as a light yellow solid.

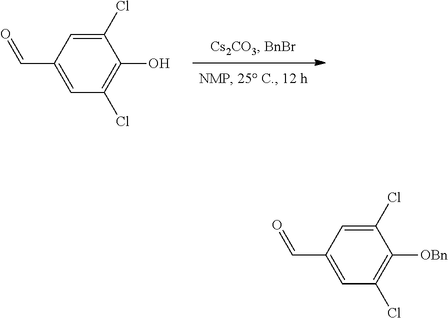

### 4-benzyloxy-3,5-dichloro-benzaldehyde

To a mixture of 3,5-dichloro-4-hydroxy-benzaldehyde (9 g, 47.12 mmol, 1 eq) and K₂CO₃ (13.02 g, 94.24 mmol, 2 eq) in DMF (100 mL) was added BnBr (9.67 g, 56.54 mmol, 6.72 mL, 1.2 eq) and it was stirred at 25° C. for 12 hr. The reaction mixture was partitioned between H₂O (500 mL) and EtOAc (800 mL). The organic layer was washed with sub-saturated brine (300 mL*3), saturated brine (300 mL), dried over Na₂SO₄ and concentrated to give a residue. The residue was purified by silica gel column (PE:EtOAc=20:1 to 5:1) to afford 4-benzyloxy-3,5-dichloro-benzaldehyde (12 g, 42.68 mmol, 90.59% yield) as a white solid.

¹H NMR: ET20960-19-P1AA (CHLOROFORM-d, 400 MHz)

δ 9.97-9.81 (m, 1H), 7.86 (s, 2H), 7.59-7.53 (m, 2H), 7.46-7.36 (m, 3H), 5.16 (s, 2H)

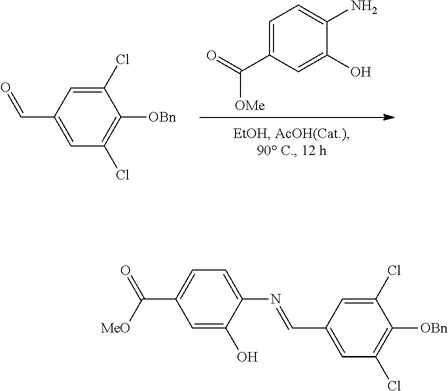 [SMILES: O=Cc1cc(Cl)c(OCc2ccccc2)c(Cl)c1 ; COC(=O)c1ccc(N)c(O)c1 ; COC(=O)c1ccc(/N=C/c2cc(Cl)c(OCc3ccccc3)c(Cl)c2)c(O)c1]

### Methyl 4-[(E)-(4-benzyloxy-3,5-dichloro-phenyl)methyleneamino]-3-hydroxy-benzoate

To a solution of methyl 4-amino-3-hydroxy-benzoate (7 g, 41.88 mmol, 1 eq) and 4-benzyloxy-3,5-dichloro-benzaldehyde (11.77 g, 41.88 mmol, 1 eq) in EtOH (180 mL) was added AcOH (125.74 mg, 2.09 mmol, 119.75 uL, 0.05 eq). The reaction mixture was stirred at 90° C. for 12 h, and then it was concentrated to give a residue. The residue was washed with MeOH (200 mL) to afford methyl 4-[(E)-(4-benzyloxy-3,5-dichloro-phenyl)methyleneamino]-3-hydroxy-benzoate (15.6 g, 36.26 mmol, 86.58% yield) as a yellow solid.

¹H NMR: ET20960-36-P1 AA (CHLOROFORM-d, 400 MHz)

δ 8.59 (s, 1H), 7.90 (s, 2H), 7.70 (d, J=1.8 Hz, 1H), 7.63 (dd, J=1.8, 8.3 Hz, 1H), 7.58 (dd, J=1.5, 7.9 Hz, 2H), 7.46-7.37 (m, 3H), 7.29 (d, J=8.4 Hz, 1H), 7.03 (s, 1H), 5.15 (s, 2H), 3.93 (s, 3H)

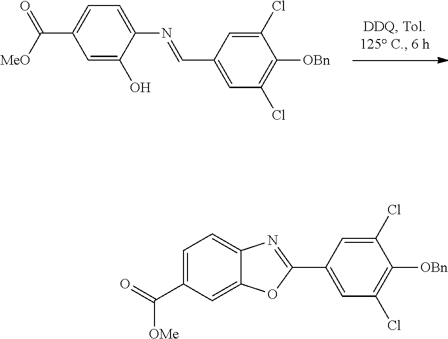 [SMILES: COC(=O)c1ccc(/N=C/c2cc(Cl)c(OCc3ccccc3)c(Cl)c2)c(O)c1 ; COC(=O)c1ccc2nc(-c3cc(Cl)c(OCc4ccccc4)c(Cl)c3)oc2c1]

### Methyl 2-(4-benzyloxy-3,5-dichloro-phenyl)-1,3-benzoxazole-6-carboxylate

To a boiling solution of methyl 4-[(E)-(4-benzyloxy-3,5-dichloro-phenyl)methylene amino]-3-hydroxy-benzoate (8 g, 18.59 mmol, 1 eq) in Tol. (0.6 L) was added DDQ (8.44 g, 37.19 mmol, 2 eq) gradually. Then the reaction mixture was stirred at 125° C. for 6 hr. The reaction mixture was filtered and concentrated to give a residue. The residue was purified by silica gel column (PE:EtOAc=20:1 to 5:1) to afford methyl 2-(4-benzyloxy-3,5-dichloro-phenyl)-1,3-benzoxazole-6-carboxylate (14 g, 32.69 mmol, 87.91% yield) as a yellow solid.

¹H NMR: ET20960-39-P1AA (CHLOROFORM-d, 400 MHz)

δ 8.32-8.22 (m, 3H), 8.12 (dd, J=1.5, 8.4 Hz, 1H), 7.79 (d, J=8.3 Hz, 1H), 7.64-7.54 (m, 2H), 7.47-7.37 (m, 3H), 5.16 (s, 2H), 3.98 (s, 3H)

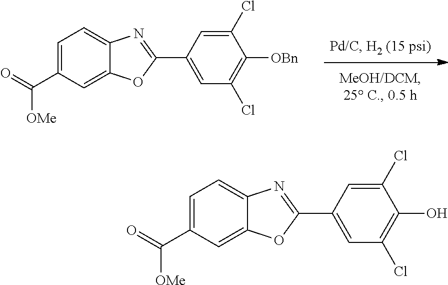 [SMILES: COC(=O)c1ccc2nc(-c3cc(Cl)c(OCc4ccccc4)c(Cl)c3)oc2c1 ; "Pd/C, H2 (15 psi)" ; COC(=O)c1ccc2nc(-c3cc(Cl)c(O)c(Cl)c3)oc2c1]

### Methyl 2-(3,5-dichloro-4-hydroxy-phenyl)-1,3-benzoxazole-6-carboxylate

To a suspension of Pd/C (1 g, 10% purity) in MeOH (30 mL) and DCM (90 mL) was added methyl 2-(4-benzyloxy-3,5-dichloro-phenyl)-1,3-benzoxazole-6-carboxylate (2.5 g, 5.84 mmol, 1 eq). The reaction mixture was degassed and purged with H₂ for 3 times and stirred under H₂ (15 psi) at 25° C. for 0.5 hr, and then it was concentrated to give a residue. The residue was washed with MeOH (20 mL) to afford methyl 2-(3,5-dichloro-4-hydroxy-phenyl)-1,3-benzoxazole-6-carboxylate (17 g) as a white solid.

The reaction was combined with another reaction ET20960-29 in 70 mg scale for work up and purification.

¹H NMR: ET20960-41-P1AA (DMSO-d6, 400 MHz)

δ 8.24 (s, 1H), 8.11 (s, 2H), 8.01 (dd, J=1.3, 8.3 Hz, 1H), 7.87 (d, J=8.3 Hz, 1H), 3.90 (s, 3H)

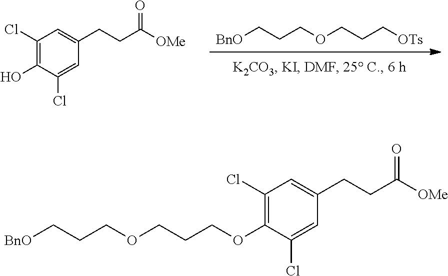 [SMILES: COC(=O)CCc1cc(Cl)c(O)c(Cl)c1 ; Cc1ccc(S(=O)(=O)OCCCOCCCOCc2ccccc2)cc1 ; COC(=O)CCc1cc(Cl)c(OCCCOCCCOCc2ccccc2)c(Cl)c1]

### Methyl 3-[4-[3-(3-benzyloxypropoxy)propoxy]-3,5-dichloro-phenyl]propanoate

To a solution of methyl 3-(3,5-dichloro-4-hydroxy-phenyl)propanoate (2.19 g, 8.81 mmol, 1 eq) and 3-(3-benzyloxypropoxy)propyl 4-methylbenzenesulfonate (4 g, 10.57 mmol, 1.2 eq) in DMF (50 mL) was added K₂CO₃ (3.65 g, 26.42 mmol, 3 eq) and KI (146.20 mg, 880.71 umol, 0.1 eq) at 25° C. The reaction mixture was stirred at 25° C. for 6 hr, and then it was diluted with EtOAc (120 mL), extracted with sub-saturated brine (80 mL*3). The organic layer was dried over Na₂SO₄ and concentrated to give a residue. The residue was purified by silica gel column (PE:EtOAc=20:1 to 8:1) to afford methyl 3-[4-[3-(3-benzyloxypropoxy) propoxy]-3,5-dichloro-phenyl]propanoate (3.3 g, 7.25 mmol, 82.28% yield) as a colorless oil.

¹H NMR: ET20960-67-P1AA (CHLOROFORM-d, 400 MHz)

δ 7.46-7.27 (m, 5H), 7.13 (s, 2H), 4.51 (s, 2H), 4.11-4.02 (m, 2H), 3.75-3.64 (m, 5H), 3.58 (dt, J=2.4, 6.3 Hz, 4H), 2.87 (t, J=7.6 Hz, 2H), 2.67-2.56 (m, 2H), 2.14-2.06 (m, 2H), 1.91 (quin, J=6.4 Hz, 2H)

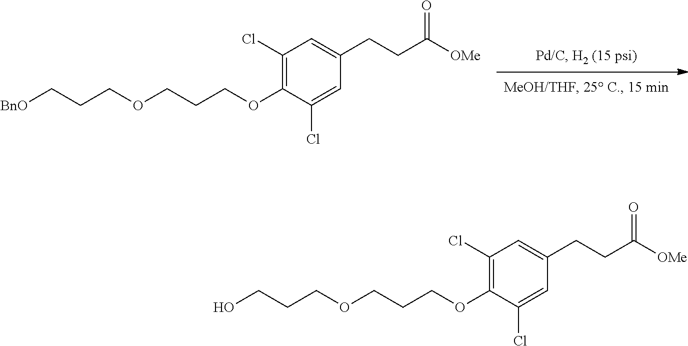 [SMILES: COC(=O)CCc1cc(Cl)c(OCCCOCCCOCc2ccccc2)c(Cl)c1 ; COC(=O)CCc1cc(Cl)c(OCCCOCCCO)c(Cl)c1]

### Methyl 3-[3,5-dichloro-4-[3-(3-hydroxypropoxy)propoxy]phenyl]propanoate

To a suspension of Pd/C (1 g, 10% purity) in MeOH (30 mL) was added a solution of methyl 3-[4-[3-(3-benzyloxypropoxy)propoxy]-3,5-dichloro-phenyl]propanoate (3.3 g, 7.25 mmol, 1 eq) in THF (30 mL), the reaction mixture was degassed and purged with H₂ for 3 times, the reaction mixture was stirred at 25° C. under H₂ at 15 psi for 15 min. The reaction mixture was filtered and the filtrate was concentrated to give a residue. The residue was purified by silica gel column (PE:EtOAc=10:1 to 4:1) to afford methyl 3-[3,5-dichloro-4-[3-(3-hydroxy propoxy)propoxy]phenyl]propanoate (2.1 g, 5.55 mmol, 76.64% yield, 96.6% purity) as a colorless oil.

¹H NMR: ET20960-68-P1AA (CHLOROFORM-d, 400 MHz)

δ 7.13 (s, 2H), 4.08 (t, J=6.1 Hz, 2H), 3.82-3.63 (m, 9H), 2.92-2.81 (m, 2H), 2.65-2.56 (m, 2H), 2.10 (quin, J=6.1 Hz, 2H), 1.85 (quin, J=5.7 Hz, 2H)

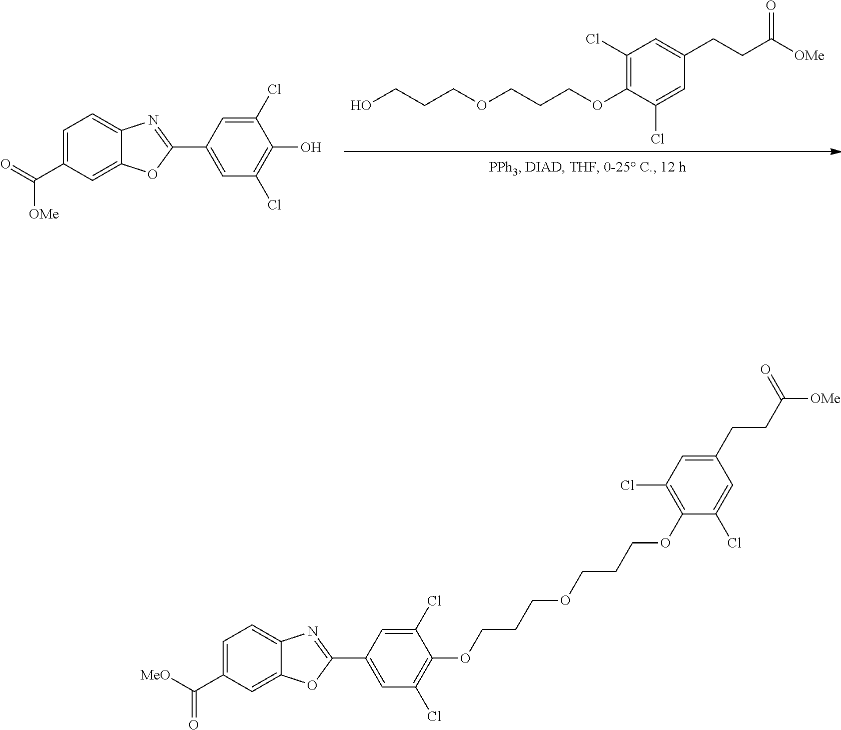 [SMILES: COC(=O)c1ccc2nc(-c3cc(Cl)c(O)c(Cl)c3)oc2c1 ; COC(=O)CCc1cc(Cl)c(OCCCOCCCO)c(Cl)c1 ; COC(=O)CCc1cc(Cl)c(OCCCOCCCOc2c(Cl)cc(-c3nc4ccc(C(=O)OC)cc4o3)cc2Cl)c(Cl)c1]

### Methyl 2-[3,5-dichloro-4-[3-[3-[2,6-dichloro-4-(3-methoxy-3-oxo-propyl)phenoxy]prop oxy]propoxy]phenyl]-1,3-benzoxazole-6-carboxylate

To a mixture of methyl 2-(3,5-dichloro-4-hydroxy-phenyl)-1,3-benzoxazole-6-carboxylate (0.6 g, 1.77 mmol, 1 eq), methyl 3-[3,5-dichloro-4-[3-(3-hydroxypropoxy)propoxy]phenyl]propanoate (797.16 mg, 2.18 mmol, 1.23 eq) and PPh₃ (698.10 mg, 2.66 mmol, 1.5 eq) in THF (10 mL) was added a solution of DIAD (538.20 mg, 2.66 mmol, 517.50 uL, 1.5 eq) in THF (5 mL) at 0° C., then the reaction mixture was stirred at 25° C. for 12 hr. The reaction mixture was concentrated to give a residue. The residue was purified by silica gel column (PE:EtOAc=50:1 to 20:1) to afford methyl 2-[3,5-dichloro-4-[3-[3-[2,6-dichloro-4-(3-meth oxy-3-oxo-propyl)phenoxy]propoxy]propoxy]phenyl]-1,3-benzoxazole-6-carboxylate (1.1 g, 1.49 mmol, 84.12% yield, 93% purity) as a white solid

¹H NMR: ET20960-70-P1AA (CHLOROFORM-d, 400 MHz)

δ 8.34-8.17 (m, 3H), 8.12 (dd, J=1.4, 8.4 Hz, 1H), 7.79 (d, J=8.4 Hz, 1H), 7.12 (s, 2H), 4.25 (t, J=6.2 Hz, 2H), 4.10 (t, J=6.2 Hz, 2H), 3.99 (s, 3H), 3.81-3.62 (m, 7H), 2.86 (t, J=7.6 Hz, 2H), 2.70-2.53 (m, 2H), 2.23-2.05 (m, 4H).

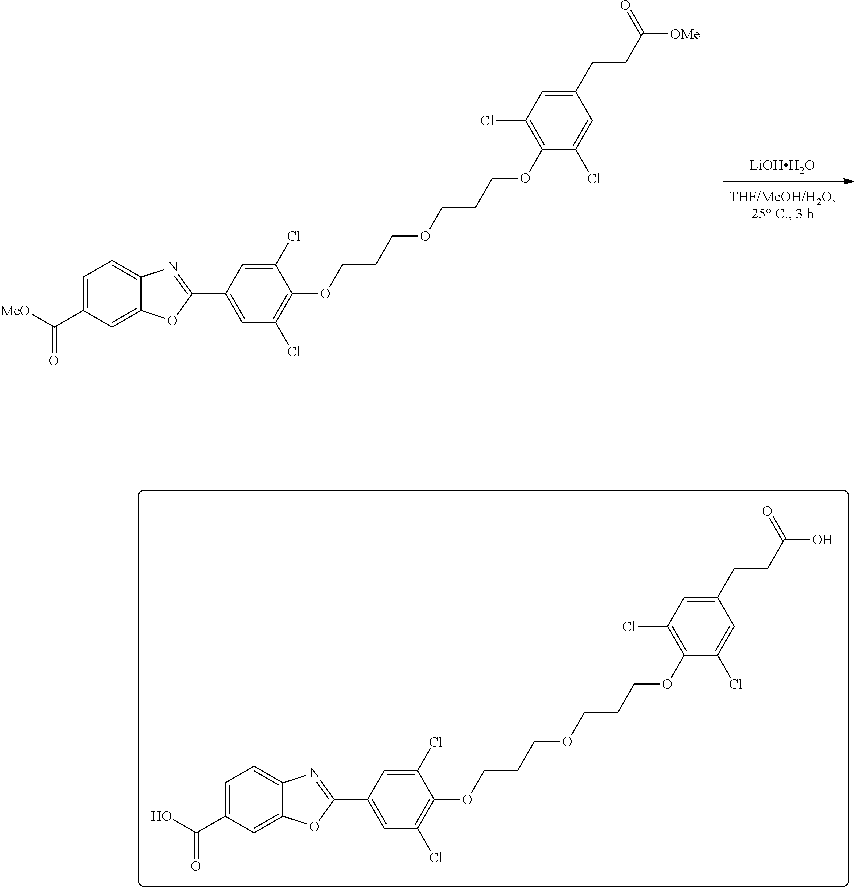 [SMILES: COC(=O)CCc1cc(Cl)c(OCCCOCCCOc2c(Cl)cc(-c3nc4ccc(C(=O)OC)cc4o3)cc2Cl)c(Cl)c1 ; "LiOH·H2O" ; O=C(O)CCc1cc(Cl)c(OCCCOCCCOc2c(Cl)cc(-c3nc4ccc(C(=O)O)cc4o3)cc2Cl)c(Cl)c1]

### 2-[4-[3-[3-[4-(2-carboxyethyl)-2,6-dichloro-phenoxy]propoxy]propoxy]-3,5-dichloro-phenyl]-1,3-benzoxazole-6-carboxylicacid

To a solution of methyl 2-[3,5-dichloro-4-[3-[3-[2,6-dichloro-4-(3-methoxy-3-oxo-propyl) phenoxy]propoxy]propoxy]phenyl]-1,3-benzoxazole-6-carboxylate (0.9 g, 1.31 mmol, 1 eq) in THF (30 mL) and MeOH (10 mL) was added a solution of LiOH·H₂O (275.52 mg, 6.57 mmol, 5 eq) in H₂O (10 mL), the reaction was stirred at 25° C. for 3 hr. The reaction mixture was diluted with H₂O (10 mL), acidified to pH=7 with HCl (2 N), extracted with EtOAc (20 mL*3). The organic layer was washed with brine (30 mL), dried over Na₂SO₄ and concentrated to give a residue. The residue was purified by FA prep-HPLC to afford 2-[4-[3-[3-[4-(2-carboxyethyl)-2,6-dichloro-phenoxy]propoxy]propoxy]-3,5-dichloro-phenyl]-1,3-benzoxazole-6-carboxylic acid (347.4 mg, 97.79% purity) as a white solid.

The reaction was combined with another reaction ET20960-73 in 0.1 g scale for work up and purification.

LCMS: ET20960-80-P1A1 (M+H⁺): 658.0 @ 3.400 min (10-80% ACN in H₂O, 4.5 min)

¹H NMR: ET20960-80-P1AA (DMSO-d6, 400 MHz)

δ 8.34-8.19 (m, 3H), 8.04 (dd, J=1.5, 8.4 Hz, 1H), 7.92 (d, J=8.4 Hz, 1H), 7.34 (s, 2H), 4.20 (t, J=6.3 Hz, 2H), 4.00 (t, J=6.2 Hz, 2H), 3.66-3.59 (m, 4H), 2.78-2.73 (m, 2H), 2.56 (br s, 2H), 2.09-1.95 (m, 4H).

### Synthesis of B8 (T-7501

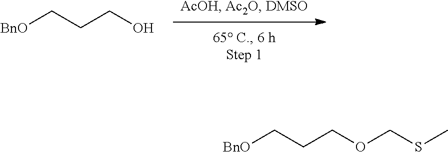

### 3-(methylsulfanylmethoxy)propoxymethylbenzene

To a mixture of 3-benzyloxypropan-1-ol (5 g, 30.08 mmol, 4.76 mL, 1 eq) in DMSO (90 mL) was added AcOH (18.97 g, 315.85 mmol, 18.06 mL, 10.5 eq) and Ac₂O (67.56 g, 661.79 mmol, 61.98 mL, 22 eq), the reaction mixture was stirred at 65° C. for 6 hr. The reaction mixture (combined ET20960-77; 1 g scale) was partitioned between H₂O (500 mL) and EtOAc (400 mL). The organic layer was washed with NaHCO₃(Sat., aq, 250 mL×3), dried over Na₂SO₄ and concentrated to give a residue. The residue was purified by silica gel column (PE) to afford 3-(methylsulfanylimethoxy)propoxymethylbenzene (2.5 g) as colorless oil.

¹H NMR: ET20960-81-P1AB (CHLOROFORM-d, 400 MHz)

δ 7.45-7.27 (m, 5H), 4.63 (s, 2H), 4.52 (s, 2H), 3.65 (t, J=6.3 Hz, 2H), 3.62-3.56 (m, 2H), 2.20-2.12 (m, 3H), 1.97-1.88 (m, 2H)

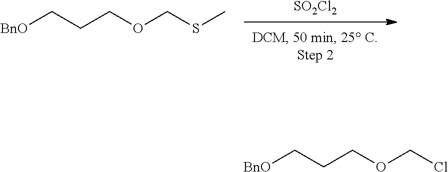

### 3-(chloromethoxy)propoxymethylbenzene

To a solution of 3-(methylsulfanylmethoxy)propoxymethylbenzene (2.2 g, 9.72 mmol, 1 eq) in DCM (22 mL) was added sulfuryl dichloride (1.31 g, 9.72 mmol, 971.80 uL, 1 eq). The mixture was stirred at 25° C. for 50 min. The reaction mixture was concentrated under reduced pressure to remove solvent to afford 3-(chloromethoxy)propoxymethylbenzene (2.2 g, crude) as yellow oil.

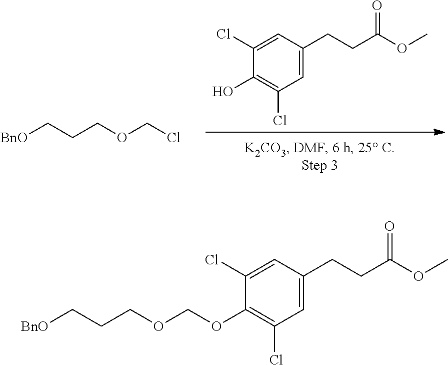 [SMILES: COC(=O)CCc1cc(Cl)c(O)c(Cl)c1 ; ClCOCCCOCc1ccccc1 ; COC(=O)CCc1cc(Cl)c(OCOCCCOCc2ccccc2)c(Cl)c1]

### Methyl 3-[4-(3-benzyloxypropoxymethoxy)-3,5-dichloro-phenyl]propanoate

To a solution of 3-(chloromethoxy)propoxymethylbenzene (2.39 g, 11.13 mmol, 1 eq) and methyl 3-(3,5-dichloro-4-hydroxy-phenyl)propanoate (2.77 g, 11.13 mmol, 1 eq) in DMF (30 mL) was added K₂CO₃ (2.31 g, 16.70 mmol, 1.5 eq). The mixture was stirred at 25° C. for 6 hr. The reaction mixture was diluted with H₂O 60 mL and extracted with EtOAc 50 mL×2. The combined organic layers were washed with brine 60 mL, dried over Na₂SO₄, filtered and concentrated under reduced pressure to give a residue. The residue was purified by flash silica gel chromatography (ISCO®; 20 g SepaFlash® Silica Flash Column, Eluent of 0˜5% Ethyl acetate/Petroleum ethergradient @ 75 mL/min) to afford methyl 3-[4-(3-benzyloxypropoxymethoxy)-3,5-dichloro-phenyl]propanoate (2.5 g, 5.85 mmol, 52.55% yield) as colorless oil.

¹H NMR: ET21585-78-P1AA (CHLOROFORM-d, 400 MHz)

δ 7.39-7.27 (m, 5H), 7.15 (s, 2H), 5.19 (s, 2H), 4.51 (s, 2H), 4.02 (t, J=6.4 Hz, 2H), 3.69 (s, 3H), 3.59 (t, J=6.3 Hz, 2H), 2.90-2.84 (m, 2H), 2.63-2.58 (m, 2H), 1.96 (quin, J=6.4 Hz, 2H)

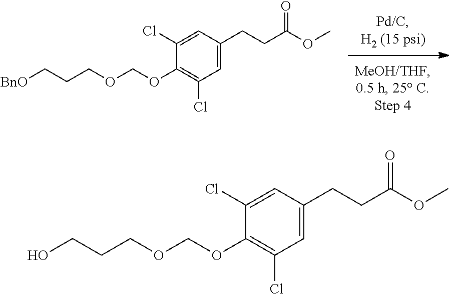 [SMILES: COC(=O)CCc1cc(Cl)c(OCOCCCOCc2ccccc2)c(Cl)c1 ; COC(=O)CCc1cc(Cl)c(OCOCCCO)c(Cl)c1]

### Methyl 3-[3,5-dichloro-4-(3-hydroxypropoxymethoxy)phenyl]propanoate

To a solution of methyl 3-[4-(3-benzyloxypropoxymethoxy)-3,5-dichloro-phenyl]propanoate (1.2 g, 2.81 mmol, 1 eq) in THF (12 mL) and MeOH (12 mL) was added Pd/C (2 g, 10% purity) under Ar. The suspension was degassed under vacuum and purged with H₂ several times. The mixture was stirred under H₂ (15 psi) at 25° C. for 0.5 hr. The suspension (combined ET21585-52; 100 mg scale) was filtered through a pad of Celite and the filter cake was washed with MeOH (800 mL). The combined filtrates were concentrated to dryness to give crude product. The crude product was purified by column chromatography (SiO₂, Petroleum ether/Ethyl acetate=10/1 to 4/1) to afford methyl 3-[3,5-dichloro-4-(3-hydroxypropoxymethoxy)phenyl]propanoate (460 mg) as yellow oil.

¹H NMR: ET21585-81-P1AA (CHLOROFORM-d, 400 MHz)

δ 7.16 (s, 2H), 5.21 (s, 2H), 4.08 (t, J=5.9 Hz, 2H), 3.79 (t, J=5.8 Hz, 2H), 3.71-3.67 (m, 3H), 2.91-2.84 (m, 2H), 2.64-2.58 (m, 2H), 1.91 (quin, J=5.9 Hz, 2H)

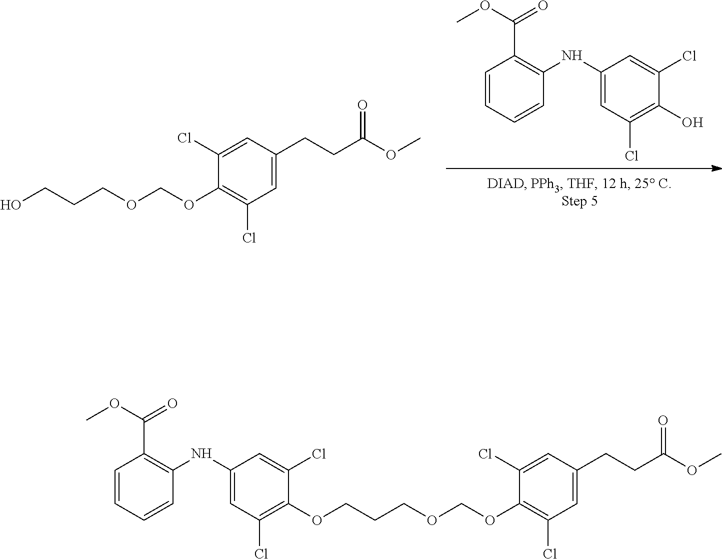

### Methyl 2-[3,5-dichloro-4-[3-[[2,6-dichloro-4-(3-methoxy-3-oxo-propyl)phenoxy]methoxy] propoxy]anilino]benzoate

To a solution of methyl 3-[3,5-dichloro-4-(3-hydroxypropoxymethoxy)phenyl]propanoate (496.91 mg, 1.47 mmol, 1 eq) and methyl 2-(3,5-dichloro-4-hydroxy-anilino)benzoate (460 mg, 1.47 mmol, 1 eq) in THF (10 mL) was added PPh₃ (579.79 mg, 2.21 mmol, 1.5 eq) and DIAD (446.98 mg, 2.21 mmol, 429.79 uL, 1.5 eq). The mixture was stirred at 25° C. for 12 hr. The reaction mixture was concentrated under reduced pressure to remove solvent. The residue was purified by flash silica gel chromatography (ISCO®; 12 g SepaFlash® Silica Flash Column, Eluent of 0˜5% Ethyl acetate/Petroleum ethergradient @ 50 mL/min) to afford methyl 2-[3,5-dichloro-4-[3-[[2,6-dichloro-4-(3-methoxy-3-oxo-propyl)phenoxy]methoxy] propoxy]anilino]benzoate (900 mg) as yellow oil.

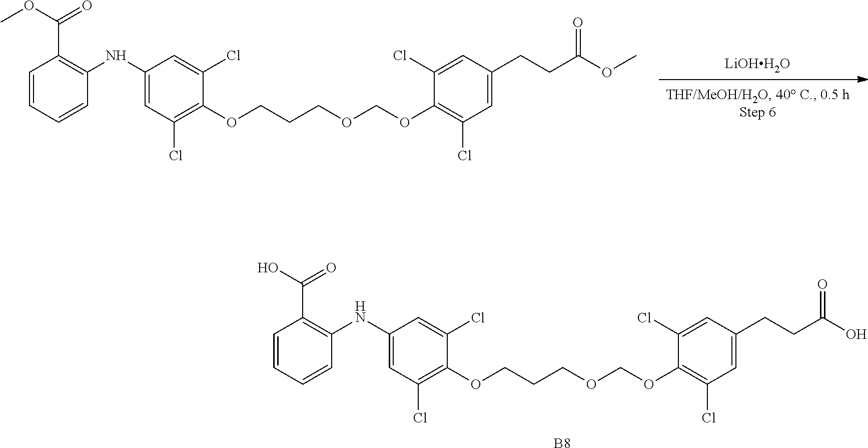

### 2-[4-[3-[[4-(2-carboxyethyl)-2,6-dichloro-phenoxy]methoxy]propoxy]-3,5-dichloro-anilino]benzoic acid

To a solution of methyl 2-[3,5-dichloro-4-[3-[[2,6-dichloro-4-(3-methoxy-3-oxo-propyl) phenoxy]methoxy]propoxy]anilino]benzoate (800.00 mg, 1.27 mmol, 1 eq) in MeOH (4 mL) and THF (4 mL) was added LiOH·H₂O (319.02 mg, 7.60 mmol, 6 eq) in H₂O (8 mL). The mixture was stirred at 40° C. for 0.5 hr. The reaction mixture (combined ET21585-86; 100 mg scale) was acidified to pH=7 with HCl (2 N) and extracted with EtOAc 20 mL×3. The combined organic layers were dried over Na₂SO₄, filtered and concentrated under reduced pressure to give a residue. The residue was purified by prep-HPLC (FA condition) to afford 2-[4-[3-[[4-(2-carboxyethyl)-2,6-dichloro-phenoxy]methoxy]propoxy]-3,5-dichloro-anilino]benzoic acid (99.16% purity) (245.0 mg) as a white solid.

LCMS: ET21585-88-P1A1 (M+H^{t}): 603.0 @ 2.895 min (10-80% ACN in H₂O, 4.5 min)

¹H NMR: ET21585-88-P1AA (DMSO-d6, 400 MHz)

δ 9.52 (br s, 1H), 7.91 (dd, J=1.6, 7.9 Hz, 1H), 7.45 (ddd, J=1.7, 7.1, 8.5 Hz, 1H), 7.38 (s, 2H), 7.34 (s, 2H), 7.25 (dd, J=0.6, 8.4 Hz, 1H), 6.91-6.85 (m, 1H), 5.20 (s, 2H), 4.09-4.00 (m, 4H), 2.81-2.74 (m, 2H), 2.55 (t, J=7.5 Hz, 2H), 2.06 (quin, J=6.4 Hz, 2H).

### Synthesis of B9 (T-751)

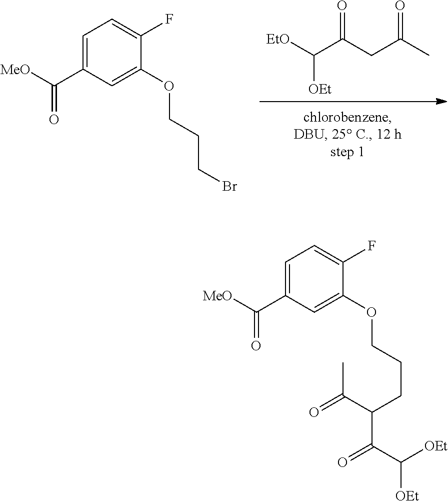 [SMILES: COC(=O)c1ccc(F)c(OCCCBr)c1 ; CCOC(OCC)C(=O)CC(C)=O ; CCOC(OCC)C(=O)C(CCCOc1cc(C(=O)OC)ccc1F)C(C)=O]

### Methyl 3-(4-acetyl-6,6-diethoxy-5-oxo-hexoxy)-4-fluoro-benzoate

To a solution of 1,1-diethoxypentane-2,4-dione (14.55 g, 77.29 mmol, 1.5 eq) and DBU (15.69 g, 103.05 mmol, 15.53 mL, 2.0 eq) in chlorobenzene (225 mL) was added methyl 3-(3-bromopropoxy)-4-fluoro-benzoate (15 g, 51.53 mmol, 1 eq) in portions. Then the mixture was stirred at 25° C. for 12 hr. The reaction was evaporated under reduced pressure. The residue was purified by flash silica gel chromatography (ISCO®; 80 g SepaFlash® Silica Flash Column, Eluent of 0˜13% Ethyl acetate/Petroleum ether gradient @ 100 mL/min) to give methyl 3-(4-acetyl-6-ethoxy-6-methoxy-5-oxo-hexoxy)-4-fluoro-benzoate (12.6 g, 32.78 mmol, 63.62% yield) as an orange oil.

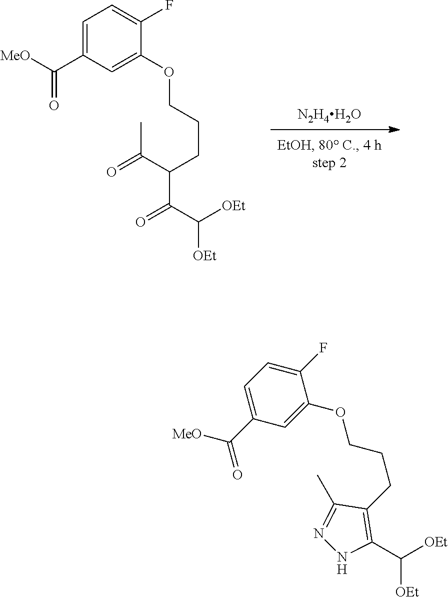 [SMILES: CCOC(OCC)C(=O)C(CCCOc1cc(C(=O)OC)ccc1F)C(C)=O ; "N2H4·H2O" ; CCOC(OCC)c1[nH]nc(C)c1CCCOc1cc(C(=O)OC)ccc1F]

### Methyl 3-[3-[5-(diethoxymethyl)-3-methyl-1H-pyrazol-4-yl]propoxy]-4-fluoro-benzoate

To a solution of methyl 3-(4-acetyl-6,6-diethoxy-5-oxo-hexoxy)-4-fluoro-benzoate (15 g, 37.65 mmol, 1 eq) in EtOH (150 mL) was added NH₂NH₂·H₂O (5.77 g, 112.95 mmol, 5.60 mL, 3 eq). The solution was stirred at 80° C. for 4 hr. The reaction was concentrated to the residue. The residue was purified by flash silica gel chromatography (ISCO®; 80 g SepaFlash® Silica Flash Column, Eluent of 0˜15% Ethylacetate/Petroleum ether gradient @100 mL/min) to give methyl 3-[3-[5-(diethoxymethyl)-3-methyl-1H-pyrazol-4-yl]propoxy]-4-fluoro-benzoate (10 g, 25.35 mmol, 67.34% yield) as a light yellow oil.

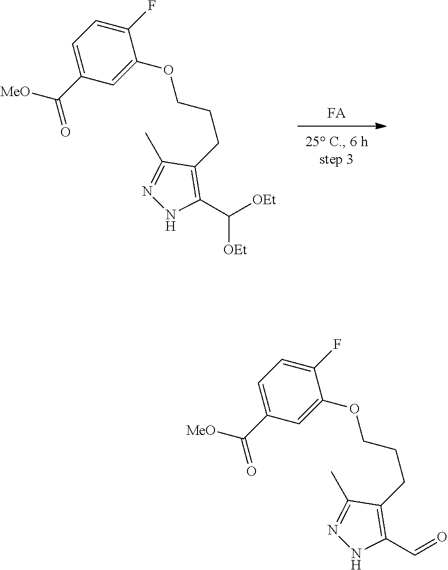 [SMILES: CCOC(OCC)c1[nH]nc(C)c1CCCOc1cc(C(=O)OC)ccc1F ; "FA" ; COC(=O)c1ccc(F)c(OCCCc2c(C)n[nH]c2C=O)c1]

### Methyl 4-fluoro-3-[3-(5-formyl-3-methyl-1H-pyrazol-4-yl)propoxy]benzoate

A solution of methyl 3-[3-[5-(diethoxymethyl)-3-methyl-1H-pyrazol-4-yl]propoxy]-4-fluoro-benzoate (8 g, 20.28 mmol, 1 eq) in methanoic acid (80 mL) was stirred at 25° C. for 6 hr. The reaction was concentrated under reduce pressure to give the residue as a yellow solid. The residue was stirred in MTBE (200 mL) at 25° C. for 0.5 h, filtered. The filter cake was dried under reduce pressure to give methyl 4-fluoro-3-[3-(5-formyl-3-methyl-1H-pyrazol-4-yl) propoxy]benzoate (5 g, crude) as a white solid. The crude product was used into the next step without further purification.

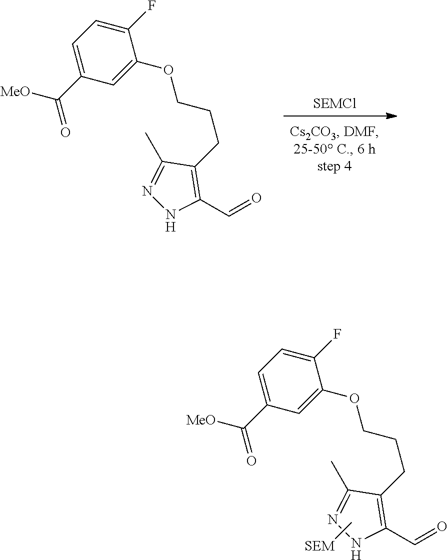

### Methyl 4-fluoro-3-(3-(5-formyl-3-methyl-1-((2-(trimethylsilyl)ethoxy)methyl)-1H-pyrazol-4-yl)propoxy)benzoate and methyl 4-fluoro-3-(3-(3-formyl-5-methyl-1-((2-(trimethyl silyl)ethoxy)methyl)-1H-pyrazol-4-yl)propoxy)benzoate (C₄₄H₆₂O₁₀N₄F₂Si₂)

To a suspension of methyl 4-fluoro-3-[3-(5-formyl-3-methyl-1H-pyrazol-4-yl)propoxy]benzoate (1 g, 3.12 mmol, 1 eq) in anhydrous DMF (10 mL) was added CS₂CO₃ (3.05 g, 9.37 mmol, 3 eq) at 25° C. The resulting mixture was stirred at 25° C. for 30 minutes. After 30 minutes, SEM-C1 (1.04 g, 6.24 mmol, 1.11 mL, 2 eq) was added into the mixture. The resulting mixture was stirred at 50° C. for 5.5 hours. The mixture (combined ET22788-11, 5 g scale) was quenched with H₂O (100 mL) and extracted with EtOAc (70 mL*3). The organic was washed with brine (30 mL), dried over Na₂SO₄, filtered and concentrated to give the residue. The residue was purified by flash silica gel chromatography (ISCO®; 20 g SepaFlash® Silica Flash Column, eluent of 0˜15% Ethyl acetate/Petroleum ethergradient @75 mL/min) to give C₄₄H₆₂O₁₀N₄F₂Si₂ (4 g) as a colorless oil. ¹H NMR was not clean, about 60% purity, byproduct was contained.

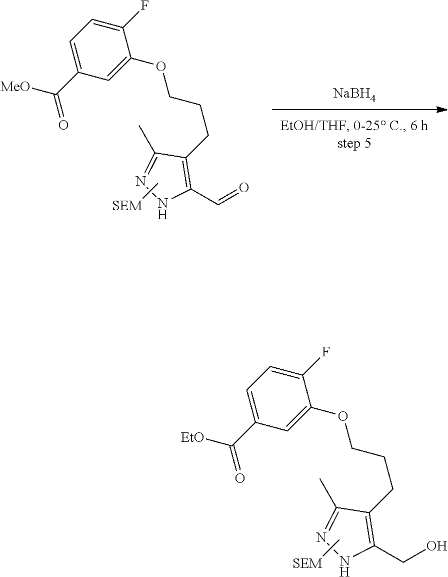

### Ethyl 4-fluoro-3-(3-(3-(hydroxymethyl)-5-methyl-1-((2-(trimethylsilyl)ethoxy)methyl)-1H-pyrazol-4-yl)propoxy)benzoate and ethyl 4-fluoro-3-(3-(5-(hydroxymethyl)-3-meth yl-1-((2-(trimethylsilyl)ethoxy)methyl)-1H-pyrazol-4-yl)propoxy)benzoate (C₄₆H₇₀O₁₀N₄F₂Si₂)

To a solution of C₄₄H₆₂O₁₀N₄F₂Si₂ (4.00 g, 5.33 mmol, 1 eq) in EtOH (30 mL)/THF (15 mL) was added NaBH₄ (604.49 mg, 15.98 mmol, 3 eq) at 0° C. The reaction was allowed to warm to 25° C. and stirred for 6 hr. The reaction was quenched with HCl (1N) to pH-7, concentrated to give a residue. The residue was partitioned between H₂O (30 mL) and EtOAc (50 mL*3). The organic layer was dried over Na₂SO₄ and concentrated to give a residue. The residue was purified by column (SiO₂, PE/EtOAc=20/1 ˜1/1) to give the crude product (3g). The crude was further purified by column (SiO₂, Petroleum ether/Ethyl acetate=20/1 ˜1/1) to give C₄₆H₇₀O₁₀N₄F₂Si₂ (1.2 g, 810.09 umol, 15.21% yield, 63% purity) as colorless oil.

¹H NMR: ET20197-189-P1AB (400 MHz, CHLOROFORM-d)

δ 7.67-7.60 (m, 2H), 7.12 (dd, J=8.4, 10.8 Hz, 1H), 5.47-5.43 (m, 1H), 5.38-5.31 (m, 1H), 4.67-4.59 (m, 2H), 4.42-4.32 (m, 2H), 4.05 (t, J=6.1 Hz, 2H), 3.58-3.50 (m, 2H), 2.67 (t, J=7.3 Hz, 2H), 2.22 (d, J=10.9 Hz, 3H), 2.09-1.97 (m, 2H), 1.45-1.34 (m, 3H), 0.92-0.84 (m, 2H), −0.01-−0.07 (m, 9H)

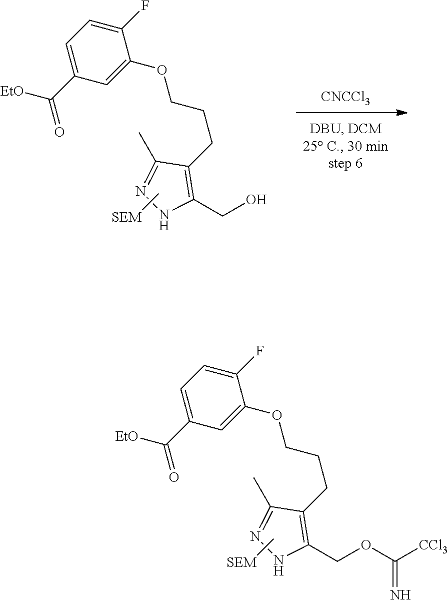

### Ethyl 4-fluoro-3-(3-(3-methyl-5-((2,2,2-trichloro-1-iminoethoxy)methyl)-1-((2-(trimethylsilyl)ethoxy)methyl)-1H-pyrazol-4-yl)propoxy)benzoate and ethyl 4-fluoro-3-(3-(5-methyl-3-((2,2,2-trichloro-1-iminoethoxy)methyl)-1-((2-(trimethylsilyl)ethoxy)methyl)-1H-pyrazol-4-yl)propoxy)benzoate (C₅₀H₇₀O₁₀N₆F₂Si₂Cl₆)

To solution of C₄₆H₇₀O₁₀N₄F₂Si₂ (1.1 g, 1.18 mmol, 1 eq) and 2,2,2-trichloroacetonitrile (1.02 g, 7.07 mmol, 709.12 uL, 6 eq) in DCM (20 mL) was added DBU (78.96 mg, 518.63 umol, 78.17 uL, 0.44 eq). The reaction was stirred at 25° C. for 0.5 hr. The reaction was concentrated to give C₅₀H₇₀O₁₀N₆F₂Si₂Cl₆ (1.5 g, crude) as a brown oil. The crude was used into the next step without further purification.

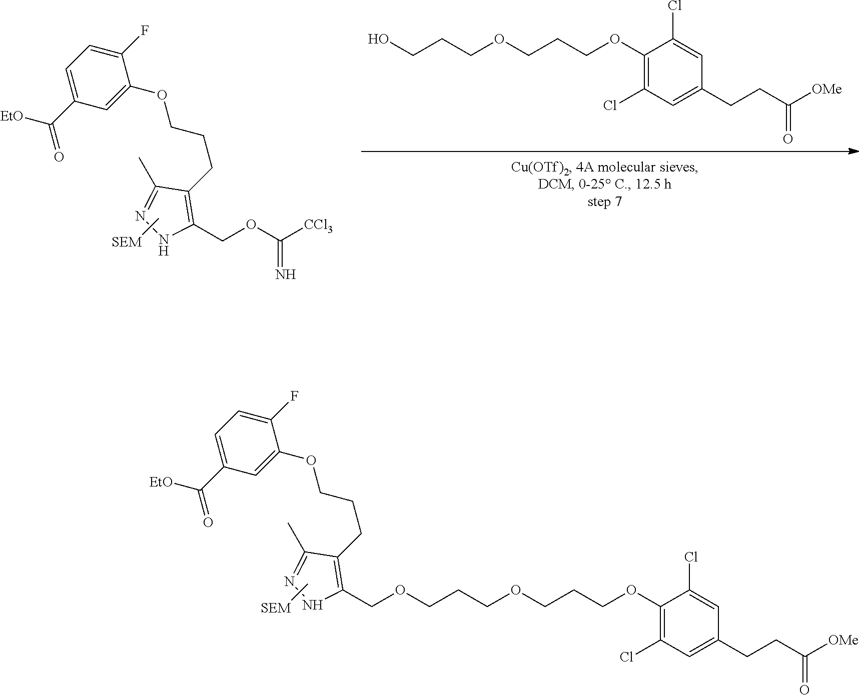

### Ethyl 3-(3-(5-((3-(3-(2,6-dichloro-4-(3-methoxy-3-oxopropyl)phenoxy)propoxy) propoxy) methyl)-3-methyl-1-((2-(trimethylsilyl)ethoxy) methyl)-1H-pyrazol-4-yl)propoxy)-4-fluorobenzoate and ethyl 3-(3-(3-((3-(3-(2,6-dichloro-4-(3-methoxy-3-oxopropyl)phenoxy) propoxy)propoxy)methyl)-5-methyl-1-((2-(trimethylsilyl)ethoxy) methyl)-1H-pyrazol-4-yl)propoxy)-4-fluorobenzoate (C₇₈H₁₁₀O₁₈Cl₄N₄F₂Si₂)

A mixture of methyl 3-[3,5-dichloro-4-[3-(3-hydroxypropoxy)propoxy]phenyl]propanoate (550 mg, 1.51 mmol, 1 eq), 4A molecular sieves (1.5 g) and C₅₀H₇₀O₁₀N₆F₂Si₂Cl₆ (1.50 g, 1.23 mmol, 0.813 eq) in DCM (5 mL) was stirred at 0° C. for 0.5 h. Then bis(trifluoromethylsulfonyloxy)copper (546.14 mg, 1.51 mmol, 1 eq) was added to the mixture. The resulting mixture was stirred at 25° C. for 12 h. The reaction was filtered through the Celite and the filtrate was washed with NaHCO₃ (sat. 20 mL*2), H₂O (20 mL*2), brine (20 mL). The organic layers were dried over Na₂SO₄, filtered and concentrated to give the residue. The residue was purified by column chromatography (SiO₂, Petroleum ether/2-methyltetrahydrofuran=5/1 to 1:1) to give C₇₈H₁₁₀O₁₈Cl₄N₄F₂Si₂ (1 g, 411.01 umol, 27.22% yield, 66.9% purity) as a light blue oil.

LCMS: ET22788-17-P1D2 (M+H⁺): 813.2 @ 1.872 min (5-95% ACN in H₂O, 2.0 min)

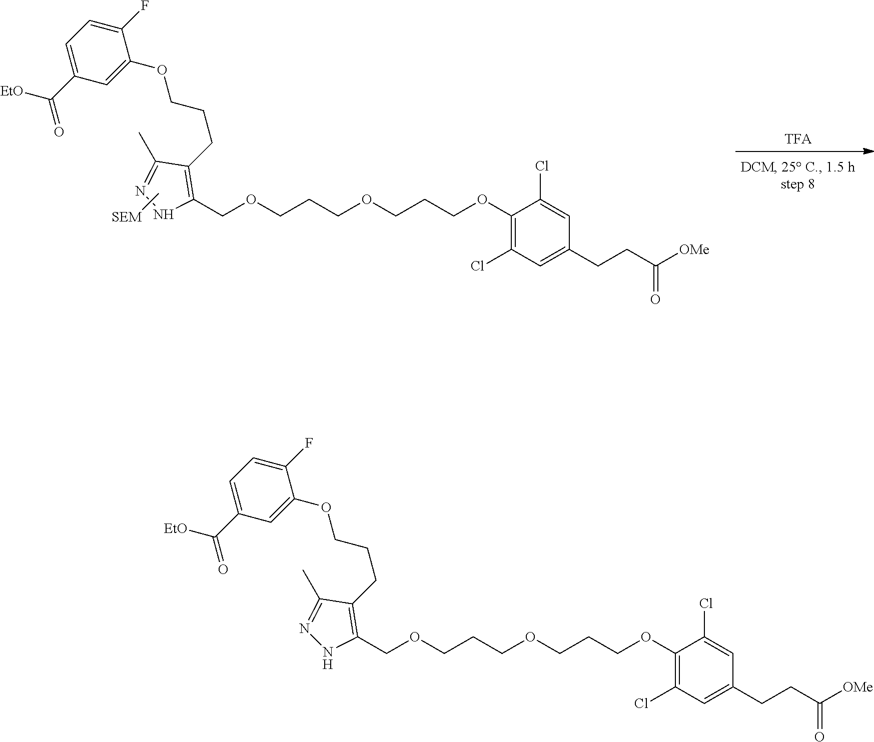

### Ethyl 3-[3-[5-[3-[3-[2,6-dichloro-4-(3-methoxy-3-oxo-propyl)phenoxy]propoxy]propoxy methyl]-3-methyl-1H-pyrazol-4-yl]propoxy]-4-fluoro-benzoate

To a solution of C₇₈H₁₁₀O₁₈Cl₄N₄F₂Si₂ (950 mg, 583.65 umol, 1 eq) in DCM (1 mL) was added TFA (7.70 g, 67.53 mmol, 5 mL, 115.71 eq). The reaction was stirred at 25° C. for 1 hr. The reaction was concentrated to give ethyl 3-[3-[5-[3-[3-[2,6-dichloro-4-(3-methoxy-3-oxo-propyl)phenoxy]propoxy]propoxymethyl]-3-methyl-1H-pyrazol-4-yl]propoxy]-4-fluoro-benzoate (1.0 g, crude) as a brown oil. The crude was used into next step without further purification.

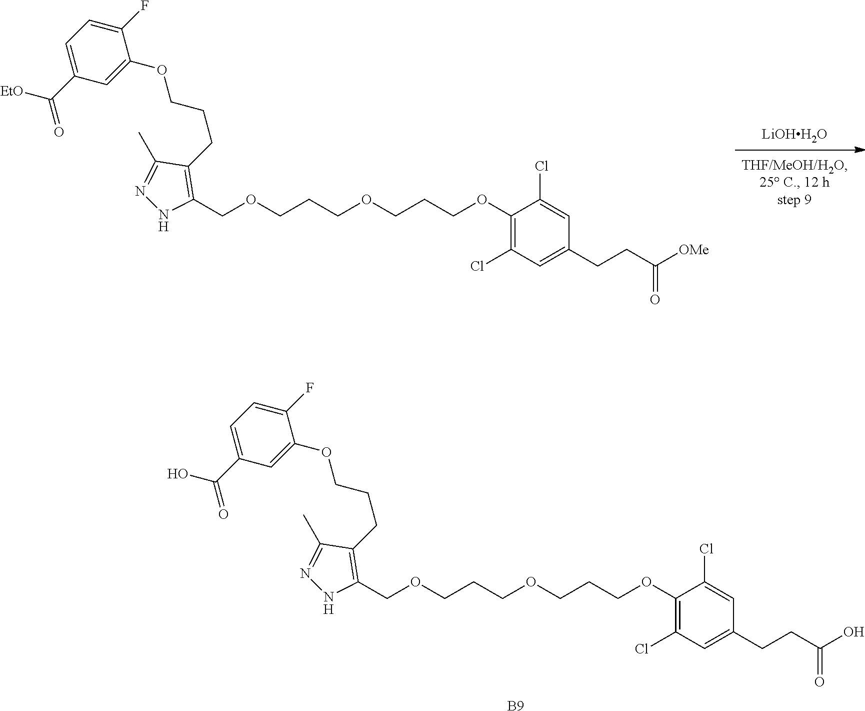 [SMILES: CCOC(=O)c1ccc(F)c(OCCCc2c(C)n[nH]c2COCCCOCCCOc2c(Cl)cc(CCC(=O)OC)cc2Cl)c1 ; "LiOH·H2O" ; Cc1n[nH]c(COCCCOCCCOc2c(Cl)cc(CCC(=O)O)cc2Cl)c1CCCOc1cc(C(=O)O)ccc1F ; "B9"]

### 3-[3-[5-[3-[3-[4-(2-carboxyethyl)-2,6-dichloro-phenoxy]propoxy]propoxymethyl]-3-methyl-1H-pyrazol-4-yl]propoxy]-4-fluorobenzoic acid

To a solution of ethyl 3-[3-[5-[3-[3-[2,6-dichloro-4-(3-methoxy-3-oxo-propyl)phenoxy]propoxy]propoxymethyl]-3-methyl-1Hpyrazol-4-yl]propoxy]-4-fluoro-benzoate (1 g, 1.46 mmol, 1 eq) in THF (3 mL)/MeOH (1 mL) was added a solution of LiOH·H₂O (306.91 mg, 7.31 mmol, 5 eq) in H₂O (1 mL) at 25° C. The mixture was stirred at 25° C. for 12 hr. The reaction was concentrated and adjusted with HCl (1N) to pH 6-7. The mixture was extracted with 2-methyltetrahydrofuran (30 mL*3). The organic layers were washed with brine (20 mL*2), filtered and concentrated to give the residue. The residue was purified by prep-HPLC (HCl condition) to give 3-[3-[5-[3-[3-[4-(2-carboxyethyl)-2,6-dichloro-phenoxy]propoxy]propoxymethyl]-3-methyl-1H-pyrazol-4-yl]propoxy]-4-fluorobenzoic acid (263.1 mg, 410.13 umol, 28.04% yield, 100% purity) as a light yellow solid.

LCMS: ET20197-205-P1P (M+H⁺): 641.1 @ 2.712 min (10-90% ACN in H₂O, 4.5 min)

¹H NMR: ET20197-205-P1AA (400 MHz, METHANOL-d4) δ 7.69-7.60 (m, 2H), 7.24-7.14 (m, 3H), 4.63 (s, 2H), 4.09 (t, J=5.6 Hz, 2H), 4.02 (t, J=6.1 Hz, 2H), 3.64 (td, J=6.2, 12.2 Hz, 4H), 3.53 (t, J=6.1 Hz, 2H), 2.87-2.80 (m, 2H), 2.77 (t, J=7.3 Hz, 2H), 2.62-2.54 (m, 2H), 2.39 (s, 3H), 2.10-1.97 (m, 4H), 1.86 (quin, J=6.2 Hz, 2H)

### Synthesis of B10 (T-752)

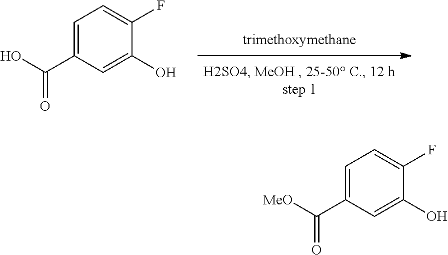 [SMILES: O=C(O)c1ccc(F)c(O)c1 ; "trimethoxymethane" ; COC(=O)c1ccc(F)c(O)c1]

### Methyl 4-fluoro-3-hydroxy-benzoate

To 4-fluoro-3-hydroxy-benzoic acid (20 g, 128.11 mmol, 1 eq) in anhydrous MeOH (160 mL) was added trimethoxymathane (23.79 g, 224.20 mmol, 24.58 mL, 1.75 eq) and concentrated sulfuric acid (5.65 g, 57.59 mmol, 3.07 mL, 0.45 eq) under nitrogen at 25° C.

The reaction mixture was stirred at 50° C. for 12 h. The solvent was removed in vacuo and the solution was poured into cooled H₂O (300 mL). A white solid was formed and dissolved by EtOAc (150 mL), extracted with EtOAc (100 mL*3). The combined organic phases were washed with a solution saturated NaHCO₃ (70 mL*3), treated with brine (70 mL*2), dried over Na₂SO₄, filtered, concentrated to give methyl 4-fluoro-3-hydroxy-benzoate (21 g, 123.43 mmol, 96.34% yield) as a white solid. The crude product was used into the next step without further purification.

¹H NMR: ET22788-1-P1A (400 MHz, DMSO-d₆)

δ 10.33 (s, 1H), 7.55 (dd, J=2.1, 8.6 Hz, 1H), 7.41 (ddd, J=2.2, 4.5, 8.5 Hz, 1H), 7.26 (dd, J=8.6, 11.0 Hz, 1H), 3.84-3.79 (m, 3H)

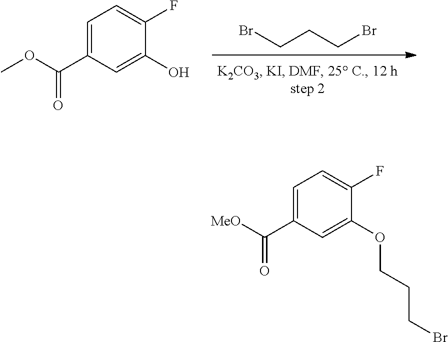 [SMILES: COC(=O)c1ccc(F)c(O)c1 ; BrCCCBr ; COC(=O)c1ccc(F)c(OCCCBr)c1]

### Methyl 3-(3-bromopropoxy)-4-fluoro-benzoate

To a solution of methyl 4-fluoro-3-hydroxy-benzoate (21 g, 123.43 mmol, 1 eq) and 1,3-dibromopropane (124.59 g, 617.15 mmol, 62.93 mL, 5 eq) in DMF (480 mL) was added K₂CO₃ (20.47 g, 148.12 mmol, 1.2 eq) and KI (1.02 g, 6.17 mmol, 0.05 eq). The reaction mixture was stirred at 25° C. for 12 h. The reaction mixture (combined ET22788-2, 5 g scale) was filtered and the filter cake was washed with DMF (40 mL*3). The filtrate was evaporated under reduced pressure. The residue was purified by flash silica gel chromatography (ISCO®; 80 g SepaFlash® Silica Flash Column, Eluent of 0˜5% Ethyl acetate/Petroleum ether gradient @ 100 mL/min) to give methyl 3-(3-bromopropoxy)-4-fluoro-benzoate (30 g) as colorless oil.

¹H NMR: ET22788-3-P1A1 (400 MHz, DMSO-d₆)

δ 7.64 (br d, J=8.2 Hz, 1H), 7.60-7.54 (m, 1H), 7.39-7.31 (m, 1H), 4.21 (t, J=6.1 Hz, 2H), 3.84 (s, 3H), 3.66 (t, J=6.6 Hz, 2H), 2.35-2.22 (m, 2H)

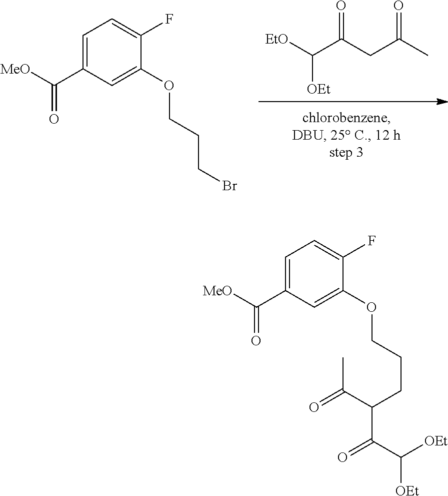 [SMILES: COC(=O)c1ccc(F)c(OCCCBr)c1 ; CCOC(OCC)C(=O)CC(C)=O ; CCOC(OCC)C(=O)C(CCCOc1cc(C(=O)OC)ccc1F)C(C)=O]

### Methyl 3-(4-acetyl-6,6-diethoxy-5-oxo-hexoxy)-4-fluoro-benzoate

To a solution of 1,1-diethoxypentane-2,4-dione (9.70 g, 51.53 mmol, 1.5 eq) and DBU (10.46 g, 68.70 mmol, 10.36 mL, 2 eq) in chlorobenzene (120 mL) was added methyl 3-(3-bromopropoxy)-4-fluoro-benzoate (10 g, 34.35 mmol, 1 eq) in portions. Then the mixture was stirred at 25° C. for 12 hr. The reaction was concentrated under reduce pressure to give the residue. The residue was purified by flash silica gel chromatography (ISCO®; 120 g SepaFlash® Silica Flash Column, Eluent of 0˜20% Ethylacetate/Petroleum ethergradient @100 mL/min) to give the crude (7.8 g). The crude was further purified by flash silica gel chromatography (ISCO®; 120 g SepaFlash® Silica Flash Column, Eluent of 0 ˜10% 2-methyltetrahydrofuran/Petroleum ethergradient @ 100 mL/min) to give methyl 3-(4-acetyl-6,6-diethoxy-5-oxo-hexoxy)-4-fluoro-benzoate (7.0 g, 17.57 mmol, 51.15% yield) as a light yellow oil.

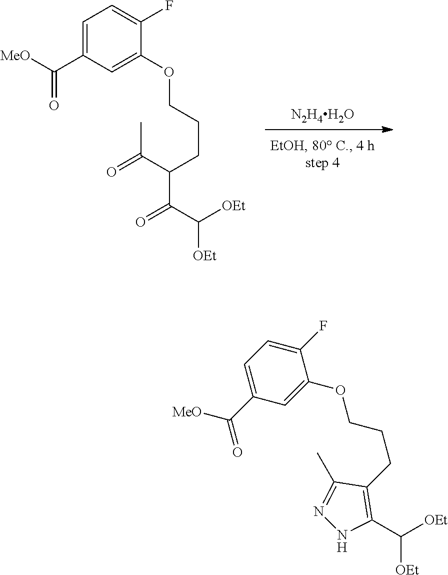 [SMILES: CCOC(OCC)C(=O)C(CCCOc1cc(C(=O)OC)ccc1F)C(C)=O ; "N2H4·H2O" ; CCOC(OCC)c1[nH]nc(C)c1CCCOc1cc(C(=O)OC)ccc1F]

### Methyl 3-[3-[5-(diethoxymethyl)-3-methyl-1H-pyrazol-4-yl]propoxy]-4-fluoro-benzoate

To a solution of methyl 3-(4-acetyl-6,6-diethoxy-5-oxo-hexoxy)-4-fluoro-benzoate (7 g, 17.57 mmol, 1 eq) in EtOH (70 mL) was added NH₂NH₂·H₂O (2.69 g, 52.71 mmol, 2.61 mL, 3 eq). The solution was stirred at 80° C. for 4 hr. The reaction was concentrated to the residue. The residue was purified by flash silica gel chromatography (ISCO®; 80 g SepaFlash® Silica Flash Column, Eluent of 0 ˜15% Ethylacetate/Petroleum ether gradient @100 mL/min) to give methyl 3-[3-[5-(diethoxymethyl)-3-methyl-1H-pyrazol-4-yl]propoxy]-4-fluoro-benzoate (5 g, 12.68 mmol, 72.15% yield) as a light yellow oil.

¹H NMR: ET20197-198-P1BB (400 MHz, CHLOROFORM-d)

δ 7.66-7.59 (m, 2H), 7.15-7.08 (m, 1H), 5.59 (s, 1H), 4.05 (t, J=6.1 Hz, 2H), 3.90 (s, 3H), 3.65-3.49 (m, 4H), 2.69 (t, J=7.5 Hz, 2H), 2.20 (s, 3H), 2.10-2.00 (m, 3H), 1.25-1.19 (m, 6H)

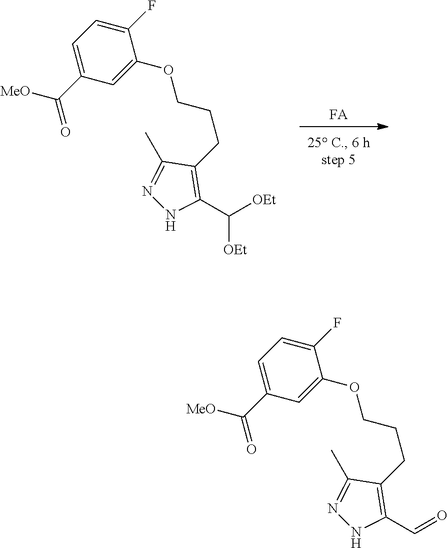 [SMILES: CCOC(OCC)c1[nH]nc(C)c1CCCOc1cc(C(=O)OC)ccc1F ; "FA" ; COC(=O)c1ccc(F)c(OCCCc2c(C)n[nH]c2C=O)c1]

### Methyl 4-fluoro-3-[3-(5-formyl-3-methyl-1H-pyrazol-4-yl)propoxy]benzoate

A solution of methyl 3-[3-[5-(diethoxymethyl)-3-methyl-1H-pyrazol-4-yl]propoxy]-4-fluoro-benzoate (5 g, 12.68 mmol, 1 eq) in formic acid (50 mL) was stirred at 25° C. for 6 hr. The reaction was concentrated under reduce pressure to give the residue as a yellow solid. The residue was stirred in MTBE (50 mL) at 25° C. for 0.5 h, filtered. The cake was dried under reduce pressure to give methyl 4-fluoro-3-[3-(5-formyl-3-methyl-1H-pyrazol-4-yl) propoxy]benzoate (3.1 g, crude) as a white solid. The crude product was used into the next step without further purification.

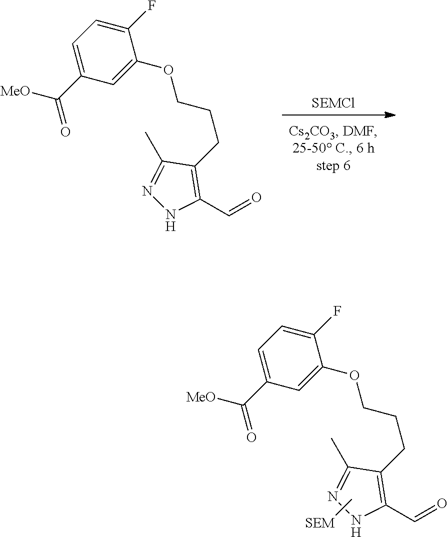

### Methyl 4-fluoro-3-(3-(5-formyl-3-methyl-1-((2-(trimethylsilyl)ethoxy)methyl)-1H-pyraz ol-4-yl)propoxy)benzoate and methyl 4-fluoro-3-(3-(3-formyl-5-methyl-1-((2-(trimethyl silyl)ethoxy)methyl)-1H-pyrazol-4-yl)propoxy)benzoate (C₄₄H₆₂O₁₀N₄F₂Si₂)

To a suspension of methyl 4-fluoro-3-[3-(5-formyl-3-methyl-1H-pyrazol-4-yl)propoxy]benzoate (3.1 g, 9.68 mmol, 1 eq) in anhydrous DMF (50 mL) was added Cs₂CO₃ (9.46 g, 29.03 mmol, 3 eq) at 25° C. The resulting mixture was stirred at 25° C. for 30 minutes. After 30 minutes, SEM-Cl (3.23 g, 19.36 mmol, 3.43 mL, 2 eq) was added into the mixture. The resulting mixture was stirred at 50° C. for 5.5 hours. The reaction mixture was quenched by addition H₂O (50 mL), and then extracted with 2-methyltetrahydrofuran (50 mL*3). The combined organic layers were washed with brine (20 mL*2), dried over Na₂SO₄, filtered and concentrated under reduced pressure to give a residue. The residue was purified by column (SiO₂, Petroleum ether/Ethyl acetate=5/1 to 10:1) to give C₄₄H₆₂O₁₀N₄F₂Si₂ (2.5 g, 2.77 mmol, 28.67% yield) as a colorless oil.

¹H NMR: ET22788-15-P1BB (400 MHz, CHLOROFORM-d)

δ 10.04-9.99 (m, 1H), 7.67-7.57 (m, 2H), 7.16-7.09 (m, 1H), 5.70 (s, 1H), 5.45 (s, 1H), 4.09-3.99 (m, 2H), 3.90 (d, J=1.0 Hz, 3H), 3.61-3.52 (m, 2H), 3.00-2.83 (m, 2H), 2.29-2.23 (m, 3H), 2.15-2.07 (m, 2H), 0.93-0.83 (m, 2H), −0.01-0.08 (m, 9H)

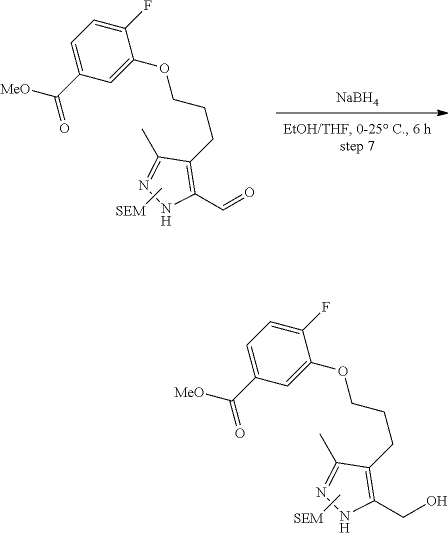

### Methyl 4-fluoro-3-(3-(3-(hydroxymethyl)-5-methyl-1-((2-(trimethylsilyl)ethoxy) methyl)-1H-pyrazol-4-yl)propoxy)benzoate and methyl 4-fluoro-3-(3-(5-(hydroxymethyl)-3-methyl-1-((2-(trimethylsilyl)ethoxy)methyl)-1H-pyrazol-4-yl) propoxy)benzoate (C₄₄H₆₆O₁₀N₄F₂Si₂)

To a solution of C₄₄H₆₂O₁₀N₄F₂Si₂ (2.5 g, 2.77 mmol, 1 eq) in EtOH (30 mL)/THF (15 mL) was added NaBH₄ (629.74 mg, 16.65 mmol, 6 eq) at 0° C. The reaction was allowed to warm to 25° C. and stirred for 6 hr. The reaction was quenched with HCl (1N) to pH˜7, concentrated to give a residue. The residue was partitioned between H₂O (30 mL) and EtOAc (50 mL*3).

The organic layer was dried over Na₂SO₄ and concentrated to give a residue. The residue was purified by column (SiO₂, Petroleum ether/Ethyl acetate=20/1 ˜1/1) to give the crude product (2.5 g). The crude was further purified by column ((SiO₂, Petroleum ether/2-methyltetrahydrofuran=20/1 ˜1/1) to give C₄₄H₆₆O₁₀N₄F₂Si₂ (2.0 g, 2.21 mmol, 79.64% yield) as colorless oil.

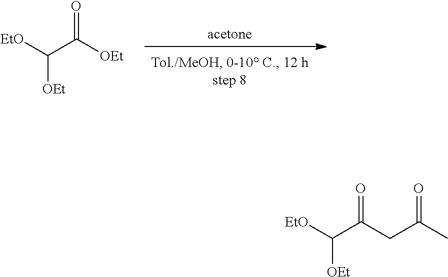

### 1,1-diethoxypentane-2,4-dione

To a suspension of NaH (5.96 g, 148.97 mmol, 60% purity, 1.05 eq) in Tol. (250 mL) was dropwise added MeOH (14 mL) at 0-10° C., then ethyl 2,2-diethoxyacetate (25 g, 141.88 mmol, 25.38 mL, 1 eq) and acetone (8.24 g, 141.88 mmol, 10.43 mL, 1 eq) was added at 0-10° C. The mixture was stirred at 25° C. for 12 hr. The reaction mixture was quenched by addition of a mixed solvent of AcOH/H₂O (1:1) to pH 7-8, and extracted with EtOAc (70 mL*3). The combined organic layers were washed with NaHCO₃ (aq. 50 mL) and brine (50 mL), dried over Na₂SO₄, filtered and concentrated under reduced pressure to give a residue. The crude product was distilled in vacuum (54° C., 300 Pa) to afford 1,1-diethoxypentane-2,4-dione (30 g, 159.39 mmol, 56.17% yield) as a colorless oil.

¹H NMR: ET20197-171-P1AA (400 MHz, CHLOROFORM-d)

δ 5.93-5.85 (m, 1H), 4.82-4.75 (m, 1H), 3.72-3.53 (m, 4H), 2.13-2.08 (m, 2H), 1.27-1.21 (m, 6H)

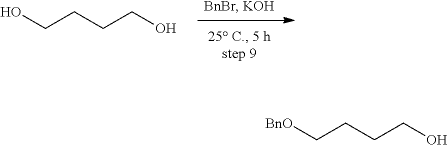

### 4-benzyloxybutan-1-ol

KOH (6.94 g, 123.77 mmol, 4.23 eq) and bromomethylbenzene (5 g, 29.23 mmol, 3.47 mL, 1 eq) were added in portions to butane-1,4-diol (11.69 g, 129.69 mmol, 11.46 mL, 4.44 eq). The mixture was stirred at 25° C. for 5 hr. The reaction mixture was diluted with H₂O (50 mL) and extracted with EtOAc (30 mL*2). The combined organic layers were washed with brine (50 mL), dried over (Na₂SO₄), filtered and concentrated under reduced pressure to give a residue. The residue was purified by flash silica gel chromatography (ISCO®; 12 g SepaFlash® Silica Flash Column, Eluent of 0 ˜25% Ethylacetate/Petroleum ethergradient @50 mL/min) to afford 4-benzyloxybutan-1-ol (5 g, 27.74 mmol, 94.89% yield) as colorless oil.

¹H NMR: ET21585-40-P1AA (400 MHz, CHLOROFORM-d)

δ 7.41-7.27 (m, 5H), 4.59-4.45 (m, 2H), 3.64 (t, J=5.9 Hz, 2H),3.52 (t, J=5.8 Hz, 2H), 2.16 (br s, 1H), 1.74-1.65 (m, 4H).

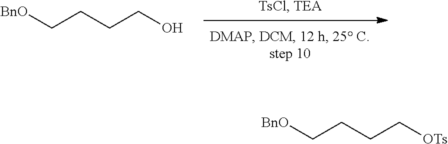 [SMILES: "TsCl, TEA" ; OCCCCOCc1ccccc1 ; Cc1ccc(S(=O)(=O)OCCCCOCc2ccccc2)cc1]

### 4-benzyloxybutyl 4-methylbenzenesulfonate

To a solution of 4-benzyloxybutan-1-ol (5 g, 27.74 mmol, 4.85 mL, 1 eq) and 4-methyl benzenesulfonyl chloride (6.35 g, 33.29 mmol, 1.2 eq) in DCM (35 mL) was added TEA (5.61 g, 55.48 mmol, 7.72 mL, 2 eq) and DMAP (338.90 mg, 2.77 mmol, 0.1 eq). The mixture was stirred at 25° C. for 12 hr. The reaction mixture was diluted with H₂O (40 mL) and partitioned. The aqueous layer was extracted with EtOAc (30 mL). The combined organic layers were washed with brine (50 mL), dried over Na₂SO₄, filtered and concentrated under reduced pressure to give a residue. The residue was purified by flash silica gel chromatography (ISCO®; 20 g SepaFlash® Silica Flash Column, Eluent of 0 ˜8% Ethylacetate/Petroleum ethergradient @ 75 mL/min) to afford 4-benzyloxybutyl 4-methylbenzenesulfonate (6 g, 17.94 mmol, 64.67% yield) as colorless oil.

¹H NMR: ET21585-45-P1 AA (400 MHz, CHLOROFORM-d)

δ 7.84-7.74 (m, 2H), 7.41-7.23 (m, 6H), 4.46 (s, 2H), 4.06 (t, J=6.4 Hz, 2H), 3.43 (t, J=6.1 Hz, 2H), 2.45 (s, 3H), 1.83-1.71 (m, 2H), 1.69-1.59 (m, 2H)

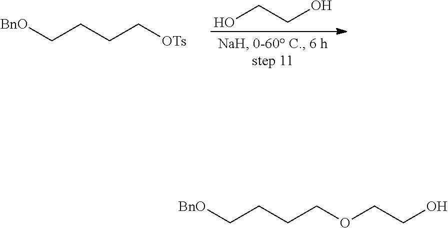

### 2-(4-benzoyloxybutoxy)ethanol

To ethylene glycol (9.28 g, 149.51 mmol, 8.36 mL, 10 eq) was added NaH (627.94 mg, 15.70 mmol, 60% purity, 1.05 eq) at 0° C. The mixture was stirred at 0° C. for 0.5 hr. Then 4-benzyloxybutyl 4-methylbenzenesulfonate (5 g, 14.95 mmol, 1 eq) was added at 0° C. The mixture was stirred at 60° C. for 5.5 hr. The reaction mixture (combined ET21585-48, 1 g scale) was diluted with H₂O (80 mL) and extracted with EtOAc (60 mL*2). The combined organic layers were washed with brine (60 mL), dried over Na₂SO₄, filtered and concentrated under reduced pressure to give a residue. The residue was purified by column chromatography (SiO₂, Petroleum ether/Ethyl acetate=10/1 to 1/1) to afford 2-(4-benzyloxybutoxy)ethanol (2 g) as a colorless oil.

¹H NMR: ET21585-51-P1AA (400 MHz, CHLOROFORM-d)

δ 7.40-7.29 (m, 4H), 4.53 (s, 2H), 3.76-3.70 (m, 2H), 3.58-3.47 (m, 6H), 2.06 (br d, J=5.1 Hz, 1H), 1.76-1.66 (m, 4H).

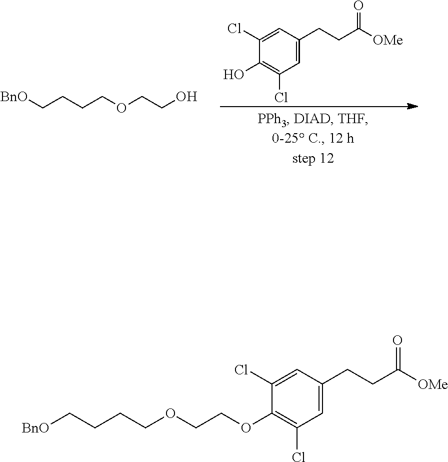 [SMILES: OCCOCCCCOCc1ccccc1 ; COC(=O)CCc1cc(Cl)c(O)c(Cl)c1 ; COC(=O)CCc1cc(Cl)c(OCCOCCCCOCc2ccccc2)c(Cl)c1]

### Methyl 3-[4-[2-(4-benzyloxybutoxy)ethoxy]-3,5-dichloro-phenyl]propanoate

To a solution of 2-(4-benzyloxybutoxy)ethanol (300 mg, 1.34 mmol, 1 eq) and methyl 3-(3,5-dichloro-4-hydroxyphenyl) propanoate (333.16 mg, 1.34 mmol, 1 eq) in THF (2 mL) was added PPh₃ (526.22 mg, 2.01 mmol, 1.5 eq) and DIAD (405.69 mg, 2.01 mmol, 390.08 uL, 1.5 eq) at 0° C. under N₂. The mixture was stirred at 25° C. for 12 hr. The reaction mixture was diluted with H₂O (30 mL) and extracted with EtOAc (20 mL*2). The combined organic layers were washed with brine (30 mL), dried over Na₂SO₄, filtered and concentrated under reduced pressure to give a residue. The residue was purified by flash silica gel chromatography (ISCO®; 4 g SepaFlash® Silica Flash Column, Eluent of 0 ˜8% Ethylacetate/Petroleum ethergradient @ 50 mL/min) to afford methyl 3-[4-[2-(4-benzyloxy butoxy)ethoxy]-3,5-dichloro-phenyl]propanoate (300 mg, 658.80 umol, 49.260/yield) as colorless oil.

¹H NMR: ET21585-52-P1AA (400 MHz, CHLOROFORM-d)

δ 7.41-7.20 (m, 4H), 7.13 (s, 2H), 4.51 (s, 2H), 4.16 (t, J=5.0 Hz, 2H), 3.81 (t, J=5.0 Hz, 2H), 3.69 (s, 3H), 3.61-3.54 (m, 2H), 3.53-3.47 (m, 2H), 2.90-2.82 (m, 2H), 2.64-2.55 (m, 2H), 1.75-1.67 (m, 4H)

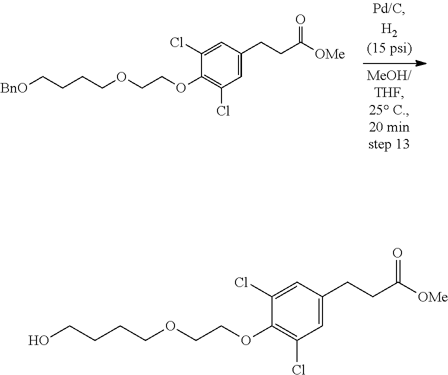 [SMILES: COC(=O)CCc1cc(Cl)c(OCCOCCCCOCc2ccccc2)c(Cl)c1 ; COC(=O)CCc1cc(Cl)c(OCCOCCCCO)c(Cl)c1]

### Methyl 3-[3,5-dichloro-4-[2-(4-hydroxybutoxy)ethoxy]phenyl]propanoate

To a solution of methyl 3-[4-[2-(4-benzyloxybutoxy)ethoxy]-3,5-dichloro-phenyl]propanoate (1.6 g, 3.51 mmol, 1 eq) in THF (10 mL) and MeOH (10 mL) was added Pd/C (1 g, 10% purity) under Ar. The suspension was degassed under vacuum and purged with H₂ several times. The mixture was stirred under H₂ (15 psi) at 25° C. for 20 min. The suspension was filtered through a pad of Celite and the pad was washed with MeOH (200 mL). The combined filtrates were concentrated to dryness to give crude product. The crude was purified by flash silica gel chromatography (ISCO®; 12 g SepaFlash® Silica Flash Column, Eluent of 0 ˜20% Ethylacetate/Petroleum ethergradient @ 50 mL/min) to afford methyl 3-[3,5-dichloro-4-[2-(4-hydroxybutoxy)ethoxy]phenyl]propanoate (800 mg) as colorless oil.

¹H NMR: ET21585-61-P1AA (400 MHz, CHLOROFORM-d)

δ 7.13 (s, 2H), 4.20-4.15 (m, 2H), 3.88-3.80 (m, 2H), 3.68 (s, 3H), 3.67-3.63 (m, 2H), 3.60 (t, J=5.9 Hz, 2H), 2.91-2.82 (m, 2H), 2.64-2.55 (m, 2H), 1.77-1.62 (m, 4H).

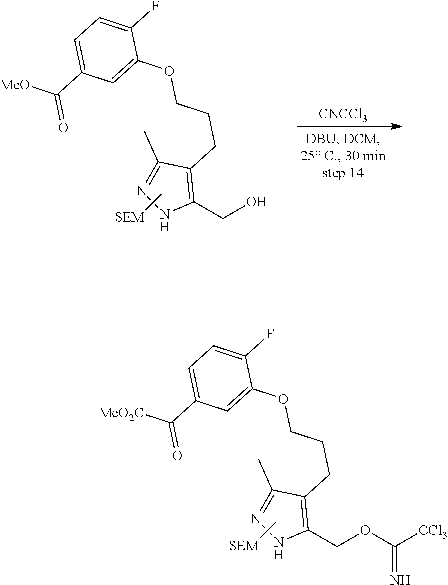

### Methyl 4-fluoro-3-(3-(3-methyl-5-((2,2,2-trichloro-1-iminoethoxy)methyl)-1-((2-(trimethylsilyl)ethoxy)methyl)-1H-pyrazol-4-yl)propoxy)benzoate and methyl 4-fluoro-3-(3-(5-methyl-3-((2,2,2-trichloro-1-iminoethoxy)methyl)-1-((2-(trimethylsilyl) ethoxy)methyl)-1H-pyrazol-4-yl)propoxy)benzoate (C₄₈H₆₆O₁₀N₆F₂Si₂Cl₆)

To solution of C₄₄H₆₆O₁₀N₄F₂Si₂ (1.2 g, 1.33 mmol, 1 eq) and 2,2,2-trichloroacetonitrile (1.15 g, 7.95 mmol, 797.56 uL, 6 eq)) in DCM (10 mL) was DBU (788.80 mg, 583.31 umol, 87.92 uL, 0.44 eq). The reaction was stirred at 25° C. for 0.5 hr. The reaction was concentrated to give C₄₈H₆₆O₁₀N₆F₂Si₂Cl₆ (1.6 g, crude) as a brown oil. The crude was used into the next step without further purification.

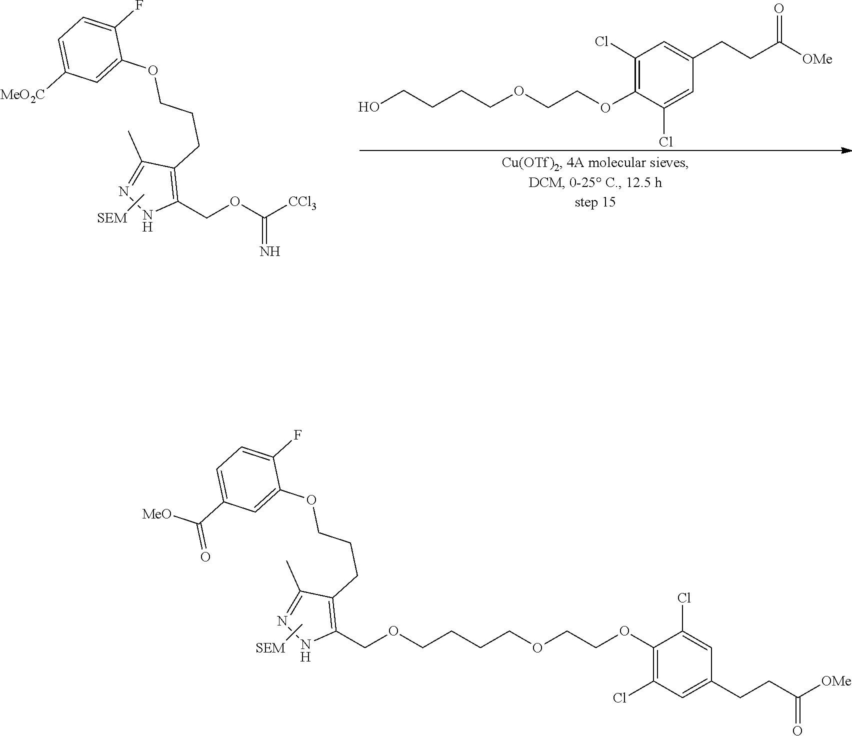

### Methyl 3-(3-(5-((4-(2-(2,6-dichloro-4-(3-methoxy-3-oxopropyl)phenoxy)ethoxy)butoxy) methyl)-3-methyl-1-((2-(trimethylsilyl)ethoxy)methyl)-1H-pyrazol-4-yl)propoxy)-4-fluorobenzoate and methyl 3-(3-(3-((4-(2-(2,6-dichloro-4-(3-methoxy-3-oxopropyl) phenoxy)ethoxy)butoxy)methyl)-5-methyl-1-((2-(trimethylsilyl)ethoxy) methyl)-1H-pyrazol-4-yl)propoxy)-4-fluorobenzoate (C₇₆H₁₀₆O₁₈Cl₄N₄F₂Si₂)

A solution of C₄₈H₆₆O₁₀N₆F₂Si₂Cl₆ (1.6 g, 1.34 mmol, 8.15 e-1 eq) in DCM (3 mL) is cooled to 0° C. A solution of methyl 3-[3,5-dichloro-4-[2-(4-hydroxybutoxy) ethoxy]phenyl]propanoate (600 mg, 1.64 mmol, 1 eq) and 4A MOLECULAR SIEVE (2 g) in DCM (3 mL) were added at 0° C. After 30 min, bis(trifluoromethylsulfonyloxy)copper (594.14 mg, 1.64 mmol, 1 eq) was added at 0° C. The reaction was stirred at 25° C. for 12 h. The reaction mixture was filtered and washed with aq.sat.NaHCO₃ (30 mL*3), H₂O (20 mL*2) and brine (20 mL). The organic layers were concentrated to give the residue. The residue was purified by flash silica gel chromatography (ISCO®; 40 g SepaFlash® Silica Flash Column, Eluent of 20-50% 2-methyltetrahydrofuran/Petroleum ethergradient @ 100 mL/min) to give C₇₆H₁₀₆O₁₈C₁₄N₄F₂Si₂ (1 g, 543.87 umol, 33.11% yield, 87% purity) as a light blue oil.

LCMS: ET20197-209-P1D3 (M+H⁺): 799.2@ 1.680 min (5-95% ACN in H₂O, 2.0 min)

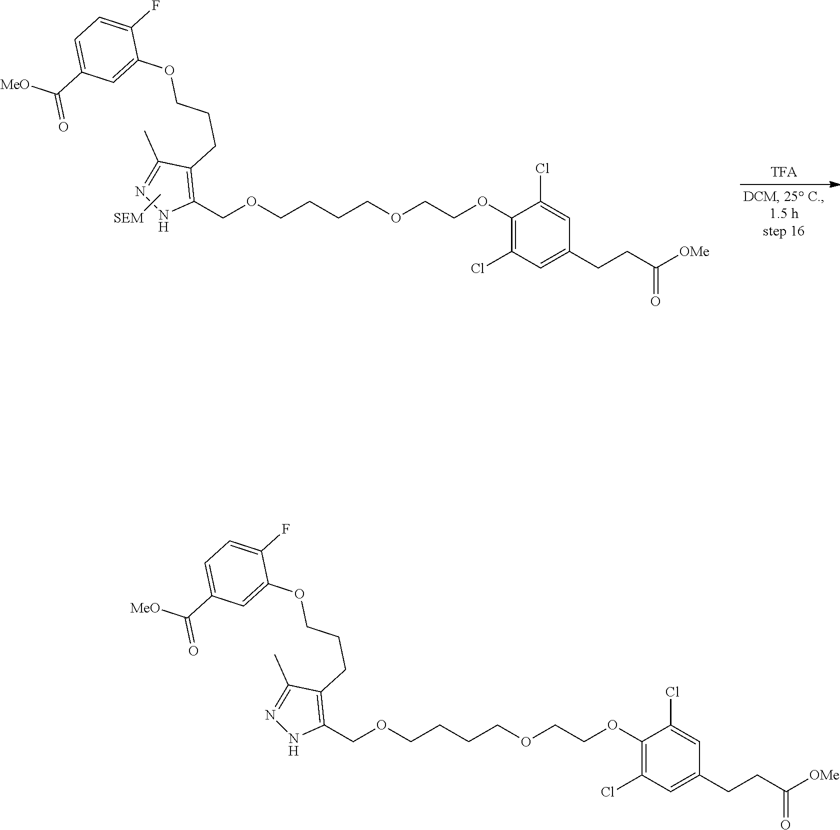

### Methyl 3-[3-[5-[4-[2-[2,6-dichloro-4-(3-methoxy-3-oxo-propyl)phenoxy]ethoxy]butoxy methyl]-3-methyl-1H-pyrazol-4-yl]propoxy]-4-fluoro-benzoate

To a solution of C₇₆H₁₀₆O₁₈Cl₄N₄F₂Si₂ (900 mg, 562.62 umol, 1 eq) in DCM (1 mL) was added TFA (7.70 g, 67.53 mmol, 5 mL, 115.71 eq). The reaction was stirred at 25° C. for 1 hr. The reaction was concentrated to give methyl 3-[3-[5-[4-[2-[2,6-dichloro-4-(3-methoxy-3-oxo-propyl)phenoxy]ethoxy]butoxymethyl]-3-methyl-1H-pyrazol-4-yl]propoxy]-4-fluoro-benzoate (1 g, crude) as a brown oil. The crude was used into next step without further purification.

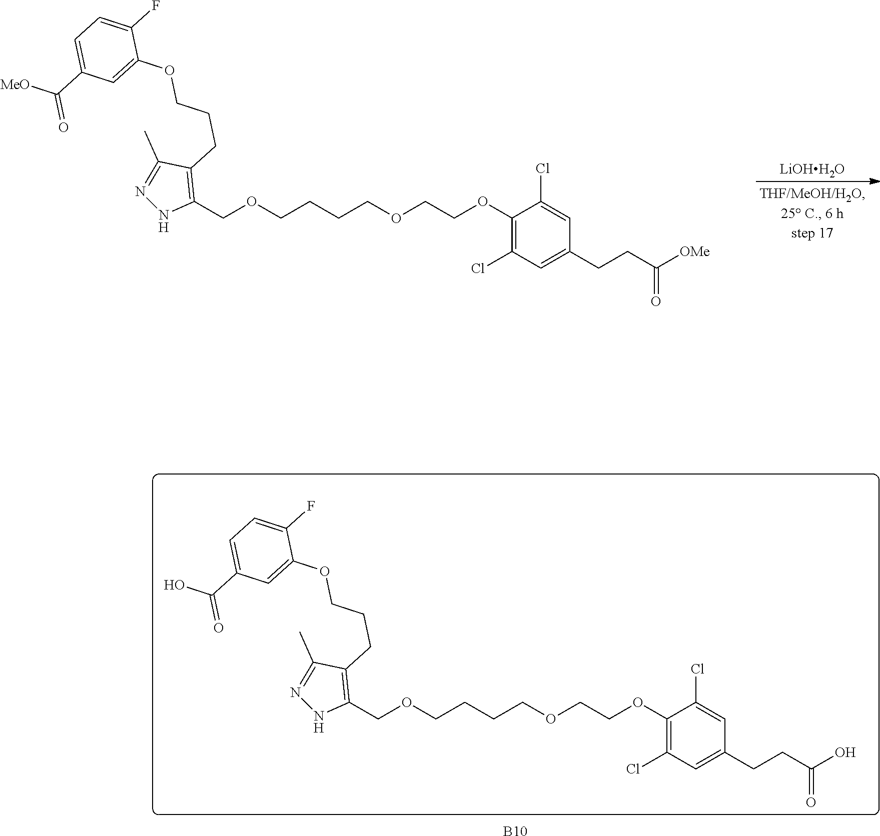 [SMILES: COC(=O)CCc1cc(Cl)c(OCCOCCCCOCc2[nH]nc(C)c2CCCOc2cc(C(=O)OC)ccc2F)c(Cl)c1 ; "LiOH·H2O" ; Cc1n[nH]c(COCCCCOCCOc2c(Cl)cc(CCC(=O)O)cc2Cl)c1CCCOc1cc(C(=O)O)ccc1F ; "B10"]

### 3-[3-[5-[4-[2-[4-(2-carboxyethyl)-2,6-dichloro-phenoxy]ethoxy]butoxymethyl]-3-methyl-1H-pyrazol-4-yl]propoxy]-4-fluoro-benzoic acid

To a solution of methyl 3-[3-[5-[4-[2-[2,6-dichloro-4-(3-methoxy-3-oxo-propyl)phenoxy]ethoxy]butoxymethyl]-3-methyl-1Hpyrazol-4-yl]propoxy]-4-fluoro-benzoate (1 g, 1.49 mmol, 1 eq) in THF (3.0 mL) and MeOH (1 mL) was added a solution of LiOH·H₂O (626.73 mg, 14.94 mmol, 10 eq) in H₂O (2 mL) at 25° C. The reaction was stirred at 25° C. for 4 hr.

The reaction (combined ET20197-211, 100 mg scale) was concentrated and adjusted with HCl (1N) to pH 5 ˜6. The mixture was extracted with 2-methyltetrahydrofuran (15 mL*3), washed with brine (20 mL*2). The organic layers were concentrated to give the residue. The residue was purified by prep-HPLC (HCl condition) to afford 3-[3-[5-[4-[2-[4-(2-carboxyethyl)-2,6-dichloro-phenoxy]ethoxy]butoxymethyl]-3-methyl-1H-pyrazol-4-yl]propoxy]-4-fluoro-benzoic acid (240.4 mg, 372.72 umol, 24.96% yield, 99.46% purity) as off-white solid.

LCMS: ET2197-213-P1P1 (M+H⁺): 641.2 @ 2.302 min (10-90% ACN in H₂O, 4.5 min)

¹H NMR: ET20197213-P1AA (400 MHz, METHANOL-d₄)

δ 7.70-7.61 (m, 2H), 7.26-7.16 (m, 3H), 4.62 (s, 2H), 4.15-4.06 (m, 4H), 3.81-3.74 (m, 2H), 3.55 (q, J=6.1 Hz, 4H), 2.87-2.80 (m, 2H), 2.76 (t, J=7.3 Hz, 2H), 2.62-2.55 (m, 2H), 2.39 (s, 3H), 2.06 (quin, J=6.4 Hz, 2H), 1.71-1.58 (m, 4H)

### Synthesis of B1 (T-753)

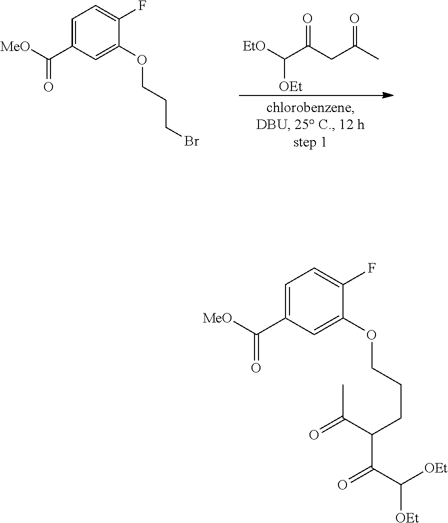 [SMILES: COC(=O)c1ccc(F)c(OCCCBr)c1 ; CCOC(OCC)C(=O)CC(C)=O ; CCOC(OCC)C(=O)C(CCCOc1cc(C(=O)OC)ccc1F)C(C)=O]

### Methyl 3-(4-acetyl-6,6-diethoxy-5-oxo-hexoxy)-4-fluoro-benzoate

To a solution of 1,1-diethoxypentane-2,4-dione (7.76 g, 41.22 mmol, 1.5 eq) and DBU (8.37 g, 54.96 mmol, 8.28 mL, 2 eq) in chlorobenzene (120 mL) was added methyl 3-(3-bromo propoxy)-4-fluoro-benzoate (8 g, 27.48 mmol, 1 eq) in portions. Then the mixture was stirred at 25° C. for 12 hr. The reaction was concentrated under reduce pressure to give the residue. The residue was purified by flash silica gel chromatography (ISCO®; 120 g Sepa Flash® Silica Flash Column, Eluent of 0 ˜20% Ethylacetate/Petroleum ether gradient @ 100 mL/min) to give methyl 3-(4-acetyl-6,6-diethoxy-5-oxo-hexoxy)-4-fluoro-benzoate (5 g, 12.55 mmol, 45.67% yield) as a light yellow oil.

¹H NMR: ET20197-154-P1AA (400 MHz, CHLOROFORM-d)

δ 7.68-7.60 (m, 2H), 7.15-7.07 (m, 1H), 4.62-4.52 (m, 1H), 4.24-4.14 (m, 1H), 4.12-4.05 (m, 2H), 3.93-3.88 (m, 3H), 3.75-3.67 (m, 2H), 3.63-3.47 (m, 2H), 2.37-2.26 (m, 3H), 2.14-1.73 (m, 4H), 1.26-1.20 (m, 6H)

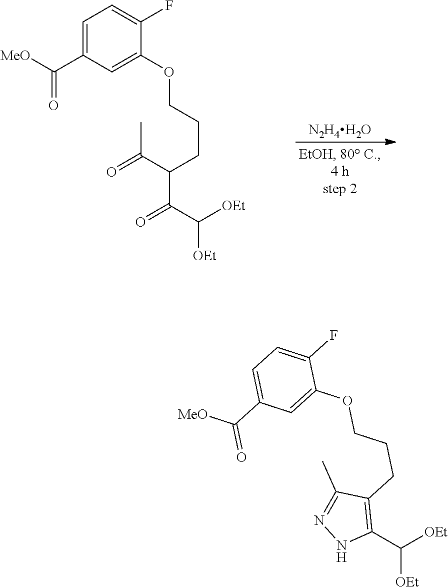 [SMILES: CCOC(OCC)C(=O)C(CCCOc1cc(C(=O)OC)ccc1F)C(C)=O ; "N2H4·H2O" ; CCOC(OCC)c1[nH]nc(C)c1CCCOc1cc(C(=O)OC)ccc1F]

### Methyl 3-[3-[5-(diethoxymethyl)-3-methyl-1H-pyrazol-4-yl]propoxy]-4-fluoro-benzoate

To a solution of methyl 3-(4-acetyl-6,6-diethoxy-5-oxo-hexoxy)-4-fluoro-benzoate (5 g, 12.55 mmol, 1 eq) in EtOH (50 mL) was added N₂H₄·H₂O (1.92 g, 37.65 mmol, 1.87 mL, 3 eq). The solution was stirred at 80° C. for 4 hr. The reaction was concentrated to the residue.

The residue was purified by flash silica gel chromatography (ISCO®; 40 g SepaFlash® Silica Flash Column, Eluent of 0 ˜15% Ethylacetate/Petroleum ethergradient @100 mL/min) to give methyl 3-[3-[5-(diethoxymethyl)-3-methyl-1H-pyrazol-4-yl]propoxy]-4-fluoro-benzoate (2.7 g, 6.85 mmol, 54.55% yield) as a light yellow oil.

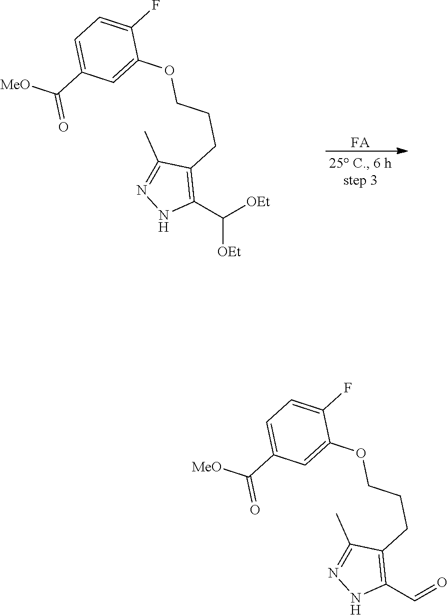 [SMILES: CCOC(OCC)c1[nH]nc(C)c1CCCOc1cc(C(=O)OC)ccc1F ; "FA" ; COC(=O)c1ccc(F)c(OCCCc2c(C)n[nH]c2C=O)c1]

### Methyl 4-fluoro-3-[3-(5-formyl-3-methyl-1H-pyrazol-4-yl)propoxy]benzoate

A solution of methyl 3-[3-[5-(diethoxymethyl)-3-methyl-1H-pyrazol-4-yl]propoxy]-4-fluoro-benzoate (3.3 g, 8.37 mmol, 1 eq) in formic acid (40 mL) was stirred at 25° C. for 6 hr. The reaction was concentrated under reduce pressure to give the residue as a yellow solid. The residue was stirred in MTBE (30 mL) at 25° C. for 0.5 h, filtered to give methyl 4-fluoro-3-[3-(5-formyl-3-methyl-1H-pyrazol-4-yl)propoxy]benzoate (2 g, 6.24 mmol, 74.63% yield) as a white solid. The crude product was used into the next step without further purification.

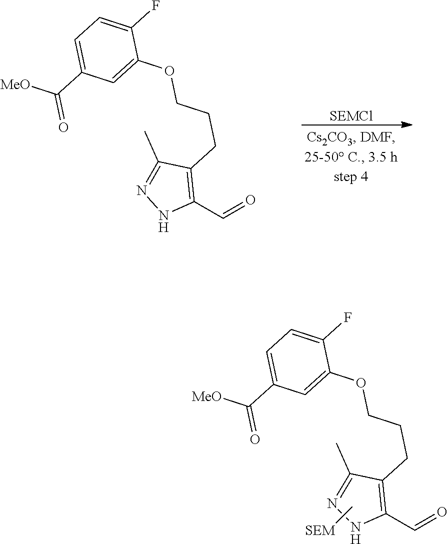

### Methyl 4-fluoro-3-(3-(5-formyl-3-methyl-1-((2-(trimethylsilyl)ethoxy)methyl)-1H-pyraz ol-4-yl)propoxy)benzoate and methyl 4-fluoro-3-(3-(3-formyl-5-methyl-1-((2-(trimethyl silyl)ethoxy)methyl)-1H-pyrazol-4-yl)propoxy)benzoate (C₄₄H₆₂O₁₀N₄F₂Si₂)

To a suspension of methyl 4-fluoro-3-[3-(5-formyl-3-methyl-1H-pyrazol-4-yl)propoxy]benz oate (1.85 g, 5.78 mmol, 1 eq) in anhydrous DMF (20 mL) was added Cs₂CO₃ (5.65 g, 17.33 mmol, 3 eq) at 25° C. The mixture was stirred at 25° C. for 30 minutes. After 30 minutes, SEM-Cl (1.93 g, 11.55 mmol, 2.04 mL, 2 eq) was added into the mixture. The resulting mixture was stirred at 50° C. for 3 hours. The reaction was quenched with H₂O (70 mL), and extracted with EtOAc (50 mL*3). The organic was washed with brine (20 mL), dried over Na₂SO₄, filtered and concentrated to give the residue. The residue was purified by flash silica gel chromatography (ISCO®; 20 g SepaFlash® Silica Flash Column, Eluent of 0 ˜15% ethylacetate/Petroleum ether gradient @ 75 mL/min) to give a mixture of two isomers C₄₄H₆₂O₁₀N₄F₂Si₂ (2.2 g, 2.21 mmol, 38.33% yield, 90.68% purity) as a colorless oil. The structure of the product was not influenced so the product was used into next step.

¹H NMR: ET20197-174-P1AA (400 MHz, CHLOROFORM-d)

δ 10.01 (s, 1H), 7.65-7.56 (m, 2H), 7.14-7.06 (m, 1H), 5.43 (s, 2H), 4.07-3.98 (m, 2H), 3.88 (d, J=0.9 Hz, 3H), 3.59-3.48 (m, 2H), 2.98-2.82 (m, 2H), 2.28-2.19 (m, 3H), 2.12-2.05 (m, 2H), 0.91-0.82 (m, 2H), 0.03-0.09 (m, 9H).

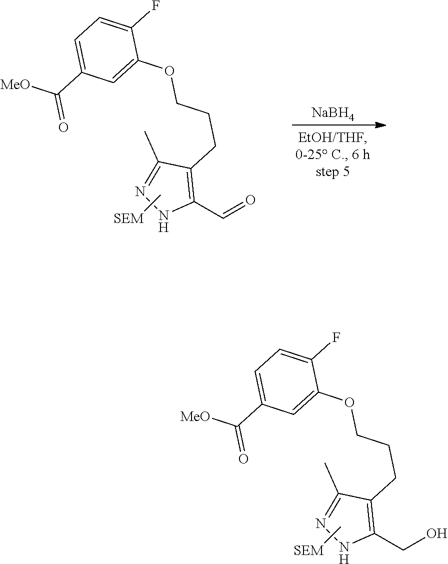

### Methyl 4-fluoro-3-(3-(3-(hydroxymethyl)-5-methyl-1-((2-(trimethylsilyl)ethoxy)methyl)-1H-pyrazol-4-yl)propoxy)benzoate and methyl 4-fluoro-3-(3-(5-(hydroxymethyl)-3-methyl-1-((2-(trimethylsilyl)ethoxy)methyl)-1H-pyrazol-4-yl)propoxy)benzoate (C₄₄H₆₆O₆₀N₄F₂Si₂)

To a solution of C₄₄H₆₂O₁₀N₄F₂Si₂ (2.2 g, 2.44 mmol, 1 eq) in EtOH (30 mL)/THF (15 mL) was added NaBH₄ (646.49 mg, 17.09 mmol, 7 eq) at 0° C. The reaction was allowed to warm to 25° C. and stirred for 6 h at 25° C. The reaction was quenched with HCl (1N) to pH˜6, concentrated to give a residue. The residue was partitioned between H₂O (40 mL) and EtOAc (50 mL*3). The organic layer was dried over Na₂SO₄ and concentrated to give a residue.

The residue was purified by column (SiO₂, PE/EtOAc=20/1 ˜1/1) to a mixture of two isomers C₄₄H₆₆O₁₀N₄F₂Si₂ (1.7 g, 1.84 mmol, 75.39%,o yield, 98% purity) as colorless oil.

¹H NMR: ET20197-175-P1BB (400 MHz, CHLOROFORM-d)

δ 7.66-7.59 (m, 2H), 7.12 (dd, J=8.8, 11.0 Hz, 1H), 5.44-5.33 (s, 1H), 4.66-4.58 (m, 2H), 4.09-4.00 (m, 2H), 3.90 (s, 3H), 3.59-3.49 (m, 2H), 2.66 (t, J=7.3 Hz, 2H), 2.22 (d, J=10.5 Hz, 3H), 2.10-1.96 (m, 2H), 0.92-0.81 (m, 2H), 0.02-0.06 (m, 9H)

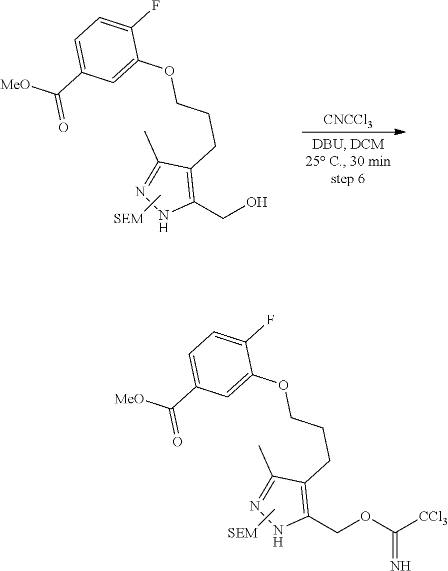

### Methyl 4-fluoro-3-(3-(3-methyl-5-((2,2,2-trichloro-1-iminoethoxy)methyl)-1-((2-(trimethylsilyl)ethoxy)methyl)-1H-pyrazol-4-yl)propoxy)benzoate and methyl 4-fluoro-3-(3-(5-methyl-3-((2,2,2-trichloro-1-iminoethoxy)methyl)-1-((2-(trimethylsilyl) ethoxy)methyl)-1H-pyrazol-4-yl)propoxy)benzoate (C₄₈H₆₆O₁₀N₆F₂Si₂Cl₆)

To a solution of C₄₄H₆₆O₁₀N₄F₂Si₂ (800 mg, 883.80 umol, 1 eq) and 2,2,2-trichloro acetonitrile (1.28 g, 8.84 mmol, 888.89 uL, 10 eq) in DCM (5 mL) was added DBU (59.22 mg, 388.96 umol, 58.63 uL, 0.44 eq) at 25° C. The reaction was stirred at 25° C. for 0.5 h. The reaction was concentrated to give C₄₈H₆₆O₁₀N₆F₂Si₂Cl₆ (1.2 g, crude) as a brown oil. The crude was used into the next step without further purification.

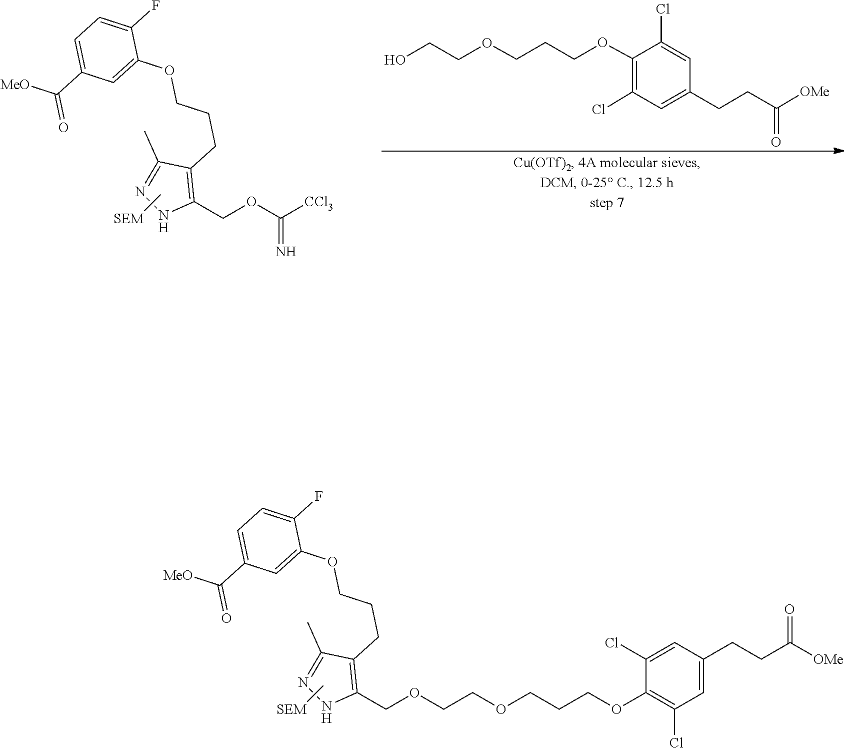

### Methyl 3-(3-(5-((2-(3-(2,6-dichloro-4-(3-methoxy-3-oxopropyl)phenoxy)propoxy) ethoxy)methyl)-3-methyl-1l-((2-(trimethylsilyl)ethoxy)methyl)-1H-pyrazol-4-yl) propoxy)-4-fluorobenzoate and methyl 3-(3-(3-((2-(3-(2,6-dichloro-4-(3-methoxy-3-oxopropyl)phenoxy)propoxy)ethoxy)methyl)-5-methyl-1-((2-(trimethylsilyl)ethoxy) methyl)-1H-pyrazol-4-yl)propoxy)-4-fluorobenzoate (C₇₄H₁₀₂O₁₈N₄F₂Cl₄Si₂)

A solution of methyl 3-[3,5-dichloro-4-[3-(2-hydroxyethoxy)propoxy]phenyl]propanoate (350 mg, 996.52 umol, 1 eq) in DCM (10 mL) is cooled to 0° C. A solution of C₄₈H₆₆O₁₀N₆F₂Cl₆Si₂ (1.2 g, 1.01 mmol, 1.01 eq) in DCM (10 mL) and 4A molecular sieves (1 g, 1.00 eq) were added. After 30 min, bis(trifluoromethylsulfonyloxy)copper (360.42 mg, 996.52 umol, 1 eq) was added at 0° C. The reaction was stirred at 25° C. for 12 h. The reaction mixture was filtered and the filtrate was washed with sat. aq. NaHCO₃ (10 mL*2), H₂O (20 mL), brine (20 mL). The organic layers were dried over Na₂SO₄, filtered and concentrated. The residue was purified by column (SiO₂, 2-methyltetrahydrofuran/Petroleum ether=20/1 ˜5/1) to afford C₇₄H₁₀₂O₁₈N₄F₂Cl₄Si₂ (0.4 g, 75.68 umol, 7.59% yield, 30% purity) as a light brown oil.

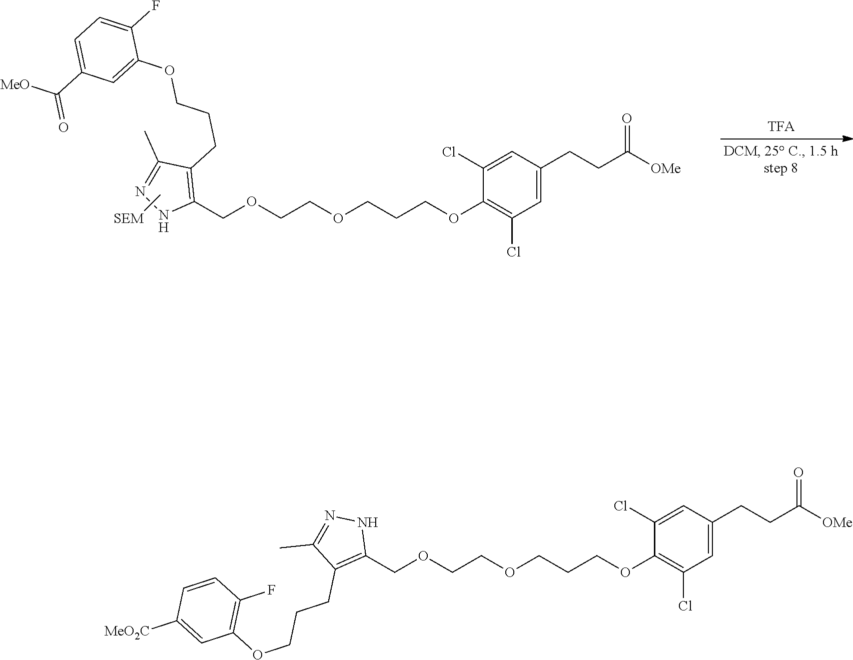

### Methyl 3-[3-[5-[2-[3-[2,6-dichloro-4-(3-methoxy-3-oxo-propyl)phenoxy]propoxy]ethoxy methyl]-3-methyl-1H-pyrazol-4-yl]propoxy]-4-fluoro-benzoate (400 mg, crude) was obtained as a brown oil

To a solution of C₇₄H₁₀₂O₁₈C₁₄N₄F₂Si₂ (0.38 g, 72.54 umol, 1 eq) in DCM (1 mL) was added TFA (7.70 g, 67.53 mmol, 5 mL, 930.97 eq). The reaction was stirred at 25° C. for 1.5 hr. The reaction was concentrated to give methyl 3-[3-[5-[2-[3-[2,6-dichloro-4-(3-methoxy-3-oxo-propyl)phenoxy]propoxy]ethoxymethyl]-3-methyl-1H-pyrazol-4-yl]propoxy]-4-fluoro-benzoate (400 mg, crude) as a brown oil. The crude was used into next step without further purification.

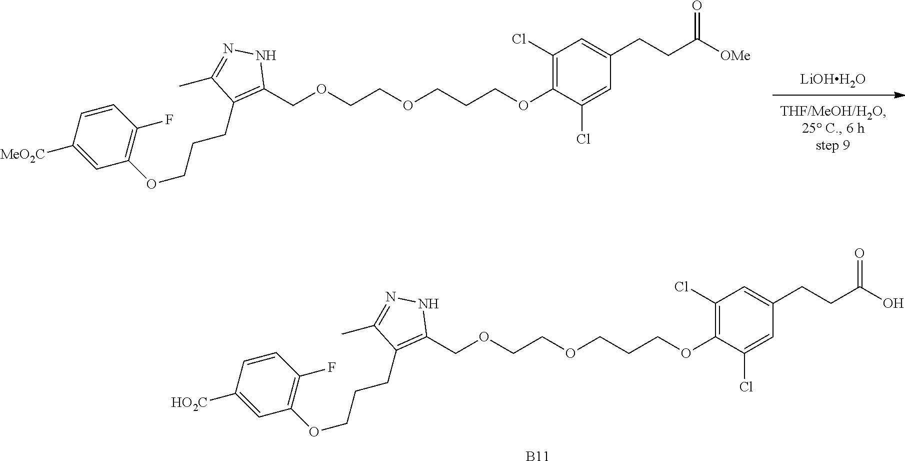 [SMILES: COC(=O)CCc1cc(Cl)c(OCCCOCCOCc2[nH]nc(C)c2CCCOc2cc(C(=O)OC)ccc2F)c(Cl)c1 ; "LiOH·H2O" ; Cc1n[nH]c(COCCOCCCOc2c(Cl)cc(CCC(=O)O)cc2Cl)c1CCCOc1cc(C(=O)O)ccc1F ; "B11"]

### 3-[3-[5-[2-[3-[4-(2-carboxyethyl)-2,6-dichloro-phenoxy]propoxy]ethoxymethyl]-3-methyl-1H-pyrazol-4-yl]propoxy]-4-fluoro-benzoic acid

To a solution of methyl 3-[3-[5-[2-[3-[2,6-dichloro-4-(3-methoxy-3-oxopropyl)phenoxy]propoxy]ethoxymethyl]-3-methyl-1H-pyrazol-4-yl]propoxy]-4-fluorobenzoate (400 mg, 610.19 umol, 1 eq) in THF (3 mL) and MeOH (1 mL) was added a solution of LiOH·H₂O (256.03 mg, 6.10 mmol, 10 eq) in H₂O (1.5 mL). The reaction was stirred at 25° C. for 3 hr. The reaction was concentrated and adjusted with HCl (1N) to pH 5 ˜6. The mixture was extracted with 2-methyltetrahydrofuran (15 mL*3), washed with brine (20 mL*2). The organic layers were concentrated to give the residue. The residue was purified by prep-HPLC (HCl condition) to give 3-[3-[5-[2-[3-[4-(2-carboxyethyl)-2,6-dichloro-phenoxy]propoxy]ethoxymethyl]-3-methyl-1H-pyrazol-4-yl]propoxy]-4-fluoro-benzoic acid (92 mg, 146.62 umol, 24.03% yield, 100% purity) as off-white solid.

LCMS: ET20197-202-P1P (M+H⁺): 627.2 @ 2.389 min (25-100% ACN in H₂O, 4.5 min)

¹H NMR: ET20197-202-P1BB (400 MHz, METHANOL-d₄)

δ 7.68-7.59 (m, 2H), 7.25-7.13 (m, 3H), 4.63 (s, 2H), 4.09-3.99 (m, 4H), 3.74-3.60 (m, 6H), 2.86-2.79 (m, 2H), 2.72 (t, J=7.3 Hz, 2H), 2.61-2.55 (m, 2H), 2.30 (s, 3H), 2.04 (quin, J=6.2 Hz, 4H)

2-(3-bromopropoxy)ethanol was prepared as described above in relation to the synthesis of Compound B1 as step 10 of the current synthesis of Compound B11

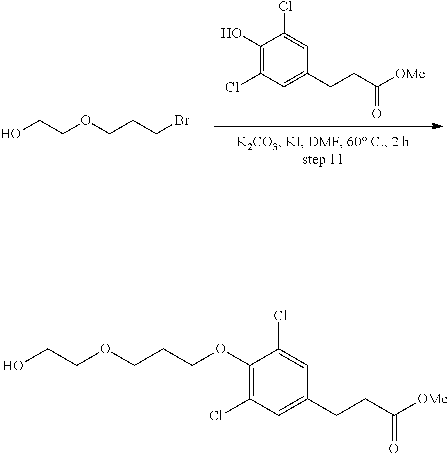 [SMILES: COC(=O)CCc1cc(Cl)c(O)c(Cl)c1 ; OCCOCCCBr ; COC(=O)CCc1cc(Cl)c(OCCCOCCO)c(Cl)c1]

### Methyl 3-[3,5-dichloro-4-[3-(2-hydroxyethoxy)propoxy]phenyl]propanoate

To a solution of methyl 3-(3,5-dichloro-4-hydroxy-phenyl)propanoate (700 mg, 2.81 mmol, 1 eq) and 2-(3-bromopropoxy)ethanol (857.32 mg, 2.81 mmol, 1 eq) in DMF (56 mL) was added K₂CO₃ (1.17 g, 8.43 mmol, 3 eq) and KI (466.50 mg, 2.81 mmol, 1 eq). The mixture was stirred at 60° C. for 2 hr. The reaction mixture was diluted with H₂O (60 mL) and extracted with EtOAc (50 mL*3). The combined organic layers were washed with brine (80 mL), dried over Na₂SO₄, filtered and concentrated under reduced pressure to give a residue. The residue was purified by flash silica gel chromatography (ISCO®; 20 g SepaFlash® Silica Flash Column, Eluent of 0 ˜30% Ethylacetate/Petroleum ether gradient @ 50 mL/min) to give methyl 3-[3,5-dichloro-4-[3-(2-hydroxyethoxy)propoxy]phenyl]propanoate (1 g, crude, 60% purity) as a yellow oil.

¹H NMR: ET21585-10-P1AA (400 MHz, CHLOROFORM-d)

δ 7.12 (s, 2H), 4.06 (t, J=12.0 Hz, 2H), 3.74-3.72 (m, 5H), 3.67 (s, 3H), 3.67-3.55 (m, 3H), 2.59 (t, J=15.2 Hz, 2H), 2.12-2.10 (m, 2H)

### Synthesis of B13 (T-747)

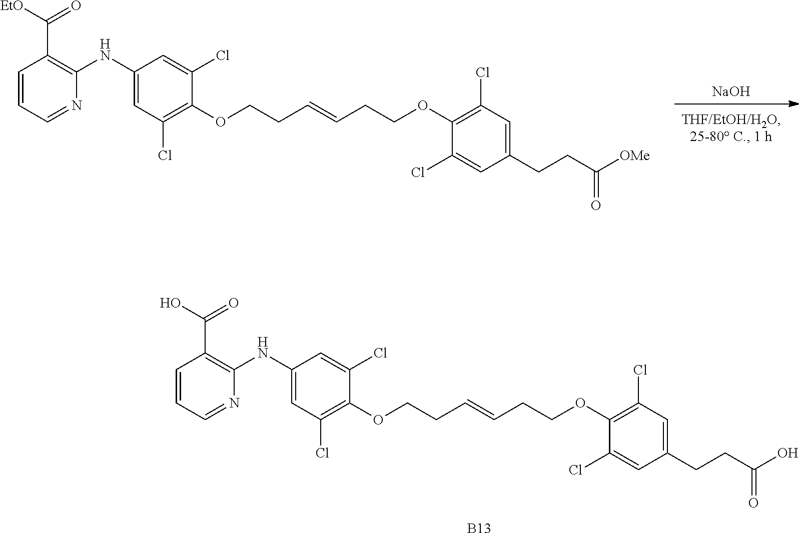 [SMILES: CCOC(=O)c1cccnc1Nc1cc(Cl)c(OCC/C=C/CCOc2c(Cl)cc(CCC(=O)OC)cc2Cl)c(Cl)c1 ; "NaOH" ; O=C(O)CCc1cc(Cl)c(OCC/C=C/CCOc2c(Cl)cc(Nc3ncccc3C(=O)O)cc2Cl)c(Cl)c1 ; "B13"]

### (E)-2-((4-((6-(4-(2-carboxyethyl)-2,6-dichlorophenoxy)hex-3-en-1-yl)oxy)-3,5-dichlorophenyl)amino)nicotinic acid

To a solution of ethyl 2-[3,5-dichloro-4-[(E)-6-[2,6-dichloro-4-(3-methoxy-3-oxo-propyl) phenoxy]hex-3-enoxy]anilino]pyridine-3-carboxylate (300 mg, 457.05 umol, 1 eq) in EtOH (4.5 mL), H₂O (2.5 mL) and THF (2 mL) was added NaOH (95.06 mg, 2.38 mmol, 5.2 eq) at 25° C., then the reaction mixture was stirred at 80° C. for 1 hr. The reaction mixture was concentrated to give a residue. The residue was suspended with H₂O (3 mL) and acidified to pH=6 with HCl (1 N). The suspension was extracted with EtOAc (10 mL*3). The organic layer was dried over Na₂SO₄ and concentrated to give a residue. The residue was purified by neutral prep-HPLC (HPLC: ET20960-56-P1Z) to afford 2-[4-[(E)-6-[4-(2-carboxyethyl)-2,6-dichloro-phenoxy]hex-3-enoxy]-3,5-dichloro-anilino]pyridine-3-carboxylic acid (134.2 mg, 215.49 umol, 47.15% yield, 98.64% purity) as a light-yellow solid.

LCMS: ET20960-56-P1A2 (M+H⁺): 615.0 @ 1.543 min (10-80% ACN in H₂O, 4.5 min)

¹H NMR: ET20960-56-P1AB (METHANOL-d4, 400 MHz)

δ 8.38-8.28 (m, 2H), 7.80 (s, 2H), 7.25 (s, 2H), 6.85 (dd, J=5.5, 7.0 Hz, 1H), 5.75 (br t, J=4.7 Hz, 2H), 4.01 (dt, J=3.4, 6.7 Hz, 4H), 2.85 (t, J=7.4 Hz, 2H), 2.64-2.50 (m, 6H).

### Synthesis of B14 (T-748)

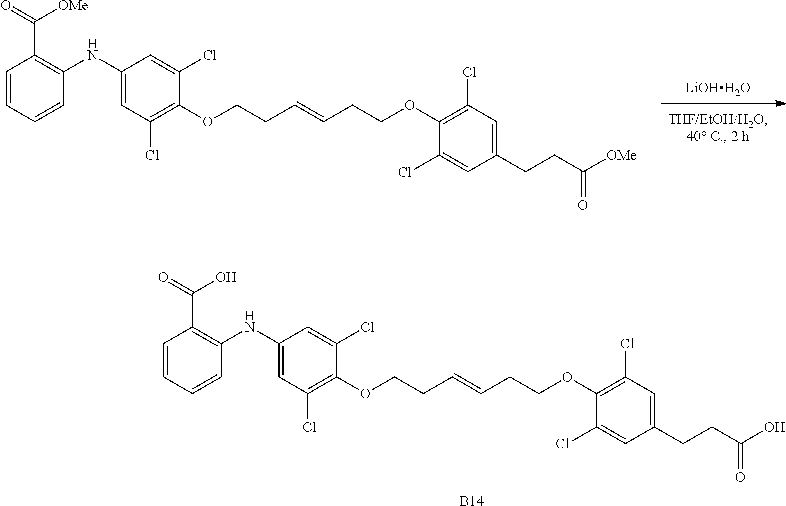 [SMILES: COC(=O)CCc1cc(Cl)c(OCC/C=C/CCOc2c(Cl)cc(Nc3ccccc3C(=O)OC)cc2Cl)c(Cl)c1 ; "LiOH·H2O" ; O=C(O)CCc1cc(Cl)c(OCC/C=C/CCOc2c(Cl)cc(Nc3ccccc3C(=O)O)cc2Cl)c(Cl)c1 ; "B14"]

### 2-[4-[(E)-6-[4-(2-carboxyethyl)-2,6-dichloro-phenoxy]hex-3-enoxy]-3,5-dichloro-anilino]benzoic acid

To a solution of methyl 2-[3,5-dichloro-4-[(E)-6-[2,6-dichloro-4-(3-methoxy-3-oxo-propyl) phenoxy]hex-3-enoxy]anilino]benzoate (300 mg, 467.75 umol, 1 eq) in THF (12.5 mL) and MeOH (12.5 mL) was added LiOH·H₂O (117.76 mg, 2.81 mmol, 6 eq) in H₂O (15 mL). The mixture was stirred at 40° C. for 2 hr. The reaction mixture was diluted with H₂O 30 mL and acidified with HCl (6 N) to pH=4, then extracted with EtOAc 30 mL×2. The combined organic layers were washed with brine 50 mL, dried over Na₂SO₄, filtered and concentrated under reduced pressure to give a residue. The residue was purified by prep-HPLC (FA condition) to give Product 150 mg, the purity was 96.9%. Then it was further purified by prep-HPLC (neutral condition) to afford 2-[4-[(E)-6-[4-(2-carboxyethyl)-2,6-dichloro-phenoxy]hex-3-enoxy]-3,5-dichloro-anilino]benzoic acid (58 mg, 93.97 umol, 20.09% yield, 99.37% purity) as a yellow solid.

LCMS: ET21585-35-P1A2 (M+H⁺): 614.0 @ 3.119 min (10-80% ACN in H₂O, 4.5 min)

¹H NMR: ET21585-35-P1BB (Acetone-d6, 400 MHz)

δ 8.04 (d, J=7.9 Hz, 1H), 7.49-7.43 (1H), 7.36-7.26 (m, 5H), 6.88 (t, J=7.5 Hz, 1H), 5.78 (t, J=3.6 Hz, 2H), 4.09-3.99 (m, 4H), 2.91-2.85 (m, 2H), 2.68-2.62 (m, 2H), 2.61-2.54 (m, 4H)

### Synthesis of B15 (T-754)

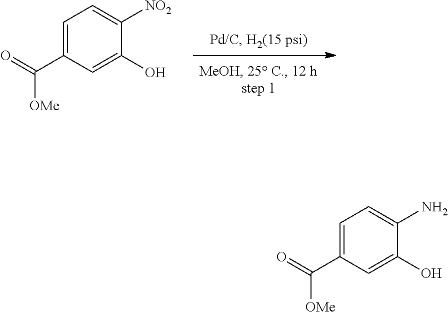 [SMILES: COC(=O)c1ccc([N+](=O)[O-])c(O)c1 ; "Pd/C, H2(15 psi)" ; COC(=O)c1ccc(N)c(O)c1]

### Methyl 4-amino-3-hydroxy-benzoate

To a solution of methyl 3-hydroxy-4-nitro-benzoate (10 g, 50.72 mmol, 1 eq) in MeOH (200 mL) was added Pd/C (10 g, 10% purity) under N₂. The suspension was degassed under vacuum and purged with H₂ several times. The mixture was stirred under H₂ (15 psi) at 25° C. for 12 hr. The reaction was filtered and concentrated to give the residue. The residue was purified by flash silica gel chromatography (ISCO®; 40 g SepaFlash® Silica Flash Column, Eluent of 0 ˜25% Ethylacetate/Petroleum ether gradient @ 100 mL/min) to afford methyl 4-amino-3-hydroxy-benzoate (8 g, 47.86 mmol, 94.35% yield) as a brown solid.

¹H NMR: ET20197-219-P1AA (400 MHz, CHLOROFORM-d)

δ 7.58 (d, J=1.8 Hz, 1H), 7.51 (dd, J=1.8, 8.2 Hz, 1H), 6.69 (d, J=8.2 Hz, 1H), 5.92 (br s, 1H), 4.20 (br s, 2H), 3.87 (s, 3H)

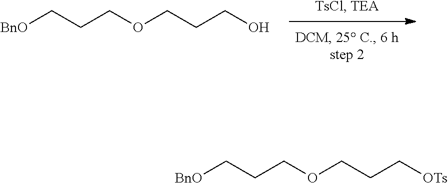 [SMILES: "TsCl, TEA" ; OCCCOCCCOCc1ccccc1 ; Cc1ccc(S(=O)(=O)OCCCOCCCOCc2ccccc2)cc1]

### 3-(3-benzyloxypropoxy)propyl 4-methylbenzenesulfonate

To a mixture of 3-(3-benzyloxypropoxy)propan-1-ol (1 g, 4.46 mmol, 1 eq) and TEA (902.29 mg, 8.92 mmol, 1.24 mL, 2 eq) in DCM (10 mL) was added a solution of TosCl (1.02 g, 5.35 mmol, 1.2 eq) in DCM (5 mL) drop wise, the reaction mixture was stirred at 25° C. for 6 hr. The reaction was concentrated to give the residue. The residue was purified by column (SiO₂, Petroleum ether/EtOAc=30:1˜5:1) to afford 3-(3-benzyloxypropoxy)propyl 4-methyl benzenesulfonate (1.3 g, 3.43 mmol, 77.04% yield) as a colorless oil.

¹H NMR: ET20197-222-P1AA (400 MHz, CHLOROFORM-d)

δ 7.79 (d, J=8.3 Hz, 2H), 7.38-7.26 (m, 7H), 4.49 (s, 2H), 4.13 (t, J=6.3 Hz, 2H), 3.51 (t, J=6.3 Hz, 2H), 3.46-3.38 (m, 4H), 2.44 (s, 3H), 1.94-1.75 (m, 4H)

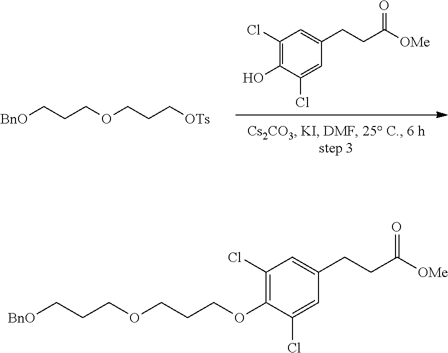 [SMILES: COC(=O)CCc1cc(Cl)c(O)c(Cl)c1 ; Cc1ccc(S(=O)(=O)OCCCOCCCOCc2ccccc2)cc1 ; COC(=O)CCc1cc(Cl)c(OCCCOCCCOCc2ccccc2)c(Cl)c1]

### Methyl 3-[4-[3-(3-benzyloxypropoxy)propoxy]-3,5-dichloro-phenyl]propanoate

To a solution of 3-(3-benzyloxypropoxy)propyl 4-methylbenzenesulfonate (1.3 g, 3.43 mmol, 1 eq) and methyl 3-(3,5-dichloro-4-hydroxy-phenyl)propanoate (1.03 g, 4.12 mmol, 1.2 eq) in DMF (20 mL) was added K₂CO₃ (1.42 g, 10.30 mmol, 3 eq) and KI (57.02 mg, 343.48 umol, 0.1 eq) at 25° C., and then the reaction mixture was stirred at 25° C. for 6 hr. The reaction mixture was diluted with H₂O (20 mL) and extracted with EtOAc (50 mL*4). The combined organic layers were washed with brine (20 mL*2), dried over Na₂SO₄, filtered and concentrated under reduced pressure to give a residue. The residue was purified by flash silica gel chromatography (ISCO®; 20 g SepaFlash® Silica Flash Column, Eluent of 0 ˜10% Ethylacetate/Petroleum ethergradient @ 75 mL/min) to afford methyl 3-[4-[3-(3-benzyloxy propoxy)propoxy]-3,5-dichloro-phenyl]propanoate (1.5 g, 3.29 mmol, 95.90% yield) as a colorless oil.

¹H NMR: ET20197-229-P1AA (400 MHz, CHLOROFORM-d)

δ 7.36-7.24 (m, 5H), 7.14-7.10 (m, 2H), 4.51 (s, 2H), 4.11-4.04 (m, 2H), 3.70-3.65 (m, 5H), 3.58 (dt, J=2.5, 6.3 Hz, 4H), 2.91-2.82 (m, 2H), 2.65-2.57 (m, 2H), 2.13-2.06 (m, 2H), 1.91 (quin, J=6.4 Hz, 2H)

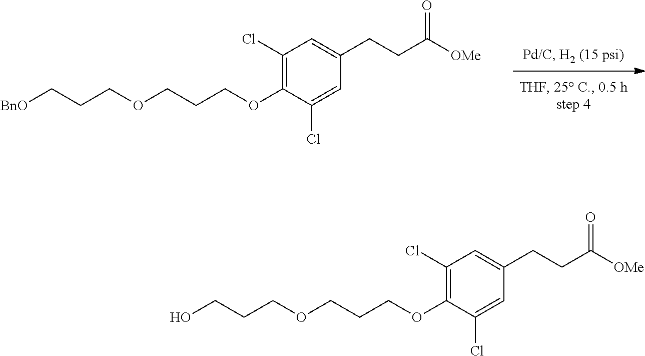 [SMILES: COC(=O)CCc1cc(Cl)c(OCCCOCCCOCc2ccccc2)c(Cl)c1 ; COC(=O)CCc1cc(Cl)c(OCCCOCCCO)c(Cl)c1]

### Methyl 3-[3,5-dichloro-4-[3-(3-hydroxypropoxy)propoxy]phenyl]propanoate

To a solution of methyl 3-[4-[3-(3-benzyloxypropoxy)propoxy]-3,5-dichloro-phenyl]propanoate (1.3 g, 2.85 mmol, 1 eq) in THF (20 mL) was added Pd/C (180 mg, 10% purity) under N₂. The suspension was degassed under vacuum and purged with H₂ several times. The mixture was stirred under H₂ (15 psi) at 25° C. for 0.5 hr. The reaction was filtered through a pad of Celite and the filtrate was concentrated to give the crude product. The crude was purified by prep-HPLC (neutral condition) to afford methyl 3-[3,5-dichloro-4-[3-(3-hydroxypropoxy)propoxy]phenyl]propanoate (700 mg, 1.92 mmol, 67.13% yield) as a white solid.

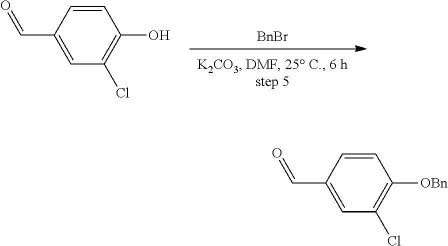 [SMILES: O=Cc1ccc(O)c(Cl)c1 ; "BnBr" ; O=Cc1ccc(OCc2ccccc2)c(Cl)c1]

### 4-benzyloxy-3-chloro-benzaldehyde

To a mixture of 3-chloro-4-hydroxy-benzaldehyde (5 g, 31.94 mmol, 1 eq) and K₂CO₃ (8.83 g, 63.87 mmol, 2 eq) in DMF (50 mL) was added bromomethylbenzene (6.55 g, 38.32 mmol, 4.55 mL, 1.2 eq), the reaction mixture was stirred at 25° C. for 12 hr. The reaction mixture was combined with a batch (ET20197-221, 1 g scale) and partitioned between H₂O (500 mL) and EtOAc (800 mL). The organic layer was washed with sub saturated brine (300 mL*3), saturated brine (300 mL), dried over Na₂SO₄ and concentrated to give a residue. The residue was purified by silica gel column (PE:EtOAc=20:1 to 5:1) to afford 4-benzyloxy-3-chloro-benzaldehyde (8.7 g) as a white solid.

¹H NMR: ET20197-227-P1AA (400 MHz, CHLOROFORM-d)

δ 9.86 (s, 1H), 7.94 (d, J=2.1 Hz, 1H), 7.74 (dd, J=2.0, 8.5 Hz, 1H), 7.51-7.33 (m, 5H), 7.09 (d, J=8.4 Hz, 1H), 5.27 (s, 2H)

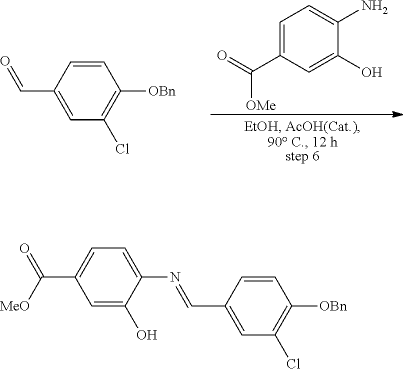 [SMILES: O=Cc1ccc(OCc2ccccc2)c(Cl)c1 ; COC(=O)c1ccc(N)c(O)c1 ; "EtOH, AcOH(Cat.),90° C., 12 hstep 6" ; COC(=O)c1ccc(/N=C/c2ccc(OCc3ccccc3)c(Cl)c2)c(O)c1]

### Methyl 4-[(E)-(4-benzyloxy-3-chloro-phenyl)methyleneamino]-3-hydroxy-benzoate

To a solution of 4-benzyloxy-3-chloro-benzaldehyde (7.7 g, 31.21 mmol, 1 eq) and methyl 4-amino-3-hydroxy-benzoate (5.22 g, 31.21 mmol, 1 eq) in EtOH (200 mL) was added AcOH (1.87 g, 31.21 mmol, 1.79 mL, 1 eq), the reaction mixture was stirred at 90° C. for 12 hr. The reaction was concentrated to give the crude product as a yellow solid. The crude product was triturated with MeOH (100 mL) at 25° C. for 30 min. The yellow suspension were filtered. The filter cake was washed with cooled MeOH (50 mL) to afford methyl 4-[(E)-(4-benzyloxy-3-chloro-phenyl)methyleneamino]-3-hydroxy-benzoate (11.5 g, 29.05 mmol, 93.08°/c yield) as a yellow solid.

¹H NMR: ET20197-233-P1AA (400 MHz, CHLOROFORM-d)

δ 8.60 (s, 1H), 8.05 (d, J=2.1 Hz, 1H), 7.74 (dd, J=2.1, 8.6 Hz, 1H), 7.69 (d, J=1.8, Hz, 1H), 7.63 (dd, J=1.8, 8.3 Hz, 1H), 7.53-7.48 (m, 2H), 7.47-7.42 (m, 2H), 7.41-7.35 (m, 1H), 7.29 (s, 1H), 7.14-7.05 (m, 2H), 5.28 (s, 2H), 3.94 (s, 3H)

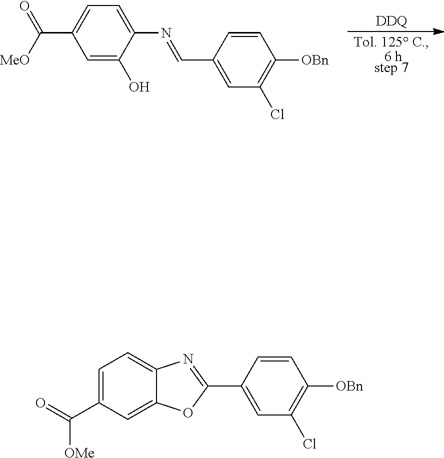

### Methyl 2-(4-benzyloxy-3-chloro-phenyl)-1,3-benzoxazole-6-carboxylate

To a yellow solution of methyl 4-[(E)-(4-benzyloxy-3-chloro-phenyl)methyleneamino]-3-hydroxy-benzoate (11.5 g, 29.05 mmol, 1 eq) in Tol. (400 mL) was added DDQ (13.19 g, 58.11 mmol, 2 eq) gradually. Then the reaction mixture was stirred at 125° C. for 6 hr. The reaction was concentrated to give the residue as a black red solid. The residue was triturated with 2-methyltetrahydrofuran (150 mL) and filtered to give the crude. The filter cake was washed with 2-methyltetrahydrofuran (100 mL) to afford methyl 2-(4-benzyloxy-3-chloro-phenyl)-1,3-benzoxazole-6-carboxylate (9 g, crude) as a light orange solid. The crude product was used into the next step without further purification.

¹H NMR: ET20197-233-P1AA (400 MHz, DMSO-d₆)

δ 8.29 (d, J=1.0 Hz, 1H), 8.26 (d, J=2.2 Hz, 1H), 8.20 (dd, J=2.2, 8.7 Hz, 1H), 8.06 (dd, J=1.5, 8.4 Hz, 1H), 7.88 (d, J=8.3 Hz, 1H), 7.57-7.51 (m, 3H), 7.47-7.33 (m, 3H), 5.38 (s, 2H), 3.93 (s, 3H)

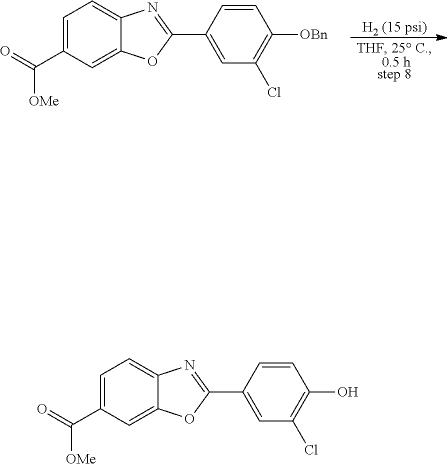 [SMILES: COC(=O)c1ccc2nc(-c3ccc(OCc4ccccc4)c(Cl)c3)oc2c1 ; "H2 (15 psi)" ; "THF, 25° C., 0.5 hstep 8" ; COC(=O)c1ccc2nc(-c3ccc(O)c(Cl)c3)oc2c1]

### Methyl 2-(3-chloro-4-hydroxy-phenyl)-1,3-benzoxazole-6-carboxylate

To a solution of methyl 2-(4-benzyloxy-3-chloro-phenyl)-1,3-benzoxazole-6-carboxylate (1 g, 2.54 mmol, 1 eq) in THF (20 mL) was added Pd/C (0.5 g, 10% purity) under N₂. The suspension was degassed under vacuum and purged with H₂ several times. The mixture was stirred under H₂ (15 psi) at 25° C. for 0.5 hr. The reaction mixture was combined with batches of ET20197-238 (100 mg) and ET20197-239 (100 mg). The reaction mixture was filtered and the filtrate was concentrated to afford methyl 2-(3-chloro-4-hydroxy-phenyl)-1,3-benzoxazole-6-carboxylate (1.07 g) as a off white solid. The crude product was used into the next step without further purification.

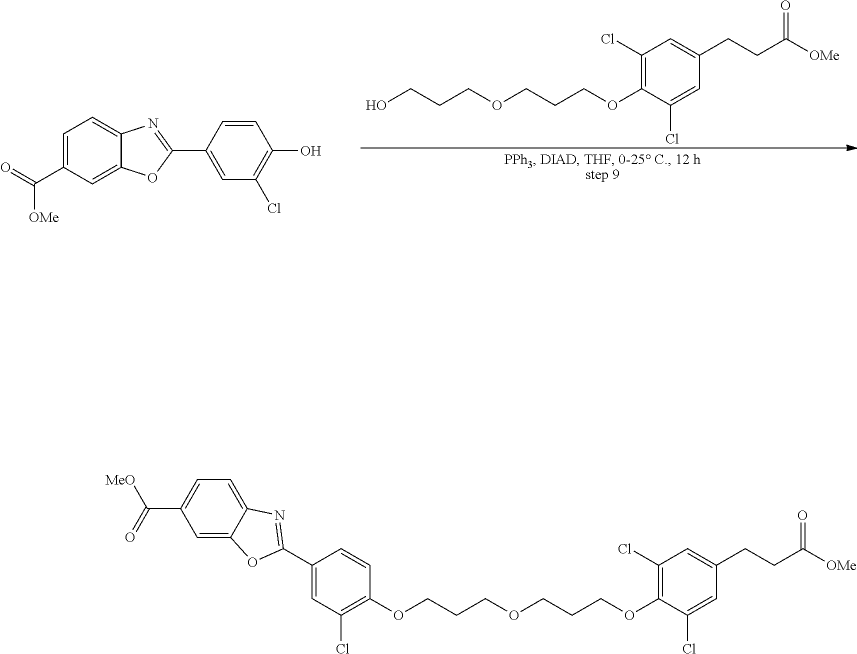 [SMILES: COC(=O)c1ccc2nc(-c3ccc(O)c(Cl)c3)oc2c1 ; COC(=O)CCc1cc(Cl)c(OCCCOCCCO)c(Cl)c1 ; "PPh3, DIAD, THF, 0-25° C., 12 hstep 9" ; COC(=O)CCc1cc(Cl)c(OCCCOCCCOc2ccc(-c3nc4ccc(C(=O)OC)cc4o3)cc2Cl)c(Cl)c1]

### Methyl 2-[3-chloro-4-[3-[3-[2,6-dichloro-4-(3-methoxy-3-oxo-propyl)phenoxy]propoxy]propoxy]phenyl]-1,3-benzoxazole-6-carboxylate

To a solution of methyl 2-(3-chloro-4-hydroxy-phenyl)-1,3-benzoxazole-6-carboxylate (370 mg, 1.22 mmol, 1 eq), methyl 3-[3,5-dichloro-4-[3-(3-hydroxypropoxy)propoxy]phenyl](533.99 mg, 1.46 mmol, 1.2 eq) in THF (20 mL) was added PPh₃ (639.10 mg, 2.44 mmol, 2 eq) at 25° C. The solution was cooled to 0° C. and then DIAD (492.71 mg, 2.44 mmol, 473.76 uL, 2 eq) was added dropwise at 0° C. The resulting mixture was warmed to 25° C. and stirred at 25° C. for 12 h. The mixture was combined with ET20197-243 (100 mg) and concentrated to give the residue. The residue was purified by column (SiO₂, Petroleum ether/Ethyl acetate=10/1 to 3:1) to afford methyl 2-[3-chloro-4-[3-[3-[2,6-dichloro-4-(3-methoxy-3-oxo-propyl)phenoxy]propoxy]propoxy]phenyl]-1,3-benzoxazole-6-carboxylate (450 mg) as a white solid.

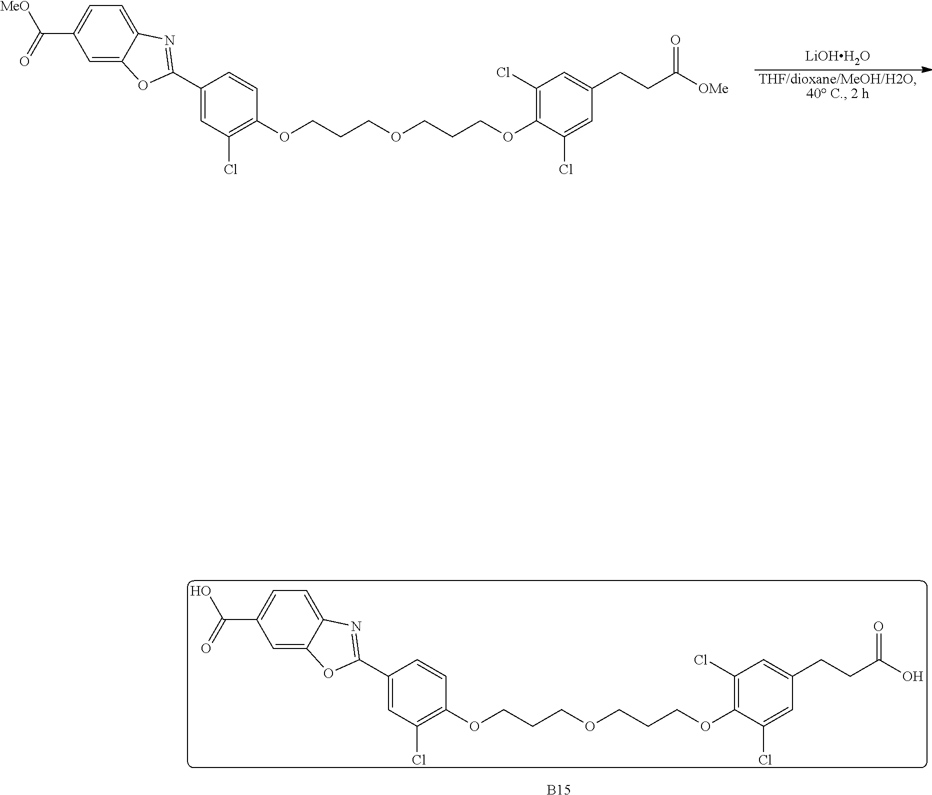

### 2-[4-[3-[3-[4-(2-carboxyethyl)-2,6-dichloro-phenoxy]propoxy]propoxy]-3-chlorophenyl]-1,3-benzoxazole-6-carboxylic acid

To a solution of methyl 2-[3-chloro-4-[3-[3-[2,6-dichloro-4-(3-methoxy-3-oxo-propyl)phen oxy]propoxy]propoxy]phenyl]-1,3-benzoxazole-6-carboxylate (450 mg, 691.32 umol, 1 eq) in THF (3 mL)/dioxane (3 mL)/MeOH (1 mL) was added a solution of LiOH·H₂O (290.10 mg, 6.91 mmol, 10 eq) in H₂O (2 mL). Then the solution was stirred at 40° C. for 2 hr. The reaction mixture was combined with ET20197-248-(50 mg) and concentrated. The residue was adjusted with HCl (1N) to pH 5 ˜6 and extracted with 2-methyltetrahydrofuran (15 mL*3), washed with brine (20 mL*2). The organic layers were concentrated to give the residue. The residue was purified by prep-HPLC (HCl condition) to afford 2-[4-[3-[3-[4-(2-carboxyethyl)-2,6-dichloro-phenoxy]propoxy]propoxy]-3-chlorophenyl]-1,3-benzoxazole-6-carboxylic acid (160.1 mg, 98.32% purity) as a white solid.

LCMS: ET20197-250-P1P (M+H⁺): 622.2 @ 2.156 min (5-100% ACN in H₂O, 3.0 min)

¹H NMR: ET20197-250-P1CC (400 MHz, DMSO-d₆)

δ 8.25 (d, J=1.0 Hz, 1H), 8.19 (d, J=2.1 Hz, 11H), 8.13 (dd, J=2.2, 8.7 Hz, 1H), 8.01 (dd, J=1.6, 8.3 Hz, 1H), 7.86 (d, J=8.3 Hz, 1H), 7.38 (d, J=8.8 Hz, 1H), 7.31 (s, 2H), 4.26 (t, J=6.1 Hz, 2H), 3.98 (t, J=6.2 Hz, 2H), 3.65-3.57 (m, 4H), 2.76-2.70 (m, 2H), 2.54-2.52 (m, 2H), 2.07-1.94 (m, 4H)

### Synthesis of B16 (T-732)

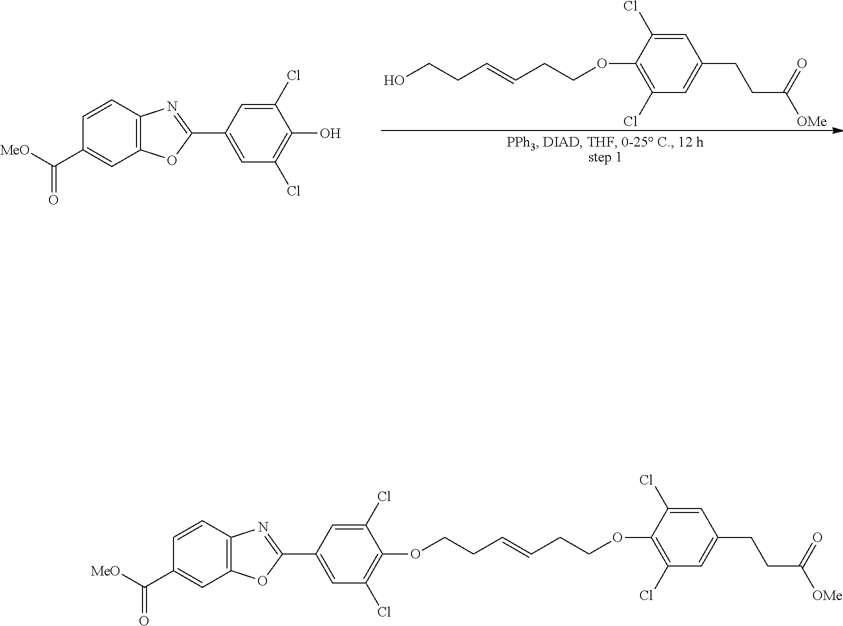

### Methyl 2-[3,5-dichloro-4-[(E)-6-[2,6-dichloro-4-(3-methoxy-3-oxo-propyl)phenoxy]hex-3-enoxy]phenyl]-1,3-benzoxazole-6-carboxylate

To a mixture of methyl 2-(3,5-dichloro-4-hydroxy-phenyl)-1,3-benzoxazole-6-carboxylate (400 mg, 1.18 mmol, 1 eq), methyl 3-[3,5-dichloro-4-[(E)-6-hydroxyhex-3-enoxy]phenyl]propanoate (616.13 mg, 1.77 mmol, 1.5 eq) and PPh₃ (620.53 mg, 2.37 mmol, 2 eq) in THF (30 mL) was added dropwise DIAD (478.40 mg, 2.37 mmol, 460.00 uL, 2 eq) at 0° C., the reaction mixture was stirred at 25° C. for 12 h. The reaction was concentrated to give the residue. The residue was purified by flash silica gel chromatography (ISCO®; 20 g SepaFlash® Silica Flash Column, Eluent of 0 ˜25% 2-MeTHF//Petroleum ethergradient @100 mL/min) to give 400 mg crude (60 purity). The crude was combined with a batch of ET20197-217 (280 mg) and further purified by prep-HPLC (neutral condition) to afford mg of methyl 2-[3,5-dichloro-4-[(E)-6-[2,6-dichloro-4-(3-methoxy-3-oxo-propyl)phenoxy]hex-3-enoxy]phenyl]-1,3-benzoxazole-6-carboxylate (300 mg) as a white solid.

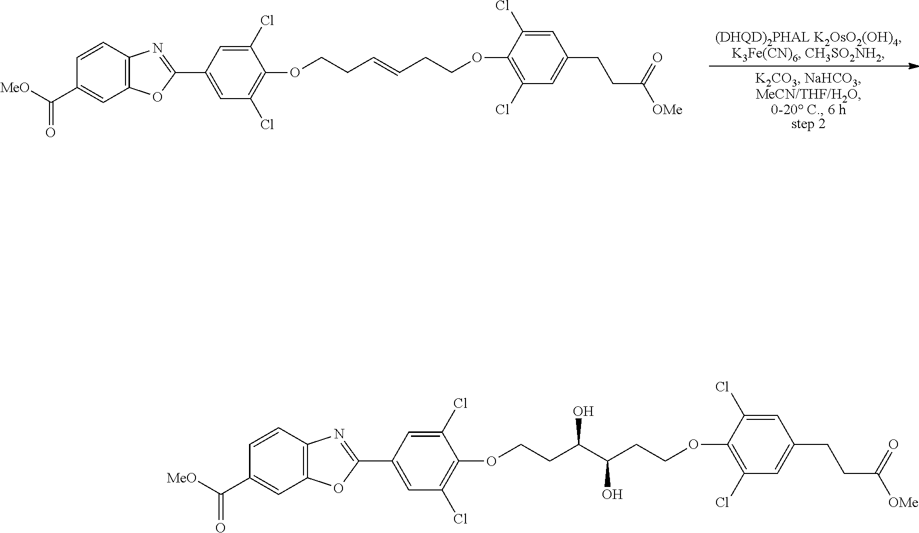 [SMILES: COC(=O)CCc1cc(Cl)c(OCC/C=C/CCOc2c(Cl)cc(-c3nc4ccc(C(=O)OC)cc4o3)cc2Cl)c(Cl)c1 ; "(DHQD)2PHAL K2OsO2(OH)4,K3Fe(CN)6, CH3SO2NH2," ; "K2CO3, NaHCO3,MeCN/THF/H2O,0-20° C., 6 hstep 2" ; COC(=O)CCc1cc(Cl)c(OCC[C@@H](O)[C@H](O)CCOc2c(Cl)cc(-c3nc4ccc(C(=O)OC)cc4o3)cc2Cl)c(Cl)c1]

### Methyl 2-[3,5-dichloro-4-[(3R,4R)-6-[2,6-dichloro-4-(3-methoxy-3-oxo-propyl) phenoxy]-3,4-dihydroxyhexoxy]phenyl]-1,3-benzoxazole-6-carboxylate

A mixture of (DHQD)₂PHAL (67.12 mg, 86.16 umol, 0.25 eq), K₂OsO₄·2H₂O (12.70 mg, 34.46 umol, 0.1 eq), K₃[Fe(CN)₆] (340.41 mg, 1.03 mmol, 283.68 uL, 3 eq), K₂CO₃ (142.89 mg, 1.03 mmol, 3 eq), NaHCO₃ (86.86 mg, 1.03 mmol, 40.21 uL, 3 eq) and MeSO₂NH₂ (32.78 mg, 344.64 umol, 1 eq) in 20 (5 mL) and MeCN (5 mL) was stirred at 20° C. for 30 min, and then the solution was cooled to 0° C., a solution of methyl 2-[3,5-dichloro-4-[(E)-6-[2,6-dichloro-4-(3-methoxy-3-oxo-propyl)phenoxy]hex-3-enoxy]phenyl]-1,3-benzoxazole-6-carboxylate (230 mg, 344.64 umol, 1 eq) in THF (25 mL) was added at 0° C. The final reaction mixture was stirred at 20° C. for 5.5 hr. Na₂SO₃ (1.0 g) in H₂O (50 mL) was added, and the mixture was stirred at 25° C. for 10 min. The mixture was extracted with 2-MeTHF (70 mL*4). The combined organic layers were washed with brine (20 mL), dried over Na₂SO₄ and concentrated under vacuum to give a crude (54% purity). The crude was purified by re-crystallization from MTBE (20 mL) at 25° C. The suspension was filtered and the cake was washed with MTBE (5 mL) to afford methyl 2-[3,5-dichloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate (200 mg, 285.15 umol, 82.74% yield, 100% purity) as a off-white solid. The product was checked by HPLC and Chiral SFC (Retention time: P1: 2.23 min; P2: 2.68 min).

HPLC: ET20197-260-P1H (M+H⁺):4.613 min (10-80% ACN in H₂O, 5.0 min

SFC: ET20197-260-P1S (Retention time: P1: 2.23 min; P2: 2.68 min).

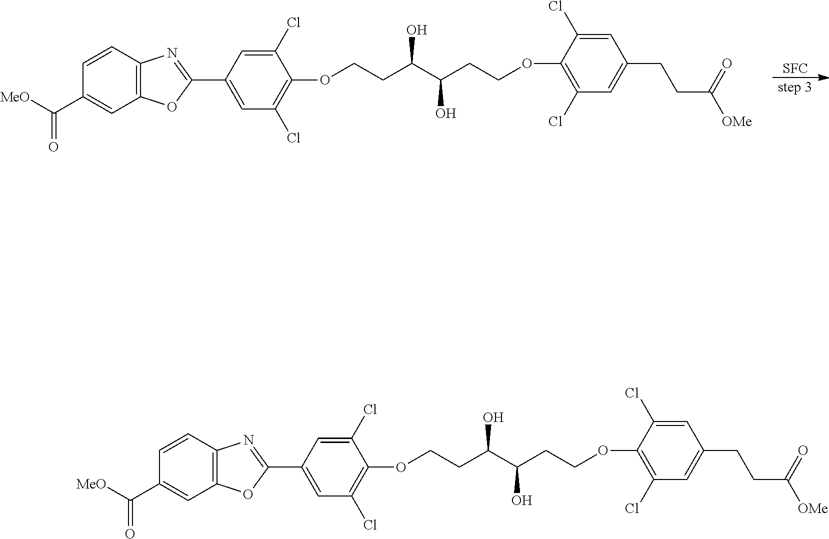

### Methyl 2-[3,5-dichloro-4-[(3R,4R)-6-[2,6-dichloro-4-(3-methoxy-3-oxo-propyl) phenoxy]-3,4-dihydroxy-hexoxy]phenyl]-1,3-benzoxazole-6-carboxylate

Methyl 2-[3,5-dichloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate was separated by SFC (Instrument: Thar SFC 80 preparative SFC; Column: Daicel Chiralpak AS, 250*25 mm i.d. 10 u; Mobile phase: A for CO₂ and B for IPA (0.1% NH₃H₂O); Gradient: B %=45%; Flow rate:75 g/min; Wavelength: 220 nm; Column temperature: 40° C.; (System back pressure: 100 bar, about 600 ml THF-MeOH-ACN) to give:

### P1:

Methyl 2-[3,5-dichloro-4-[(E)-7-[2,6-dichloro-4-(3-methoxy-3-oxo-propyl)phenoxy]hept-4-enyl]phenyl]-1,3-benzoxazole-6-carboxylate (50 mg, 66.13 umol, 33.13% yield, 88% purity) as an off-white solid.

LCMS: ET20197-265-P1A (M+H⁺): 667.9 @ 3.256 min (50-100% ACN in H₂O, 4.25 min)

### P2:

Methyl 2-[3,5-dichloro-4-[(3R,4R)-6-[2,6-dichloro-4-(3-methoxy-3-oxo-propyl)phenoxy]-3,4-dihydroxy-hexoxy]phenyl]-1,3-benzoxazole-6-carboxylate (120 mg, 167.67 umol, 84.00% yield, 98% purity) as an off-white solid.

LCMS: ET20197-265-P2A (M+H⁻): 699.8 @ 3.671 min (50-100% ACN in H₂O, 4.25 min)

SFC: ET20197-265-P2S (Retention time: 2.68; 100% ee)

¹H NMR: ET20197-265-P2P (400 MHz, DMSO-d₆)

δ 8.31 (d, J=0.9 Hz, 1H), 8.23 (s, 2H), 8.05 (dd, J=1.3, 8.4 Hz, 1H), 7.94 (d, J=8.4 Hz, 1H), 7.36 (s, 2H), 4.60 (dd, J=5.7, 11.5 Hz, 2H), 4.31-4.21 (m, 2H), 4.13-4.04 (m, 2H), 3.91 (s, 3H), 3.75-3.61 (m, 2H), 3.58 (s, 3H), 2.83-2.77 (m, 2H), 2.68-2.62 (m, 2H), 2.071.95 (m, 2H), 1.89-1.78 (m, 2H)

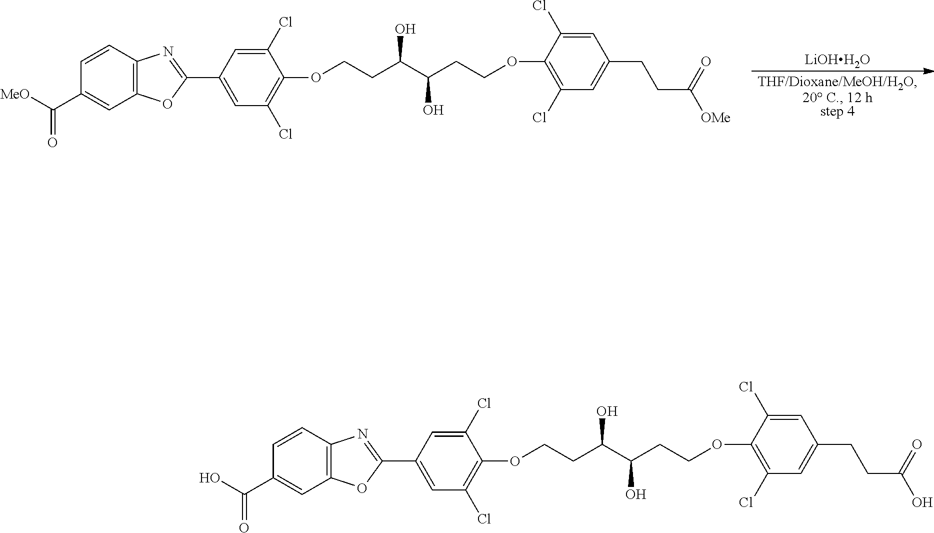 [SMILES: COC(=O)CCc1cc(Cl)c(OCC[C@@H](O)[C@H](O)CCOc2c(Cl)cc(-c3nc4ccc(C(=O)OC)cc4o3)cc2Cl)c(Cl)c1 ; "LiOH·H2O" ; "THF/Dioxane/MeOH/H2O,20° C., 12 hstep 4" ; O=C(O)CCc1cc(Cl)c(OCC[C@@H](O)[C@H](O)CCOc2c(Cl)cc(-c3nc4ccc(C(=O)O)cc4o3)cc2Cl)c(Cl)c1]

### 2-[4-[(3R,4R)-6-[4-(2-carboxyethyl)-2,6-dichloro-phenoxy]-3,4-dihydroxy-hexoxy]-3,5-dichloro-phenyl]-1,3-benzoxazole-6-carboxylic acid

To a solution of methyl 2-[3,5-dichloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate (120 mg, 171.09 umol, 1 eq) in THF (2 mL)/dioxane (2 mL)/MeOH (2 mL) was added a solution of LiOH·H₂O (35.90 mg, 855.46 umol, 5 eq) in H₂O (2 mL) at 20° C. The light yellow solution was stirred at 20° C. for 12 hr. The reaction was adjusted with HCl (1N) to pH-6 and a white suspension was formed. The suspension was concentrated to remove the organic solvent, filtered to give the cake. The cake was washed with H₂O (2 mL) to afford the crude product. The crude was purified by prep-HPLC (HCl condition, in a mixture of DMSO/THF/ACN/H₂O solvent) to afford 2-[4-[(3R,4R)-6-[4-(2-carboxyethyl)-2,6-dichloro-phenoxy]-3,4-dihydroxy-hexoxy]-3,5-dichloro-phenyl]-1,3-benzoxazole-6-carboxylic acid (79.3 mg, 117.57 umol, 68.72% yield, 99.83% purity) as a white solid. The product was checked by QC LCMS and Chiral SFC (Retention time: P1: 5.09 min; P2: 7.74 min).

LCMS: ET20197-282-P1P (M+H⁺): 674.0 @ 1.976 min (5-100% ACN in H₂O, 4.5 min)

SFC: ET20197-282-P1S (Retention time: P1: 5.09 min; P2: 7.74 min)

DSC: ET20197-282-P1D (a range, no detected data)

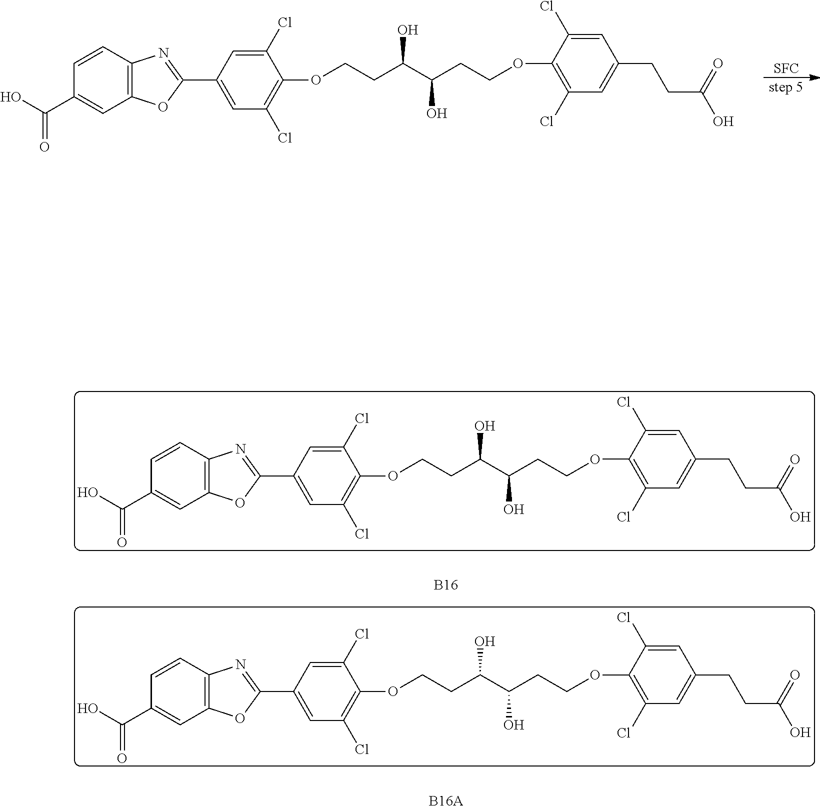

### 2-[4-[(3R,4R)-6-[4-(2-carboxyethyl)-2,6-dichloro-phenoxy]-3,4-dihydroxy-hexoxy]-3,5-dichloro-phenyl]-1,3-benzoxazole-6-carboxylic acid

2-[4-[(3R,4R)-6-[4-(2-carboxyethyl)-2,6-dichloro-phenoxy]-3,4-dihydroxy-hexoxy]-3,5-dichloro-phenyl]-1,3-benzoxazole-6-carboxylic acid was separated by SFC (Instrument: Waters SFC 80Q preparative SFC; Column: Chiralpak AD-H, 250*30 mm i.d., Sum; Mobile phase: A for CO₂ and B for MEOH:ACN=7:3 (0.1% NH₄OH); Gradient: B %=50% Flow rate:70 g/min; Column temperature: 40′C; System back pressure:100 bar) to give:

### P1:

2-[4-[(3R,4R)-6-[4-(2-carboxyethyl)-2,6-dichloro-phenoxy]-3,4-dihydroxy-hexoxy]-3,5-dichloro-phenyl]-1,3-benzoxazole-6-carboxylic acid (21.2 mg, 31.18 umol, 26.41% yield, 99.02% purity) as a white solid.

LCMS: ET20197-296-P1P (M+H⁺): 674.2 @ 2.016 min (5-100% ACN in H₂O, 4.5 min)

SFC: ET20197-296-P1S (Retention time: P1: 4.87 min, 100% ee)

¹H NMR: ET20197-296-P1AA (400 MHz, DMSO-d₆)

δ 8.33-8.22 (m, 3H), 8.04 (br d, J=8.3 Hz, 1H), 7.90 (d, J=8.1 Hz, 1H), 7.36 (s, 2H), 4.59 (br s, 2H), 4.33-4.18 (m, 2H), 4.15-4.02 (m, 2H), 3.67 (br s, 2H), 2.82-2.73 (m, 2H), 2.58-2.54 (m, 2H), 2.11-1.94 (m, 2H), 1.91-1.73 (m, 2H)

### P2:

2-[4-[(3S,4S)-6-[4-(2-carboxyethyl)-2,6-dichloro-phenoxy]-3,4-dihydroxy-hexoxy]-3,5-dichloro-phenyl]-1,3-benzoxazole-6-carboxylic acid (13.2 mg, 19.33 umol, 16.37% yield, 98.61% purity) as a mixture.

LCMS: ET20197-296-P2P (M+H⁻): 673.9 @ 3.291 min (5-100% ACN in H₂O, 4.5 min)

SFC: ET20197-296-P2S (Retention time: P1: 4.81 min, Retention time: 7.23; 50.76% ee)

¹H NMR: ET20197-296-P2AA (400 MHz, DMSO-d₆)

δ 8.24 (s, 2H), 8.04 (br d, J=7.5 Hz, 1H), 7.86 (br d, J=7.7 Hz, 1H), 7.36 (s, 2H), 4.59 (br s, 2H), 4.33-4.19 (m, 2H), 4.14-3.99 (m, 2H), 3.67 (br s, 2H), 2.83-2.72 (m, 2H), 2.58-2.53 (m, 2H), 2.11-1.96 (m, 2H), 1.91-1.74 (m, 2H)

### Synthesis of B17 (T-756)

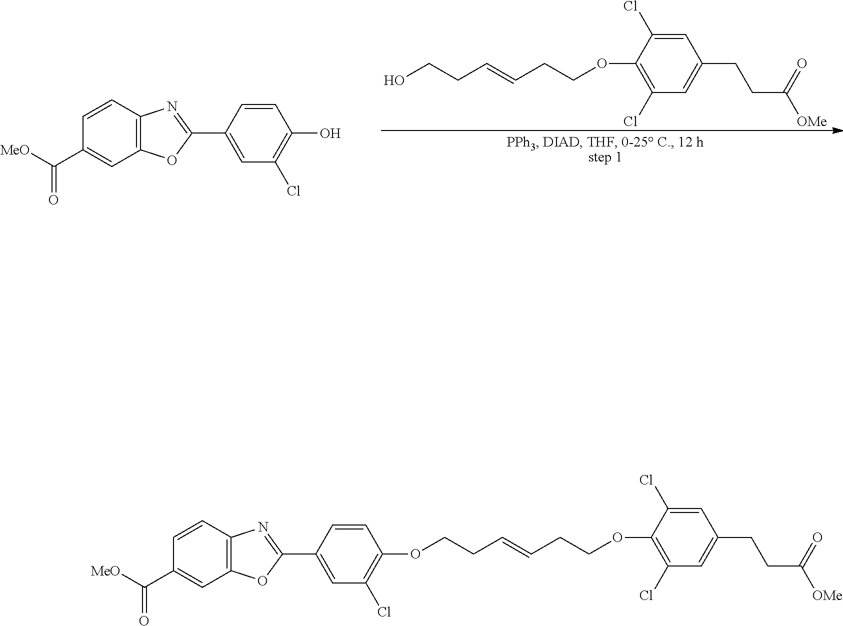

### Methyl 2-[3-chloro-4-[(E)-6-[2,6-dichloro-4-(3-methoxy-3-oxo-propyl)phenoxy]hex-3-enoxy]phenyl]-1,3-benzoxazole-6-carboxylate

To a solution of methyl 2-(3-chloro-4-hydroxy-phenyl)-1,3-benzoxazole-6-carboxylate (600 mg, 1.98 mmol, 1 eq), methyl 3-[3,5-dichloro-4-[(E)-6-hydroxyhex-3-enoxy]phenyl]propanoate (823.21 mg, 2.37 mmol, 1.2 eq) in THF (20 mL) was added PPh₃ (1.04 g, 3.95 mmol, 2 eq) at 25° C. The solution was cooled to 0° C. And then DIAD (798.99 mg, 3.95 mmol, 768.26 uL, 2 eq) was added dropwise at 0° C. The resulting mixture was warmed to 25° C. and stirred at 25° C. for 12 h. The reaction was combined with a batch of ET20197-242 (100 mg). The mixture was concentrated to give the residue. The residue was purified by column chromatography (SiO₂, Petroleum ether/Ethyl acetate=201 to 5:1) to give a crude (900 mg). The crude was further purified by prep-HPLC (neutral condition) to afford methyl 2-[3-chloro-4-[(E)-6-[2,6-dichloro-4-(3-methoxy-3-oxo-propyl)phenoxy]hex-3-enoxy]phenyl]-1,3-benzoxazole-6-carboxylate (370 mg) as a light yellow solid.

¹H NMR: ET20197-245-P1AA (400 MHz, DMSO-d₆)

δ 8.33-8.23 (m, 2H), 8.18-8.05 (m, 2H), 7.76 (d, J=8.2 Hz, 1H), 7.13 (s, 2H), 7.06 (d, J=8.7 Hz, 1H), 5.85-5.68 (m, 2H), 4.17 (br t, J=6.7 Hz, 2H), 4.08-3.93 (m, 5H), 3.69 (s, 3H), 2.86 (t, J=7.6 Hz, 2H), 2.69-2.55 (m, 6H)

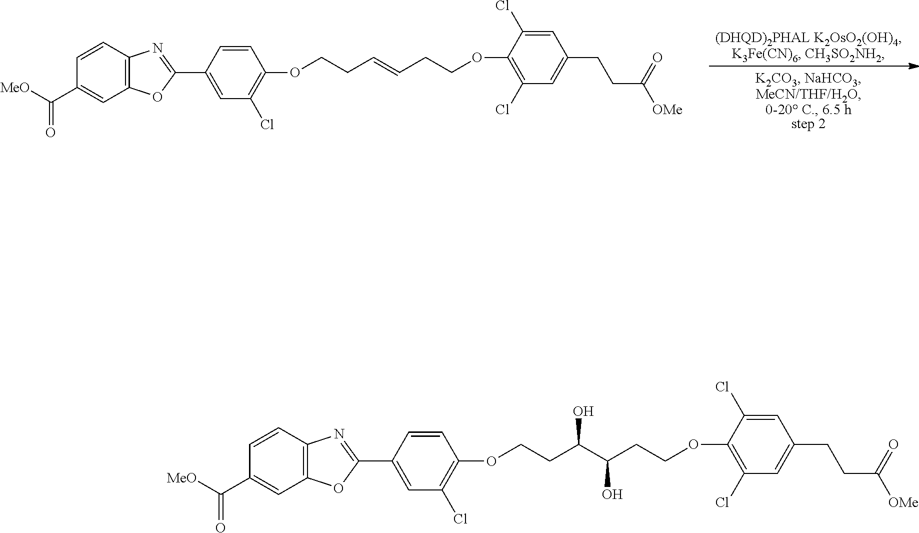 [SMILES: COC(=O)CCc1cc(Cl)c(OCC/C=C/CCOc2ccc(-c3nc4ccc(C(=O)OC)cc4o3)cc2Cl)c(Cl)c1 ; "(DHQD)2PHAL K2OsO2(OH)4,K3Fe(CN)6, CH3SO2NH2," ; "K2CO3, NaHCO3,MeCN/THF/H2O,0-20° C., 6.5 hstep 2" ; COC(=O)CCc1cc(Cl)c(OCC[C@@H](O)[C@H](O)CCOc2ccc(-c3nc4ccc(C(=O)OC)cc4o3)cc2Cl)c(Cl)c1]

### Methyl 2-[3-chloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate

A mixture of (DHQD)₂PHAL (92.31 mg, 118.50 umol, 0.25 eq), K₂OsO₄.2H₂O (17.46 mg, 47.40 umol, 0.1 eq), K₃[Fe(CN)₆] (468.18 mg, 1.42 mmol, 390.15 uL, 3 eq), K₂CO₃ (196.53 mg, 1.42 mmol, 3 eq), NaHCO₃ (119.46 mg, 1.42 mmol, 55.31 uL, 3 eq) and MeSO₂NH₂ (45.09 mg, 474.00 umol, 1 eq) in H₂O (5 mL) and THF (5 mL) was stirred at 20° C. for 30 min, then methyl 2-[3-chloro-4-[(E)-6-[2,6-dichloro-4-(3-methoxy-3-oxo-propyl)phenoxy]hex-3-enoxy]phenyl]-1,3-benzoxazole-6-carboxylate (300 mg, 474.00 umol, 1 eq) in THF (10 mL) was added at 0° C., the final reaction mixture was stirred at 20° C. for 6 hr. Na₂SO₃ (1 g) in H₂O (20 mL) was added, and the mixture was stirred at 25° C. for 10 min. The mixture was extracted with 2-MeTHF (70 mL*4). The combined organic layers were washed with brine (20 mL), dried over Na₂SO₄ and concentrated under vacuum to give a residue. The crude was purified by re-crystallization from MTBE (20 mL) at 25° C. The suspension was filtered and the cake was washed with MTBE (5 mL) to afford methyl 2-[3-chloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate (300 mg, crude) as an off-white solid. The residue was checked by HPLC and Chiral SFC (Retention time: 3.66 min).

HPLC: ET20197-261-P1H (M+H⁺):4.337 min (10-80% ACN in H₂O, 5.0 min)

SFC: ET20197-261-P1S (Retention time:3.66 min, 100% ee).

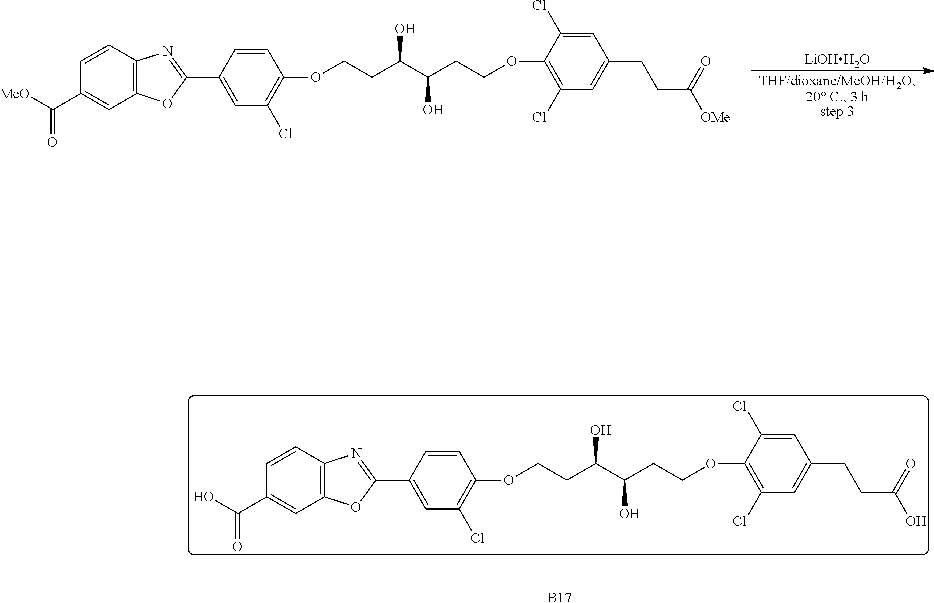

### 2-[4-[(3R,4R)-6-[4-(2-carboxyethyl)-2,6-dichloro-phenoxy]-3,4-dihydroxy-hexoxy]-3-chloro-phenyl]-1,3-benzoxazole-6-carboxylic acid

To a solution of methyl 2-[3-chloro-4-[(3R,4R)-6-[2,6-dichloro-4-(3-methoxy-3-oxo-propyl)phenoxy]-3,4-dihydroxyhexoxy]phenyl]-1,3-benzoxazole-6-carboxylate (280 mg, 419.83 umol, 1 eq) in THF (3 mL)/dioxane (3 mL)/MeOH (2 mL) was added a solution of LiOH·H₂O (176.16 mg, 4.20 mmol, 10 eq) in H₂O (1 mL) at 20° C. The light yellow solution was stirred at 20° C. for 3 hr. The reaction mixture was combined with a batch of ET20197-266 (20 mg) and adjusted with HCl (1N) to pH-6. A white suspension was formed and filtered to give the cake. The cake was washed with H₂O (2 mL) to afford the crude (79% purity). The crude was purified by prep-HPLC (HCl condition, NH₃·H₂O/ACN/H₂O) to give the product (200 mg 100% purity) as a salt. The product was dissolved in a solution of DMSO/MeOH, adjusted with HCl (1N) to pH-5 and further purified by prep-HPLC (MeOH/H₂O condition) to afford 2-[4-[(3R,4R)-6-[4-(2-carboxyethyl)-2,6-dichloro-phenoxy]-3,4-dihydroxy-hexoxy]-3-chloro-phenyl]-1,3-benzoxazole-6-carboxylic acid (27 mg, 42.26 umol, 10.07% yield, 100% purity) as a white solid.

LCMS: ET20197-270-P1L1 (M+H⁺): 638.2 @ 2.460 min (5-90% ACN in H₂O, 4.5 min)

SFC: ET20197-270-P1S (Retention time: 2.73 min, 100% ee)

¹H NMR: ET20197-270-P1N₂ (400 MHz, DMSO-d₆)

δ 8.24 (s, 1H), 8.22-8.15 (m, 2H), 8.01 (d, J=8.3 Hz, 1H), 7.85 (d, J=8.3 Hz, 1H), 7.43 (d, J=8.8 Hz, 1H), 7.36 (s, 2H), 4.71-4.53 (m, 2H), 4.33 (br d, J=6.4 Hz, 2H), 4.14-4.02 (m, 2H), 3.67 (br s, 2H), 2.80-2.74 (m, 2H), 2.57-2.54 (m, 2H), 2.00 (br s, 2H), 1.84 (br s, 2H) DSC: ET20197-270-P1D (Peak: 140.6° C., Onset: 130.7° C., End: 156.0° C.)

### Synthesis of B18 (T-757)

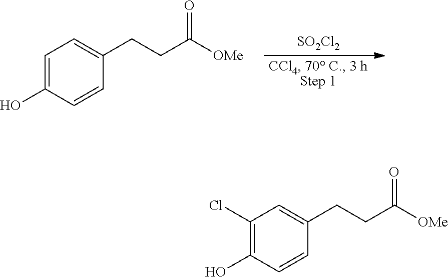 [SMILES: COC(=O)CCc1ccc(O)cc1 ; "SO2Cl2" ; "CCl4, 70° C., 3 hStep 1" ; COC(=O)CCc1ccc(O)c(Cl)c1]

### Methyl 3-(3-chloro-4-hydroxy-phenyl)propanoate

To a solution of methyl 3-(4-hydroxyphenyl)propanoate (5 g, 27.75 mmol, 1 eq) in CCl₄ (50 mL) was added sulfuryl chloride (4.12 g, 30.52 mmol, 3.05 mL, 1.1 eq). The mixture was stirred at 70° C. for 3 hr. The reaction mixture was quenched by addition H₂O 80 mL and partitioned; the aqueous layer was extracted with EtOAc 40 mL×2. The combined organic layers were washed with brine 80 mL, dried over Na₂SO₄, filtered and concentrated under reduced pressure to give a residue. The residue was purified by flash silica gel chromatography (ISCO®; 40 g SepaFlash® Silica Flash Column, Eluent of 0 ˜7% Ethyl acetate/Petroleum ethergradient @ 100 mL/min) to afford methyl 3-(3-chloro-4-hydroxy-phenyl)propanoate (6 g, crude) as a yellow solid.

¹H NMR: ET21585-95-P1AA (CHLOROFORM-d, 400 MHz)

δ 7.16 (d, J=1.8 Hz, 1H), 7.04-6.99 (m, 1H), 6.96-6.91 (m, 1H), 5.52 (s, 1H), 3.68 (s, 3H), 2.91-2.83 (m, 2H), 2.63-2.57 (m, 2H)

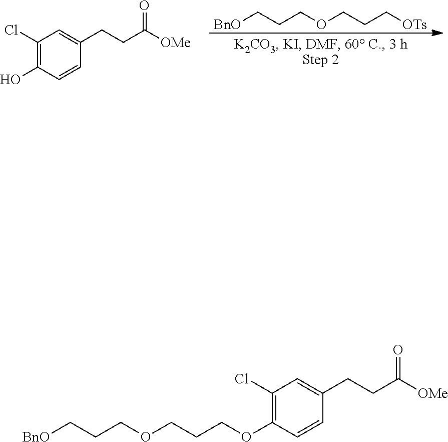

### Methyl 3-[4-[3-(3-benzyloxypropoxy)propoxy]-3-chloro-phenyl]propanoate

To a solution of methyl 3-(3-chloro-4-hydroxy-phenyl)propanoate (4.04 g, 15.06 mmol, 1 eq) and 3-(3-benzyloxypropoxy)propyl 4-methylbenzenesulfonate (5.7 g, 15.06 mmol, 1 eq) in DMF (60 mL) was added K₂CO₃ (6.24 g, 45.18 mmol, 3 eq) and KI (250.00 mg, 1.51 mmol, 0.1 eq). The mixture was stirred at 60° C. for 3 hr. The reaction mixture was diluted with H₂O 100 mL and partitioned; the aqueous layer was extracted with EtOAc 80 mL×2. The combined organic layers were washed with brine 100 mL, dried over Na₂SO₄, filtered and concentrated under reduced pressure to give a residue. The residue was purified by flash silica gel chromatography (ISCO®; 40 g SepaFlash® Silica Flash Column, Eluent of 0 ˜5% Ethyl acetate/Petroleum ethergradient @ 100 mL/min) to afford methyl 3-[4-[3-(3-benzyloxypropoxy)propoxy]-3-chloro-phenyl]propanoate (4.2 g, crude) as a yellow solid.

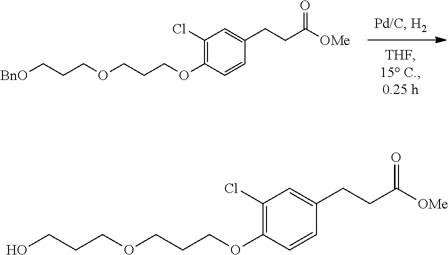 [SMILES: COC(=O)CCc1ccc(OCCCOCCCOCc2ccccc2)c(Cl)c1 ; COC(=O)CCc1ccc(OCCCOCCCO)c(Cl)c1]

### Methyl 3-[3-chloro-4-[3-(3-hydroxypropoxy)propoxy]phenyl]propanoate

To a solution of methyl 3-[4-[3-(3-benzyloxypropoxy)propoxy]-3-chloro-phenyl]propanoate (3.7 g, 8.79 mmol, 1 eq) in THF (40 mL) was added Pd/C (3.7 g, 10% purity) under Ar. The suspension was degassed under vacuum and purged with H₂ several times. The mixture was stirred under H₂ (15 psi) at 15° C. for 15 min. The suspension was filtered through a pad of Celite and the pad was washed with THF 300 mL and EtOAc 500 mL. The combined filtrates were concentrated to dryness to give a residue. The residue was purified by flash silica gel chromatography (ISCO®; 40 g SepaFlash® Silica Flash Column, Eluent of 0˜50% Ethyl acetate/Petroleum ethergradient @ 100 mL/min). Then it was purified by prep-HPLC (neutral condition) to afford methyl 3-[3-chloro-4-[3-(3-hydroxypropoxy)propoxy]phenyl]propanoate (1.3 g) as yellow oil.

The reaction was combined with another reaction (ET21585-101) in 500 mg scale for work up and purification.

¹H NMR: ET21585-101-P1AA (CHLOROFORM-d, 400 MHz)

δ 7.20 (d, J=2.1 Hz, 1H), 7.03 (dd, J=2.1, 8.3 Hz, 1H), 6.86 (d, J=8.4 Hz, 1H), 4.10 (t, J=6.1 Hz, 2H), 3.76 (br t, J=5.4 Hz, 2H), 3.70-3.62 (m, 7H), 2.90-2.84 (m, 2H), 2.63-2.57 (m, 2H), 2.27 (br s, 1H), 2.12-2.06 (m, 2H), 1.84 (quin, J=5.7 Hz, 2H)

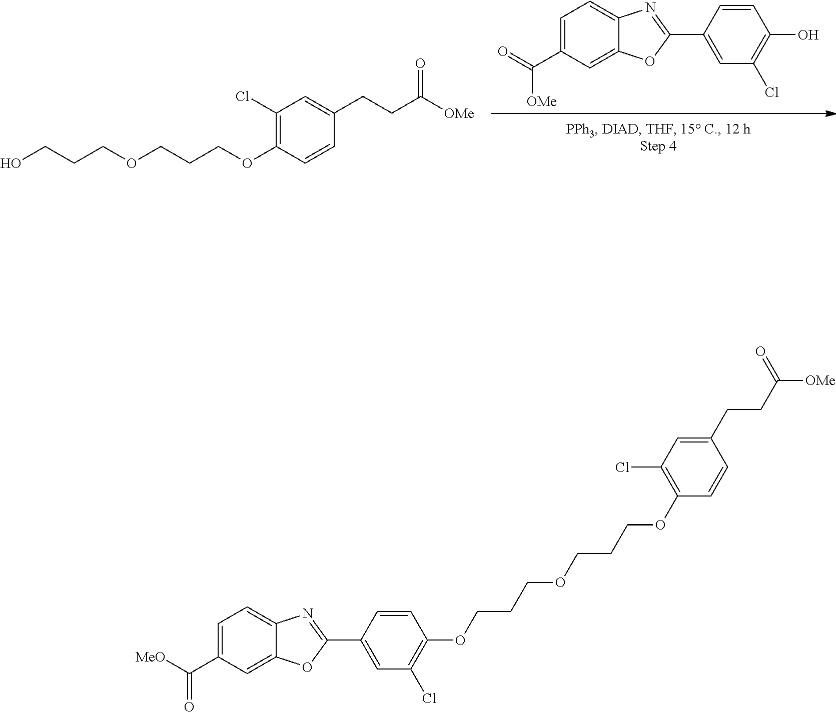 [SMILES: COC(=O)CCc1ccc(OCCCOCCCO)c(Cl)c1 ; COC(=O)c1ccc2nc(-c3ccc(O)c(Cl)c3)oc2c1 ; COC(=O)CCc1ccc(OCCCOCCCOc2ccc(-c3nc4ccc(C(=O)OC)cc4o3)cc2Cl)c(Cl)c1]

### Methyl 2-[3-chloro-4-[3-[3-[2-chloro-4-(3-methoxy-3-oxo-propyl)phenoxy]propoxy]propoxy]phenyl]-1,3-benzoxazole-6-carboxylate

To a solution of methyl 3-[3-chloro-4-[3-(3-hydroxypropoxy)propoxy]phenyl]propanoate (500 mg, 1.51 mmol, 1 eq), methyl 2-(3-chloro-4-hydroxy-phenyl)-1,3-benzoxazole-6-carboxylate (459.03 mg, 1.51 mmol, 1 eq) and PPh₃ (594.67 mg, 2.27 mmol, 1.5 eq) in THF (10 mL) was added DIAD (458.45 mg, 2.27 mmol, 440.82 uL, 1.5 eq). The mixture was stirred at 25° C. for 12 hr. The reaction mixture was concentrated under reduced pressure to remove solvent. The residue was purified by flash silica gel chromatography (ISCO®; 20 g SepaFlash® Silica Flash Column, Eluent of 0 ˜25% Ethyl acetate/Petroleum ethergradient @75 mL/min) to afford methyl 2-[3-chloro-4-[3-[3-[2-chloro-4-(3-methoxy-3-oxo-propyl) phenoxy]propoxy] propoxy]phenyl]-1,3-benzoxazole-6-carboxylate (1 g) as a yellow solid. The reaction was combined with another reaction (ET21585-107) in 50 mg scale for work up and purification.

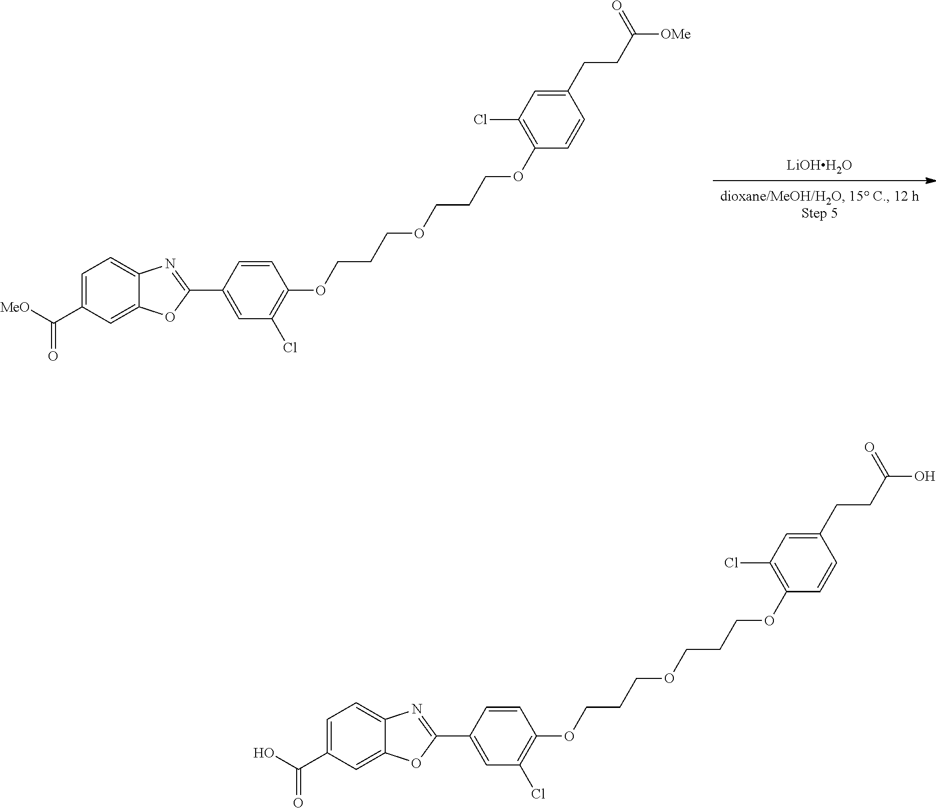 [SMILES: COC(=O)CCc1ccc(OCCCOCCCOc2ccc(-c3nc4ccc(C(=O)OC)cc4o3)cc2Cl)c(Cl)c1 ; "LiOH·H2O" ; O=C(O)CCc1ccc(OCCCOCCCOc2ccc(-c3nc4ccc(C(=O)O)cc4o3)cc2Cl)c(Cl)c1]

### 2-[4-[3-[3-[4-(2-carboxyethyl)-2-chloro-phenoxy]propoxy]propoxy]-3-chloro-phenyl]-1,3-benzoxazole-6-carboxylic acid

To a solution of methyl 2-[3-chloro-4-[3-[3-[2-chloro-4-(3-methoxy-3-oxo-propyl)phenoxy]propoxy]propoxy]phenyl]-1,3-benzoxazole-6-carboxylate (900 mg, 1.46 mmol, 1 eq) in dioxane (10 mL) and MeOH (5 mL) was added LiOH·H₂O (367.54 mg, 8.76 mmol, 6 eq) in H₂O (5 mL). The mixture was stirred at 15° C. for 12 hr. The reaction mixture was diluted with H₂O 20 mL and extracted with EtOAc 20 mL. The aqueous layer was acidified to pH=3 with HCl (6 N). Then it was filtered and the filter cake was washed with 30 mL of H₂O, dried in vacuum to give crude product. The crude product was dissolved in the mixture solvent (THF, DMF, dioxane, NaOH in H₂O) 10 mL. It was purified by basic condition prep-HPLC (NH₄HCO₃ condition) to afford 2-[4-[3-[3-[4-(2-carboxyethyl)-2-chloro-phenoxy]propoxy]propoxy]-3-chloro-phenyl]-1,3-benzoxazole-6-carboxylic acid (225 mg, 98.76% purity) as a white solid.

The reaction was combined with another reaction (ET21585-116) in 100 mg scale for work up and purification.

LCMS: ET21585-119-P1A2 (M+H⁺): 588.1@ 2.513 min (10-80% ACN in H₂O, 4.5 min)

¹H NMR: ET21585-119-P1AA (DMSO-d₆, 400 MHz)

δ 8.20-8.14 (m, 2H), 8.09 (dd, J=1.8, 8.6 Hz, 1H), 7.98 (br d, J=8.3 Hz, 1H), 7.75 (d, J=8.2 Hz, 1H), 7.32 (br d, J=8.8 Hz, 1H), 7.23 (d, J=2.0 Hz, 1H), 7.06 (dd, J=2.0, 8.4 Hz, 1H), 6.95 (d, J=8.4 Hz, 1H), 4.22 (br t, J=6.0 Hz, 2H), 4.03 (t, J=6.2 Hz, 2H), 3.58 (q, J=6.3 Hz, 4H), 2.73-2.65 (m, 2H), 2.48-2.43 (m, 2H), 2.06-1.91 (m, 4H)

DSC: ET21585-119-P1S (Peak: 198.5° C., Onset: 193.5° C., End: 205.7° C.)

### Synthesis of B19 (T-758)

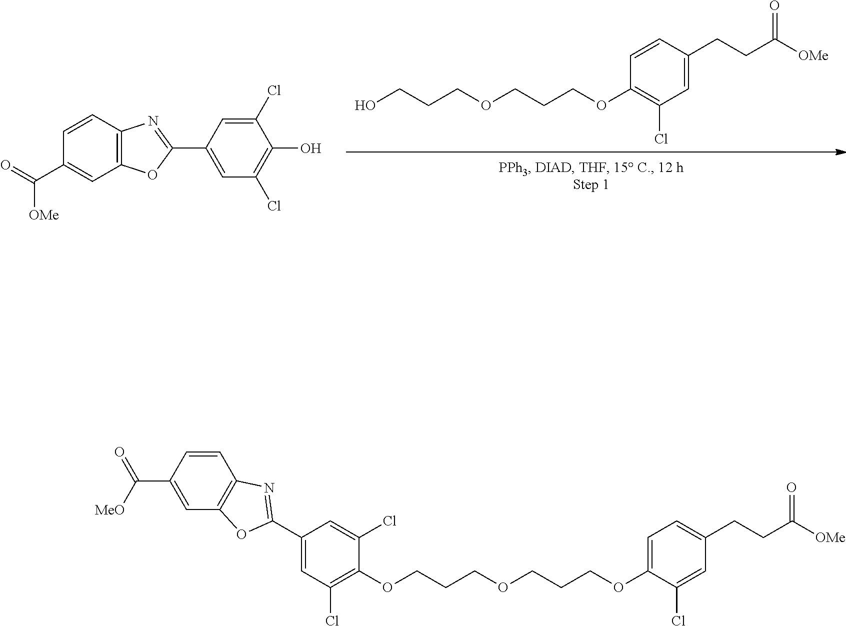 [SMILES: COC(=O)c1ccc2nc(-c3cc(Cl)c(O)c(Cl)c3)oc2c1 ; COC(=O)CCc1ccc(OCCCOCCCO)c(Cl)c1 ; COC(=O)CCc1ccc(OCCCOCCCOc2c(Cl)cc(-c3nc4ccc(C(=O)OC)cc4o3)cc2Cl)c(Cl)c1]

### Methyl 2-[3,5-dichloro-4-[3-[3-[2-chloro-4-(3-methoxy-3-oxo-propyl)phenoxy]propoxy]propoxy]phenyl]-1,3-benzoxazole-6-carboxylate

To a solution of methyl 3-[3-chloro-4-[3-(3-hydroxypropoxy)propoxy]phenyl]propanoate (567.41 mg, 1.72 mmol, 1 eq), methyl 2-(3,5-dichloro-4-hydroxy-phenyl)-1,3-benzoxazole-6-carboxylate (580 mg, 1.72 mmol, 1 eq) and PPh₃ (674.84 mg, 2.57 mmol, 1.5 eq) in THF (20 mL) was added DIAD (520.26 mg, 2.57 mmol, 500.25 uL, 1.5 eq). The mixture was stirred at 15° C. for 12 hr. The reaction mixture was concentrated under reduced pressure to remove solvent. The residue was purified by flash silica gel chromatography (ISCO®; 20 g SepaFlash® Silica Flash Column, Eluent of 0 ˜24% Ethyl acetate/Petroleum ethergradient @75 mL/min) to afford methyl 2-[3,5-dichloro-4-[3-[3-[2-chloro-4-(3-methoxy-3-oxo-propyl) phenoxy]propoxy]propoxy]phenyl]-1,3-benzoxazole-6-carboxylate (1.2 g) as a white solid. The reaction was combined with another reaction (ET21585-108 and ET21585-110) in 100 mg scale for purification and work up.

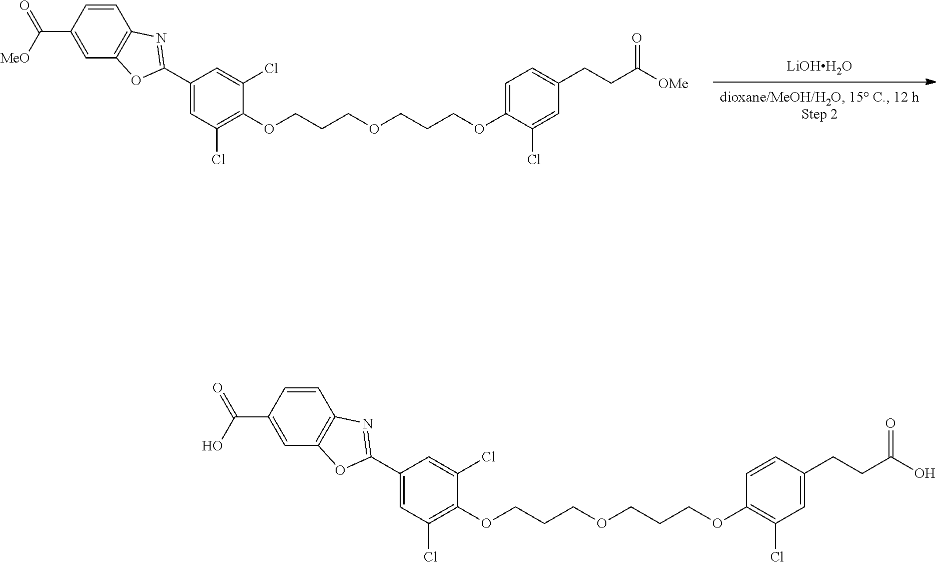 [SMILES: COC(=O)CCc1ccc(OCCCOCCCOc2c(Cl)cc(-c3nc4ccc(C(=O)OC)cc4o3)cc2Cl)c(Cl)c1 ; "LiOH·H2O" ; O=C(O)CCc1ccc(OCCCOCCCOc2c(Cl)cc(-c3nc4ccc(C(=O)O)cc4o3)cc2Cl)c(Cl)c1]

### 2-[4-[3-[3-[4-(2-carboxyethyl)-2-chloro-phenoxy]propoxy]propoxy]-3,5-dichloro-phenyl]-1,3-benzoxazole-6-carboxylic acid

To a solution of methyl 2-[3,5-dichloro-4-[3-[3-[2-chloro-4-(3-methoxy-3-oxo-propyl) phenoxy]propoxy]propoxy]phenyl]-1,3-benzoxazole-6-carboxylate (1 g, 1.54 mmol, 1 eq) in dioxane (10 mL) and MeOH (5 mL) was added LiOH·H₂O (386.77 mg, 9.22 mmol, 6 eq) in H₂O (5 mL). The mixture was stirred at 15° C. for 12 hr. The reaction mixture was diluted with H₂O 20 mL and extracted with EtOAc 20 mL. The aqueous layer was acidified to pH=3 with HCl (6 N). Then it was filtered and the filter cake was washed with 30 mL of H₂O, dried in vacuum to give crude product. The crude product was purified by prep-HPLC (HCl condition) to afford 2-[4-[3-[3-[4-(2-carboxyethyl)-2-chloro-phenoxy]propoxy]propoxy]-3,5-dichloro-phenyl]-1,3-benzoxazole-6-carboxylic acid (123 mg, 99.74% purity) as a white solid.

The reaction was combined with another reaction (ET21585-121) in 200 mg scale for purification and work up.

LCMS: ET21585-121-P1A1 (M+H⁺): 624.0@ 2.822 min (10-80% ACN in H₂O, 4.5 min)

¹H NMR: ET21585-121-P1AA (DMSO-d₆, 400 MHz)

δ 8.25 (d, J=0.9 Hz, 1H), 8.16 (s, 2H), 8.02 (dd, J=1.3, 8.3 Hz, 1H), 7.88 (d, J=8.3 Hz, 1H), 7.25 (d, J=2.0 Hz, 1H), 7.09 (dd, J=1.9, 8.4 Hz, 1H), 7.03-6.97 (m, 1H), 4.14 (t, J=6.2 Hz, 2H), 4.07 (t, J=6.2 Hz, 2H), 3.60 (td, J=6.2, 16.2 Hz, 4H), 2.75-2.68 (m, 2H), 2.49-2.44 (m, 2H), 2.08-1.93 (m, 4H)

DSC: ET21585-121-P1S (Peak: 184.8° C., Onset: 170.9° C., End: 190.9° C.)

### Synthesis of B20 (T-606/T-616)

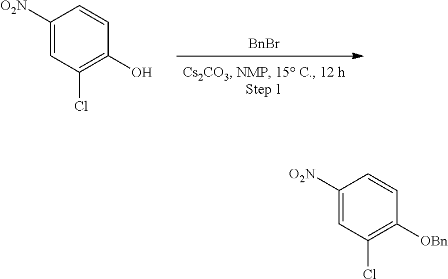 [SMILES: O=[N+]([O-])c1ccc(O)c(Cl)c1 ; "BnBr" ; O=[N+]([O-])c1ccc(OCc2ccccc2)c(Cl)c1]

### 1-benzyloxy-2-chloro-4-nitro-benzene

To a mixture of 2-chloro-4-nitro-phenol (25 g, 144.05 mmol, 1 eq) and Cs₂CO₃ (117.34 g, 360.13 mmol, 2.5 eq) in NMP (400 mL) was added bromomethylbenzene (24.64 g, 144.05 mmol, 17.11 mL, 1 eq) dropwise at 15° C., and then the reaction mixture was stirred at 15° C. for 12 hr. The reaction was diluted with H₂O 1000 mL and extracted with EtOAc 500 mL×3. The organic layer was washed with brine 200 mL and concentrated to give the residue. The crude product was purified by re-crystallization from MTBE (100 mL) at 15° C. to give 1-benzyloxy-2-chloro-4-nitro-benzene (31 g, 117.57 mmol, 81.62% yield) as a yellow solid.

¹H NMR: ET20197-257-P1AA (CHLOROFORM-d, 400 MHz)

δ 8.31 (d, J=2.8 Hz, 1H), 8.23 (dd, J=2.8, 9.2 Hz, 1H), 7.57-7.27 (m, 6H), 5.37 (s, 2H)

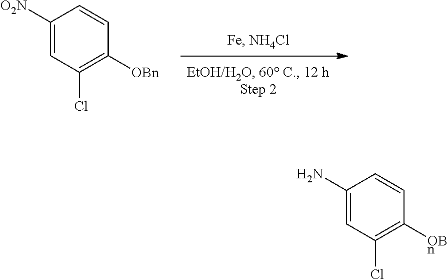

### 4-benzyloxy-3-chloro-aniline

To a solution of 1-benzyloxy-2-chloro-4-nitro-benzene (31 g, 117.57 mmol, 1 eq) and NH₄Cl (31.44 g, 587.84 mmol, 5 eq) in EtOH (150 mL) and H₂O (150 mL) was added Fe (32.83 g, 587.84 mmol, 5 eq) in portions. The mixture was stirred at 60° C. for 12 hr. The suspension was filtered through a pad of Celite and the filter cake was washed with EtOAc (1.5 L). The combined filtrates were concentrated to dryness to give crude product. The crude product was purified by flash silica gel chromatography (ISCO®; 220 g SepaFlash® Silica Flash Column, Eluent of 0 ˜30% Ethyl acetate/Petroleum ethergradient @ 100 mL/min) to afford 4-benzyloxy-3-chloro-aniline (25.4 g, 108.69 mmol, 92.45% yield) as a brown solid.

¹H NMR: ET21585-105-P1AA (CHLOROFORM-d, 400 MHz)

δ 7.49-7.43 (m, 2H), 7.42-7.35 (m, 2H), 7.35-7.29 (m, 1H), 6.80 (d, J=8.7 Hz, 1H), 6.76 (d, J=2.7 Hz, 1H), 6.51 (dd, 1=2.8, 8.6 Hz, 1H), 5.06 (s, 2H), 3.49 (br s, 2H)

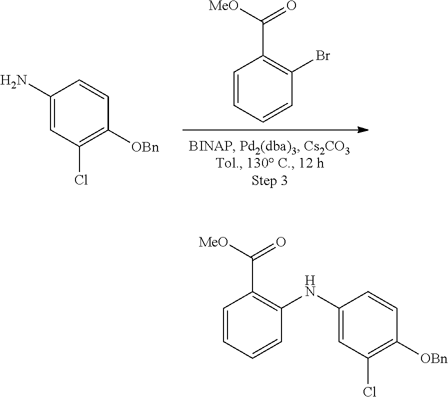 [SMILES: Nc1ccc(OCc2ccccc2)c(Cl)c1 ; COC(=O)c1ccccc1Br ; COC(=O)c1ccccc1Nc1ccc(OCc2ccccc2)c(Cl)c1]

### Methyl 2-(4-benzyloxy-3-chloro-anilino)benzoate

A mixture of 4-benzyloxy-3-chloro-aniline (5 g, 21.40 mmol, 1 eq), methyl 2-bromobenzoate (4.60 g, 21.40 mmol, 3.01 mL, 1 eq), BINAP (999.18 mg, 1.60 mmol, 0.075 eq), Pd₂(dba)₃ (979.62 mg, 1.07 mmol, 0.05 eq) and Cs₂CO₃ (17.43 g, 53.49 mmol, 2.5 eq) in Tol. (70 mL) was degassed and purged with N₂ for 3 times, and then the mixture was stirred at 130° C. for 12 hr under N₂ atmosphere. The reaction mixture was diluted with H₂O 200 mL and partitioned; the aqueous layer was extracted with EtOAc 100 mL×2. The combined organic layers were washed with brine 200 mL, dried over Na₂SO₄, filtered and concentrated under reduced pressure to give a residue. The residue was purified by flash silica gel chromatography (ISCO®; 40 g SepaFlash® Silica Flash Column, Eluent of 0 ˜2% Ethyl acetate/Petroleum ethergradient @ 100 mL/min) to afford methyl 2-(4-benzyloxy-3-chloro-anilino)benzoate (6.5 g, 17.67 mmol, 82.59% yield) as a white solid.

¹H NMR: ET21585-112-P1AA (CHLOROFORM-d, 400 MHz)

δ 9.31 (s, 1H), 7.96 (dd, J=1.5, 8.0 Hz, 1H), 7.52-7.46 (m, 2H), 7.41 (t, J=7.4 Hz, 2H), 7.37-7.28 (m, 3H), 7.09-7.03 (m, 2H), 6.96 (d, J=8.7 Hz, 1H), 6.75-6.69 (m, 1H), 5.17 (s, 2H), 3.91 (s, 3H)

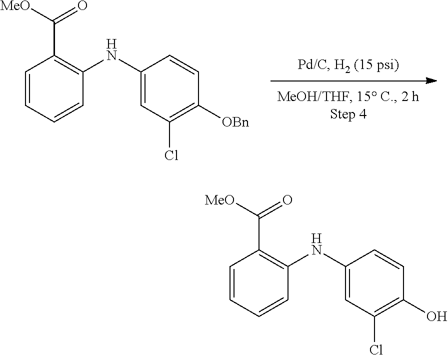 [SMILES: COC(=O)c1ccccc1Nc1ccc(OCc2ccccc2)c(Cl)c1 ; COC(=O)c1ccccc1Nc1ccc(O)c(Cl)c1]

### Methyl 2-(3-chloro-4-hydroxy-anilino)benzoate

To a solution of methyl 2-(4-benzyloxy-3-chloro-anilino)benzoate (6 g, 16.31 mmol, 1 eq) in MeOH (60 mL) and THF (60 mL) was added Pd/C (1.5 g, 16.31 mmol, 10% purity) under Ar. The suspension was degassed under vacuum and purged with H₂ several times. The mixture was stirred under H₂ (15 psi) at 15° C. for 2 hours. The suspension (combined with ET21585-117, 500 mg scale) was filtered through a pad of Celite and the filter cake was washed with EtOAc 1000 mL. The combined filtrates were concentrated to dryness to give crude product. The crude product was purified by flash silica gel chromatography (ISCO®; 40 g SepaFlash® Silica Flash Column, Eluent of 0 ˜3% Ethyl acetate/Petroleum ethergradient @ 100 mL/min) to afford methyl 2-(3-chloro-4-hydroxy-anilino)benzoate (4.3 g) as a yellow solid.

¹H NMR: ET21585-118-P1AA (CHLOROFORM-d, 400 MHz)

δ 9.29 (br s, 1H), 7.96 (dd, J=1.4, 8.0 Hz, 1H), 7.34-7.28 (m, 1H), 7.25 (d, J=2.4 Hz, 1H), 7.11-7.05 (m, 1H), 7.04-6.96 (m, 2H), 6.75-6.70 (m, 1H), 5.45 (s, 1H), 3.95-3.88 (m, 3H)

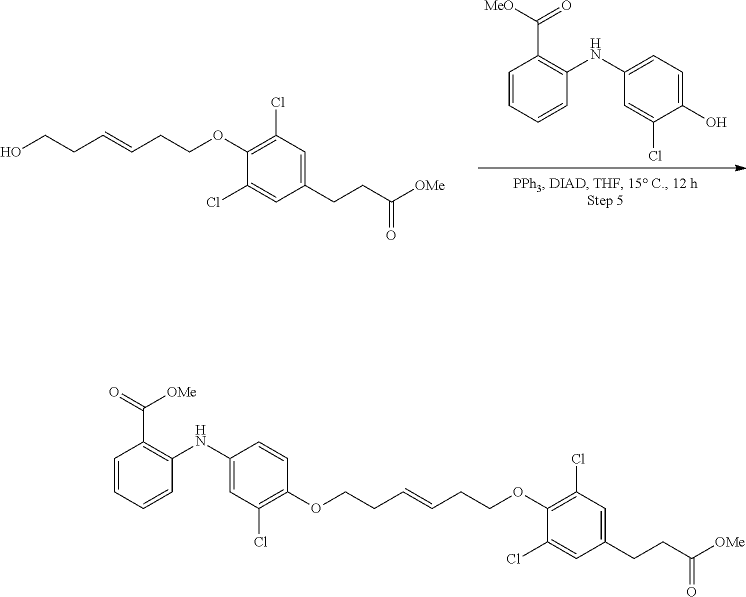 [SMILES: COC(=O)CCc1cc(Cl)c(OCC/C=C/CCO)c(Cl)c1 ; COC(=O)c1ccccc1Nc1ccc(O)c(Cl)c1 ; COC(=O)CCc1cc(Cl)c(OCC/C=C/CCOc2ccc(Nc3ccccc3C(=O)OC)cc2Cl)c(Cl)c1]

### Methyl 2-[3-chloro-4-[(E)-6-[2,6-dichloro-4-(3-methoxy-3-oxo-propyl)phenoxy]hex-3-enoxy]anilino]benzoate

To a solution of methyl 2-(3-chloro-4-hydroxy-anilino)benzoate (1.20 g, 4.32 mmol, 1 eq), methyl 3-[3,5-dichloro-4-[(E)-6-hydroxyhex-3-enoxy]phenyl]propanoate (1.5 g, 4.32 mmol, 1 eq), PPh₃ (1.70 g, 6.48 mmol, 1.5 eq) in THF (25 mL) was added DIAD (1.31 g, 6.48 mmol, 1.26 mL, 1.5 eq). The mixture was stirred at 15° C. for 12 hr. The reaction mixture (combined with ET21585-124, 500 mg scale) was concentrated under reduced pressure to remove solvent. The crude product was purified by reversed MPLC (HCl condition, SiO2, 50˜100% H2O/MeOH) to afford methyl 2-[3-chloro-4-[(E)-6-[2,6-dichloro-4-(3-methoxy-3-oxo-propyl)phenoxy]hex-3-enoxy]anilino]benzoate (1 g) as a yellow solid.

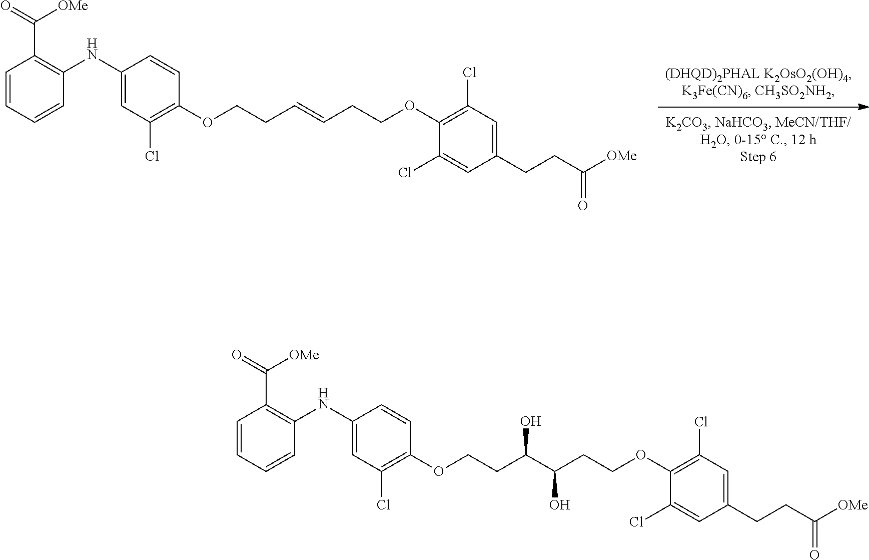 [SMILES: COC(=O)CCc1cc(Cl)c(OCC/C=C/CCOc2ccc(Nc3ccccc3C(=O)OC)cc2Cl)c(Cl)c1 ; COC(=O)CCc1cc(Cl)c(OCC[C@@H](O)[C@H](O)CCOc2ccc(Nc3ccccc3C(=O)OC)cc2Cl)c(Cl)c1]

### Methyl 2-[3-chloro-4-[(3R,4R)-6-[2,6-dichloro-4-(3-methoxy-3-oxo-propyl)phenoxy]-3,4-dihydroxy-hexoxy]anilino]benzoate

A mixture of K₂OsO₄·2H₂O (11.53 mg, 31.31 umol, 0.02 eq), (DHQD)₂PHAL (60.97 mg, 78.26 umol, 0.05 eq), NaHCO₃ (131.49 mg, 1.57 mmol, 60.88 uL, 1 eq), MeSO₂NH₂ (148.89 mg, 1.57 mmol, 1 eq), K₂CO₃ (540.83 mg, 3.91 mmol, 2.5 eq) and K₃[Fe(CN)₆] (1.29 g, 3.91 mmol, 1.07 mL, 2.5 eq) in H₂O (20 mL) and MeCN (20 mL) was stirred for 15 min at 15° C. The mixture was cooled to 0° C. and methyl 2-[3-chloro-4-[(E)-6-[2,6-dichloro-4-(3-methoxy-3-oxo-propyl)phenoxy]hex-3-enoxy]anilino]benzoate (950 mg, 1.57 mmol, 1 eq) in THF (120 mL) was added. The reaction was stirred at 0° C. for 3 hr and 15° C. for 8 hr 45 min. The reaction mixture was quenched by addition Na₂SO₃ 10 g in H₂O 70 mL and extracted with EtOAc 60 mL×3. The combined organic layers were washed with brine 70 mL, dried over Na₂SO₄, filtered and concentrated under reduced pressure to give a residue The residue was purified by flash silica gel chromatography (ISCO®; 20 g SepaFlash® Silica Flash Column, Eluent of 0 ˜50% Ethyl acetate/Petroleum ethergradient @ 75 mL/min) to afford methyl 2-[3-chloro-4-[(3R,4R)-6-[2,6-dichloro-4-(3-methoxy-3-oxo-propyl)phenoxy]-3,4-dihydroxy-hexoxy]anilino]benzoate (760 mg, 1.17 mmol, 75.00% yield) as a yellow solid.

SFC: ET21585-133-P1A1 (Retention time: 2.22 min; 100% ee value)

¹H NMR: ET21585-133-P1AA (DMSO-d₆, 400 MHz)

δ 9.13 (s, 1H), 7.87 (dd, J=1.4, 8.0 Hz, 1H), 7.42-7.34 (m, 3H), 7.32 (d, J=2.2 Hz, 1H), 7.22-7.13 (m, 2H), 7.00 (d, J=8.4 Hz, 1H), 6.77 (t, J=7.5 Hz, 1H), 4.57 (dd, J=5.7, 17.0 Hz, 2H), 4.17 (br t, J=6.4 Hz, 2H), 4.13-4.03 (m, 2H), 3.85 (s, 3H), 3.65 (br s, 2H), 3.58 (s, 3H), 2.84-2.77 (m, 2H), 2.69-2.61 (m, 2H), 2.05-1.91 (m, 2H), 1.88-1.74 (m, 2H)

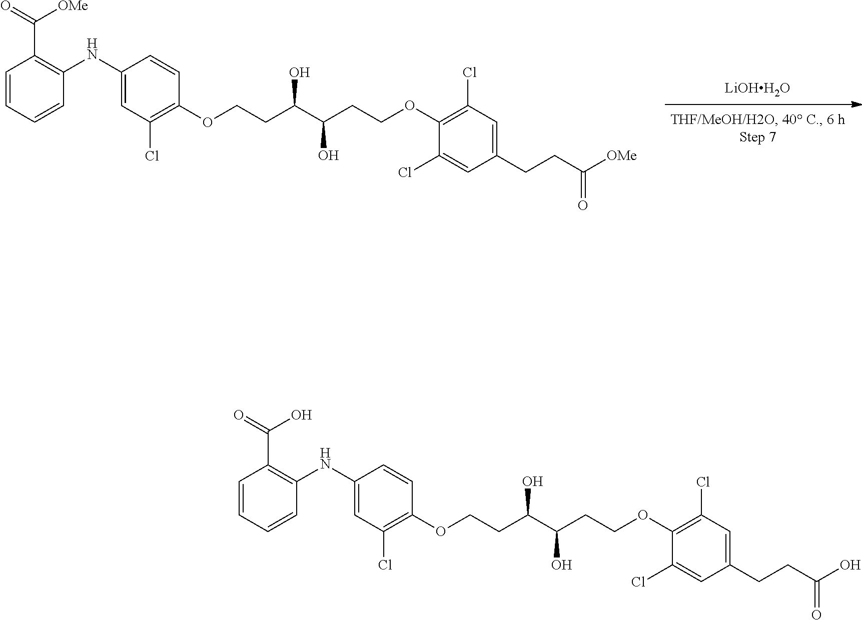 [SMILES: COC(=O)CCc1cc(Cl)c(OCC[C@@H](O)[C@H](O)CCOc2ccc(Nc3ccccc3C(=O)OC)cc2Cl)c(Cl)c1 ; "LiOH·H2O" ; O=C(O)CCc1cc(Cl)c(OCC[C@@H](O)[C@H](O)CCOc2ccc(Nc3ccccc3C(=O)O)cc2Cl)c(Cl)c1]

### 2-[4-[(3R,4R)-6-[4-(2-carboxyethyl)-2,6-dichloro-phenoxy]-3,4-dihydroxy-hexoxy]-3-chloro-anilino]benzoic acid

To a solution of methyl 2-[3-chloro-4-[(3R,4R)-6-[2,6-dichloro-4-(3-methoxy-3-oxo-propyl) phenoxy]-3,4-dihydroxy-hexoxy]anilino]benzoate (690 mg, 1.08 mmol, 1 eq) in THF (30 mL) and MeOH (15 mL) was added LiOH·H₂O (271.03 mg, 6.46 mmol, 6 eq) in H₂O (15 mL). The mixture was stirred at 40° C. for 6 hr. The reaction mixture (combined with ET21585-136, 70 mg scale) was diluted with H₂O 40 mL and acidified to pH=6 with HCl (1 N), the mixture was extracted with EtOAc 30 mL×3. The combined organic layers were washed with brine 60 mL, dried over Na₂SO₄, filtered and concentrated under reduced pressure to give a residue. The residue was purified by prep-HPLC (Phenomenex Luna C18 200*40 mm*10 um; mobile phase: [water (0.05% HCl)-ACN]; B %: 35%-65%, 10 min) to afford 2-[4-[(3R,4R)-6-[4-(2-carboxyethyl)-2,6-dichloro-phenoxy]-3,4-dihydroxy-hexoxy]-3-chloro-anilino]benzoic acid (547 mg) as a light yellow solid.

LCMS: ET21585-138-P1A1 (M+H⁺): 612.1@ 2.603 min (10-80% ACN in H₂O, 4.5 min)

¹H NMR: ET21585-138-P1AA (DMSO-d6, 400 MHz)

δ 13.26-11.93 (m, 2H), 9.44 (s, 1H), 7.88 (dd, J=1.6, 7.9 Hz, 1H), 7.40-7.31 (m, 4H), 7.22-7.13 (m, 2H), 7.00 (d, J=8.6 Hz, 1H), 6.78-6.71 (m, 1H), 4.57 (br s, 2H), 4.17 (br t, J=6.4 Hz, 2H), 4.13-4.02 (m, 2H), 3.65 (br d, J=7.6 Hz, 2H), 2.81-2.73 (m, 2H), 2.55 (t, J=7.6 Hz, 2H), 2.05-1.92 (m, 2H), 1.88-1.73 (m, 2H)

SFC: ET21585-138-P1A3 (Retention time: 4.56 min; 100% ee value)

DSC: ET21585-138-P1A2 (Peak: 138.8° C., onset: 130.4° C., end: 143.0° C.)

### Synthesis of B21 (T-672/T-673)

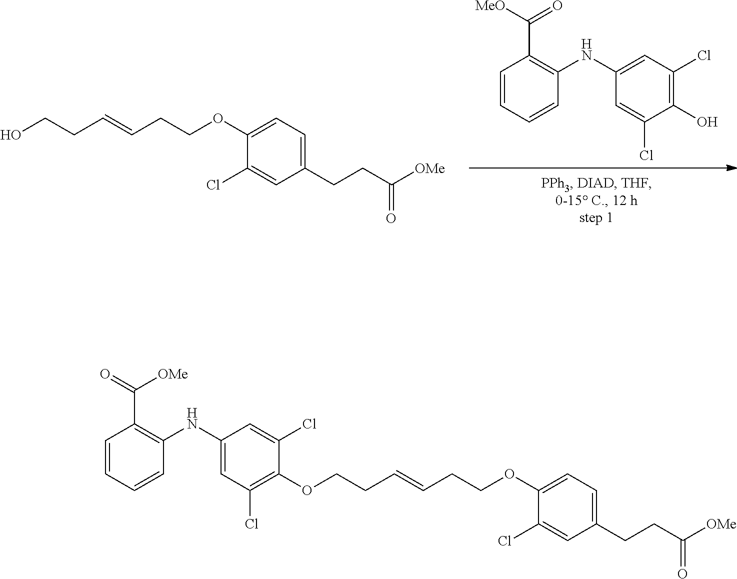 [SMILES: COC(=O)CCc1ccc(OCC/C=C/CCO)c(Cl)c1 ; COC(=O)c1ccccc1Nc1cc(Cl)c(O)c(Cl)c1 ; COC(=O)CCc1ccc(OCC/C=C/CCOc2c(Cl)cc(Nc3ccccc3C(=O)OC)cc2Cl)c(Cl)c1]

### Methyl 2-[3,5-dichloro-4-[(E)-6-[2-chloro-4-(3-methoxy-3-oxo-propyl)phenoxy]hex-3-enoxy]anilino]benzoate

To a mixture of methyl 3-[3-chloro-4-[(E)-6-hydroxyhex-3-enoxy]phenyl]propanoate (1 g, 3.20 mmol, 1 eq), methyl 2-(3,5-dichloro-4-hydroxy-anilino)benzoate (997.95 mg, 3.20 mmol, 1 eq) and PPh₃ (1.68 g, 6.39 mmol, 2 eq) in THF (10 mL) was added DIAD (1.29 g, 6.39 mmol, 1.24 mL, 2 eq) at 0° C., and then the reaction mixture was stirred at 15° C. for 12 h. The reaction was concentrated to give the residue. The crude product was purified by reversed-phase HPLC (MeOH/0.1% TFA condition) to afford methyl 2-[3,5-dichloro-4-[(E)-6-[2-chloro-4-(3-methoxy-3-oxo-propyl)phenoxy]hex-3-enoxy]anilino]benzoate (1 g, 1.59 mmol, 49.66% yield, 96.35% purity) as a yellow solid.

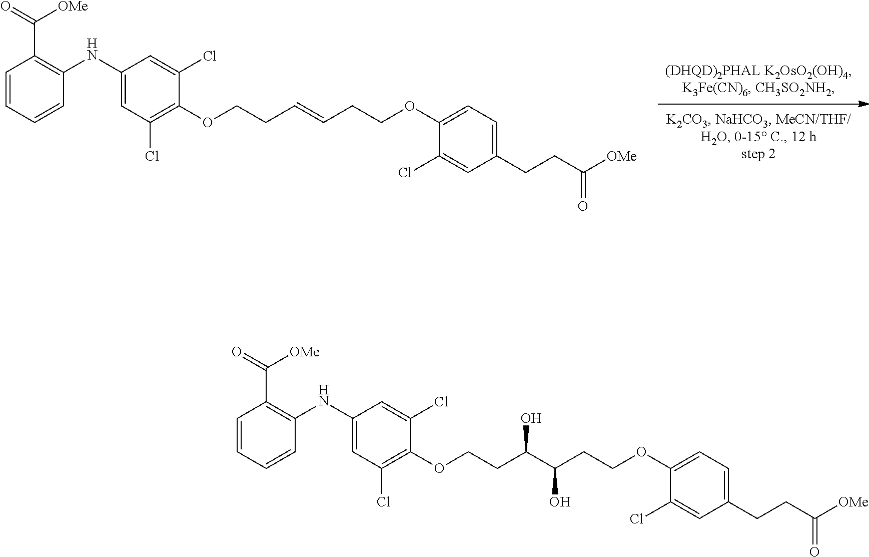 [SMILES: COC(=O)CCc1ccc(OCC/C=C/CCOc2c(Cl)cc(Nc3ccccc3C(=O)OC)cc2Cl)c(Cl)c1 ; COC(=O)CCc1ccc(OCC[C@@H](O)[C@H](O)CCOc2c(Cl)cc(Nc3ccccc3C(=O)OC)cc2Cl)c(Cl)c1]

### Methyl 2-[3,5-dichloro-4[(3R,4R)-6-[2-chloro-4-(3-methoxy-3-oxo-propyl)phenoxy]-3,4-dihydroxyhexoxy]anilino]benzoate

A solution of NaHCO₃ (166.10 mg, 1.98 mmol, 76.90 uL, 1 eq), (DHQD)₂PHAL (77.01 mg, 98.86 umol, 0.05 eq), K₂OsO₄·2H₂O (14.57 mg, 39.54 umol, 0.02 eq), K₂CO₃ (683.17 mg, 4.94 mmol, 2.5 eq), K₃[Fe(CN)₆] (1.63 g, 4.94 mmol, 1.36 mL, 2.5 eq) and MeSO₂NH₂ (188.07 mg, 1.98 mmol, 1 eq) in Water (20 mL) and MeCN (20 mL) at 15° C. was stirred for 15 min. The clear solution was cooled to 0° C. and a solution of methyl 2-[3,5-dichloro-4-[(E)-6-[2-chloro-4-(3-methoxy-3-oxo-propyl)phenoxy]hex-3-enoxy]anilino]benzoate (1.2 g, 1.98 mmol, 1 eq) in THF (20 mL) was added. The suspension was diluted with THF (200 mL) to clear solution. The solution was stirred at 15° C. for 12 h. Na₂SO₃ (5 g) in H₂O (50 mL) was added, and the mixture was stirred at 15° C. for 10 min. The mixture was extracted with EtOAc (4×70 mL). The combined organic layers were washed with brine (30 mL), dried over Na₂SO₄ and concentrated under vacuum to give a residue. The residue was purified by column (SiO₂, PE/EtOAc=20/1 ˜1/1) to afford methyl 2-[3,5-dichloro-4-[(3R,4R)-6-[2-chloro-4-(3-methoxy-3-oxo-propyl)phenoxy]-3,4-dihydroxyhexoxy]anilino]benzoate (1.0 g, 1.51 mmol, 76.54% yield, 97% purity) as a light yellow solid. The product was checked by ¹H NMR and Chiral SFC (Retention time: P1: 3.242 min, P2: 3.584 min)

¹H NMR: ET20197-301-P1AA (400 MHz, DMSO-d₆)

δ 9.12 (s, 1H), 7.89 (dd, J=1.5, 8.0 Hz, 1H), 7.50-7.43 (m, 1H), 7.31 (s, 2H), 7.28-7.22 (m, 2H), 7.14-7.10 (m, 1H), 7.06-7.02 (m, 1H), 6.93-6.87 (m, 1H), 4.55 (dd, J=5.8, 13.9 Hz, 2H), 4.16-4.05 (m, 4H), 3.84 (s, 3H), 3.67-3.59 (m, 2H), 3.56 (s, 3H), 2.80-2.73 (m, 2H), 2.62-2.56 (m, 2H), 1.99-1.72 (m, 4H)

SFC: ET20197-301-P1S (Retention time:3.242 min, 97.8% ee).

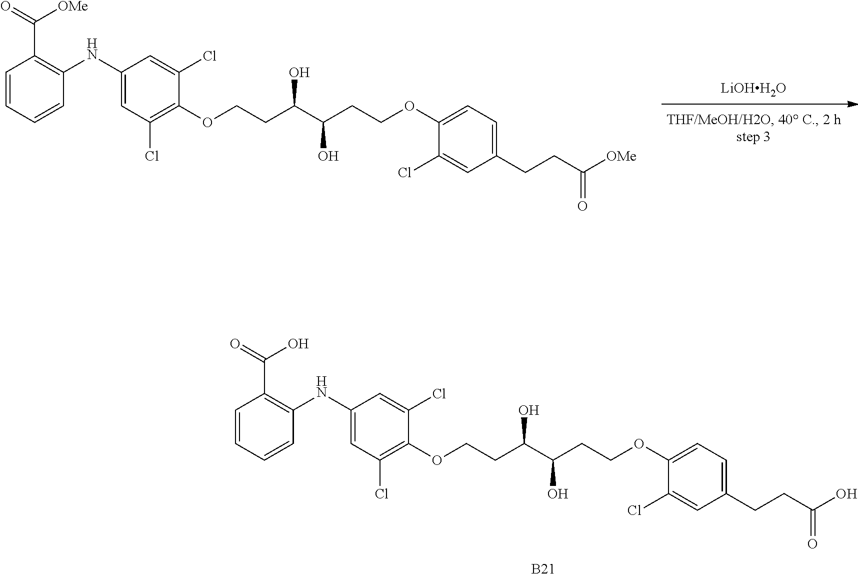

### 2-[4-[6-[4-(2-carboxyethyl)-2-chloro-phenoxy]-3,4-dihydroxy-hexoxy]-3,5-dichloro-anilino]benzoic acid

To a solution of methyl 2-[3,5-dichloro-4-[6-[2-chloro-4-(3-methoxy-3-oxo-propyl)phenoxy]-3,4-dihydroxyhexoxy]anilino]benzoate (1 g, 1.56 mmol, 1 eq) in THF (30 mL) was added LiOH·H₂O (327.36 mg, 7.80 mmol, 5 eq). The mixture was stirred at 40° C. for 2.0 h. The reaction was diluted with H₂O (10 mL) and extracted with EtOAc (10 mL*2). The aqueous layers were acidified with HCl (1N) to pH 5 ˜6. The suspension was extracted with 2-methyltetrahydrofuran (50 mL*3) and washed with brine (20 mL), dried over Na₂SO₄ and filtered and concentrated to give the residue. The residue was purified by prep-HPLC (HCl condition) to afford 2-[4-[6-[4-(2-carboxyethyl)-2-chloro-phenoxy]-3,4-dihydroxy-hexoxy]-3,5-dichloro-anilino]benzoic acid (661.1 mg, 1.08 mmol, 99.73% purity, 69.14% yield) as a white solid.

LCMS: ET2019-306-P1P1 (M+H⁺): 614.1 @ 1.986 min (5-90% ACN in H₂O, 4.5 min)

SFC: ET20197-306-P1S (Retention time: 3.10 min, 100% ee)

¹H NMR: ET20197-306-P1BB, (400 MHz, METHANOL-d₄)

δ 8.00 (dd, J=1.6, 7.9 Hz, 1H), 7.41 (dt, J=1.7, 7.8 Hz, 1H), 7.26-7.20 (m, 4H), 7.11 (dd, J=2.0, 8.4 Hz, 1H), 7.00 (d, J=8.4 Hz, 1H), 6.84 (t, J=7.6 Hz, 1H), 4.27-4.11 (m, 4H), 3.93-3.86 (m, 2H), 2.83 (t, J=7.5 Hz, 2H), 2.60-2.51 (m, 2H), 2.19-1.93 (m, 4H)

DSC: ET20197-306-P1D (Peak: 172.3° C., Onset: 169.9° C., End: 174.0° C.)

### Synthesis of B22 (T-643)

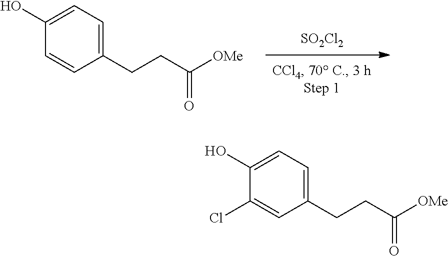 [SMILES: COC(=O)CCc1ccc(O)cc1 ; "SO2Cl2" ; COC(=O)CCc1ccc(O)c(Cl)c1]

### Methyl 3-(3-chloro-4-hydroxy-phenyl)propanoate

To a solution of methyl 3-(4-hydroxyphenyl)propanoate (10 g, 55.49 mmol, 1 eq) in CCl₄ (100 mL) was added sulfuryl chloride (8.24 g, 61.04 mmol, 6.10 mL, 1.1 eq). The mixture was stirred at 70° C. for 3 hr. The reaction mixture was quenched by addition H₂O 150 mL and partitioned, the aqueous layer was extracted with EtOAc 80 mL×2. The combined organic layers were washed with brine 150 mL, dried over Na₂SO₄, filtered and concentrated under reduced pressure to give a residue. The residue was purified by flash silica gel chromatography (ISCO®; 40 g SepaFlash® Silica Flash Column, Eluent of 0 ˜7% Ethyl acetate/Petroleum ethergradient @ 100 mL/min) to give crude product 12 g. Then the crude product was purified by reversed-phase MPLC (HCl condition, ET21585-114-P1H) to afford methyl 3-(3-chloro-4-hydroxy-phenyl)propanoate (9 g, 37.74 mmol, 68.00% yield, 90% purity) as a yellow solid.

¹H NMR: ET21585-114-P1AB (CHLOROFORM-d, 400 MHz)

δ 7.16 (d, J=1.8 Hz, 1H), 7.03-6.97 (m, 1H), 6.96-6.90 (m, 1H), 5.56 (br s, 1H), 3.68 (s, 3H), 2.91-2.83 (m, 2H), 2.63-2.56 (m, 2H)

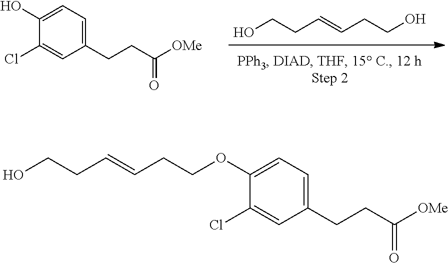 [SMILES: COC(=O)CCc1ccc(O)c(Cl)c1 ; OCC/C=C/CCO ; COC(=O)CCc1ccc(OCC/C=C/CCO)c(Cl)c1]

### Methyl 3-[3-chloro-4-[(E)-6-hydroxyhex-3-enoxy]phenyl]propanoate

To a solution of (E)-hex-3-ene-1,6-diol (5.41 g, 46.58 mmol, 2 eq), methyl 3-(3-chloro-4-hydroxy-phenyl)propanoate (5 g, 23.29 mmol, 1 eq) and PPh₃ (12.22 g, 46.58 mmol, 2 eq) in THF (100 mL) was added DIAD (9.42 g, 46.58 mmol, 9.06 mL, 2 eq). The mixture was stirred at 15° C. for 12 hr. The reaction mixture was concentrated under reduced pressure to remove solvent. The residue was purified by flash silica gel chromatography (ISCO®; 80 g SepaFlash® Silica Flash Column, Eluent of 0 ˜16% Ethyl acetate/Petroleum ethergradient @100 mL/min) to give crude product 12 g. Then the crude product was purified by reversed MPLC (HCl condition) to afford 3-[3-chloro-4-[(E)-6-hydroxyhex-3-enoxy]phenyl]propanoate (3 g, 9.59 mmol, 41.18% yield) as yellow oil.

¹H NMR: ET21585-123-P1AA (CHLOROFORM-d, 400 MHz)

δ 7.20 (d, J=2.0 Hz, 1H), 7.02 (dd, J=2.1, 8.3 Hz, 1H), 6.83 (d, J=8.4 Hz, 1H), 5.70-5.53 (m, 2H), 4.03 (t, J=6.5 Hz, 2H), 3.69-3.63 (m, 5H), 2.90-2.83 (m, 2H), 2.63-2.52 (m, 4H), 2.30 (q, J=6.4 Hz, 2H)

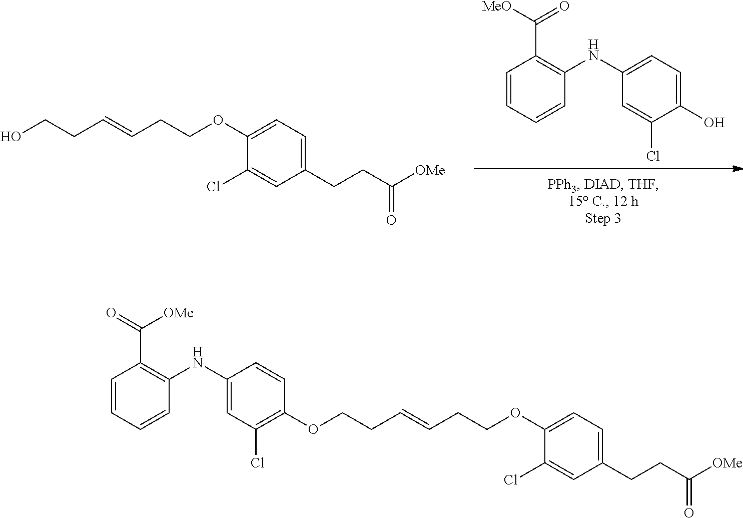 [SMILES: COC(=O)CCc1ccc(OCC/C=C/CCO)c(Cl)c1 ; COC(=O)c1ccccc1Nc1ccc(O)c(Cl)c1 ; COC(=O)CCc1ccc(OCC/C=C/CCOc2ccc(Nc3ccccc3C(=O)OC)cc2Cl)c(Cl)c1]

### Methyl 2-[3-chloro 4-[(E)-6-[2-chloro 4-(3-methoxy-3-oxo-propyl)phenoxy]hex-3-enoxy]anilino]benzoate

To a solution of methyl 2-(3-chloro-4-hydroxy-anilino)benzoate (887.83 mg, 3.20 mmol, 1 eq), methyl 3-[3-chloro-4-[(E)-6-hydroxyhex-3-enoxy]phenyl]propanoate (1 g, 3.20 mmol, 1 eq) and PPh₃ (1.26 g, 4.80 mmol, 1.5 eq) in THF (20 mL) was added DIAD (969.72 mg, 4.80 mmol, 932.42 uL, 1.5 eq). The mixture was stirred at 15° C. for 12 hr. The reaction mixture (combined with ET21585-126, 100 mg scale) was concentrated under reduced pressure to remove solvent. The crude product was purified by reversed MPLC (HCl condition, SiO2, 50 ˜100% H2O/MeOH) to afford methyl 2-[3-chloro-4-[(E)-6-[2-chloro-4-(3-methoxy-3-oxo-propyl)phenoxy]hex-3-enoxy]anilino]benzoate (1.15 g) as a yellow solid.

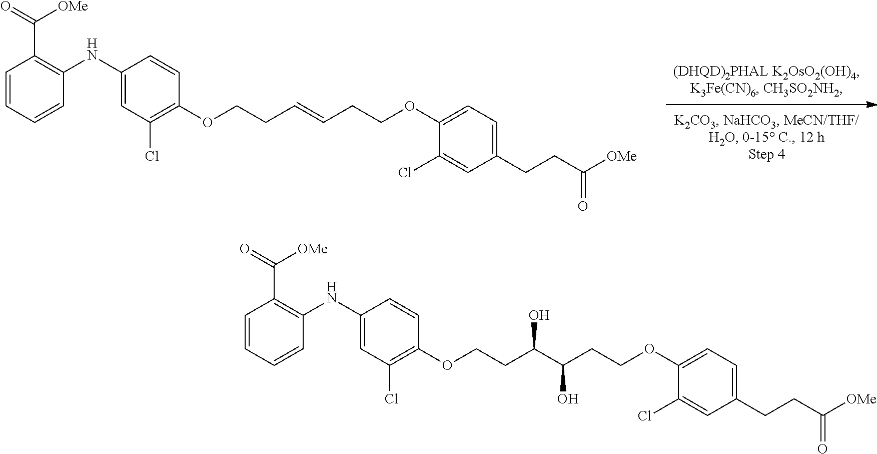 [SMILES: COC(=O)CCc1ccc(OCC/C=C/CCOc2ccc(Nc3ccccc3C(=O)OC)cc2Cl)c(Cl)c1 ; COC(=O)CCc1ccc(OCC[C@@H](O)[C@H](O)CCOc2ccc(Nc3ccccc3C(=O)OC)cc2Cl)c(Cl)c1]

### Methyl 2-[3-chloro-4-[(3R,4R)-6-[2-chloro-4-(3-methoxy-3-oxo-propyl)phenoxy]-3,4-dihydroxy-hexoxy]anilino]benzoate

A mixture of K₂OsO₄·2H₂O (12.90 mg, 35.00 umol, 0.02 eq), (DHQD)₂PHAL (68.16 mg, 87.50 umol, 0.05 eq), NaHCO₃ (147.02 mg, 1.75 mmol, 68.06 uL, 1 eq), MeSO₂NH₂ (166.46 mg, 1.75 mmol, 1 eq), K₂CO₃ (604.67 mg, 4.38 mmol, 2.5 eq) and K₃[Fe(CN)₆] (1.44 g, 4.38 mmol, 1.20 mL, 2.5 eq) in H₂O (40 mL) and MeCN (40 mL) was stirred for 15 min at 15° C. The mixture was cooled to 0° C. and methyl 2-[3-chloro-4-[(E)-6-[2-chloro-4-(3-methoxy-3-oxo-propyl)phenoxy]hex-3-enoxy]anilino]benzoate (1 g, 1.75 mmol, 1 eq) in THF (80 mL) was added. The reaction was stirred at 0° C. for 3 hr and 15° C. for 8 hr 45 min. The reaction mixture was quenched by addition Na₂SO₃ 10 g in H₂O 70 mL and extracted with EtOAc 60 mL×3. The combined organic layers were washed with brine 70 mL, dried over Na₂SO₄, filtered and concentrated under reduced pressure to give a residue. The residue was purified by flash silica gel chromatography (ISCO®; 20 g SepaFlash® Silica Flash Column, Eluent of 0 ˜50% Ethyl acetate/Petroleum ethergradient @ 75 mL/min) to afford methyl 2-[3-chloro-4-[(3R,4R)-6-[2-chloro-4-(3-methoxy-3-oxo-propyl)phenoxy]-3,4-dihydroxy-hexoxy]anilino]benzoate (880 mg, 1.42 mmol, 81.25% yield, 98% purity) as a yellow solid.

SFC: ET21585-132-P1A1 (Retention time: 2.22 min; 98.8% ee value)

¹H NMR: ET21585-132-P1AA ET20197-144-P2AA (DMSO-d6, 400 MHz)

δ 9.13 (s, 1H), 7.87 (dd, J=1.5, 8.0 Hz, 1H), 7.41-7.35 (m, 1H), 7.32 (d, J=2.3 Hz, 1H), 7.27 (d, J=2.1 Hz, 1H), 7.21-7.11 (m, 3H), 7.06-7.03 (m, 1H), 7.00 (d, J=8.4 Hz, 1H), 6.79-6.74 (m, 1H), 4.59 (dd, J=2.3, 5.9 Hz, 2H), 4.16 (td, J=6.5, 13.0 Hz, 4H), 3.85 (s, 3H), 3.66-3.60 (m, 2H), 3.57 (s, 3H), 2.80-2.74 (m, 2H), 2.62-2.57 (m, 2H), 1.98-1.89 (m, 2H), 1.85-1.75 (m, 2H)

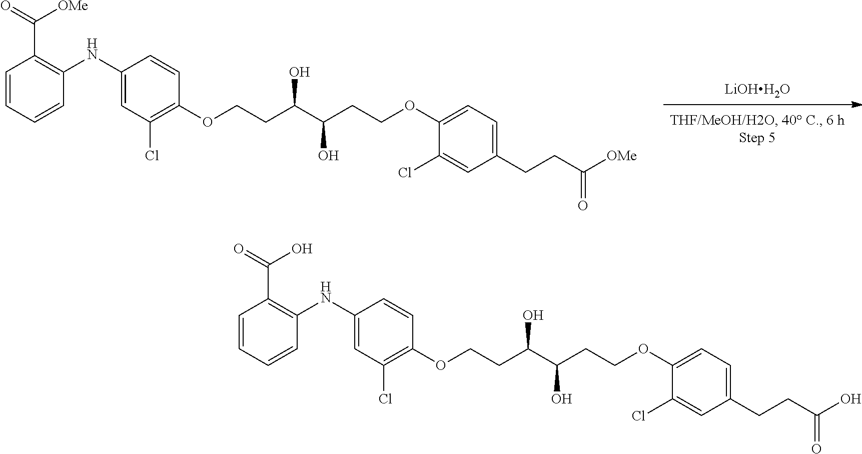 [SMILES: COC(=O)CCc1ccc(OCC[C@@H](O)[C@H](O)CCOc2ccc(Nc3ccccc3C(=O)OC)cc2Cl)c(Cl)c1 ; "LiOH·H2O" ; O=C(O)CCc1ccc(OCC[C@@H](O)[C@H](O)CCOc2ccc(Nc3ccccc3C(=O)O)cc2Cl)c(Cl)c1]

### 2-[4-[(3R,4R)-6-[4-(2-carboxyethyl)-2-chloro-phenoxy]-3,4-dihydroxy-hexoxy]-3-chloro-anilino]benzoic acid

To a solution of methyl 2-[3-chloro-4-[(3R,4R)-6-[2-chloro-4-(3-methoxy-3-oxo-propyl) phenoxy]-3,4-dihydroxy-hexoxy]anilino]benzoate (800.00 mg, 1.32 mmol, 1 eq) in THF (30 mL) and MeOH (15 mL) was added LiOH·H₂O (332.09 mg, 7.91 mmol, 6 eq) in H₂O (15 mL). The mixture was stirred at 40° C. for 6 hr. The reaction mixture (combined with ET21585-137, 80 mg scale) was diluted with H₂O 40 mL and acidified to pH=6 with HCl (1 N), the mixture was extracted with EtOAc 30 mL×3. The combined organic layers were washed with brine 60 mL, dried over Na₂SO₄, filtered and concentrated under reduced pressure to give a residue. The residue was purified by prep-HPLC (Phenomenex Luna C18 200*40 mm*10 um; mobile phase: [water (0.05% HCl)-ACN]; B %: 35%-55%, 10 min,) to afford 2-[4-[(3R,4R)-6-[4-(2-carboxyethyl)-2-chloro-phenoxy]-3,4-dihydroxy-hexoxy]-3-chloro-anilino]benzoic acid (420 mg) as a white solid.

LCMS: ET21585-139-P1A4 (M+H+): 578.0@ 2.860 min (10-80% ACN in H2O, 4.5 min)

¹H NMR: ET21585-139-P1AC (METHANOL-d₄, 400 MHz)

δ 7.96 (dd, J=1.5, 8.0 Hz, 1H), 7.31 (dt, J=1.6, 7.8 Hz, 1H), 7.24-7.18 (m, 2H), 7.14-7.06 (m, 3H), 7.00 (dd, J=8.4, 14.0 Hz, 2H), 6.74-6.67 (m, 1H), 4.26-4.15 (m, 4H), 3.90-3.82 (m, 2H), 2.86-2.78 (m, 2H), 2.55 (t, J=7.5 Hz, 2H), 2.16-2.05 (m, 2H), 2.04-1.93 (m, 2H)

SFC: ET21585-139-P1A3 (Retention time: 5.24 min; 100% ee value)

DSC: ET21585-139-P1A2 (Peak: 182.5° C., onset: 180.7° C., end: 183.9° C.).

### Synthesis of B23 (T-762)

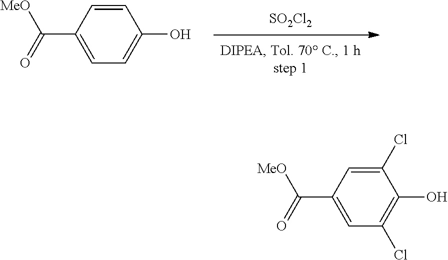 [SMILES: COC(=O)c1ccc(O)cc1 ; "SO2Cl2" ; COC(=O)c1cc(Cl)c(O)c(Cl)c1]

### Methyl 3,5-dichloro-4-hydroxy-benzoate

A solution of methyl 4-hydroxybenzoate (10 g, 65.73 mmol, 1 eq) and DIPA (665.08 mg, 6.57 mmol, 928.88 uL, 0.1 eq) in tol. (500 mL) was heated to 70° C., and then sulfuryl chloride (22.18 g, 164.31 mmol, 16.43 mL, 2.5 eq) was added drop wise at 70° C. The reaction mixture was stirred at 70° C. for 1 hr. The reaction mixture was cooled to 15° C., quenched with H₂O (500 mL) and separated. The organic layer was washed with water (100 mL*4). The organic layer was dried with Na₂SO₄, filtered and concentrated in vacuo to give methyl 3,5-dichloro-4-hydroxy-benzoate (15 g, crude) as a white solid.

¹H NMR: ET20197-262-P1AA (400 MHz, CHLOROFORM-d)

δ 7.90 (s, 2H), 6.26 (br s, 1H), 3.83 (s, 3H)

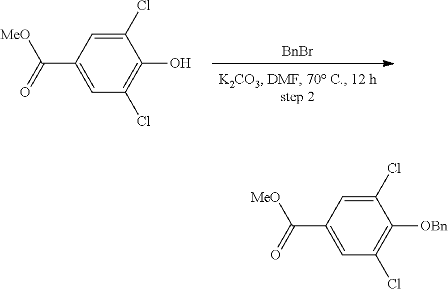 [SMILES: COC(=O)c1cc(Cl)c(O)c(Cl)c1 ; "BnBr" ; COC(=O)c1cc(Cl)c(OCc2ccccc2)c(Cl)c1]

### Methyl 4-benzyloxy-3,5-dichloro-benzoate

To a solution of methyl 3,5-dichloro-4-hydroxy-benzoate (15 g, 67.86 mmol, 1 eq) in DMF (100 mL) were added K₂CO₃ (16.58 g, 119.99 mmol, 1.77 eq) and bromomethylbenzene (21.59 g, 126.22 mmol, 14.99 mL, 1.86 eq). The mixture was stirred at 70° C. for 12 hr. The reaction mixture was filtered and diluted with EtOAc (200 mL), washed with sub-saturated brine (100 mL*3). The organic layer was dried over Na₂SO₄ and concentrated to give a residue. The residue was purified by column (SiO₂, Petroleum ether/Ethyl acetate=1-20/1) to give a crude. The crude product was further purified by re-crystallization from MeOH (100 mL) at 15° C. to afford methyl 4-benzyloxy-3,5-dichloro-benzoate (11 g, 35.35 mmol, 52.09% yield) as a white solid.

¹H NMR: ET20197-269-P1AA (400 MHz, DMSO-d₆)

δ 7.97 (s, 2H), 7.52 (br d, J=6.8 Hz, 2H), 7.45-7.36 (m, 3H), 5.11 (s, 2H), 3.86 (s, 3H)

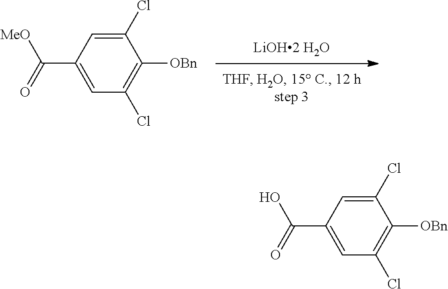 [SMILES: COC(=O)c1cc(Cl)c(OCc2ccccc2)c(Cl)c1 ; "LiOH·2 H2O" ; O=C(O)c1cc(Cl)c(OCc2ccccc2)c(Cl)c1]

### 4-benzyloxy-3,5-dichloro-benzoic acid

To a solution of methyl 4-benzyloxy-3,5-dichloro-benzoate (11 g, 35.35 mmol, 1 eq) in THF (15 mL)/H₂O (5 mL) was added LiOH·H₂O (5.93 g, 141.41 mmol, 4 eq). The reaction was stirred at 15° C. for 12 h. The reaction was adjusted with HCl (1N) to pH 5 ˜6 and extracted with EtOAc (100 mL*3). The organic layer was washed with brine (50 mL), dried Na₂SO₄, filtered and concentrated to give 4-benzyloxy-3,5-dichloro-benzoic acid (8.5 g, 28.61 mmol, crude) as a white solid.

¹H NMR: ET20197-276-P1AA (400 MHz, DMSO-d₆)

δ 7.95 (s, 2H), 7.53 (br d, J=6.7 Hz, 2H), 7.45-7.32 (m, 3H), 5.10 (s, 2H)

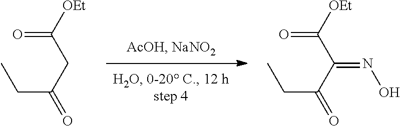

### Ethyl (2E)-2-hydroxyimino-3-oxo-pentanoate

A solution of NaNO₂ (7.18 g, 104.05 mmol, 1.5 eq) in H₂O (20 mL) was added drop wise to a solution of ethyl 3-oxopentanoate (10 g, 69.36 mmol, 1 eq) in AcOH (70 mL) at 0° C. The reaction mixture was stirred at 20° C. for 12 hr. Saturated aqueous NaHCO₃ solution (200 mL) was added to the reaction mixture, extracted with EtOAc (70 mL*3), dried over Na₂SO₄, filtered and concentrated to ethyl (2E)-2-hydroxyimino-3-oxo-pentanoate (12 g, crude) as a light yellow oil.

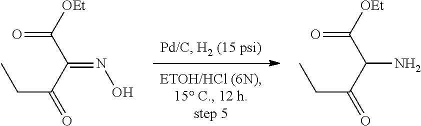

### Ethyl 2-amino-3-oxo-pentanoate

To a solution of ethyl (2E)-2-hydroxyimino-3-oxo-pentanoate (10 g, 57.75 mmol, 1 eq) in EtOH (140 mL)/HCl (6 M, 30 mL, 3.12 eq) was added Pd/C (3 g, 10% purity, 1.00 eq). The mixture was stirred at 15° C. for 12 hr under H₂ (15 psi). The reaction was filtered and concentrated to give the residue. The residue was washed with MTBE (100 mL) and filtered. The crude ethyl 2-amino-3-oxo-pentanoate (10 g, crude, HCl) was obtained as a white solid.

¹H NMR: ET20197-279-P1AA (400 MHz, DMSO-d₆)

δ 8.98 (br s, 2H), 5.31-5.18 (m, 1H), 4.32-4.17 (m, 2H), 2.89-2.69 (m, 2H), 1.25 (t, J=7.2 Hz, 3H), 0.99 (t, J=7.2 Hz, 3H)

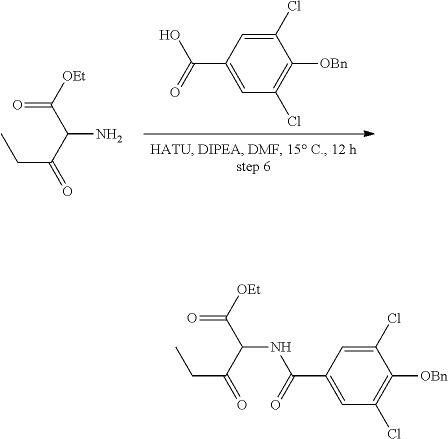 [SMILES: CCOC(=O)C(N)C(=O)CC ; O=C(O)c1cc(Cl)c(OCc2ccccc2)c(Cl)c1 ; CCOC(=O)C(NC(=O)c1cc(Cl)c(OCc2ccccc2)c(Cl)c1)C(=O)CC]

### Ethyl 2-[(4-benzyloxy-3,5-dichloro-benzoyl)amino]-3-oxo-pentanoate

To a solution of 4-benzyloxy-3,5-dichloro-benzoic acid (7.75 g, 26.07 mmol, 1 eq) and ethyl 2-amino-3-oxo-pentanoate (5.1 g, 26.07 mmol, 1 eq, HCl) in DMF (3 mL) was added HATU (14.87 g, 39.10 mmol, 1.5 eq), DIPEA (10.11 g, 78.20 mmol, 13.62 mL, 3 eq) at 15° C. The reaction was stirred at 15° C. for 12 hr. The reaction was diluted with H₂O (100 mL) and extracted with EtOAc (50 mL*4). The combined organic layers were washed with brine (50 mL*2), dried over [Na₂SO₄], filtered and concentrated under reduced pressure to give a residue. The residue was purified by column chromatography (SiO₂, Petroleum ether/Ethyl acetate=20/1 to 5:1) to afford ethyl 2-[(4-benzyloxy-3,5-dichloro-benzoyl)amino]-3-oxo-pentanoate (5.2 g, 11.86 mmol, 45.51% yield) as a white solid.

¹H NMR: ET20197-275-P1AA (400 MHz, DMSO-d₆)

δ 9.42 (d, J=7.7 Hz, 1H), 8.05 (s, 2H), 7.53 (br d, J=6.6 Hz, 2H), 7.47-7.35 (m, 3H), 5.43 (d, J=7.5 Hz, 1H), 5.11 (s, 2H), 4.27-4.12 (m, 2H), 2.72-2.65 (m, 2H), 1.22 (t, J=7.1 Hz, 3H), 0.98 (t, J=7.2 Hz, 3H)

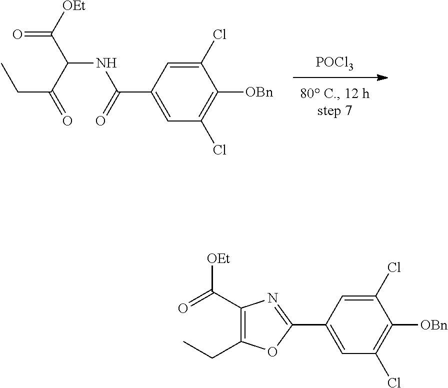 [SMILES: CCOC(=O)C(NC(=O)c1cc(Cl)c(OCc2ccccc2)c(Cl)c1)C(=O)CC ; "POCl3" ; CCOC(=O)c1nc(-c2cc(Cl)c(OCc3ccccc3)c(Cl)c2)oc1CC]

### Ethyl 2-(4-benzyloxy-3,5-dichloro-phenyl)-5-ethyl-oxazole-4-carboxylate

A solution of ethyl 2-[(4-benzyloxy-3,5-dichloro-benzoyl)amino]-3-oxo-pentanoate (5 g, 11.41 mmol, 1 eq) in POCl₃ (16.50 g, 107.61 mmol, 10 mL, 9.43 eq) was stirred at 80° C. for 12 hr. The reaction was concentrated to remove POCl₃, and the residue was dropped into an ice-cold saturated NaHCO₃ aqueous solution (200 mL) and extracted with 2-methyl tetrahydrofuran (70 mL*3), dried over Na₂SO₄ and concentrated. Ethyl 2-(4-benzyloxy-3,5-dichloro-phenyl)-5-ethyl-oxazole-4-carboxylate (1.2 g, 2.86 mmol, 25.03% yield) was obtained as a light yellow solid.

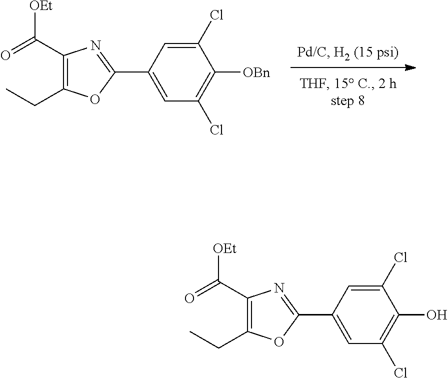 [SMILES: CCOC(=O)c1nc(-c2cc(Cl)c(OCc3ccccc3)c(Cl)c2)oc1CC ; "Pd/C, H2 (15 psi)" ; CCOC(=O)c1nc(-c2cc(Cl)c(O)c(Cl)c2)oc1CC]

### Ethyl 2-(3,5-dichloro-4-hydroxy-phenyl)-5-ethyl-oxazole-4-carboxylate

To a solution of ethyl 2-(4-benzyloxy-3,5-dichloro-phenyl)-5-ethyl-oxazole-4-carboxylate (1.7 g, 4.04 mmol, 1 eq) in THF (20 mL) was added Pd/C (0.5 g, 4.04 mmol, 10% purity, 1 eq) under N₂. The suspension was degassed under vacuum and purged with H₂ several times. The mixture was stirred under H₂ (15 psi) at 15° C. for 2 hr. The reaction was filtered through a layer of Celite and concentrated to give the crude. The crude was purified by column (SiO₂, PE/EtOAc=10:1-2:1) to afford ethyl 2-(3,5-dichloro-4-hydroxy-phenyl)-5-ethyl-oxazole-4-carboxylate (0.8 g, 2.42 mmol, 59.90% yield) as a white solid.

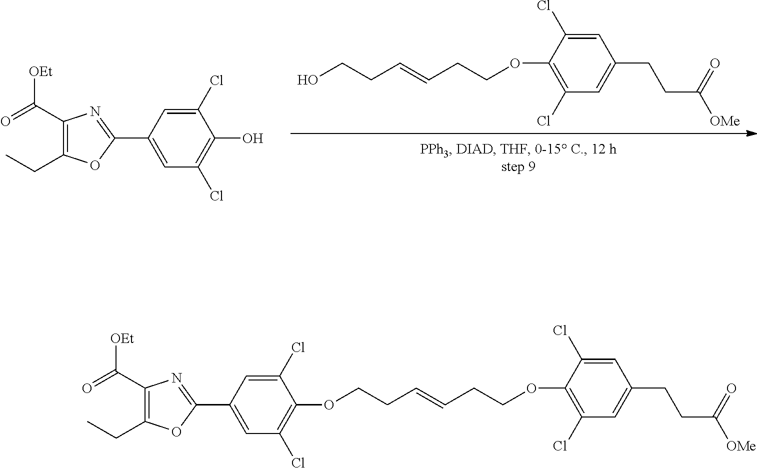 [SMILES: CCOC(=O)c1nc(-c2cc(Cl)c(O)c(Cl)c2)oc1CC ; COC(=O)CCc1cc(Cl)c(OCC/C=C/CCO)c(Cl)c1 ; CCOC(=O)c1nc(-c2cc(Cl)c(OCC/C=C/CCOc3c(Cl)cc(CCC(=O)OC)cc3Cl)c(Cl)c2)oc1CC]

### Ethyl 2-[3,5-dichloro-4-[(E)-6-[2,6-dichloro-4-(3-methoxy-3-oxo-propyl)phenoxy]hex-3-enoxy]phenyl]-5-ethyl-oxazole-4-carboxylate

To a mixture of ethyl 2-(3,5-dichloro-4-hydroxy-phenyl)-5-ethyl-oxazole-4-carboxylate (0.8 g, 2.42 mmol, 1 eq), methyl 3-[3,5-dichloro-4-[(E)-6-hydroxyhex-3-enoxy]phenyl]propanoate (1.05 g, 2.42 mmol, 1 eq) and PPh₃ (1.27 g, 4.85 mmol, 2 eq) in THF (2 mL) was added dropwise DIAD (979.92 mg, 4.85 mmol, 942.23 uL, 2 eq) at 0° C., the reaction mixture was stirred at 15° C. for 12 h. The reaction was concentrated under vacuum to give a residue. The crude product was purified by reversed-phase HPLC (0.1% HCl condition/ACN/H₂O) to afford ethyl 2-[3,5-dichloro-4-[(E)-6-[2,6-dichloro-4-(3-methoxy-3-oxo-propyl)phenoxy] hex-3-enoxy]phenyl]-5-ethyl-oxazole-4-carboxylate (1.0 g, 1.52 mmol, 62.59% yield) as a white solid.

¹H NMR: ET20197-297-P1AA (400 MHz, DMSO-d₆)

δ 7.95 (s, 2H), 7.31 (s, 2H), 5.69 (td, J=2.4, 4.7 Hz, 2H), 4.30 (q, J=7.1 Hz, 2H), 4.08 (t, J=6.7 Hz, 2H), 3.94 (t, J=6.7 Hz, 2H), 3.58 (s, 3H), 3.07 (q, J=7.5 Hz, 2H), 2.83-2.74 (m, 2H), 2.66-2.58 (m, 2H), 2.56-2.51 (m, 2H), 2.49-2.45 (m, 2H), 1.36-1.23 (m, 6H)

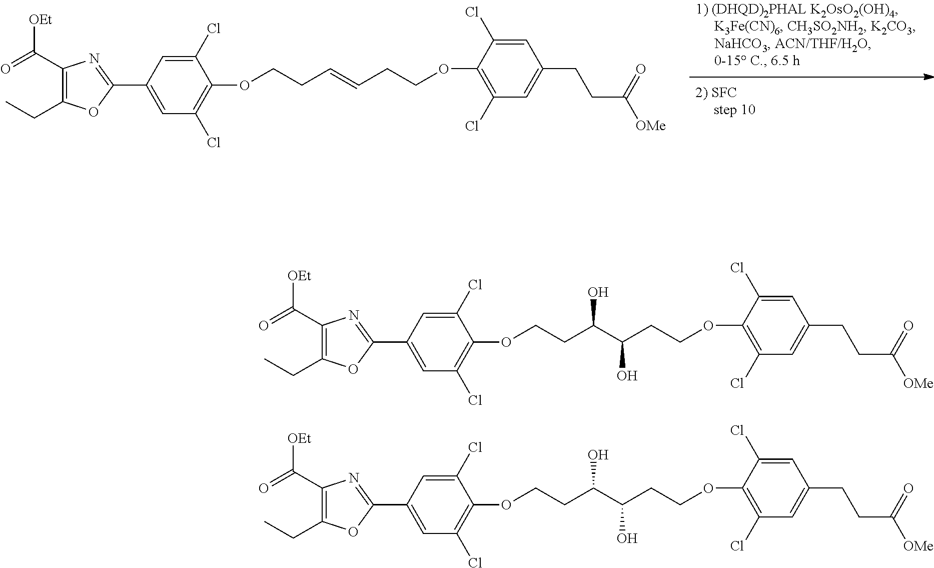 [SMILES: CCOC(=O)c1nc(-c2cc(Cl)c(OCC/C=C/CCOc3c(Cl)cc(CCC(=O)OC)cc3Cl)c(Cl)c2)oc1CC ; CCOC(=O)c1nc(-c2cc(Cl)c(OCC[C@@H](O)[C@H](O)CCOc3c(Cl)cc(CCC(=O)OC)cc3Cl)c(Cl)c2)oc1CC ; CCOC(=O)c1nc(-c2cc(Cl)c(OCC[C@H](O)[C@@H](O)CCOc3c(Cl)cc(CCC(=O)OC)cc3Cl)c(Cl)c2)oc1CC]

### Ethyl 2-[3,5-dichloro-4-[(3R,4R)-6-[2,6-dichloro-4-(3-methoxy-3-oxo-propyl)phenoxy]-3,4-dihydroxyhexoxy]phenyl]-5-ethyl-oxazole-4-carboxylate

A solution of NaHCO₃ (89.19 mg, 1.06 mmol, 41.29 uL, 1 eq), (DHQD)₂PHAL (165.39 mg, 212.32 umol, 0.2 eq), K₂OsO₄·2H₂O (7.82 mg, 21.23 umol, 0.02 eq), K₂CO₃ (366.81 mg, 2.65 mmol, 2.5 eq), K₃[Fe(CN)] (873.81 mg, 2.65 mmol, 728.17 uL, 2.5 eq) and MeSO₂NH₂ (100.98 mg, 1.06 mmol, 1 eq) in Water (20 mL) and MeCN (20 mL) at 15° C. was stirred for 30 min. The clear solution was cooled to 0° C. and a solution of ethyl 2-[3,5-dichloro-4-[(E)-6-[2,6-dichloro-4-(3-methoxy-3-oxo-propyl)phenoxy]hex-3-enoxy]phenyl]-5-ethyl-oxazole-4-carboxylate (700 mg, 1.06 mmol, 1 eq) in THF (20 mL) was added. The suspension was diluted with THF (200 mL) to turn clear. The resulting solution was stirred at 15° C. for 6 h. Na₂SO₃ (3 g) in H₂O (50 mL) was added, and the mixture was stirred at 15° C. for 10 min. The mixture was extracted with EtOAc (4×100 mL). The combined organic layers were washed with brine (50 mL), dried over Na₂SO₄ and concentrated under vacuum to give a residue. The residue was purified by column (SiO₂, PE/EtOAc=10/1 ˜1/1)(Plate 1) to afford a mixture of P1 and P2 (400 mg). The mixture was detected by HPLC and SFC (Retention time: P1: 2.03 min; P2: 2.63 min)

The mixture (combined with a batch of ET20197-304, 30 mg scale and ET20197-305, 30 mg scale) was separated by SFC (Instrument: Waters SFC80Q preparative SFC; Column: Chiralpak AD-H, 250*30 mm i.d., Sum; Mobile phase: A for CO₂ and B for MeOH:ACN=4:1(Neu); Gradient: B %=50% Flow rate:70 g/min; Column temperature: 40° C.; System back pressure:100 bar) to give:

### P1:

Ethyl 2-[3,5-dichloro-4-[(3R,4R)-6-[2,6-dichloro-4-(3-methoxy-3-oxo-propyl)phenoxy]-3,4-dihydroxyhexoxy]phenyl]-5-ethyl-oxazole-4-carboxylate (310 mg, 441.68 umol, 41.61% yield, 98.794% purity, 99.78% ee) as a off-white solid.

SFC: ET20197-307-P1S1 (Retention time: P1: 1.794 min; 99.78% ee).

### P2:

Ethyl 2-[3,5-dichloro-4-[(3S,4S)-6-[2,6-dichloro-4-(3-methoxy-3-oxo-propyl)phenoxy]-3,4-dihydroxy-hexoxy]phenyl]-5-ethyloxazole-4-carboxylate (30 mg, 40.12 umol, 3.78% yield, 92.727% purity, 89.96%) as a off-white solid.

SFC: ET20197-307-P2S1 (Retention time: P2: 2.036 min; 89.96% ee).

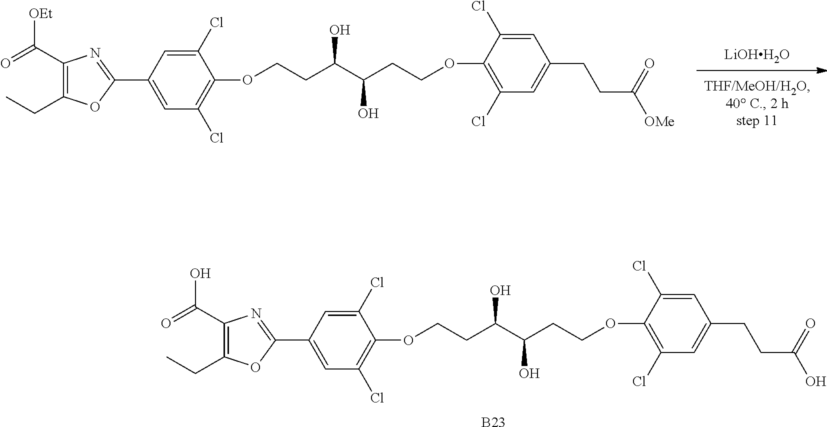

### 2-[4-[(3R,4R)-6-[4-(2-carboxyethyl)-2,6-dichloro-phenoxy]-3,4-dihydroxy-hexoxy]-3,5-dichloro-phenyl]-5-ethyl-oxazole-4-carboxylic acid

To a solution of ethyl 2-[3,5-dichloro-4-[(3R,4R)-6-[2,6-dichloro-4-(3-methoxy-3-oxo-propyl)phenoxy]-3,4-dihydroxy-hexoxy]phenyl]-5-ethyl-oxazole-4-carboxylate (310.00 mg, 447.08 umol, 1 eq) in THF (20 mL)/MeOH (10 mL) was added LiOH·H₂O (93.80 mg, 2.24 mmol, 5 eq). The mixture was stirred at 40° C. for 2 hr. The reaction was adjusted with HCl (1N) to pH 5 ˜6 and extracted with EtOAc (50 mL*3). The combined organic layers were washed with brine (20 mL), dried over [Na₂SO₄], filtered and concentrated under reduced pressure to give a residue. The residue was purified by prep-HPLC (column: Luna C18 100*30 5 u; mobile phase: [water (0.04% HCl)-ACN]; B %: 35%-60%, 10 min) to afford 2-[4-[(3R,4R)-6-[4-(2-carboxyethyl)-2,6-dichloro-phenoxy]-3,4-dihydroxy-hexoxy]-3,5-dichloro-phenyl]-5-ethyl-oxazole-4-carboxylic acid (185.9 mg, 285.42 umol, 63.84% yield, 100% purity) was obtained as a white solid.

LCMS: ET20197-308-P1P2 (M+H): 652.0 @ 3.217 min (10-100% ACN in H₂O, 4.5 min)

SFC: ET20197-308-P1S (Retention time:2.01 min, 100% ee)

¹H NMR: ET20197-308-P1AA (400 MHz, METHANOL-d₄)

δ 8.03 (s, 2H), 7.25 (s, 2H), 4.37-4.09 (m, 4H), 3.94 (tt, J=3.6, 7.6 Hz, 2H), 3.15 (q, J=7.6 Hz, 2H), 2.89-2.80 (m, 2H), 2.63-2.54 (m, 2H), 2.24-2.08 (m, 2H), 2.07-1.92 (m, 2H), 1.35 (t, J=7.6 Hz, 3H)

DSC: ET20197-308-P1D (Peak: 165.53° C., Onset: 163.38° C., End: 168.38° C.)

### Synthesis of B24

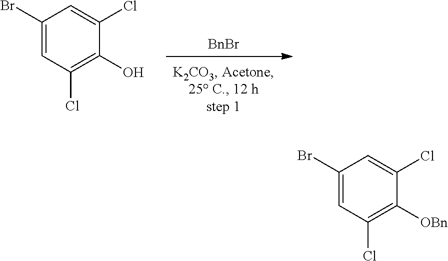

### 2-benzyloxy-1,3-dichloro-5-nitro-benzene

4-bromo-2,6-dichloro-phenol (1 g, 4.13 mmol, 1 eq) was dissolved in ACETONE (20 mL), K₂CO₃ (571.34 mg, 4.13 mmol, 1 eq) was suspended in this solution. BnBr (707.05 mg, 4.13 mmol, 491.01 uL, 1 eq) was dosed to the suspension at 25° C. The reaction was stirred at 25° C. for 6 hr. The suspension was filtered and he cake was washed with acetone (100 mL). The filtrate was concentrated to give 2-benzyloxy-5-bromo-1,3-dichloro-benzene (1.2 g, crude) as a slight yellow solid.

¹H NMR: ET20197-366-P1BB (400 MHz, DMSO-d₆)

δ 7.82 (s, 2H), 7.53-7.49 (m, 2H), 7.45-7.37 (m, 3H), 5.02 (s, 2H)

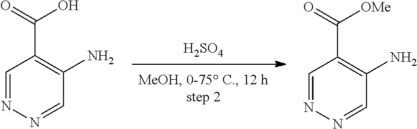 [SMILES: Nc1cnncc1C(=O)O ; "H2SO4" ; COC(=O)c1cnncc1N]

### 2-benzyloxy-5-bromo-1,3-dichloro-benzene

The solution of H₂SO₄ (6.44 g, 65.66 mmol, 3.5 mL, 18.27 eq) in MeOH (15 mL) was cooled to 0° C. The suspension of 5-aminopyridazine-4-carboxylic acid (500 mg, 3.59 mmol, 1 eq) in MeOH (2 mL) slowly added 0° C. And then the mixture was heated at 75° C. for 12 h. The mixture was cooled to r.t. and was poured into ice-water (100 mL) and neutralized with solid Na₂CO₃ to pH 7˜8. The aqueous mixture was extracted with 2-Me THF (100 mL*10) and the organic extracts were dried over Na₂SO₄, filtered, and concentrated to give the crude. The crude was purified by flash silica gel chromatography (ISCO®; 20 g SepaFlash® Silica Flash Column, Eluent of 0 ˜20% MeOH/Ethylacetate@ 75 mL/min) to give methyl 5-aminopyridazine-4-carboxylate (500 mg, 3.27 mmol, 90.84% yield) as a slight yellow solid.

¹H NMR: ET20197-377-P1AB (400 MHz, METHANOL-d4)

δ 8.90 (s, 1H), 8.70 (d, J=0.6 Hz, 1H), 3.94 (s, 3H).

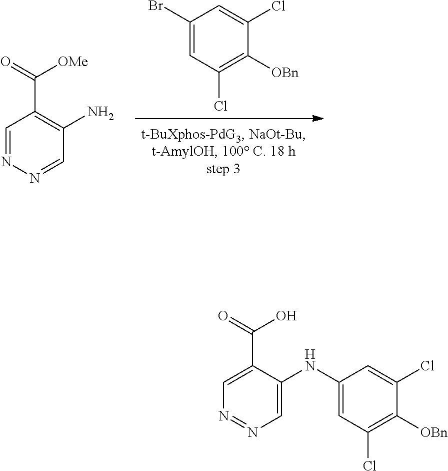 [SMILES: COC(=O)c1cnncc1N ; Clc1cc(Br)cc(Cl)c1OCc1ccccc1 ; O=C(O)c1cnncc1Nc1cc(Cl)c(OCc2ccccc2)c(Cl)c1]

### Methyl 5-(4-benzyloxy-3,5-dichloro-anilino)pyridazine-4-carboxylate

Methyl 5-aminopyridazine-4-carboxylate (170 mg, 1.11 mmol, 1 eq), 2-benzyloxy-5-bromo-1,3-dichloro-benzene (368.58 mg, 1.11 mmol, 1 eq), [2-(2-aminophenyl)phenyl]-methyl sulfonyloxy-palladium; ditert-butyl-[2-(2,4,6-triisopropylphenyl)phenyl]phosphane (88.18 mg, 111.01 umol, 0.1 eq) was taken up into a sealed bottle in 2-methyl-2-butanol (5.5 mL). t-NaOBu (2 M, 1.11 mL, 2 eq) was added into the suspension. The operation was taken in glove box, the sealed bottle was moved out glove box. The sealed bottle was heated in 100° C. for 18 hr. The reaction was (combined with ET20197-382, 40 mg scale) diluted with H₂O (100 mL) and acidified with HCl (1M) to pH 5 ˜6 then extracted with 2-MeTHF (70 mL*5). The combined organic phase was washed with saturated brine (50 mL*2), dried over anhydrous Na₂SO₄, filtered and concentrated. Methyl 5-(4-benzyloxy-3,5-dichloro-anilino)pyridazine-4-carboxylate (1.6 g, crude, 11.9% purity) was obtained as a brown-black oil without further purification.

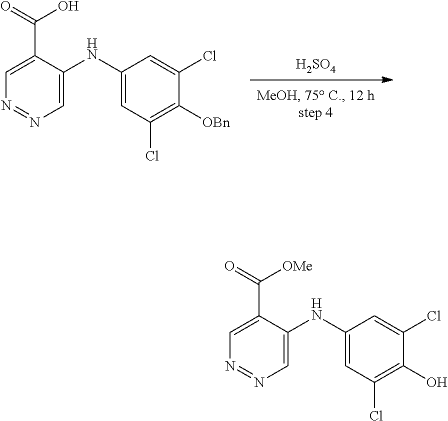 [SMILES: O=C(O)c1cnncc1Nc1cc(Cl)c(OCc2ccccc2)c(Cl)c1 ; "H2SO4" ; COC(=O)c1cnncc1Nc1cc(Cl)c(O)c(Cl)c1]

### Methyl 5-(3,5-dichloro-4-hydroxy-anilino)pyridazine-4-carboxylate

H₂SO₄ (18.40 g, 187.60 mmol, 10 mL, 45.75 eq) in MeOH (15 mL) was cooled to 0° C. A solution of 5-(4-benzyloxy-3,5-dichloro-anilino)pyridazine-4-carboxylic acid (1.6 g, 4.10 mmol, 1 eq) in THF (10 mL) was added into the cooled solution at 0° C. The mixture was stirred at 75° C. for 12 h. The mixture was cooled to rt and was poured into ice-water (100 mL) and neutralized with NaOH (1M) to pH 7-8. The aqueous mixture was extracted with 2-Me THF (100 mL*10) and the organic extracts were dried over Na₂SO₄, filtered, and concentrated to give the crude. The residue was purified by flash silica gel chromatography (ISCO®; 12 g SepaFlash® Silica Flash Column, Eluent of 50-100% Ethylacetate/Petroleum ether gradient @ 75 mL/min, 10% MeOH added) to give methyl 5-(4-benzyloxy-3,5-dichloro-anilino)pyridazine-4-carboxylate (170 mg, 365.87 umol, 8.92% yield, 87% purity) as a brown solid.

LCMS: ET20197-386-P1P (M+H⁺): 314.0 @ 0.656 min (5-95% ACN in H₂O, 2.0 min)

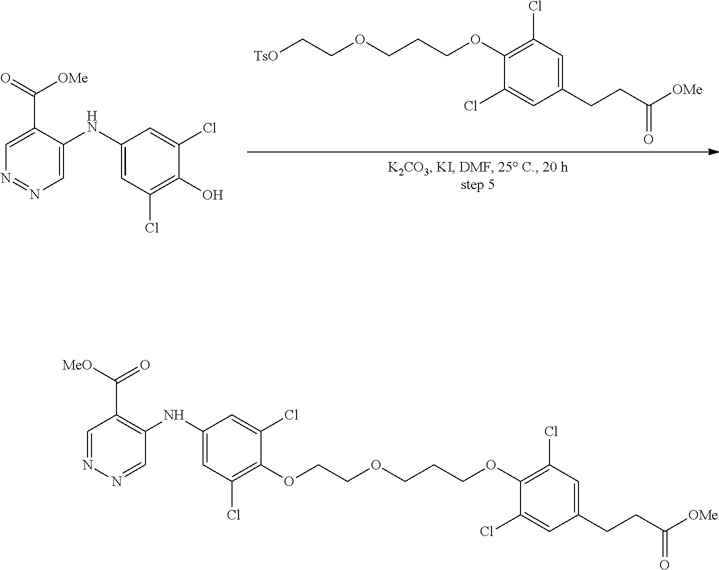 [SMILES: COC(=O)c1cnncc1Nc1cc(Cl)c(O)c(Cl)c1 ; COC(=O)CCc1cc(Cl)c(OCCCOCCOS(=O)(=O)c2ccc(C)cc2)c(Cl)c1 ; COC(=O)CCc1cc(Cl)c(OCCCOCCOc2c(Cl)cc(Nc3cnncc3C(=O)OC)cc2Cl)c(Cl)c1]

### Methyl 5-[3,5-dichloro-4-[2-[3-[2,6-dichloro-4-(3-methoxy-3-oxo-propyl)phenoxy]propoxy]ethoxy]anilino]pyridazine-4-carboxylate

A mixture of methyl 5-(3,5-dichloro-4-hydroxy-anilino)pyridazine-4-carboxylate (180 mg, 573.02 umol, 1 eq), methyl (E)-3-[3,5-dichloro-4-[3-[2-(p-tolylsulfonyloxy)ethoxy]propoxy]phenyl]prop-2-enoate (288.45 mg, 573.02 umol, 1 eq), KI (95.12 mg, 573.02 umol, 1 eq), K₂CO₃ (237.59 mg, 1.72 mmol, 3 eq) in DMF (5 mL) was degassed and purged with N₂ for 3 times, and then the mixture was stirred at 25° C. for 20 hr under N₂ atmosphere. The reaction was filtered and concentrated to give the residue. The reaction was diluted with H₂O (50 mL) and then extracted with EtOAc (50 mL*3). The combined organic phase was washed with saturated brine (50 mL*2), dried over anhydrous Na₂SO₄, filtered and concentrated in vacuo. The residue was purified by flash silica gel chromatography (ISCO®; 12 g SepaFlash® Silica Flash Column, Eluent of 0 ˜50% Ethylacetate/Petroleum ether gradient @ 75 mL/min) to give methyl 5-[3,5-dichloro-4-[2-[3-[2,6-dichloro-4-(3-methoxy-3-oxo-propyl)phenoxy]propoxy] ethoxy]anilino]pyridazine-4-carboxylate (120 mg, 94.54 umol, 16.50% yield, 51% purity) as slight yellow solid.

LCMS: ET20197-389-P1D (M+H⁺): 648.0 @ 1.944 min (5-95% ACN in H₂O, 4.5 min)

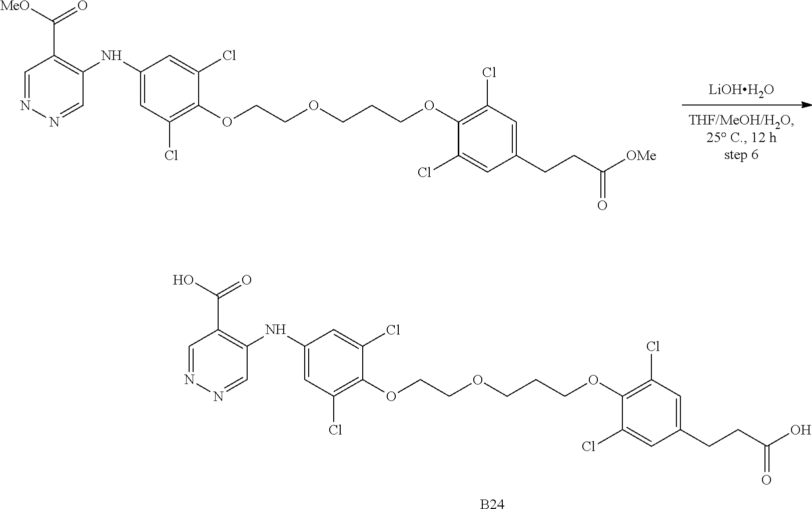

### 5-[4-[2-[3-[4-(2-carboxyethyl)-2,6-dichloro-phenoxy]propoxy]ethoxy]-3,5-dichloro-anilino]pyridazine-4-carboxylic acid

To a solution of methyl 5-[3,5-dichloro-4-[2-[3-[2,6-dichloro-4-(3-methoxy-3-oxopropyl) phenoxy]propoxy]ethoxy]anilino]pyridazine-4-carboxylate (120 mg, 185.38 umol, 1 eq) in MeOH (3 mL)/H₂O (3 mL)/THF (3 mL) was added LiOH·H₂O (77.78 mg, 1.85 mmol, 10 eq). The reaction was stirred at 25° C. for 12 hr. The reaction was acidified with HCl (1M) to pH 5 ˜6 and extracted with EtOAc (50 mL*3). The combined organic phase was washed with saturated brine (50 mL*2), dried over anhydrous Na₂SO₄, filtered and concentrated in vacuo. The residue was purified by prep-HPLC (column: Phenomenex Luna C18 150*30 mm*5 um; mobile phase: [water (0.04% HCl)-ACN]; B %: 35%-65%, 10 min;) to give 5-[4-[2-[3-[4-(2-carboxyethyl)-2,6-dichloro-phenoxy]propoxy]ethoxy]-3,5-dichloro-anilino]pyridazine-4-carboxylic acid (23.2 mg, 37.09 umol, 20.01% yield, 99% purity) as a yellow solid.

LCMS: ET20197-390-PIP (M+H⁺): 620.1 @ 2.11 min (5-95% ACN in H₂O, 6.0 min)

¹H NMR: ET20197-390-P1AA (400 MHz, DMSO-d₆)

δ 11.20 (br s, 1H), 9.27 (s, 1H), 8.88 (s, 1H), 7.65 (s, 2H), 7.36 (s, 2H), 4.22-4.17 (m, 2H), 4.03 (t, J=6.4 Hz, 2H), 3.81-3.77 (m, 2H), 3.72-3.68 (m, 3H), 2.79-2.74 (m, 2H), 2.57-2.54 (m, 2H), 2.00 (quin, J=6.3 Hz, 2H)

DSC: ET20197-347-P1D (Min:169.7° C., Max: 210.9° C.)

### Synthesis of B25 (T-764)

In Step 1 of this synthesis, 2-benzyloxy-1,3-dichloro-5-nitro-benzene was prepared as described above in the synthesis of B4 and B4A.

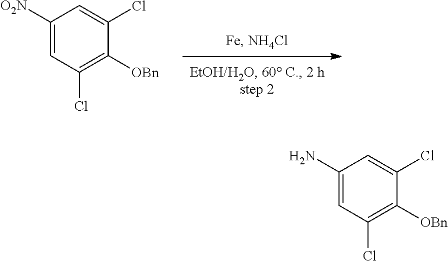

### Methyl 3-[4-[3-[2-(4-amino-2,6-dichloro-phenoxy)ethoxy]propoxy]-3,5-dichloro-phenyl]propanoate

To a solution of 2-benzyloxy-1,3-dichloro-5-nitro-benzene (6 g, 20.13 mmol, 1 eq) in EtOH (40 mL)/H₂O (8 mL) was added NH₄Cl (1.08 g, 20.13 mmol, 1 eq). And Fe (5.62 g, 100.63 mmol, 5 eq) was added in batches. The reaction was stirred at 60° C. for 2 h. The reaction was filtered through the Celite, washed with MeOH (50 mL) and concentrated. The residue was purified by flash silica gel chromatography (ISCO®; 40 g SepaFlash® Silica Flash Column, eluent of 0˜20% Ethyl acetate/Petroleum ether gradient @ 100 mL/min) to afford 4-benzyloxy-3,5-dichloro-aniline (4.2 g, 15.66 mmol, 77.83% yield) as a white solid.

¹H NMR: ET20197-325-P1AA (400 MHz, CHLOROFORM-d)

δ 7.56 (d, J=7.1 Hz, 2H), 7.44-7.33 (m, 3H), 6.64 (s, 2H), 4.96 (s, 2H), 3.64 (br s, 2H)

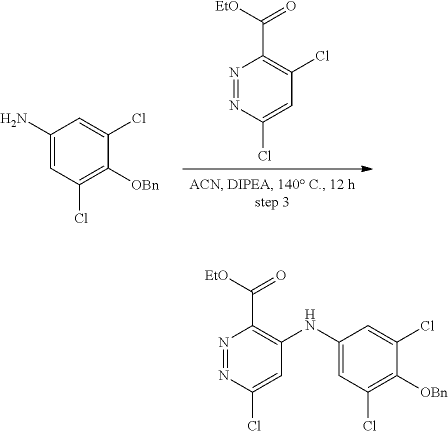 [SMILES: Nc1cc(Cl)c(OCc2ccccc2)c(Cl)c1 ; CCOC(=O)c1nnc(Cl)cc1Cl ; CCOC(=O)c1nnc(Cl)cc1Nc1cc(Cl)c(OCc2ccccc2)c(Cl)c1]

### Ethyl 4-(4-benzyloxy-3,5-dichloro-anilino)-6-chloro-pyridazine-3-carboxylate

Ethyl 4,6-dichloropyridazine-3-carboxylate (350 mg, 1.58 mmol, 1 eq), 4-benzyloxy-3,5-dichloro-aniline (424.58 mg, 1.58 mmol, 1 eq) and DIPEA (409.28 mg, 3.17 mmol, 551.59 uL, 2 eq) were taken up into a microwave tube in ACN (10 mL). The sealed tube was heated at 140° C. for 12 hr under microwave. The reaction was concentrated to give the residue. The residue was purified by flash silica gel chromatography (ISCO®; 20 g SepaFlash® Silica Flash Column, Eluent of 0˜25% Ethyl acetate/Petroleum ether gradient @ 75 mL/min) to afford ethyl 4-(4-benzyloxy-3,5-dichloro-anilino)-6-chloro-pyridazine-3-carboxylate (200 mg, 397.60 umol, 25.11% yield, 90% purity) as a brown solid.

¹H NMR: ET20197-355-P1AA (400 MHz, DMSO-d₆)

δ 9.49 (s, 1H), 7.59 (s, 2H), 7.55 (br d, J=6.6 Hz, 2H), 7.47-7.36 (m, 3H), 7.19 (s, 1H), 5.05 (s, 2H), 4.48-4.35 (m, 2H), 1.37 (t, J=7.1 Hz, 3H)

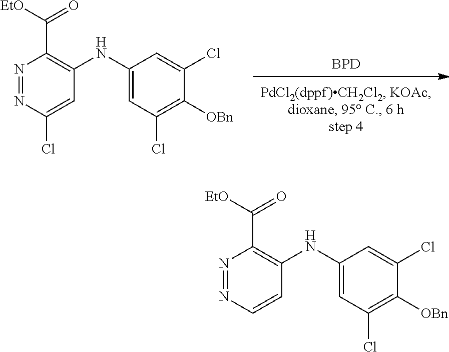 [SMILES: CCOC(=O)c1nnc(Cl)cc1Nc1cc(Cl)c(OCc2ccccc2)c(Cl)c1 ; "BPD" ; CCOC(=O)c1nnccc1Nc1cc(Cl)c(OCc2ccccc2)c(Cl)c1]

### Ethyl 4-(4-benzyloxy-3,5-dichloro-anilino)pyridazine-3-carboxylate

To a solution of ethyl 4-(4-benzyloxy-3,5-dichloro-anilino)-6-chloro-pyridazine-3-carboxylate (200 mg, 441.78 umol, 1 eq), 4,4,5,5-tetramethyl-2-(4,4,5,5-tetramethyl-1,3,2-dioxaborolan-2-yl)-1,3,2-dioxaborolane (224.37 mg, 883.55 umol, 2 eq) in dioxane (20 mL) was added Pd(dppf)Cl₂·CH₂Cl₂ (72.15 mg, 88.36 umol, 0.2 eq) and KOAc (130.07 mg, 1.33 mmol, 3 eq). The reaction was stirred at 90° C. for 6 h. The reaction was (combined with ET20197-357, 80 mg) filtered and concentrated to give the residue. The residue was purified by flash silica gel chromatography (ISCO®; 20 g SepaFlash® Silica Flash Column, Eluent of 0 ˜70% Ethylacetate/Petroleum ether gradient @ 75 mL/min) and further purified by prep-TLC (SiO₂, Petroleum ether/Ethylacetate=2:3) to ethyl 4-(4-benzyloxy-3,5-dichloro-anilino)pyridazine-3-carboxylate (110 mg) as a brown solid.

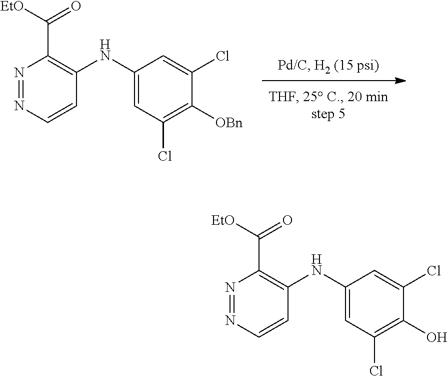 [SMILES: CCOC(=O)c1nnccc1Nc1cc(Cl)c(OCc2ccccc2)c(Cl)c1 ; "Pd/C, H2 (15 psi)" ; CCOC(=O)c1nnccc1Nc1cc(Cl)c(O)c(Cl)c1]

### Ethyl 4-(3,5-dichloro-4-hydroxy-anilino)pyridazine-3-carboxylate

To a solution of ethyl 4-(4-benzyloxy-3,5-dichloro-anilino)pyridazine-3-carboxylate (55 mg, 131.49 umol, 1 eq) in THF (10 mL) was added Pd/C (5 mg, 10% purity) under N₂. The suspension was degassed under vacuum and purged with H₂ several times. The mixture was stirred under H₂ (15 psi) at 25° C. for 20 min. The reaction mixture was filtered and the filter was concentrated to give 75 mg (60.9% purity). The crude product was triturated with MTBE (10 mL) at 25° C. for 2 min and filtered to give ethyl 4-(3,5-dichloro-4-hydroxy-anilino)pyridazine-3-carboxylate (54 mg, 116.84 umol, 44.43% yield, 71% purity) as a light yellow solid.

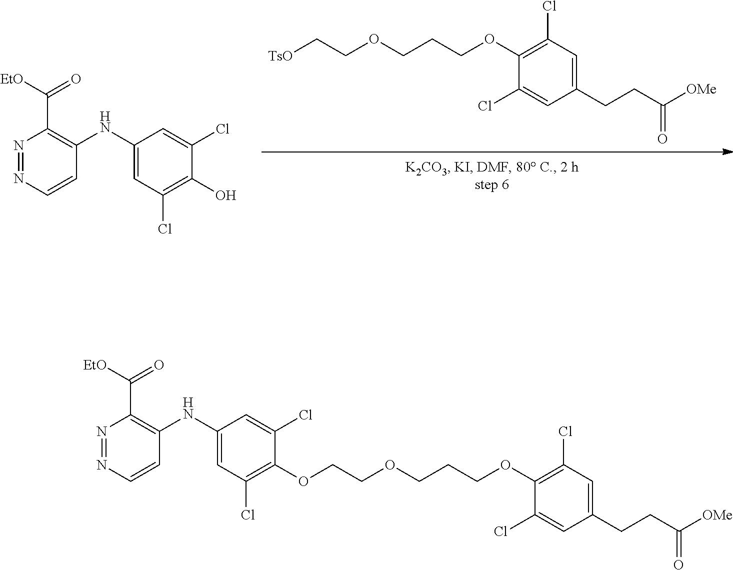 [SMILES: CCOC(=O)c1nnccc1Nc1cc(Cl)c(O)c(Cl)c1 ; COC(=O)CCc1cc(Cl)c(OCCCOCCOS(=O)(=O)c2ccc(C)cc2)c(Cl)c1 ; CCOC(=O)c1nnccc1Nc1cc(Cl)c(OCCOCCCOc2c(Cl)cc(CCC(=O)OC)cc2Cl)c(Cl)c1]

### Ethyl 4-[3,5-dichloro-4-[2-[3-[2,6-dichloro-4-(3-methoxy-3-oxo-propyl)phenoxy]propoxy] ethoxy]anilino]pyridazine-3-carboxylate

A mixture of ethyl 4-(3,5-dichloro-4-hydroxy-anilino)pyridazine-3-carboxylate (56 mg, 170.65 umol, 1 eq), methyl 3-[3,5-dichloro-4-[3-[2-(p-tolylsulfonyloxy)ethoxy]propoxy]phenyl]propanoate (103.50 mg, 204.78 umol, 1.2 eq), KI (28.33 mg, 170.65 umol, 1 eq), K₂CO₃ (70.76 mg, 511.96 umol, 3 eq) in DMF (1 mL) was degassed and purged with N₂ for 3 times, and then the mixture was stirred at 80° C. for 2 h under N₂ atmosphere. The reaction was (combined with ET20197-364, 8 mg scale) filtered and concentrated to give the residue. The reaction was diluted with H₂O (50 mL) and then extracted with 2-Me THF (50 mL*5). The combined organic phase was washed with saturated brine (50 mL*2), dried over anhydrous Na₂SO₄, filtered and concentrated in vacuo to give the residue (200 mg, 17% purity). The residue was purified by flash silica gel chromatography (ISCO®; 4 g SepaFlash® Silica Flash Column, Eluent of 0 ˜100% Ethylacetate/Petroleum ether gradient @ 50 mL/min) and further purified by prep-TLC (SiO₂, DCM:MeOH=20:1) to afford:
batch 1: ethyl 4-[3,5-dichloro-4-[2-[3-[2,6-dichloro-4-(3-methoxy-3-oxo-propyl)phenoxy]propoxy]ethoxy]anilino]pyridazine-3-carboxylate (˜10 mg, 83% purity) as slight yellow solid;
batch 2: ethyl 4-[3,5-dichloro-4-[2-[3-[2,6-dichloro-4-(3-methoxy-3-oxo-propyl)phenoxy]propoxy]ethoxy]anilino]pyridazine-3-carboxylate (˜20 mg, 31% purity) as a brown oil.

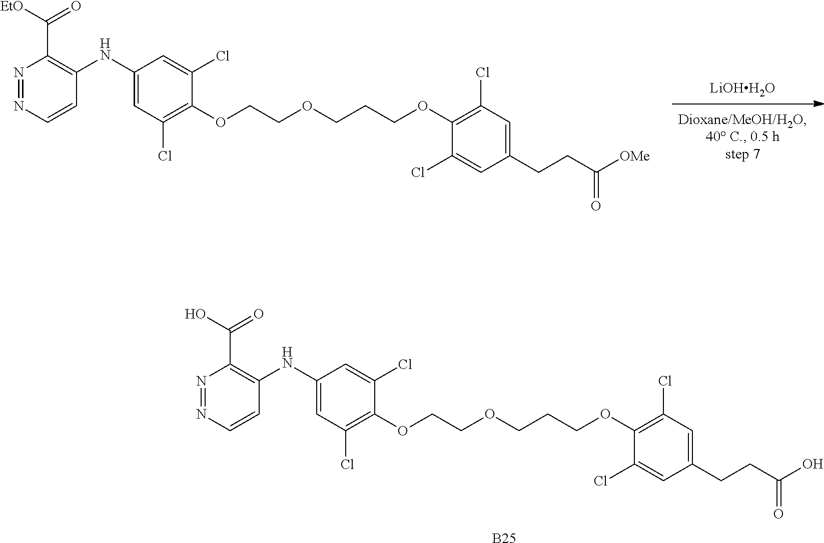

### 4-[4-[2-[3-[4-(2-carboxyethyl)-2,6-dichloro-phenoxy]propoxy]ethoxy]-3,5-dichloro-anilino]pyridazine-3-carboxylic acid

To a solution of ethyl 4-[3,5-dichloro-4-[2-[3-[2,6-dichloro-4-(3-methoxy-3-oxopropyl) phenoxy]propoxy]ethoxy]anilino]pyridazine-3-carboxylate (10 mg, 15.12 umol, 1 eq) in MeOH (1 mL)/H₂O (1 mL)/dioxane (1 mL) was added LiOH·H₂O (6.35 mg, 151.20 umol, 10 eq). The reaction was stirred at 40° C. for 0.5 hr. The reaction was acidified with HCl (1M) to pH 5 ˜6 and directly purified by prep-HPLC (column: Phenomenex Luna C18 150*30 mm*5 um; mobile phase: [water (0.04% HCl)-ACN]; B %: 20%-50%, 10 min;) to afford 4-[4-[2-[3-[4-(2-carboxyethyl)-2,6-dichloro-phenoxy]propoxy]ethoxy]-3,5-dichloro-anilino]pyridazine-3-carboxylic acid (combined with ET20197-367, 20 mg*2 scale and ET20197-368, 10 mg scale) (6.6 mg, 98.8% purity) as an off-white solid.

LCMS: ET20197-368-P1P1 (M+H⁺): 620.0 @ 2.123 min (15-100% ACN in H₂O, 4.5 min)

¹H NMR: ET20197-368-P1BB (400 MHz, METHANOL-d₄)

δ 8.77 (d, J=7.2 Hz, 1H), 7.53 (s, 2H), 7.41 (d, J=7.3 Hz, 1H), 7.25 (s, 2H), 4.33-4.27 (m, 2H), 4.08 (t, J=6.3 Hz, 2H), 3.92-3.85 (m, 2H), 3.79 (t, J=6.2 Hz, 2H), 2.88-2.81 (m, 2H), 2.63-2.55 (m, 2H), 2.08 (quin, J=6.2 Hz, 2H)

### Synthesis of B26 (T-765)

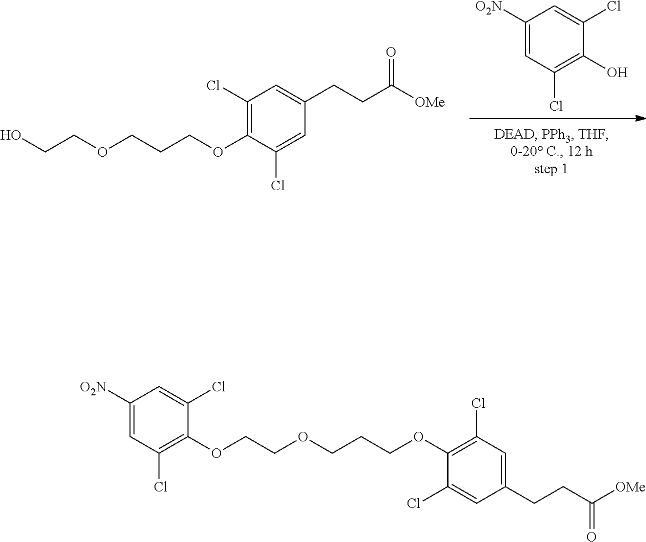 [SMILES: COC(=O)CCc1cc(Cl)c(OCCCOCCO)c(Cl)c1 ; O=[N+]([O-])c1cc(Cl)c(O)c(Cl)c1 ; COC(=O)CCc1cc(Cl)c(OCCCOCCOc2c(Cl)cc([N+](=O)[O-])cc2Cl)c(Cl)c1]

### Methyl 3-[3,5-dichloro-4-[3-[2-(2,6-dichloro-4-nitro-phenoxy)ethoxy]propoxy]phenyl]propanoate

To a solution of methyl 3-[3,5-dichloro-4-[3-(2-hydroxyethoxy)propoxy]phenyl]propanoate (2.5 g, 7.12 mmol, 1 eq), 2,6-dichloro-4-nitro-phenol (1.78 g, 8.54 mmol, 1.2 eq), PPh₃ (2.80 g, 10.68 mmol, 1.5 eq) in THF (20 mL) was added dropwise DIAD (2.16 g, 10.68 mmol, 2.08 mL, 1.5 eq) at 0° C. The reaction was stirred at 20° C. for 12 h. The reaction was concentrated to give the residue. The crude product was purified by reversed-phase HPLC (0.1% HCl condition, MeOH/H₂O) to give methyl 3-[3,5-dichloro-4-[3-[2-(2,6-dichloro-4-nitro-phenoxy)ethoxy]propoxy]phenyl]propanoate (1.5 g, 2.77 mmol, 38.94% yield) as a brown oil.

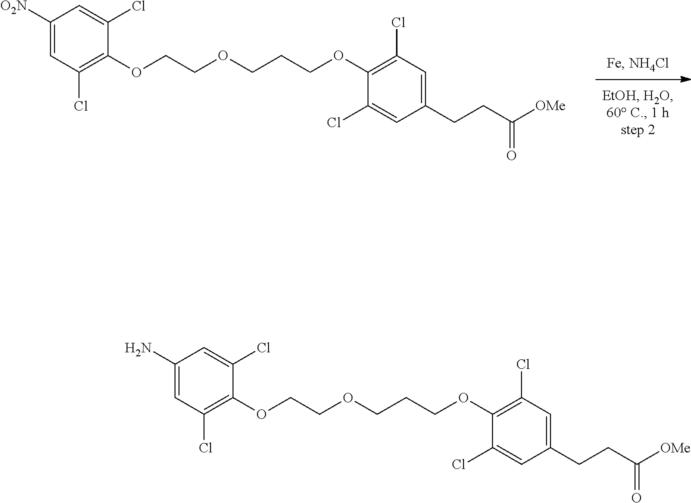 [SMILES: COC(=O)CCc1cc(Cl)c(OCCCOCCOc2c(Cl)cc([N+](=O)[O-])cc2Cl)c(Cl)c1 ; COC(=O)CCc1cc(Cl)c(OCCCOCCOc2c(Cl)cc(N)cc2Cl)c(Cl)c1]

### Methyl 3-[4-[3-[2-(4-amino-2,6-dichloro-phenoxy)ethoxy]propoxy]-3,5-dichloro-phenyl] propanoate

To a solution of methyl 3-[3,5-dichloro-4-[3-[2-(2,6-dichloro-4-nitro-phenoxy)ethoxy]propoxy]phenyl]propanoate (1.5 g, 2.77 mmol, 1 eq) in EtOH (50 mL)/H₂O (10 mL) was added NH₄Cl (148.26 mg, 2.77 mmol, 1 eq). And Fe (773.90 mg, 13.86 mmol, 5 eq) was added in batches. The reaction was stirred at 60° C. for 2 hr. The reaction was filtered through the Celite and washed with MeOH (50 mL), filtered and concentrated to give the residue. The residue was purified by flash silica gel chromatography (ISCO®; 12 g SepaFlash® Silica Flash Column, Eluent of 0 ˜10% Ethyl acetate/Petroleum ether gradient @ 75 mL/min) to give methyl 3-[4-[3-[2-(4-amino-2,6-dichloro-phenoxy)ethoxy]propoxy]-3,5-dichloro-phenyl]propanoate (1.4 g, 2.74 mmol, 98.81% yield) as a brown oil.

¹H NMR: ET20197-339-P1AA (400 MHz, CHLOROFORM-d)

δ 7.12 (s, 2H), 6.63-6.52 (m, 2H), 4.10 (br d, J=3.4 Hz, 2H), 3.88-3.76 (m, 4H), 3.73-3.59 (m, 5H), 2.85 (t, J=7.6 Hz, 2H), 2.65-2.55 (m, 2H), 2.13 (quin, J=6.3 Hz, 2H)

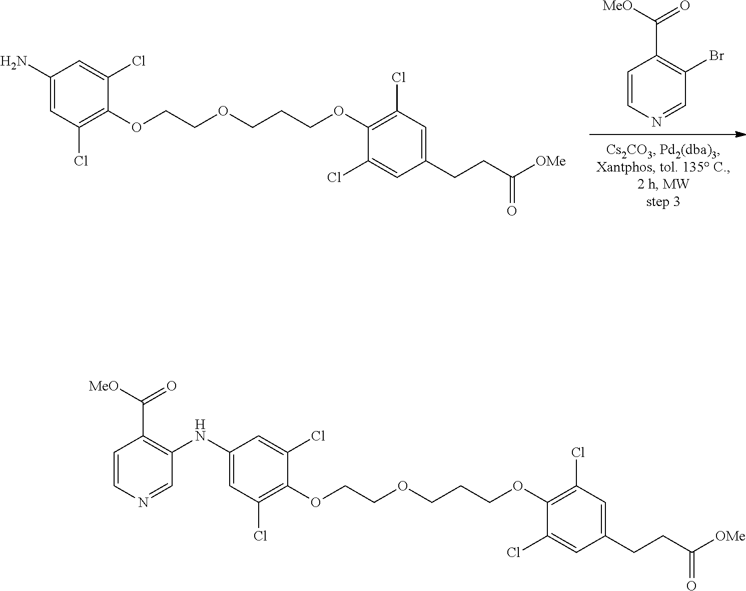 [SMILES: COC(=O)CCc1cc(Cl)c(OCCCOCCOc2c(Cl)cc(N)cc2Cl)c(Cl)c1 ; COC(=O)c1ccncc1Br ; COC(=O)CCc1cc(Cl)c(OCCCOCCOc2c(Cl)cc(Nc3cnccc3C(=O)OC)cc2Cl)c(Cl)c1]

### Methyl 3-[3,5-dichloro-4-[2-[3-[2,6-dichloro-4-(3-methoxy-3-oxo-propyl)phenoxy]propoxy]ethoxy]anilino]pyridine-4-carboxylate

A suspension of methyl 3-bromopyridine-4-carboxylate (101.42 mg, 469.46 umol, 1.2 eq), methyl 3-[4-[3-[2-(4-amino-2,6-dichloro-phenoxy)ethoxy]propoxy]-3,5-dichloro-phenyl]propanoate (200 mg, 391.22 umol, 1 eq), Cs₂CO₃ (178.45 mg, 547.71 umol, 1.4 eq), Pd₂(dba)₃ (71.65 mg, 78.24 umol, 0.2 eq) and Xantphos (135.82 mg, 234.73 umol, 0.6 eq) in anhydrous Tol. (10 mL)) was heated to 135° C. for 2 hr under Microwave irradiation. The reaction (combined with ET20197-328, 20 mg scale) was filtered and concentrated to give the residue. The residue was diluted with H₂O (50 mL) and then extracted with EtOAc (50 mL*3). The combined organic phase was washed with saturated brine (50 mL*2), dried over anhydrous Na₂SO₄, filtered and concentrated in vacuo. The residue was purified by flash silica gel chromatography (ISCO®; 12 g SepaFlash® Silica Flash Column, Eluent of 0 ˜25% Ethylacetate/Petroleum ether gradient @ 75 mL/min) to give methyl 3-[3,5-dichloro-4-[2-[3-[2,6-dichloro-4-(3-methoxy-3-oxo-propyl)phenoxy]propoxy]ethoxy]anilino]pyridine-4-carboxylate (310 mg) as a light brown oil.

¹H NMR: ET20197-341-P1AA (400 MHz, CHLOROFORM-d)

δ 9.01 (s, 1H), 8.66 (s, 1H), 8.11 (d, J=5.1 Hz, 1H), 7.70 (d, J=5.1 Hz, 1H), 7.21 (s, 2H), 7.12 (s, 2H), 4.25-4.17 (m, 2H), 4.12 (t, J=6.3 Hz, 2H), 3.95 (s, 3H), 3.91-3.86 (m, 2H), 3.85-3.80 (m, 2H), 3.68 (s, 3H), 2.90-2.82 (m, 2H), 2.64-2.55 (m, 2H), 2.19-2.09 (m, 2H)

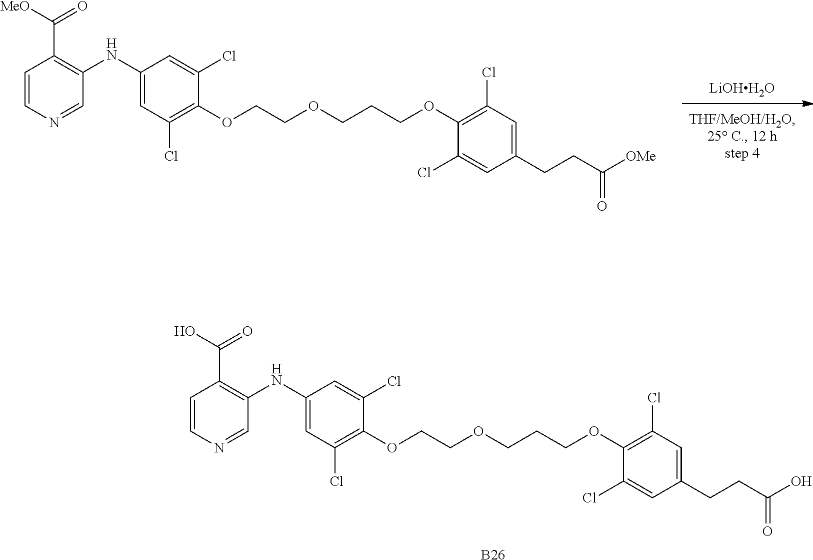

### 3-[4-[2-[3-[4-(2-carboxyethyl)-2,6-dichloro-phenoxy]propoxy]ethoxy]-3,5-dichloro-anilino]pyridine-4-carboxylic acid

To a solution of methyl 3-[3,5-dichloro-4-[2-[3-[2,6-dichloro-4-(3-methoxy-3-oxopropyl) phenoxy]propoxy]ethoxy]anilino]pyridine-4-carboxylate (310 mg, 479.62 umol, 1 eq) in THF (5 mL)/MeOH (5 mL)/H₂O (2 mL) was added LiOH·H₂O (201.27 mg, 4.80 mmol, 10 eq). The reaction was stirred at 25° C. for 12 hr. The reaction was acidified with HCl (1M) to pH 5 ˜6 and a solid was formed. The suspension was filtered to give the cake. The cake was dissolved in DMSO/MeOH/THF/H₂O (1:1:1:1, 10 mL), adjusted with NaHCO₃ (sat., aq.) to pH 8-9. The residue was purified by prep-HPLC (column: Phenomenex Luna C18 150*30 mm*5 um; mobile phase:[water (0.04% HCl)-ACN]; B %: 40%-70%, 10 min; column: Phenomenex Luna C18 150*30 mm*5 um; mobile phase: [water (0.04% HCl)-ACN]; B %: 40%-70%, 10 min;) to give 3-[4-[2-[3-[4-(2-carboxyethyl)-2,6-dichloro-phenoxy]propoxy]ethoxy]-3,5-dichloro-anilino]pyridine-4-carboxylic acid (100.5 mg, 161.68 umol, 33.71% yield, 99.47% purity) as a yellow solid.

LCMS: ET20197-347-P1P1 (M+H): 618.9 @ 1.815 min (15-100% ACN in H₂O, 4.5 min)

¹H NMR: ET20197-347-P1AA (400 MHz, DMSO-d₆)

δ 9.10 (s, 1H), 8.59 (s, 1H), 8.15 (d, J=5.1 Hz, 1H), 7.78 (d, J=5.1 Hz, 1H), 7.40 (s, 2H), 7.35 (s, 2H), 4.15-4.09 (m, 2H), 4.02 (t, J=6.3 Hz, 2H), 3.79-3.75 (m, 2H), 3.68 (br t, J=6.2 Hz, 2H), 2.79-2.72 (m, 2H), 2.56-2.54 (m, 2H), 2.00 (quin, J=6.2 Hz, 2H)

DSC: ET20197-347-P1D (Peak: 86.7° C., Onset: 74.2° C., End:10.3.0° C.)

### Synthesis of B27 (T-766)

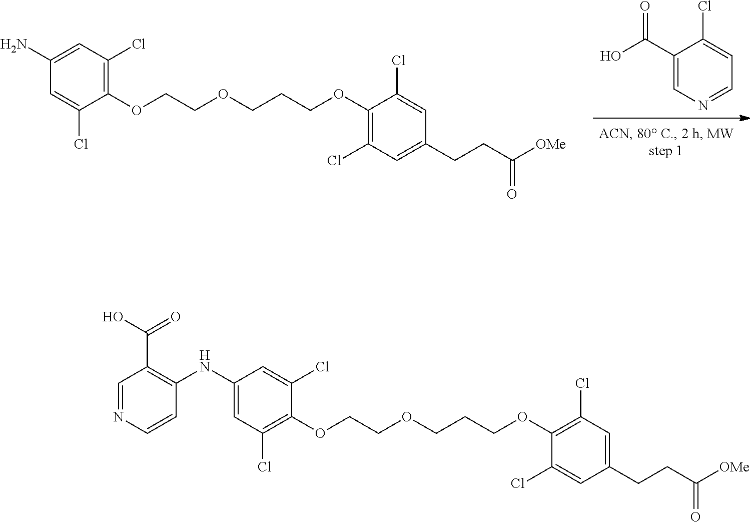 [SMILES: COC(=O)CCc1cc(Cl)c(OCCCOCCOc2c(Cl)cc(N)cc2Cl)c(Cl)c1 ; O=C(O)c1cnccc1Cl ; COC(=O)CCc1cc(Cl)c(OCCCOCCOc2c(Cl)cc(Nc3ccncc3C(=O)O)cc2Cl)c(Cl)c1]

### 4-[3,5-dichloro-4-[2-[3-[2,6-dichloro-4-(3-methoxy-3-oxo-propyl)phenoxy]propoxy]ethoxy]anilino]pyridine-3-carboxylic acid

Methyl 3-[4-[3-[2-(4-amino-2,6-dichloro-phenoxy)ethoxy]propoxy]-3,5-dichloro-phenyl]propanoate (200 mg, 391.22 umol, 1 eq), 4-chloropyridine-3-carboxylic acid (92.46 mg, 586.83 umol, 1.5 eq) were taken up into a microwave tube in ACN (5 mL). And then the solution was stirred at 80° C. for 1 hr under microwave. The reaction (combined with ET20197-333, 20 mg scale) was concentrated to remove the solvent. 4-[3,5-dichloro-4-[2-[3-[2,6-dichloro-4-(3-methoxy-3-oxo-propyl)phenoxy]propoxy]ethoxy]anilino]pyridine-3-carboxylic acid (210 mg) as a yellow solid. The crude was used into next step without further purification.

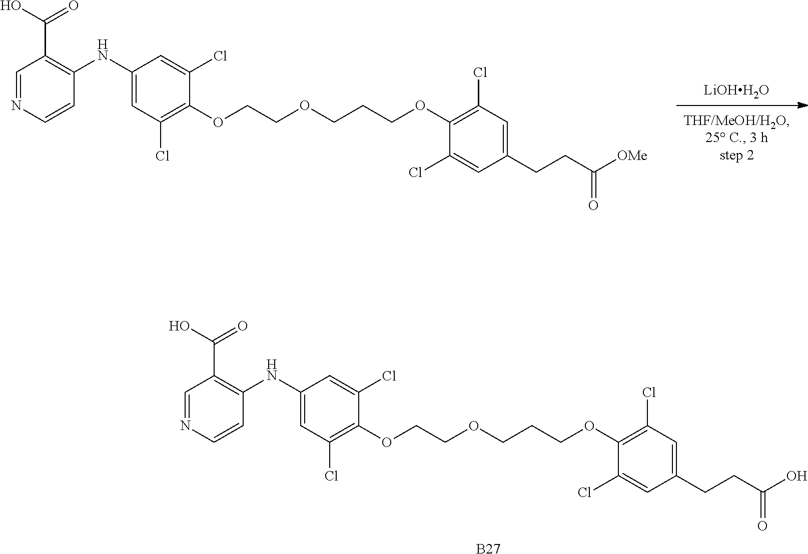

### 4-[4-[2-[3-[4-(2-carboxyethyl)-2,6-dichloro-phenoxy]propoxy]ethoxy]-3,5-dichloro-anilino]pyridine-3-carboxylic acid

To a solution of 4-[3,5-dichloro-4-[2-[3-[2,6-dichloro-4-(3-methoxy-3-oxo-propyl)phenoxy]propoxy]ethoxy]anilino]pyridine-3-carboxylic acid (190 mg, 300.48 umol, 1 eq) in dioxane (5 mL)/MeOH (5 mL)/H₂O (3 mL) was added LiOH·H₂O (12.61 mg, 300.48 umol, 1 eq) at 25° C. The reaction was stirred at 25° C. for 3 hr. The reaction (combined with ET20197-342, 20 mg scale) was acidified with HCl (1M) to pH 5 ˜6 and a solid were formed. The suspension was filtered to give the cake. The cake was dissolved in DMSO/MeOH/THF/H₂O (1:1:1:1, 10 mL), adjusted with NaHCO₃ (sat., aq.) to pH 8-9. The residue was purified by prep-HPLC (column: Phenomenex Luna C18 150*30 mm*5 um; mobile phase: [water (0.04% HCl)-ACN]; B %: 30%-55%, 10 min column: Phenomenex Luna C18 150*30 mm*5 um; mobile phase: [water (0.04% HCl)-ACN]; B %: 30%-55%, 10 min) to give 4-[4-[2-[3-[4-(2-carboxyethyl)-2,6-dichloro-phenoxy]propoxy]ethoxy]-3,5-dichloro-anilino]pyridine-3-carboxylic acid (65.1 mg, 99.2% purity) as a white solid.

LCMS: ET20197-345-P1P1 (M+H⁺): 618.9 @ 2.792 min (0-100% ACN in H₂O, 4.5 min)

¹H NMR: ET20197-345-P1AA (400 MHz, DMSO-d₆)

δ 11.21 (br s, 1H), 8.90 (s, 1H), 8.27 (br d, J=6.9 Hz, 1H), 7.66-7.53 (m, 2H), 7.36 (s, 2H), 7.16-6.99 (m, 1H), 4.22-4.16 (m, 2H), 4.02 (t, J=6.4 Hz, 2H), 3.82-3.74 (m, 2H), 3.69 (t, J=6.2 Hz, 2H), 2.81-2.72 (m, 2H), 2.57-2.54 (m, 2H), 2.00 (quin, J=6.3 Hz, 2H)

DSC: ET20197-345-P1D (Peak: 187.4° C., Onset: 183.4° C., End: 195.0° C.)

### Synthesis of B28 (T-767)

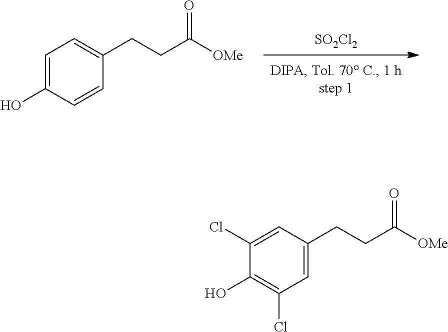 [SMILES: COC(=O)CCc1ccc(O)cc1 ; "SO2Cl2" ; COC(=O)CCc1cc(Cl)c(O)c(Cl)c1]

### Methyl 3-(3,5-dichloro-4-hydroxy-phenyl)propanoate

To a solution of methyl 3-(4-hydroxyphenyl)propanoate (10 g, 55.49 mmol, 1 eq) and DIPA (561.54 mg, 5.55 mmol, 784.28 uL, 0.1 eq) in toluene (1 L) was added sulfuryl chloride (18.72 g, 138.73 mmol, 13.87 mL, 2.5 eq) at 70° C. The reaction was stirred at 70° C. for 1 hr. The reaction was washed with H₂O (100 mL*4), brine (100 mL*2) and dried over Na₂SO₄, filtered and concentrated to give the residue. The residue was purified by flash silica gel chromatography (ISCO®; 40 g SepaFlash® Silica Flash Column, Eluent of 0 ˜25% Ethylacetate/Petroleum ether gradient @ 100 mL/min) to give methyl 3-(3,5-dichloro-4-hydroxy-phenyl)propanoate (9.5 g, 38.14 mmol, 68.73% yield) as a white solid.

¹H NMR: ET20197-312-P1AA (400 MHz, CHLOROFORM-d)

δ 7.17-7.05 (m, 2H), 5.84 (s, 1H), 3.73-3.60 (m, 3H), 2.91-2.80 (m, 2H), 2.67-2.52 (m, 2H)

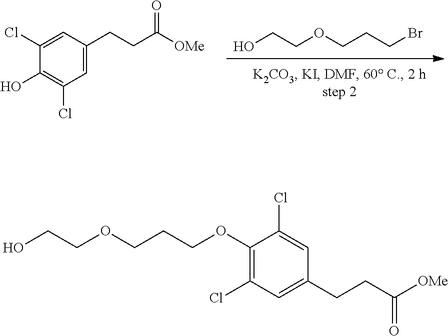 [SMILES: COC(=O)CCc1cc(Cl)c(O)c(Cl)c1 ; OCCOCCCBr ; COC(=O)CCc1cc(Cl)c(OCCCOCCO)c(Cl)c1]

### Methyl 3-[3,5-dichloro-4-[3-(2-hydroxyethoxy)propoxy]phenyl]propanoate

To a solution of methyl 3-(3,5-dichloro-4-hydroxy-phenyl)propanoate (7.5 g, 30.11 mmol, 1 eq) and 2-(3-bromopropoxy)ethanol (6.06 g, 33.12 mmol, 1.1 eq) in DMF (120 mL) was added K₂CO₃ (12.48 g, 90.33 mmol, 3 eq) and KI (5.00 g, 30.11 mmol, 1 eq). The mixture was stirred at 60° C. for 2 hr. The reaction mixture was diluted with H₂O (100 mL) and extracted with EtOAc (80 mL*3). The combined organic layers were washed with brine 100 mL, dried over Na₂SO₄, filtered and concentrated under reduced pressure to give a residue. The residue was purified by flash silica gel chromatography (ISCO®; 40 g SepaFlash® Silica Flash Column, Eluent of 0 ˜25% Ethyl acetate/Petroleum ether gradient @100 mL/min) to afford methyl 3-[3,5-dichloro-4-[3-(2-hydroxyethoxy)propoxy]phenyl]propanoate (6.5 g, 15.92 mmol, 52.86% yield, 86% purity on 220 nm) as yellow oil.

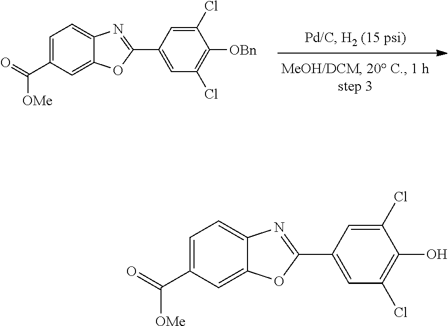 [SMILES: COC(=O)c1ccc2nc(-c3cc(Cl)c(OCc4ccccc4)c(Cl)c3)oc2c1 ; "Pd/C, H2 (15 psi)" ; COC(=O)c1ccc2nc(-c3cc(Cl)c(O)c(Cl)c3)oc2c1]

### Methyl 2-(3,5-dichloro-4-hydroxy-phenyl)-1,3-benzoxazole-6-carboxylate

To a solution of methyl 2-(4-benzyloxy-3,5-dichloro-phenyl)-1,3-benzoxazole-6-carboxylate (3 g, 7.01 mmol, 1 eq) in DCM (150 mL) and MeOH (50 mL) was added Pd/C (3 g, 10%, wet) under N₂. The suspension was degassed under vacuum and purged with H₂ several times. The mixture was stirred under H₂ (15 psi) at 15° C. for 1 hr. The reaction was filtered through the Celite and concentrated to give the crude methyl 2-(3,5-dichloro-4-hydroxy-phenyl)-1,3-benzoxazole-6-carboxylate (2.5 g, crude) as a light-brown solid. The crude product was used into the next step without further purification.

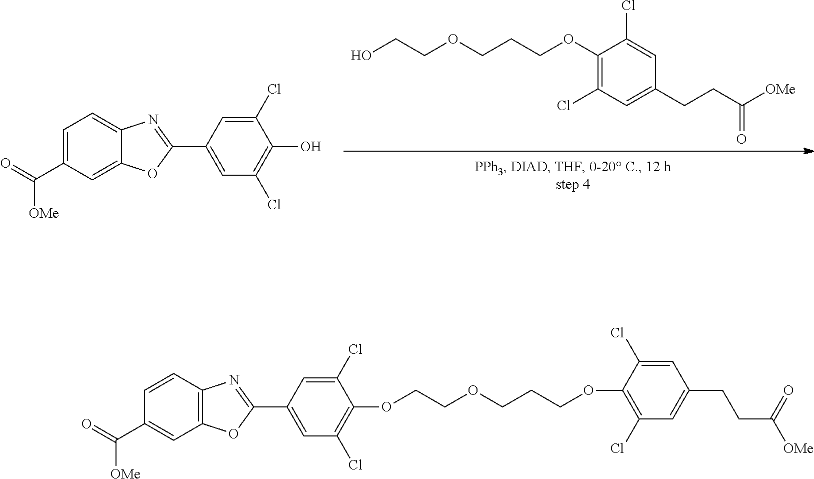 [SMILES: COC(=O)c1ccc2nc(-c3cc(Cl)c(O)c(Cl)c3)oc2c1 ; COC(=O)CCc1cc(Cl)c(OCCCOCCO)c(Cl)c1 ; COC(=O)CCc1cc(Cl)c(OCCCOCCOc2c(Cl)cc(-c3nc4ccc(C(=O)OC)cc4o3)cc2Cl)c(Cl)c1]

### Methyl 2-[3,5-dichloro-4-[2-[3-[2,6-dichloro-4-(3-methoxy-3-oxo-propyl)phenoxy]propoxy]ethoxy]phenyl]-1,3-benzoxazole-6-carboxylate

To a mixture of methyl 2-(3,5-dichloro-4-hydroxy-phenyl)-1,3-benzoxazole-6-carboxylate (200 mg, 591.47 umol, 1 eq), methyl 3-[3,5-dichloro-4-[3-(2-hydroxyethoxy) propoxy]phenyl]propanoate (249.28 mg, 709.76 umol, 1.2 eq) and PPh₃ (232.70 mg, 887.20 umol, 1.5 eq) in THF (15 mL) was added dropwise DIAD (179.40 mg, 887.20 umol, 172.50 uL, 1.5 eq) at 0° C., then the black brown solution was stirred at 20° C. for 12 h. The yellow solution was concentrated to give the residue. The residue was purified by flash silica gel chromatography (ISCO®; 12 g SepaFlash® Silica Flash Column, Eluent of 0˜15% Ethyl acetate/Petroleum ether gradient @ 75 mL/min) to give methyl 2-[3,5-dichloro-4-[2-[3-[2,6-dichloro-4-(3-methoxy-3-oxo-propyl)phenoxy]propoxy]ethoxy]phenyl]-1,3-benzoxazole-6-carboxylate (170 mg, 207.64 umol, 35.11% yield, 82% purity) as an off-white solid.

¹H NMR: ET20197-318-P1AA (400 MHz, CHLOROFORM-d)

δ 8.30-8.25 (m, 1H), 8.22 (s, 2H), 8.15-8.09 (m, 1H), 7.79 (d, J=8.2 Hz, 1H), 7.11 (s, 2H), 4.39-4.30 (m, 2H), 4.09 (t, J=6.3 Hz, 2H), 3.99 (s, 3H), 3.94-3.88 (m, 2H), 3.82 (t, J=6.2 Hz, 2H), 3.68 (s, 3H), 2.85 (t, J=7.7 Hz, 2H), 2.65-2.53 (m, 2H), 2.12 (quin, J=6.3 Hz, 2H)

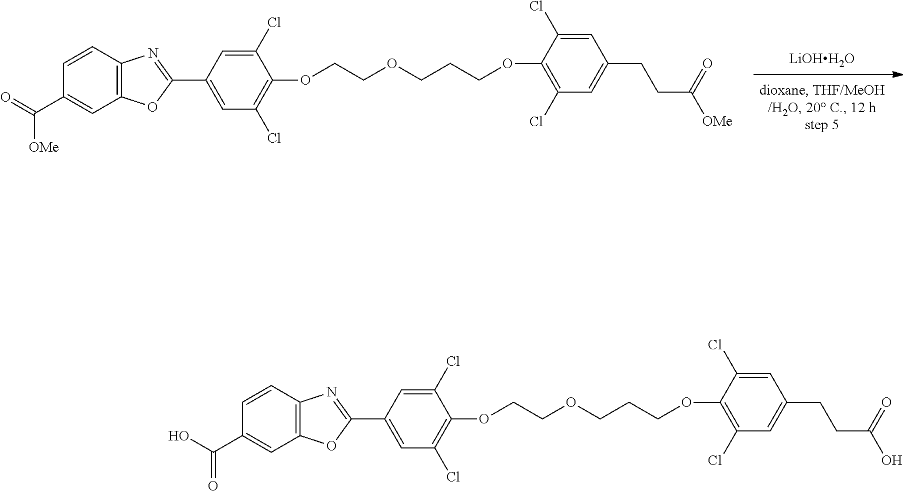 [SMILES: COC(=O)CCc1cc(Cl)c(OCCCOCCOc2c(Cl)cc(-c3nc4ccc(C(=O)OC)cc4o3)cc2Cl)c(Cl)c1 ; "LiOH·H2O" ; O=C(O)CCc1cc(Cl)c(OCCCOCCOc2c(Cl)cc(-c3nc4ccc(C(=O)O)cc4o3)cc2Cl)c(Cl)c1]

### 2-[4-[2-[3-[4-(2-carboxyethyl)-2,6-dichloro-phenoxy]propoxy]ethoxy]-3,5-dichloro-phenyl]-1,3-benzoxazole-6-carboxylic acid

To a solution of methyl 2-[3,5-dichloro-4-[2-[3-[2,6-dichloro-4-(3-methoxy-3-oxo-propyl) phenoxy]propoxy]ethoxy]phenyl]-1,3-benzoxazole-6-carboxylate (150 mg, 223.43 umol, 1 eq) in THF (3 mL)/dioxane (3 mL)/H₂O (3 mL)/MeOH (3 mL) was added LiOH·H₂O (93.75 mg, 2.23 mmol, 10 eq). The reaction was stirred at 20° C. for 12 hr. The reaction was (combined with ET20197-323, 20 mg scale) acidified with HCl (1M) to pH 5 ˜6 and a solid was formed. The suspension was filtered to give the cake. The cake was dissolved in DMSO/MeOH/THF/H₂O (1:1:1:1, 4 mL), adjusted with NaHCO₃ (sat., aq.) to pH 8-9. The solution was purified by prep-HPLC (column: Phenomenex Luna C18 100*30 mm*5 um; mobile phase: [water (0.04% HCl)-ACN]; B %: 70%-95%, 10 min) to afford 2-[4-[2-[3-[4-(2-carboxyethyl)-2,6-dichloro-phenoxy]propoxy]ethoxy]-3,5-dichloro-phenyl]-1,3-benzoxazole-6-carboxylic acid (30.8 mg, 96.8% purity) as an off-white solid.

LCMS: ET20197-324-PIP (M+H⁺): 643.8 @ 2.292 min (5-100% ACN in H₂O, 4.5 min)

¹H NMR: ET20197-324-P1BB (400 MHz, DMSO-d₆)

δ 8.28 (s, 1H), 8.21 (s, 2H), 8.04 (dd, J=1.4, 8.4 Hz, 1H), 7.91 (d, J=8.3 Hz, 1H), 7.30 (s, 2H), 4.36-4.28 (m, 2H), 3.94 (t, J=6.5 Hz, 2H), 3.82-3.76 (m, 2H), 3.64 (t, J=6.2 Hz, 2H), 2.76-2.69 (m, 2H), 2.54-2.52 (m, 2H), 1.95 (quin, J=6.4 Hz, 2H)

DSC: ET20197-324-P1D (Peak: 192.2° C., Onset: 188.1° C., End: 197.2° C.)

### Synthesis of B29 (T-768)

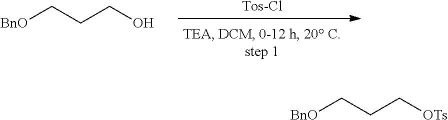 [SMILES: OCCCOCc1ccccc1 ; "Tos-Cl" ; Cc1ccc(S(=O)(=O)OCCCOCc2ccccc2)cc1]

### 3-benzyloxypropyl 4-methylbenzenesulfonate

To a solution of 3-benzyloxypropan-1-ol (10 g, 60.16 mmol, 9.52 mL, 1 eq) in DCM (100 mL) was added TEA (6.09 g, 60.16 mmol, 8.37 mL, 1 eq) and Tos-Cl (11.47 g, 60.16 mmol, 1 eq) at 0° C. The reaction was stirred at 20° C. for 12 hr. The reaction was concentrated to give the residue. The residue was purified by flash silica gel chromatography (ISCO®; 40 g SepaFlash® Silica Flash Column, Eluent of 0 ˜50% Ethyl acetate/Petroleum ether gradient @100 mL/min) to give 3-benzyloxypropyl 4-methylbenzenesulfonate (8.7 g, 27.15 mmol, 45.13% yield) as a colorless oil.

¹H NMR: ET20197-311-P1AA (400 MHz, CHLOROFORM-d)

δ 7.71 (d, J=8.3 Hz, 2H), 7.32-7.10 (m, 7H), 4.33 (s, 2H), 4.09 (t, J=6.2 Hz, 2H), 3.42 (t, J=5.9 Hz, 2H), 2.35 (s, 3H), 1.87 (quin, J=6.1 Hz, 2H)

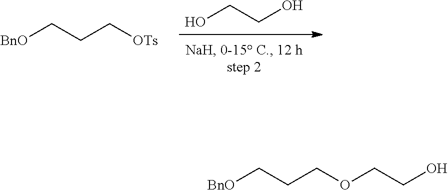

### 2-(3-benzyloxypropoxy)ethanol

To ethylene glycol (16.85 g, 271.53 mmol, 15.18 mL, 10 eq) was added NaH (1.19 g, 29.87 mmol, 60% purity, 1.1 eq) at 0° C. The reaction was stirred at 0° C. for 0.5 h. And then 3-benzyloxypropyl 4-methylbenzenesulfonate (8.7 g, 27.15 mmol, 1 eq) was added at 0° C. The reaction was stirred at 15° C. for 11.5 h. The reaction mixture was quenched and extracted with EtOAc (100 mL*3). The combined organic layers were washed with brine (50 mL*2), dried over [Na₂SO₄], filtered and concentrated under reduced pressure to give a residue. The residue was purified by flash silica gel chromatography (ISCO®; 40 g SepaFlash® Silica Flash Column, Eluent of 0 ˜50% Ethyl acetate/Petroleum ether gradient @ 100 mL/min) to give compound 2-(3-benzyloxypropoxy)ethanol (4.1 g, 18.33 mmol, 67.50% yield, 94% purity) was obtained as a light yellow oil.

¹H NMR: ET20197-314-P1AA (400 MHz, DMSO-d₆)

δ 7.41-7.27 (m, 5H), 4.53 (s, 2H), 3.76-3.68 (m, 2H), 3.65-3.59 (m, 4H), 3.56 (dd, J=4.9, 9.8 Hz, 2H), 2.26 (br t, J=5.9 Hz, 1H), 1.92 (quin, J=6.3 Hz, 2H)

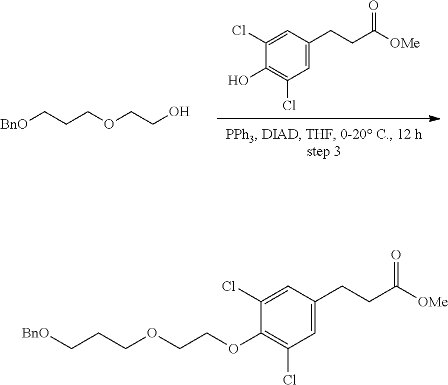 [SMILES: COC(=O)CCc1cc(Cl)c(O)c(Cl)c1 ; OCCOCCCOCc1ccccc1 ; COC(=O)CCc1cc(Cl)c(OCCOCCCOCc2ccccc2)c(Cl)c1]

### Methyl 3-[4-[2-(3-benzyloxypropoxy)ethoxy]-3,5-dichloro-phenyl]propanoate

To a mixture of 2-(3-benzyloxypropoxy)ethanol (1.01 g, 4.82 mmol, 1.2 eq), methyl 3-(3,5-dichloro-4-hydroxyphenyl) propanoate (1 g, 4.01 mmol, 1 eq) and PPh₃ (1.05 g, 4.01 mmol, 1 eq) in THF (20 mL) was added DIAD (811.79 mg, 4.01 mmol, 780.56 uL, 1 eq) at 0° C., the reaction mixture was stirred at 20° C. for 12 hr. The reaction was concentrated to give the residue. The residue was purified by flash silica gel chromatography (ISCO®; 20 g SepaFlash® Silica Flash Column, Eluent of 0 ˜20% Ethyl acetate/Petroleum ether gradient @75 mL/min) to give methyl 3-[4-[2-(3-benzyloxypropoxy)ethoxy]-3,5-dichloro-phenyl]propanoate (1.1 g, 2.24 mmol, 55.87% yield, 90% purity) as a white solid.

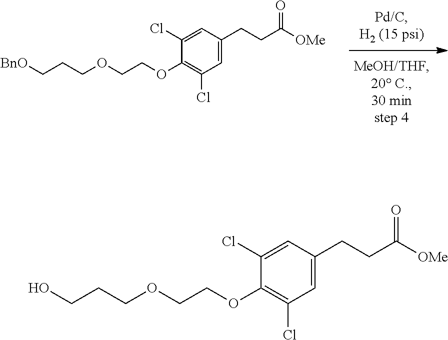 [SMILES: COC(=O)CCc1cc(Cl)c(OCCOCCCOCc2ccccc2)c(Cl)c1 ; COC(=O)CCc1cc(Cl)c(OCCOCCCO)c(Cl)c1]

### Ethyl (2E)-2-hydroxyimino-3-oxo-pentanoate

To a suspension of Pd/C (0.3 g, 10,o purity) in MeOH (10 mL) was added a solution of methyl 3-[4-[2-(3-benzyloxypropoxy)ethoxy]-3,5-dichloro-phenyl]propanoate (500 mg, 1.13 mmol, 1 eq) in THF (10 mL). The reaction mixture was degassed and purged with H₂ for 3 times, the reaction mixture was stirred at 20° C. under H₂ at 15 psi for 30 min. The reaction was detected by TLC. The reaction was filtered through the Celite and washed with MeOH (100 mL). The filtrate was concentrated to give the crude methyl 3-[3,5-dichloro-4-[2-(3-hydroxypropoxy)ethoxy]phenyl]propanoate (400 mg, 990.83 umol, 87.46% yield, 87% purity) as a light yellow solid. The crude was used into next step without further purification.

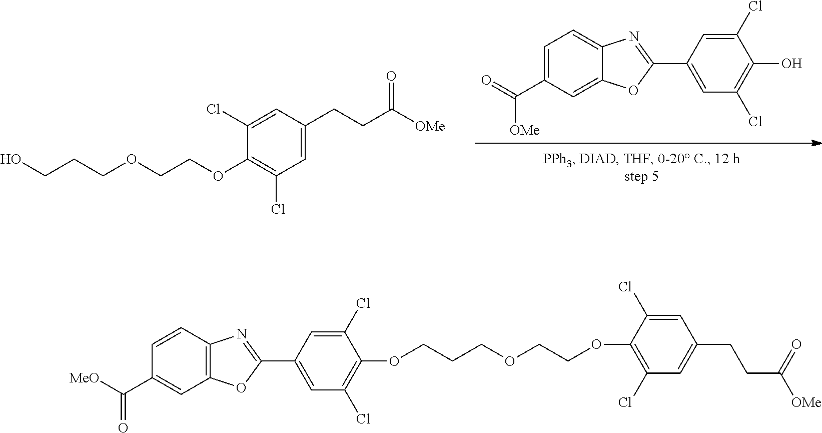 [SMILES: COC(=O)CCc1cc(Cl)c(OCCOCCCO)c(Cl)c1 ; COC(=O)c1ccc2nc(-c3cc(Cl)c(O)c(Cl)c3)oc2c1 ; COC(=O)CCc1cc(Cl)c(OCCOCCCOc2c(Cl)cc(-c3nc4ccc(C(=O)OC)cc4o3)cc2Cl)c(Cl)c1]

### Methyl 2-[3,5-dichloro-4-[3-[2-[2,6-dichloro-4-(3-methoxy-3-oxo-propyl)phenoxy]ethoxy] propoxy]phenyl]-1,3-benzoxazole-6-carboxylate

To a mixture of methyl 3-[3,5-dichloro-4-[2-(3-hydroxypropoxy)ethoxy]phenyl]propanoate (400 mg, 1.14 mmol, 1 eq), methyl 2-(3,5-dichloro-4-hydroxy-phenyl)-1,3-benzoxazole-6-carboxylate (385.10 mg, 1.14 mmol, 1 eq) and PPh₃ (448.07 mg, 1.71 mmol, 1.5 eq) in THF (10 mL) was added dropwise DIAD (345.44 mg, 1.71 mmol, 332.15 uL, 1.5 eq) at 0° C., then the reaction solution was stirred at 20° C. for 12 hr. The solution was concentrated to give the residue. The residue was purified by flash silica gel chromatography (ISCO®; 12 g SepaFlash® Silica Flash Column, Eluent of 0 ˜15% Ethyl acetate/Petroleum ether gradient @75 mL/min) to give methyl 2-[3,5-dichloro-4-[3-[2-[2,6-dichloro-4-(3-methoxy-3-oxo-propyl)phenoxy]ethoxy]propoxy]phenyl]-1,3-benzoxazole-6-carboxylate (300 mg, 285.10 umol, 25.03% yield, 63.8% purity) as an off-white solid.

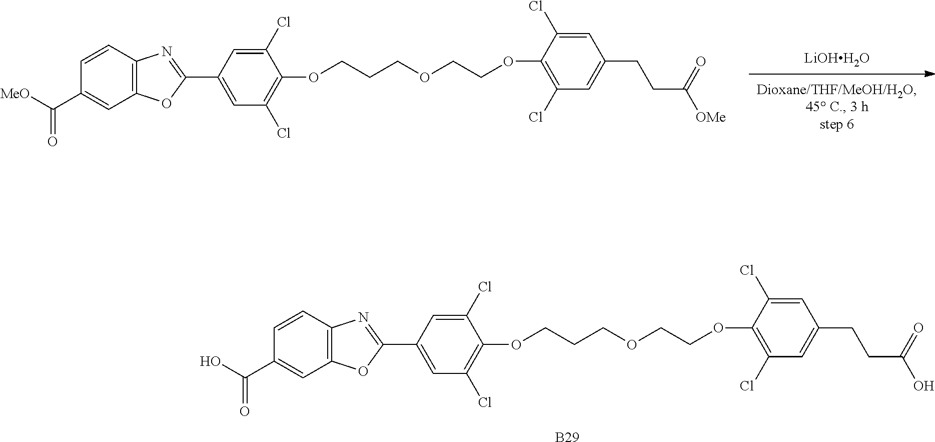

### 2-[4-[3-[2-[4-(2-carboxyethyl)-2,6-dichloro-phenoxy]ethoxy]propoxy]-3,5-dichloro-phenyl]-1,3-benzoxazole-6-carboxylic acid

To a solution of methyl 2-[3,5-dichloro-4-[3-[2-[2,6-dichloro-4-(3-methoxy-3-oxo-propyl) phenoxy]ethoxy]propoxy]phenyl]-1,3-benzoxazole-6-carboxylate (150 mg, 223.43 umol, 1 eq) in THF (2 mL)/dioxane (2 mL)/H₂O (2 mL)/MeOH (2 mL) was added LiOH·H₂O (9.38 mg, 223.43 umol, 1 eq). The reaction was stirred at 45° C. for 3 hr. The reaction was acidified with HCl (1N) and a solid was formed. The suspension was filtered and the cake was washed with H₂O (1 mL). The cake was dissolved in a mixture of DMSO/MeOH/THF/H₂O (1:1:1:1, 4 mL) and adjusted with NaHCO₃ (sat. aq.) to pH˜8 to afford a clean solution. The solution was purified by prep-HPLC (column: Phenomenex Luna C18 100*30 mm*5 um; mobile phase: [water (0.04% HCl)-ACN]; B %: 70%-95%, 10 min]) to give 2-[4-[3-[2-[4-(2-carboxyethyl)-2,6-dichloro-phenoxy]ethoxy]propoxy]-3,5-dichloro-phenyl]-1,3-benzoxazole-6-carboxylic acid (20.6 mg, 31.50 umol, 14.10% yield, 98.38% purity) as a off-white solid.

LCMS: ET20197-321-P1P (M+H⁺): 643.8 @ 2.305 min (5-100% ACN in H₂O, 4.5 min)

¹H NMR: ET20197-321-P1BB (400 MHz, DMSO-d₆)

δ 8.29 (s, 1H), 8.25 (s, 2H), 8.05 (dd, J=1.5, 8.3 Hz, 1H), 7.93 (d, J=8.4 Hz, 1H), 7.33 (s, 2H), 4.20 (t, J=6.4 Hz, 2H), 4.13-4.07 (m, 2H), 3.79-3.74 (m, 2H), 3.70 (t, J=6.1 Hz, 2H), 2.76-2.69 (m, 2H), 2.45-2.39 (m, 2H), 2.10-2.01 (m, 2H)

DSC: ET20197-321-P1D1 (Peak: 199.2° C., Onset: 196.4° C., End: 203.0° C.).

3. Evaluation of Ligand Activity Based on Capacity to Displace ¹²⁵I-Thyroxine (T4) from TTR in Buffer/Human Plasma
3.1 Displacement of ¹²⁵I-T4 from Isolated Wild Type TR.

Competition of ligands with T4 for binding by TTR was assayed quantitatively by a previously described procedure (Kolstoe et al., 2010). Briefly, a solution of 125 nM TTR in 0.1 M Tris-HCl, 0.1 M NaCl and 0.001 M EDTA buffer, pH 8.0, was incubated overnight at 4° C. in the presence of 1.07 nM ¹²⁵I T4 (1,200 μCi/μg, 200 μCi/ml, Perkin Elmer) and increasing concentrations of inhibitor (0-5 μM) in 40 μl final volume. ¹²⁵I T4 bound by TTR was separated from unbound T4 by gel filtration chromatography using Micro Bio Spin 6 columns (Bio-Rad) previously equilibrated with the reaction buffer containing 1% w/v BSA. After counting with a Wizard2 gamma counter (Perkin-Elmer) for 60 s, percentage binding was plotted against the logarithm of the inhibitor concentration, and D₅₀ (concentration of ligand reducing the binding of T4 by TTR by 50%) was determined using a four parameters dose-response curve with GraphPad Prism 5 (Table 1).

3.2 Displacement of ¹²⁵-T4 from TTR in Normal Human Plasma.

The displacement of ¹²⁵I-T4 from TTR in whole plasma, from individuals homozygous for wild type TTR, was studied by incubating plasma with each ligand at increasing concentrations for 30 min at 37° C., further incubation with ¹²³I-T4 followed by measurement of the radioactivity in the immunoprecipitate obtained with anti-TTR antibodies. Briefly 5 μl of plasma were incubated for 30 min at 37° C. with 1 μl of ligand at different final molar concentrations (0-250 μM) followed by a 15 min incubation with 1 μl of ¹²⁵I-T4 (1,200 μCi/μg, 200 μCi/ml, Perkin-Elmer) in a 1/10 dilution with 0.25 μg/ml cold T4 (Sigma-Aldrich) in 0.1 M Tris-HCl, 0.1 M NaCl, pH 8.0 (reaction buffer). Immunoprecipitation was carried out overnight at 4° C. with 2.5 μl of affinity purified sheep polyclonal anti-human TTR antibody (6.26 mg/ml, The Binding Site), 4 μl of 20% w/v PEG6000 in 0.1 M Tris-HCl, 0.1 M NaCl, pH 8.0 and 10 μl Sepharose G15 beads in the same buffer. The immunoprecipitate was washed twice with the reaction buffer containing 2% PEG6000 by centrifugation (11,7000 g for 15 min) and then counted with a Wizard 2 gamma counter (Perkin-Elmer) for 60 s. Percentage binding was plotted against the logarithm of the inhibitor concentration, and D₅₀ (concentration of ligand reducing the binding of T4 by TTR by 50%) was determined using a four parameter dose-response curve with GraphPad Prism 5 (Table 1).

TABLE 1
Ligand concentrations reducing the binding of ¹²⁵I-T4 by TTR in buffer and by
whole human plasma. Values are mean (SD) of at least 3 replicates.

| Ligand | D₅₀ buffer (μM) | | D₅₀ plasma (μM) | |
|---|---|---|---|---|
| T-304 | 0.074 | (0.004) | 1.7 | (0.1) |
| tafamidis | 0.4 | (0.1) | 19.2 | (3.9) |
| AG10 | 0.17 | (0.01) | 1.5 | (0.2) |
| B1 | 0.1 | (0.027) | 1.14 | (0.11) |
| B2 | 0.1 | (0.002) | 2.59 | (0.59) |
| B3 | 0.07 | (0.0095) | 1.7 | (0.2) |
| B4 | 0.07 | (0.012) | 4.4, | (1.2) |
| B4A | 0.066 | (0.009) | 2.3 | (0.5) |
| B5 | 4.6 | (0.4) | 66.4 | (15.7) |
| B6 | 1.17 | (0.41) | 39.7 | (13) |
| B7 | 0.10 | (0.01) | 18.4 | (3.2) |
| B8 | 2.06 | (0.21) | no binding | |
| B9 | 1.35 | (0.4) | no binding | |
| B10 | 5.6 | (3.0) | no binding | |
| B11 | 0.5 | (0.2) | no binding | |
| B15 | 0.10 | (0.014) | 61.0 | (5.0) |
| B16 | 0.09 | (0.012) | 14.1 | (0.8) |
| B17 | 0.10 | (0.03) | 17.3 | (7.6) |
| B18 | 0.21 | (0.04) | >100 | |
| B19 | 0.16 | (0.04) | >100 | |
| B20 | 0.07 | (0.004) | 7.0 | (2.6) |
| B21 | 0.07 | (0.004) | 4.1 | (2.1) |
| B22 | 0.07 | (0.01) | 6.4 | (2.9) |
| B23 | 0.11 | (0.01) | 9.8 | (2.7) |
| B24 | 0.14 | (0.005) | 7.6 | (1.3) |
| B25 | 0.07 | (0.01) | 3.2 | (1.8) |
| B26 | 0.09 | (0.002) | 1.4 | (0.3) |
| B27 | 0.10 | (0.02) | 2.2 | (0.9) |
| B28 | 0.08 | (0.01) | 20.0 | (5.2) |
| B29 | 0.08 | (0.01) | 3.6 | (1.5) |
| B30 | 0.18 | (0.02) | 26.2 | (1.5) |
| B31 | 0.13 | (0.02) | 13.8 | (3.0) |

| | | |
|---|---|---|
| B32* | Weak binding | na |
| B33** | No binding | na |

*B32 shows only weak binding on the short timescale of the present assay, but shows good inhibition of fibrillogenesis on the longer, more physiological timescale described below.
**B33 is a reference compound having the following formula:

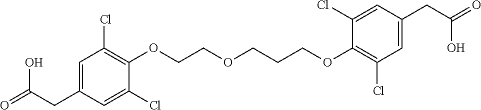 [SMILES: O=C(O)Cc1cc(Cl)c(OCCCOCCOc2c(Cl)cc(CC(=O)O)cc2Cl)c(Cl)c1]

### 4. Inhibition of Mechano-Enzymatic Fibrillogenesis

### 4.1 Quantitative Analysis of TTR Aggregation by Turbidity

The assay for testing the potency of TTR ligands as inhibitors of TTR amyloidogenesis mediated by the selective proteolytic cleavage was carried out as previously described (Marcoux et al 2015; Verona et al 2017).

Briefly, fibrillogenesis of recombinant Val122Ile TTR at 0.5 mg/ml (9 μM) in PBS, pH 7.4 was carried out in glass vials stirred at 1,400 rpm (IKA magnetic stirrer) and 37° C. in the presence and in the absence of trypsin (5 ng/μl). Glass vials had a diameter of 1.4 cm and an air-water interface of 1.5 cm² mimicking the hydrophobic surfaces to which the protein is exposed in vivo. Each ligand was tested at 4 different concentrations (4.5, 9, 18, 36 μM respectively) corresponding to the following drug/TTR molar ratios of 0.5:1, 1:1, 2:1 and 4:1. Several dilutions of the drug stock solutions were made in order to add the same volume of DMSO to each sample. Control samples without drug were added with the same volume of DMSO only. To allow complete binding of ligand by TTR each sample was incubated for 30 min at 37° C. before addition of trypsin. Spectrophotometric turbidity at 400 nm was used to monitor fibril formation over time until it reached a plateau at 96 h. Aggregation of TTR in the presence of equimolar ligand concentrations was quantified as spectrophotometric turbidity at 400 nm as a proportion of 100% for aggregation of the protein in the absence of ligands (Table 2).

### 4.2 Quantitative Analysis of TTR Amyloid Fibrillogenesis by ThT Emission Fluorescence

At the end of the 96 h aggregation, pellets were harvested from each protein sample by 20 min centrifugation at 11,600 g, thoroughly rinsed with PBS to remove non-bound ligand, resuspended with 100 μl PBS, pH 7.4 containing 10 μM ThT in Costar 96-well black-wall plates. Bottom fluorescence values (excitation 440 nm, emission 480 nm) were recorded using a BMG LABTECH FLUostar Omega plate reader. Each value was normalized to the ThT signal of the TTR sample without ligand (Table 2)

TABLE 2
Aggregation of TTR in the presence of equimolar
concentrations of each ligand. Spectrophotometric
turbidity at 400 nm was quantified and ThT emission
fluorescence was normalized to 100 % for aggregation
of the protein without ligands. Mean (SD) values are
shown for at least three independent experiments.

| | % Aggregation | % Aggregation |
|---|---|---|
| Compound | (Turbidity method) | (Fluorescence method) |
| tafamidis | 24.2 (15.0) | 39.0 (14.9) |
| T-304 | 8.64 (5.16) | 5.74 (3.50) |
| T-402 | 16.8 (13.4) | 13.7 (5.82) |
| T-659 | 13.4 (9.55) | 16.0 (12.2) |
| T-205 (B5) | 17.3 (14.8) | 28.8 (13.4) |
| T-540 (B1) | 5.76 (2.79) | 5.21 (3.55) |
| T-449 (B2) | 5.25 (1.68) | 4.36 (1.51) |
| T-663 (B3) | 5.11 (1.58) | 4.49 (2.08) |
| B6 | 6.24 (1.71) | 12.0 (4.44) |
| T-670 (B4) | 3.52 (1.14) | 4.74 (2.93) |
| B3A | 5.76 (2.32) | 8.63 (5.17) |
| B4A | 4.99 (0.46) | 3.30 (1.19) |
| B7 | 4.99 (1.88) | 8.23 (2.61) |
| B8 | 6.35 (2.73) | 11.7 (3.67) |
| B13 | 5.31 (0.62) | 9.76 (4.50) |
| B14 | 5.73 (0.50) | 12.6 (3.17) |
| B9 | 17.1 (0.66) | 9.34 (4.36) |
| B10 | 14.88 (nd) | 16.6 (nd) |
| B15 | 5.56 (0.75) | 12.7 (5.73) |
| B17 | 4.52 (1.08) | 3.86 (2.29) |
| B16 | 4.42 (1.45) | 4.76 (3.13) |
| B18 | 3.31 (0.68) | 2.45 (1.39) |
| B19 | 3.99 (0.928) | 7.27 (1.17) |
| B20 | 11.6 (8.30) | 8.54 (6.10) |
| B21 | 6.43 (3.09) | 8.31 (6.09) |
| B22 | 6.43 (2.12) | 15.5 (3.25) |
| B23 | 4.41 (1.99) | 12.6 (4.42) |
| B28 | 9.30 (4.32) | 24.5 (8.90) |
| B29 | 7.62 (3.39) | 19.2 (13.0) |
| B27 | 4.24 (1.96) | 12.0 (1.06) |
| B25 | 3.96 (1.04) | 8.96 (7.24) |
| B26 | 3.93 (1.73) | 4.62 (2.37) |
| B24 | 6.60 (2.40) | 10.6 (10.5) |
| B30 | Not measured | 15 (5) |
| B31 | Not measured | 7 (2) |
| B32 | Not measured | 18 (6) |
| B33** | Not measured | 75 (30) |

**Reference Compound (see above)

### 5. Ligand Binding Strength Determined by Native Mass Spectrometry (Marcoux et al, 2015)

Recombinant human wild type TTR was prepared for ligand experiments as previously described (Lashuell et al 1999). Immediately before analysis, fully reduced recombinant TTR preparations were buffer-exchanged into 20 mM ammonium acetate, pH 7.0 (Micro-Bio spin 6 column; Bio-Rad). TTR at 3.5 μM final concentration was incubated with two equivalents of T-304 or four equivalents of tafamidis respectively for 2 h at room temperature and excess of any unbound ligand was removed before MS analysis. Spectra were recorded in non-dissociating conditions to highlight the complex (holo TTR); sample cone voltage was then increased to induce ligand dissociation and determine the dissociation voltage (lowest voltage at which the intensity of the TTR alone, apo TTR, corresponded to at least 10% of the holo TTR). To test whether ligands were displaced from the complex, four equivalents of T4 were added and MS analysis was performed immediately and repeated after 24 h if no initial displacement was observed.

Mass spectra were recorded using an LCT mass spectrometer with Z-spray source (Waters) with the following parameters: capillary voltage 1.7 kV, extraction cone voltage 20 V, Pirani pressure ˜5.5 mbar, Penning pressure ˜1.9×10⁻⁶ mbar. Calibration was performed using caesium iodide at 100 mg ml⁻¹, and mass spectra were analysed with MassLynx 4.1 software (Waters). Each solution, 2 μl, was electro-sprayed from gold-coated borosilicated capillaries prepared in house (Hernandez & Robinson 2007).

The mass spectra of TTR/T304 and TTR/tafamidis recorded at low voltage show essentially only peaks corresponding to holo TTR. At increasing cone voltage conditions, tetramer peaks start splitting into corresponding apo and holo TTR as the ligand begins to dissociate. The dissociation voltage, which may be considered as a measure of stability of each complex in the gas phase, is higher for T-304 (110 V) than for tafamidis (70 V) suggesting that the bivalent ligand forms a more stable complex with TTR in the gas phase than tafamidis. Analysis of the spectra confirms stoichiometry of the complexes. Indeed, for the bivalent ligand, the mass difference between the holo (56528±5 Da) and apo (55842±1 Da) species confirms the stoichiometry of 1:1 for T-304/TTR with a 686 Da difference. For tafamidis, the peaks observed at 70 V, corresponds to the two holo forms, h1 (56142±2 Da) and h2 (56467±3 Da) and the apo TTR (55854±34 Da) respectively thus confirming that TTR can be bound by up to two molecules of monovalent ligand.

Analysis of the mass spectra of TTR samples following addition of four-fold molar excess of T4 to the preformed complexes shows that only a small amount of T-304 is displaced from TTR after 24 h incubation. In contrast, tafamidis is mostly displaced within two min of adding T4. This is consistent with the ¹²⁵I-T4 displacement results.

In conclusion, T-304 forms a stable complex with TTR with a protein; ligand ratio of 1:1. The ligand is very slightly displaced by a fourfold molar excess of T4 consistent with a pseudo-irreversible mode. In contrast, the monovalent ligand tafamidis forms a much weaker complex with TTR, with the ligand readily displaced by T4.

### 6. Kinetics of Ligand Binding by TTR

Ligand binding by native TTR quenches the intrinsic fluorescence of the protein. After mixing of ligands with TTR in solution, monitoring emission of the intrinsic fluorescence over time defines the rate at which the ligand is bound by the protein. The fast phase of these binding kinetics was characterized using an SFM 3000 stopped flow device coupled to a MOS500 spectrometer with a fluorescence detection system (Bio Logic, Claix, France) and a cell path length of 1.5 mm. TTR and ligands at 37° C. were mixed at final concentrations of 1 μM each in PBS pH 7.4 containing 0.9% v/v DMSO, and were excited at 280 nm for measurement of total fluorescence emission over 325 nm using a cut off filter. Kinetic data were acquired as the average of at least 3 experimental mixings and fitted to a first order reaction using the general formula y(t)=at +b+C exp (−kt), where y(t) is the observed fluorescence, a (slope) and b (offset) corresponding to the baseline. C and k are, respectively, amplitude and rate constant of the observed fluorescence change. Data were analysed using the Biokine software and values of half-life time (t½) correspond to ln 2/k. (Table 3).

TABLE 3
Binding kinetics of TTR ligands. Fluorescence emission above
325 nm after excitation at 280 nm of native recombinant wild
type TTR at 1 μM with equimolar ligands.
Values of t½ calculated as described in the text.

| TTR ligand | T 1/2 (s) |
|---|---|
| Mds84 | 0.202 |
| B16 | 1.415 |
| B23 | 1.216 |
| B27 | 0.866 |
| B28 | 0.163 |
| B29 | 0.122 |
| B31 | 0.0111 |
| T-304 | 0.0123 |

The kinetics of these typical ligands illustrate the dramatic differences between them, in particular the much faster binding of some of the compounds of the present invention than the prototypic palindromic ligand, mds84, of the prior art (WO2009/040405).

The superiority of the optimal compounds of the present invention is further specifically demonstrated by the much faster quenching of intrinsic protein fluorescence by T-304 than by mds84. The very early changes of fluorescence take place within the dead time of measurement (<5 ms) for both ligands. Burst phases occur in the millisecond time scale with different amplitude for T-304 (13%) and mds84 (8%) reflecting the faster kinetics of T-304 compared to the palindromic compound. The constant rate determined for the ligand-induced-quenching reaction was approximately 15 fold faster for T-304 (k=0.0123 s⁻¹) than for mds84 (k=0.202 s⁻¹). Removal of an aryl group from one of the head groups of the bivalent ligand clearly remarkably accelerates the binding kinetics, which is most desirable for the therapeutic and prophylactic inhibition of TTR amyloid fibrillogenesis.

### 7. 2D NMR of TTR-T304 TTR-B23 Complexes

We have recently reported (Corazza et al 2019) the first evidence that ligands occupying the natural binding site of thyroxine can induce long distance conformational changes that we were able to single out throughout the measurement of chemical shift changes occurring on every single residue. This finding is particularly interesting because it has not previously been observed in the many published X-ray structures of TTR-ligand complexes and it demonstrates the presence in solution of different populations that are not present in the crystals. Moreover, the structural effects of ligand binding were much greater with mds84 than with tafamidis.

Using the same methodology as in Corazza 2019, we have studied the effect of equimolar addition of T304 or B23 to a sample of 90 μM (²H, ¹³C,¹⁵N) TTR in PBS buffer, pH 7.4. 2D NMR [¹H,⁴N] TROSY spectra were acquired with a Bruker AVANCE 800 MHz Spectrometer at 310K. After the addition of T304 or B23 to TTR some of the amide peaks of the apo form vanish and new peaks of the holo species appear indicating the binding of T304 or B23 by TTR. The changes occur for specific TTR residues where the protein senses the chemical effect of the ligands or where the ligands induce structural changes in the protein. The symmetry of tetrameric TTR and the asymmetry of T304 or B23 induce the holo TTR to show peaks with multiple forms, mainly double, a clear indication of the entrance of T304 or B23 into the TTR channel and of occupancy of both binding sites. Analysis of the chemical shift perturbations in the holo TTR structure confirms the involvement of the halogen binding pocket in the binding but also highlights an effect that is well beyond the ligand binding site, involving the outer helix and the outer beta strands.

### 8. Pharmacokinetic Properties

The following properties were determined for selected compounds: Rat oral Cmax in ng/ml at 5 mpk; Rat IV PK at 1 mpk, t½ h; and calculated log P. The results were as follows, with tafamidis for comparison:

TABLE 4

| | Rat Oral | Rat IV PK | |
|---|---|---|---|
| | Cmax | t½ | |
| Compound | (ng/ml) | (h) | logP |
| Tafamidis | 20066 | 16.4 | 4.21 |
| B1 (T-540) | 1630 | 23 | 8.37 |
| B23 (T-762) | 2578 | 5.11 | 5.55 |
| B25 | 217 | 6.97 | 5.36 |
| B26 (T-765) | 1447 | 5.5 | 6.68 |
| B27 | 316 | 5.34 | 5.36 |

It can be seen that the compounds according to the present invention exhibit acceptable pharmacokinetic properties in comparison with tafamidis. Taken in conjunction with the superior TTR binding properties of the compounds of the invention relative to tafamidis, it can be seen that they provide a promising route to TTR stabilization. Crucially, the properties of the present compounds are pharmaceutically acceptable in contrast to the properties of mds84 (WO2009/040405) that made it impossible to develop as a drug.

Each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

### REFERENCES

Corazza, A., Verona, G., Waudby, C. A., Mangione, P. P., Bingham, R., Uings, I., Canetti, D., Nocerino, P., Taylor, G. W., Pepys, M. B., Christodoulou, J., Bellotti, V. Binding of Monovalent and Bivalent Ligands by Transthyretin Causes Different Short- and Long-Distance Conformational Changes. J. Med. Chem. 2019; 62: 8274-8283.
Green, N. S., Palaninathan, S. K., Sacchettini, I C, Kelly, J. W. Synthesis and characterization of potent bivalent amyloidosis inhibitors that bind prior to transthyretin tetramerization. J. Am. Chem. Soc. 2003; 125: 13404-13414.
Hernandez, H., Robinson, C. V. Determining the stoichiometry and interactions of macromolecular assemblies from mass spectrometry. Nat. Protocol., 2007; 2: 715-726.
Jacobson, D. R., Pastore, R. D., Yaghoubian, R., Kane, L5 GaIIo, G., Buck, F. S., Buxbaum, J. N. Variant-sequence transthyretin (isoleucine 122) in late-onset cardiac amyloidosis in black Americans. N. Engl. J. Med., 1997; 336: 466-473.
Kolstoe, S. E., Mangione, P. P., Bellotti, V., Taylor, G. W., Tennent, G. A., Deroo, S., Morrison, A. J., Cobb, A. J., Coyne, A., McCammon, M. G., Warner, T. D., Mitchell, J., Gill R., Smith, M. D., Ley, S. V., Robinson, C. V., Wood, S. P., Pepys, M. B. Trapping of palindromic ligands within native transthyretin prevents amyloid formation. Proc. Natl. Acad. Sci. USA, 2010; 107: 20483-20488.
Lashuel, H. A., Wurth, C., Woo, L., Kelly, J. W. The most pathogenic transthyretin variant, L55P, forms amyloid fibrils under acidic conditions and protofilaments under physiological conditions. Biochemistry, 1999; 38: 13560-13573.
Mangione, P. P., Verona, G., Corazza A., Marcoux, J., Canetti, D., Giorgetti, S., Raimondi, S., Stoppini, M., Esposito, M., Relini, A., Canale, C., Valli, M., Marchese, L., Faravelli, G., Obici, L., Hawkins, P. N., Taylor, G. W., Gillmore, J. D., Pepys, M. B., Bellotti, V. Plasminogen activation triggers transthyrettin amyloidogenesis in vitro. J. Biol. Chem. 2018; 293:14192-14199.
Mangione P P, Porcari R, Gillmore J D, et al. Proteolytic cleavage of Ser52Pro variant transthyretin triggers its amyloid fibrillogenesis. Proc Natd Acad Sci USA 2014; 111:1539-1544:
Mangione P P, Verona G, Corazza A, et al. Plasminogen activation triggers transthyretin amyloidogenesis in vitro. J Biol Chem 2018; 293:14192-14199.
Marcoux, J., Mangione, P. P., Porcari, R., Degiacomi, M. T., Verona, G., Taylor, G. W., Giorgetti, S., Raimondi, S., Sanglier-Cianferani, S., Benesch, J. L., Cecconi, C., Naqvi, M. M., Gillmore, J. D., Hawkins, P. N., Stoppini, M., Robinson, C. V., Pepys, M. B., Bellotti, V. A novel mechano-enzymatic cleavage mechanism underlies transthyretin amyloidogenesis. EMBO Mol. Med. 2015; 7: 1337-1349.
Oza, V. B., Smith, C, Raman, P., Koepf, E. K., Lashuel, H. A., Petrassi, H. M., Chiang, K. P., Powers, E. T., Sachettinni, J., Kelly, J. W. Synthesis, structure, and activity of diclofenac analogues as transthyretin amyloid fibril formation inhibitors. J. Med. Chem. 2002; 45, 321-323.
Pepys, M. B. Amyloidosis. Annu. Rev. Med. 2006; 57: 223-241.
Raimondi S, Mangione P P, Verona G, et al. Comparative study of the stabilities of synthetic in vitro and natural ex vivo transthyretin amyloid fibrils. J Biol Chem 2020; 295:11379-11387.
Robbins, J. Transthyretin from discovery to now. Clin. Chem. Lab. Med. 2002; 40:1183-1190.
Sunde, M., Serpell, L. C., Bartlam, M., Fraser, P. E., Pepys, M. B., Blake, CCF. Common core structure of amyloid fibrils by synchrotron X-ray diffraction. J. MoI. Biol. 1997; 273: 729-739.
Verona, G., Verona, G., Mangione, P. P., Raimondi, S., Giorgetti, S., Faravelli, G., Porcari, R., Corazza, A., Gillmore, J. D., Hawkins, P. N., Pepys, M. B., Taylor, G. W., Bellotti, V. Inhibition of the mechano-enzymatic amyloidogenesis of transthyretin: role of ligand affinity, binding cooperativity and occupancy of the inner channel. Sci. Rep. 2017; 7: 182.
Wiseman, R. L., Johnson, S. M., Kelker, M. S., Foss, T., Wilson, LA., Kelly, J. W. Kinetic stabilization of an oligomeric protein by a single ligand binding event. JACS 2005; 127:5540-5551.

1. Agent for stabilising the native tetrameric form of transthyretin and thereby protecting it from proteolytic cleavage, which comprises a compound of the general formula (I) or a pharmaceutically acceptable salt, ester or prodrug thereof:
A-L-B (I)
wherein:
A is a group of formula (II): 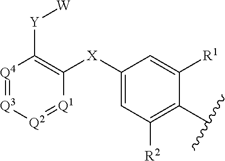
or of formula (III): 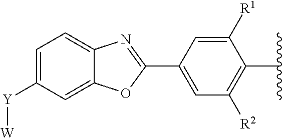
or of formula (IV): 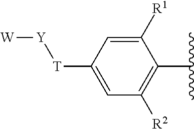
or of formula (V): 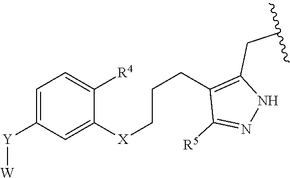
wherein: Y is independently a direct bond or a C1-C4 alkylene group which may be linear, branched, or may include a cyclopropyl group;
W is —COOH or a tetrazole group;
Q¹, Q², Q³ and Q⁴ are independently CH or N, provided that no more than two of Q¹, Q², Q³ and Q⁴ are N;
X is independently —NH—, —O—, —S—, —CH₂—, —NR—, —CO—, —CONH—, —CONR—, —C═N—O—, —NHCO—, —NRCO—, —O—N═C—, —SO—, —SO₂— or a direct bond, wherein R is C1-C3 alkyl optionally substituted by one or more halogen atoms;
each of R¹, R² and R⁴ is independently selected from H, F, Cl, Br, I, CN, CF₃, OCF₃, R′, OR′, NR′R′, SOR′ or SO₂R′, wherein R′ are each independently C1-C3 alkyl optionally substituted by one or more halogen atoms;
R⁵ is selected from C1-C3 alkyl or C1-C3 alkoxy optionally substituted by one or more halogen atoms or —OH groups; and
T is selected from groups of the following formulas (IVa) to (IVf): 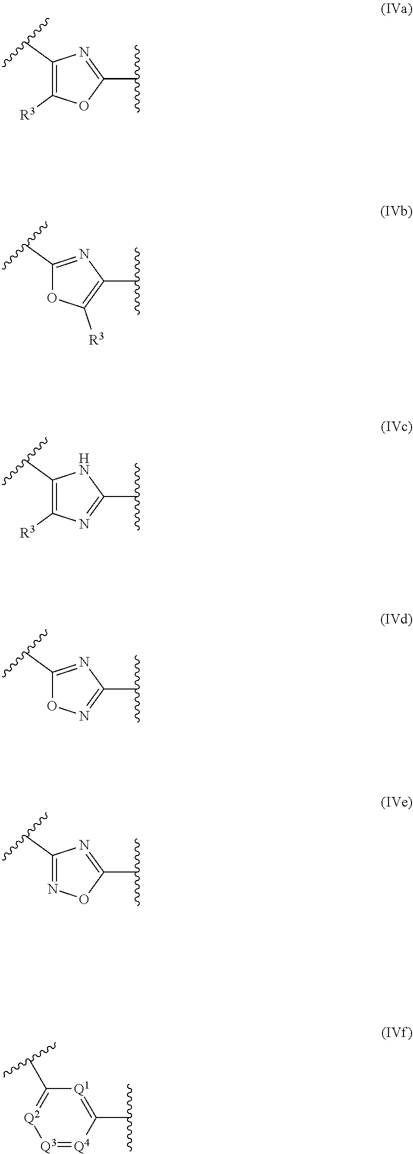
wherein R³ is selected from C1-C3 alkyl or C1-C3 alkoxy optionally substituted by one or more halogen atoms or —OH groups;
B is a group of formula (III), (IV) or (V), or a group of formula (VI): 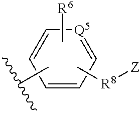
or a group of formula —R¹⁰Z,
wherein: Q⁵ is N or CR⁷;
each of R⁶ and R⁷ is independently selected from H, F, Cl, Br, I, CF₃, CN, OCF₃, R′, OR′, NR′R′, SOR′ or SO₂R′, wherein R and R′ are each independently C₁-C₃ alkyl optionally substituted by one or more halogen atoms, and provided that R⁶ and R⁷ are not both H;
R⁸ is -Alk-, —CONH(Alk)-, or —COO(Alk)-, where Alk is a C1-C4 alkylene or alkenylene group which may be linear or branched or may include a cyclopropyl group, or Re is a group of formula (VII): 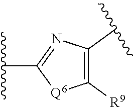
wherein Q⁶ is selected from O or S and R⁹ is C1-C4 alkyl or alkoxy;
Z is selected from —CO₂R′, —CONR′R″, —SO₂R′ wherein R′ and R″ are independently H or C1-C4 alkyl; and
R¹⁰ is a C1-C4 alkylene or alkenylene group;
and
L represents a linker group which is a saturated or unsaturated chain of 5 to 13 carbon atoms in which optionally from one to three of the carbon atoms are replaced by O, S, NR′, SO, SO₂, or CONR′, wherein R′ is H or C1-C3 alkyl, and wherein the said chain is unsubstituted or substituted by one or more groups comprising halogen, OH, C1-C3 alkyl, C2-C3 alkenyl, C2-C3 alkynyl or C1-C3 alkoxy.

2. Agent according to claim 1, wherein A is a group of formula (II) or (III), and: Y is a direct bond, and/or W is —COOH, and/or X is —NH—, and/or R¹ is Cl or H, and/or R² is Cl.

3. Agent according to claim 1, wherein the groups of formula (II) are selected from: 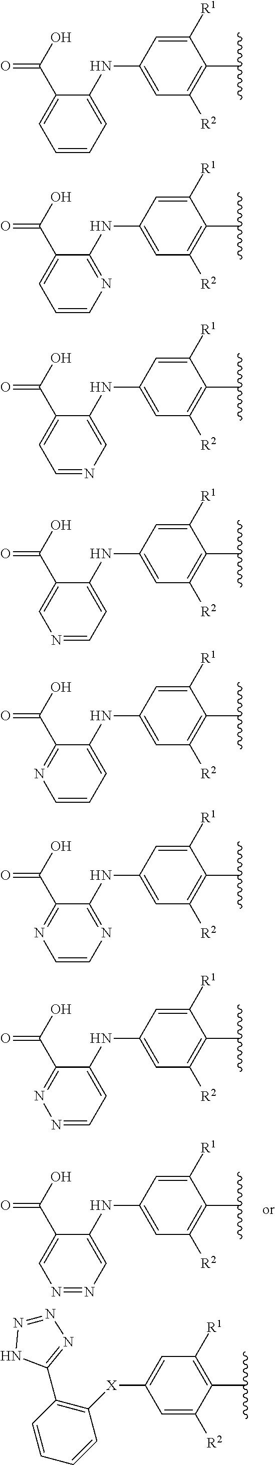
wherein X, R¹ and R² are as defined in claim 1, suitably wherein R¹ and R² are independently selected from H and C₁.

4. Agent according to claim 1, wherein the groups of formula (III) are selected from: 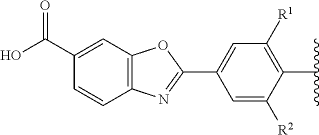
wherein R¹ and R² are as defined in claim 1, suitably wherein R¹ and R² are independently selected from H and Cl.

5. Agent according to claim 1, wherein, in the groups of formula (IV): Y is a direct bond, and/or W is —COOH, and/or R¹ is Cl or H, and/or R² is Cl, and/or R³ is —CH₃ or —C₂H₅.

6. Agent according to claim 1, wherein the groups of formula (IV) have the following formula: 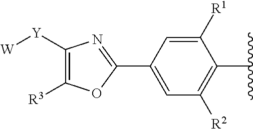
and more suitably the following formula: 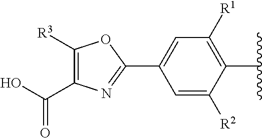
wherein the substituents are as defined in claim 1, and R³ is H, methyl or ethyl.

7. Agent according to claim 1, wherein in the groups of formula (V): Y is a direct bond, and/or W is —COOH, and/or X is —O—, and/or R⁴ is F, and/or R⁵ is —CH₃ or —C₂H₅.

8. Agent according to any preceding claim, wherein B is a group of formula (VI) having the following structure: 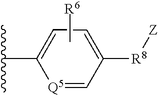
for example wherein B has the following structure:

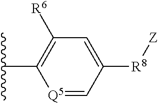

9. Agent according to any of claims 1 to 7, wherein B is a group of formula (VI) and wherein Q⁵ is CH, Z is COOH, and R⁸ is a direct bond, CH₂ (methylene), C₂H₄ (ethylene) or C₃H₆ (n-propylene).

10. Agent according to any of claims 1 to 8, wherein B is a group of formula (VI), and Q⁵ is CR⁷, and/or each of R⁶ and R⁷ is Cl, and/or Z is —COOH, and/or R⁸ is selected from —CH₂—, —C₂H₄—, or —CONHCH(CH₃)—, or wherein the group R⁸Z is selected from:

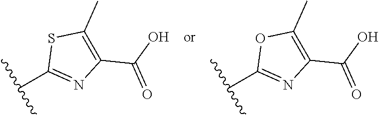

11. Agent according to any preceding claim, wherein R¹⁰ is —CH₂— or —C₂H₄— and/or Z is —COOH.

12. Agent according to any preceding claim, wherein the linker group L is linked to the groups A and B by ether linkages at the terminal ends of the linker group.

13. Agent according to claim 12, wherein the linker group L is a dialkylene oxide group or a trialkylene oxide group of formula (VII):
—O—R¹¹—X—R¹²—O—(R¹³—O)ₘ
wherein: m is 0 or 1; X is O, NH, CH(OH), C(═O)NH, SO, or SO₂; and R¹¹, R¹² and R¹³ are independently methylene, ethylene, n-propylene or n-butylene groups optionally substituted by one or more groups selected from the group consisting of halogen, OH, C1-C3 alkyl, C2-C3 alkenyl, C2-C3 alkynyl or C1-C3 alkoxy.

14. Agent according to claim 13, wherein: m is 0, R¹¹ and R¹² are independently ethylene or n-propylene groups, and X is O, NH, CH(OH), C(═O)NH, SO, or SO₂.

15. Agent according to any claim 13, wherein the linker group is one of the following linker groups:

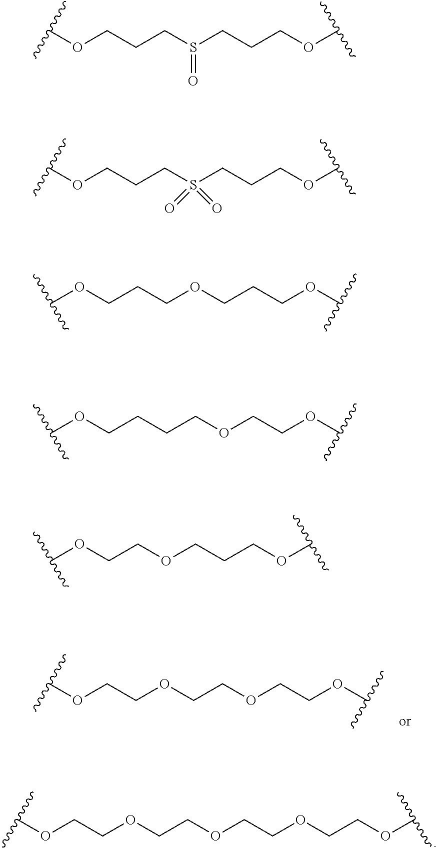

16. Agent according to claim 12, wherein the linker group is a linear or branched chain of 5 to 13 carbon atoms substituted with one, two or three —OH groups.

17. Agent according to claim 16, wherein the linker group is one of the following linker groups:

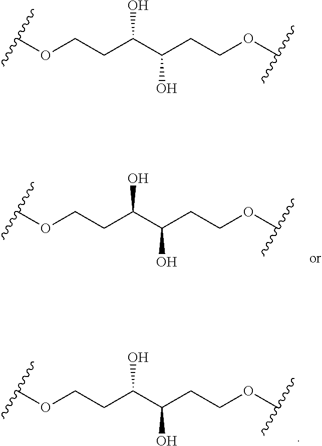 [SMILES: *OCC[C@H](O)[C@@H](O)CCO* ; *OCC[C@@H](O)[C@H](O)CCO* ; *OCC[C@@H](O)[C@@H](O)CCO*]

18. Agent according to claim 12, wherein the linker group is a group of formula (VII):
—O—R¹⁰—O—
wherein R¹⁰ is an alkylene or alkenylene group comprising from 4 to 10 carbon atoms in the chain, optionally substituted by one or more groups selected from the group consisting of halogen, OH, C1-C3 alkyl, C2-C3 alkenyl, C2-C3 alkynyl or C1-C3 alkoxy.

19. Agent according to claim 18, wherein the linker group is as follows: 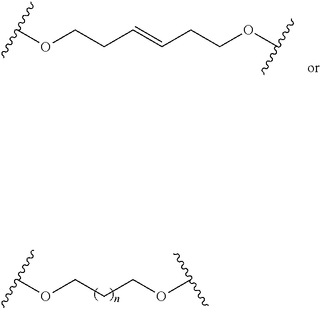
wherein n=2, 3, 4, 5, 6, 7 or 8.

20. Agent according to claim 1, wherein the agent comprises one or more of the following compounds according to general formula (I): 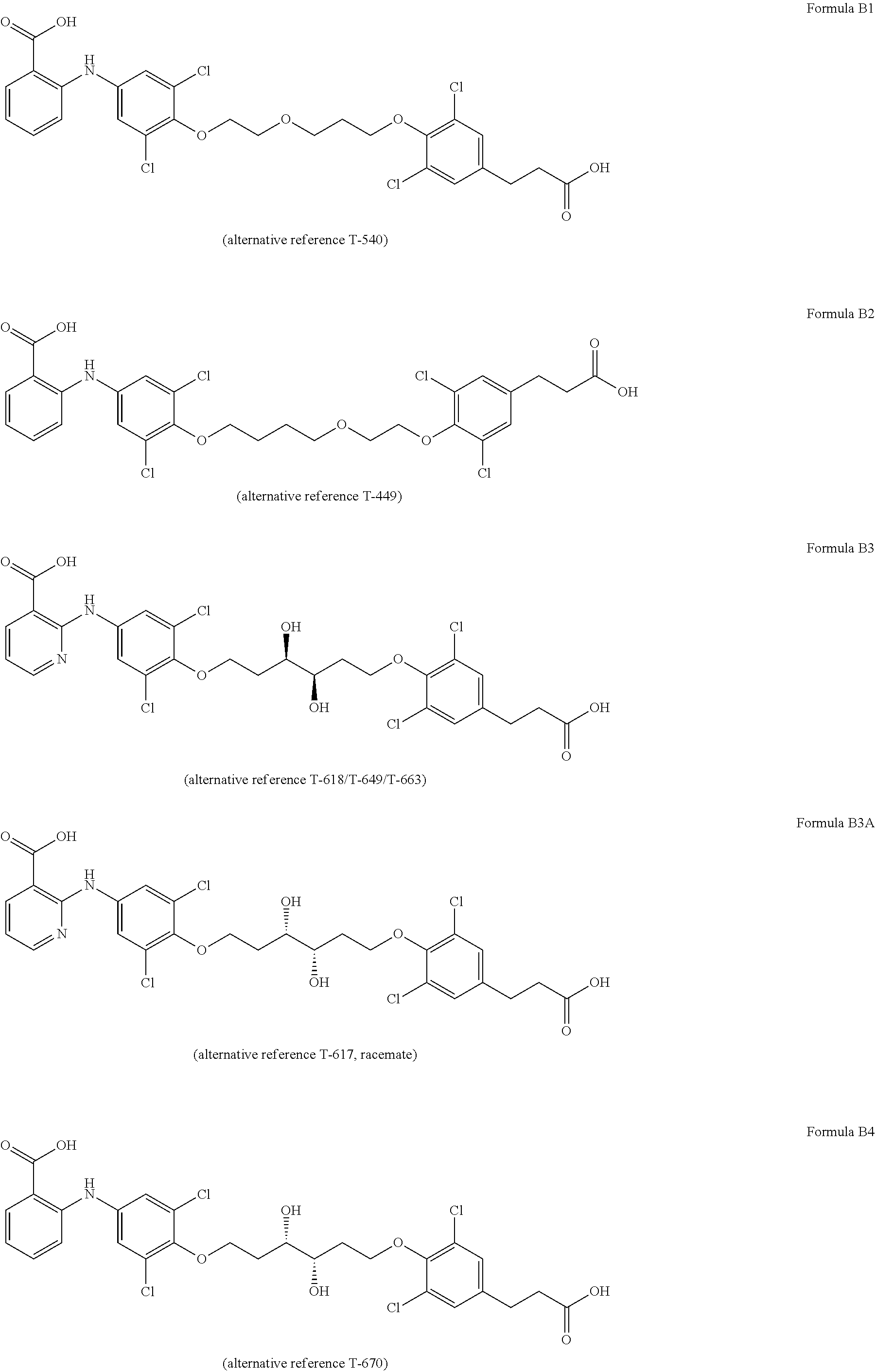 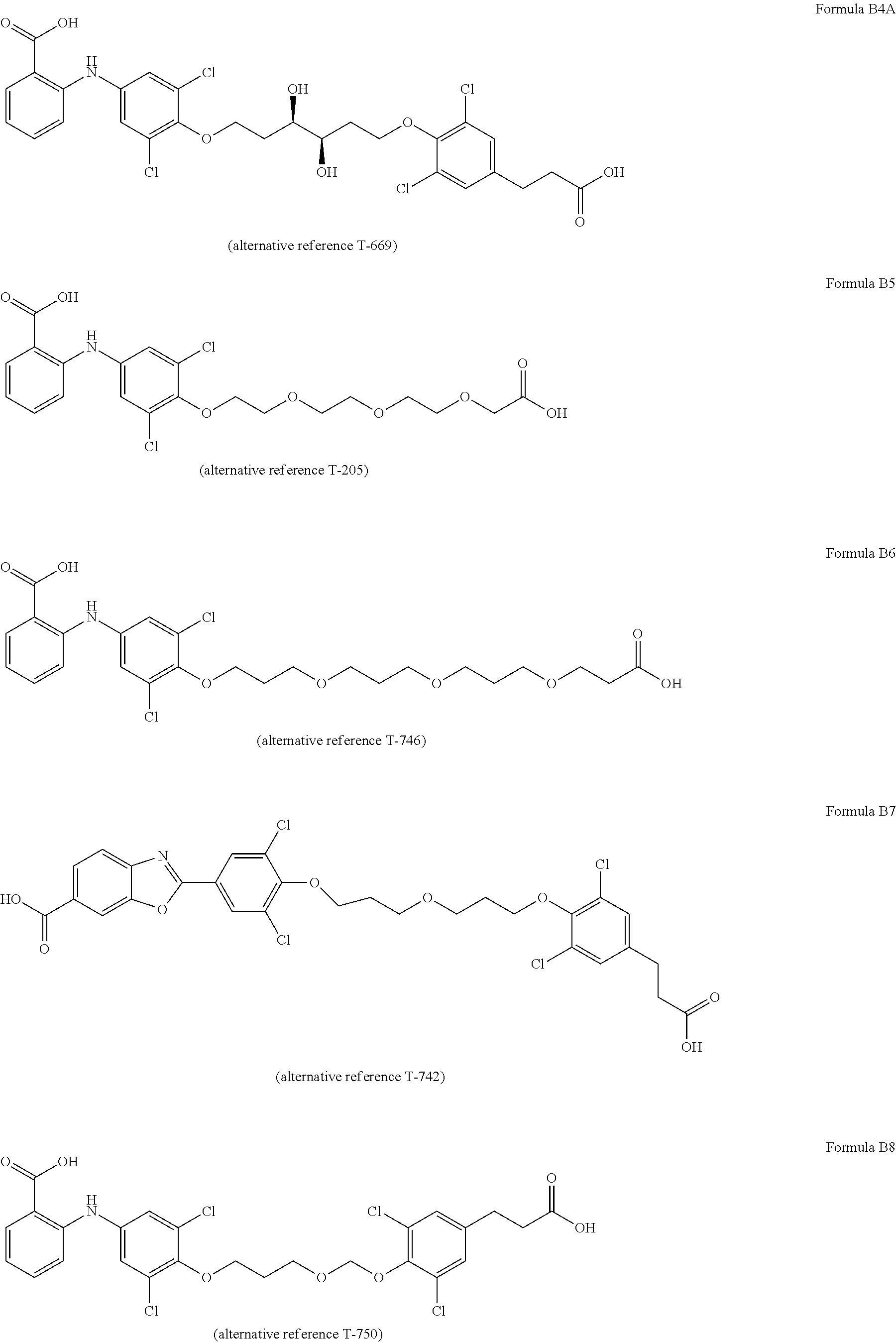 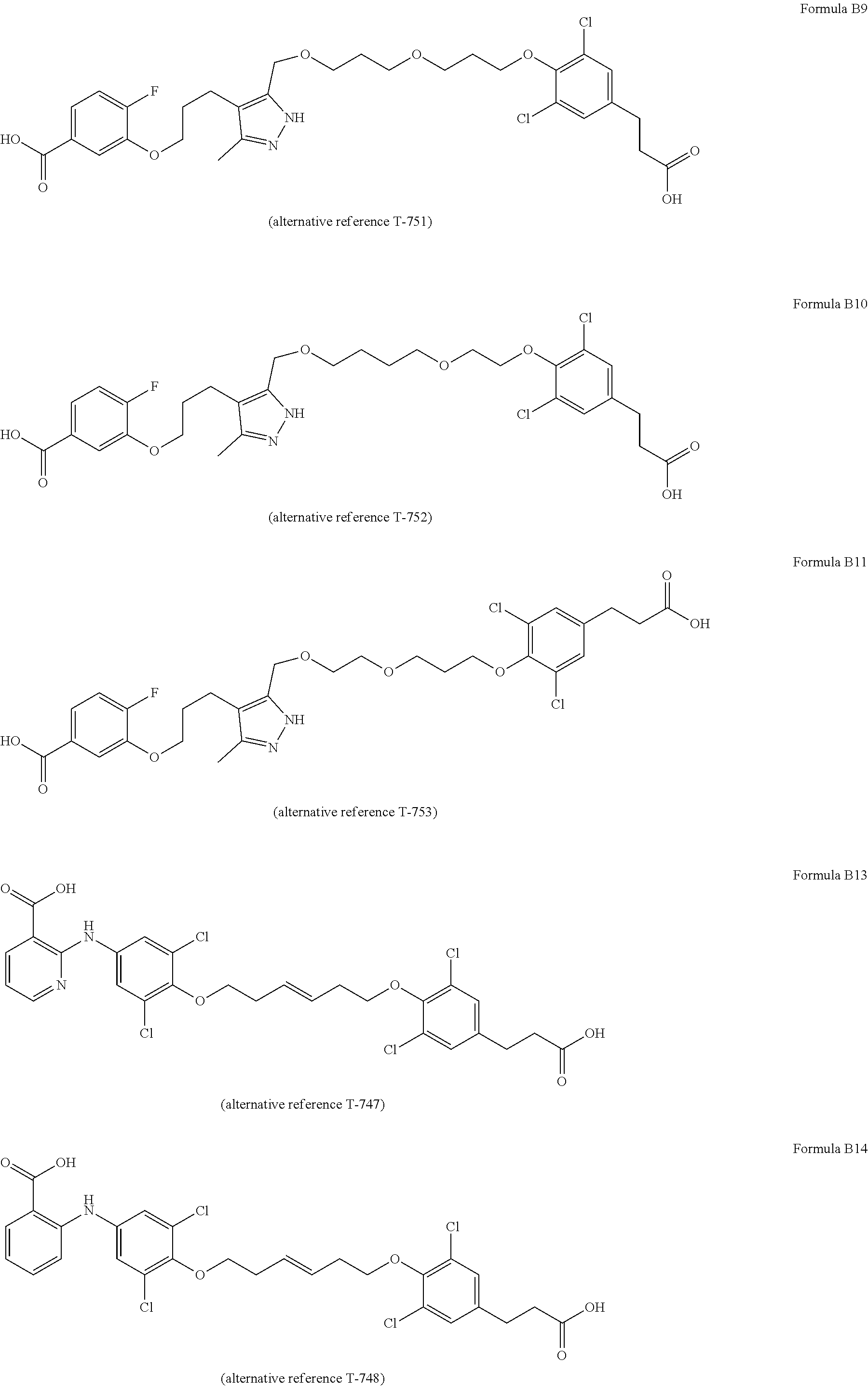 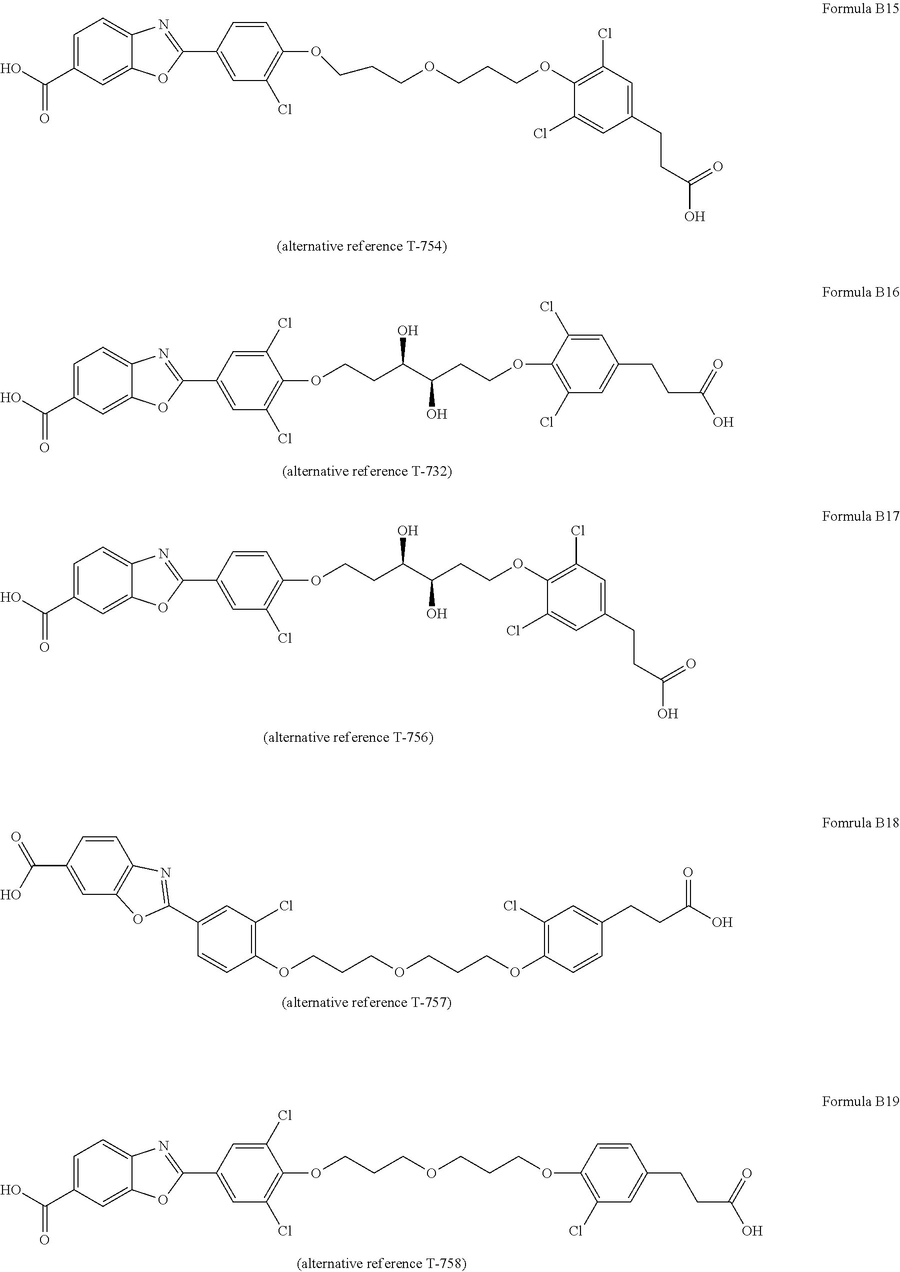 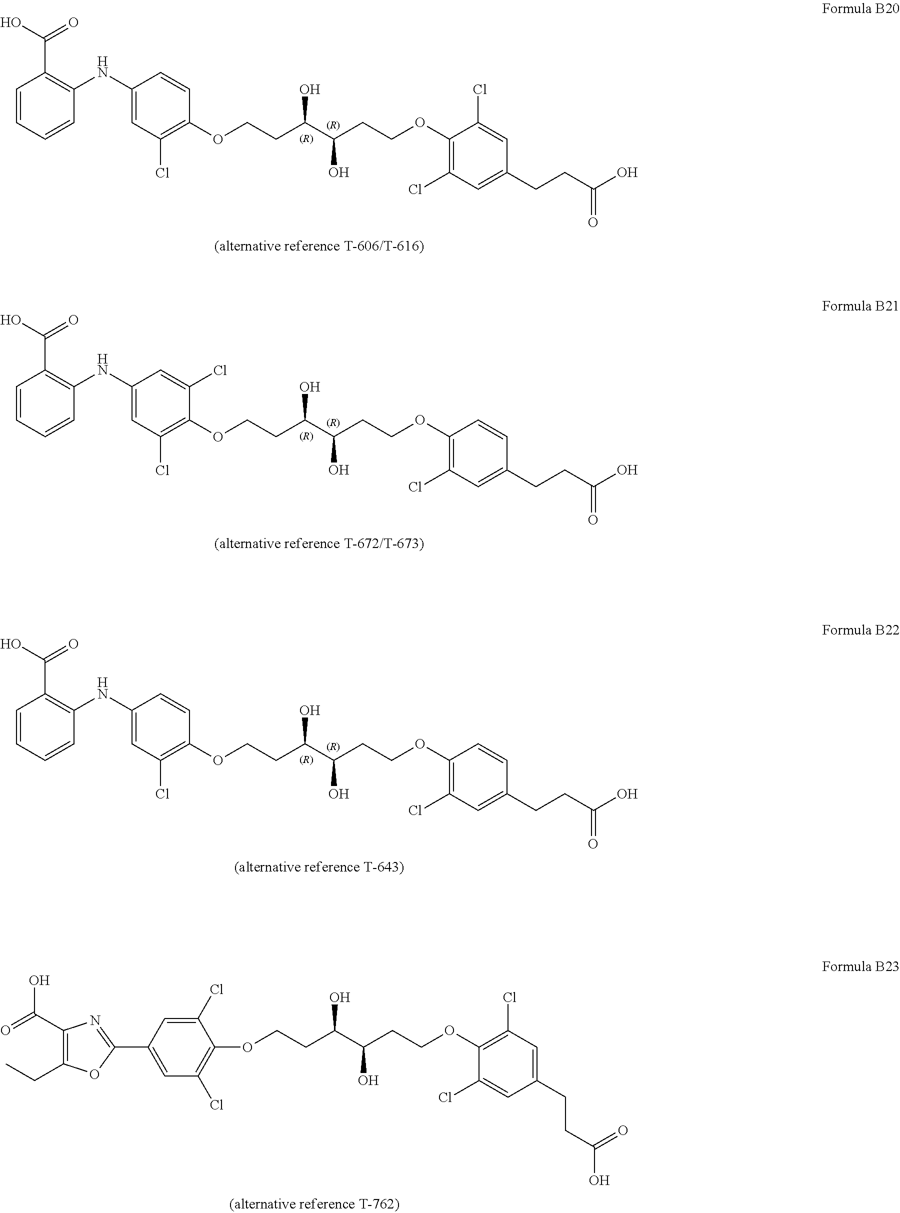 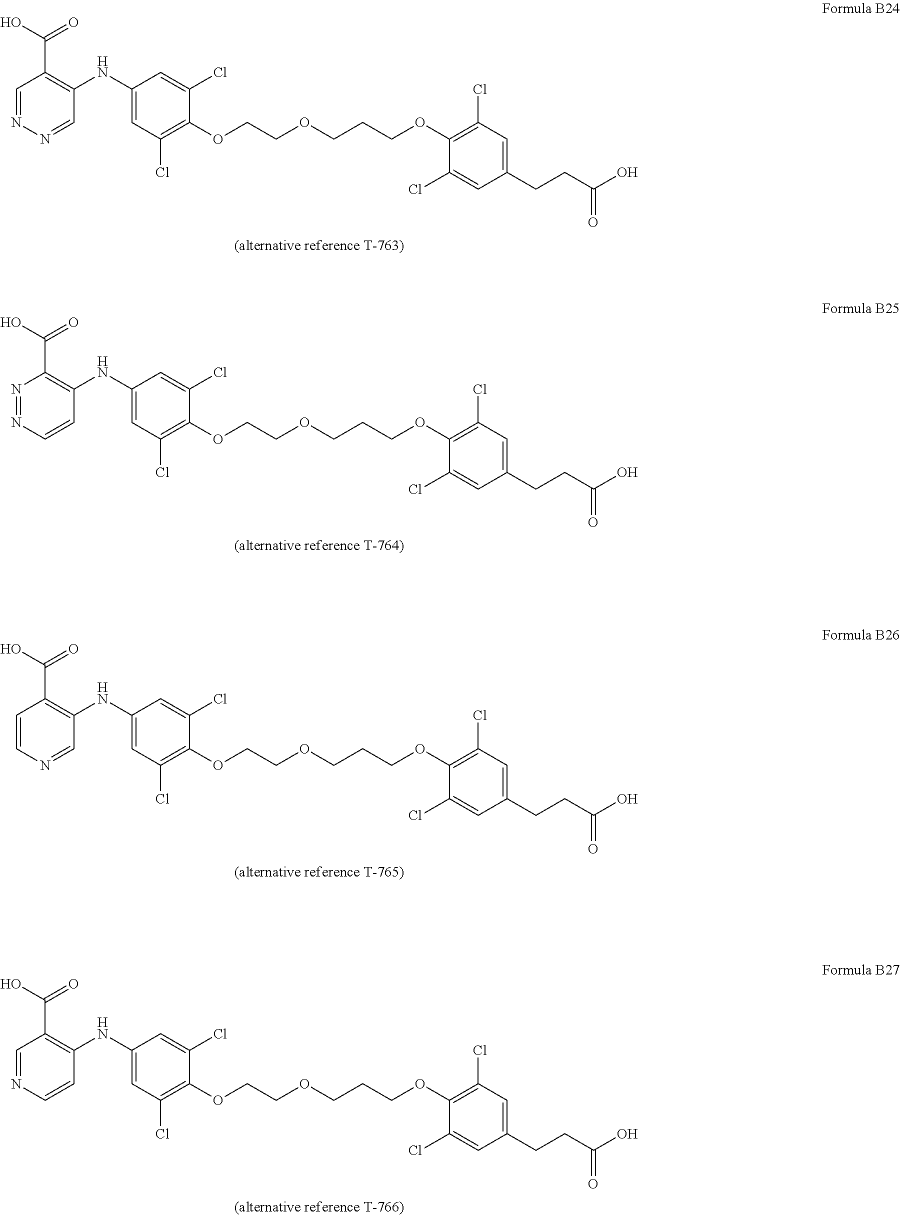 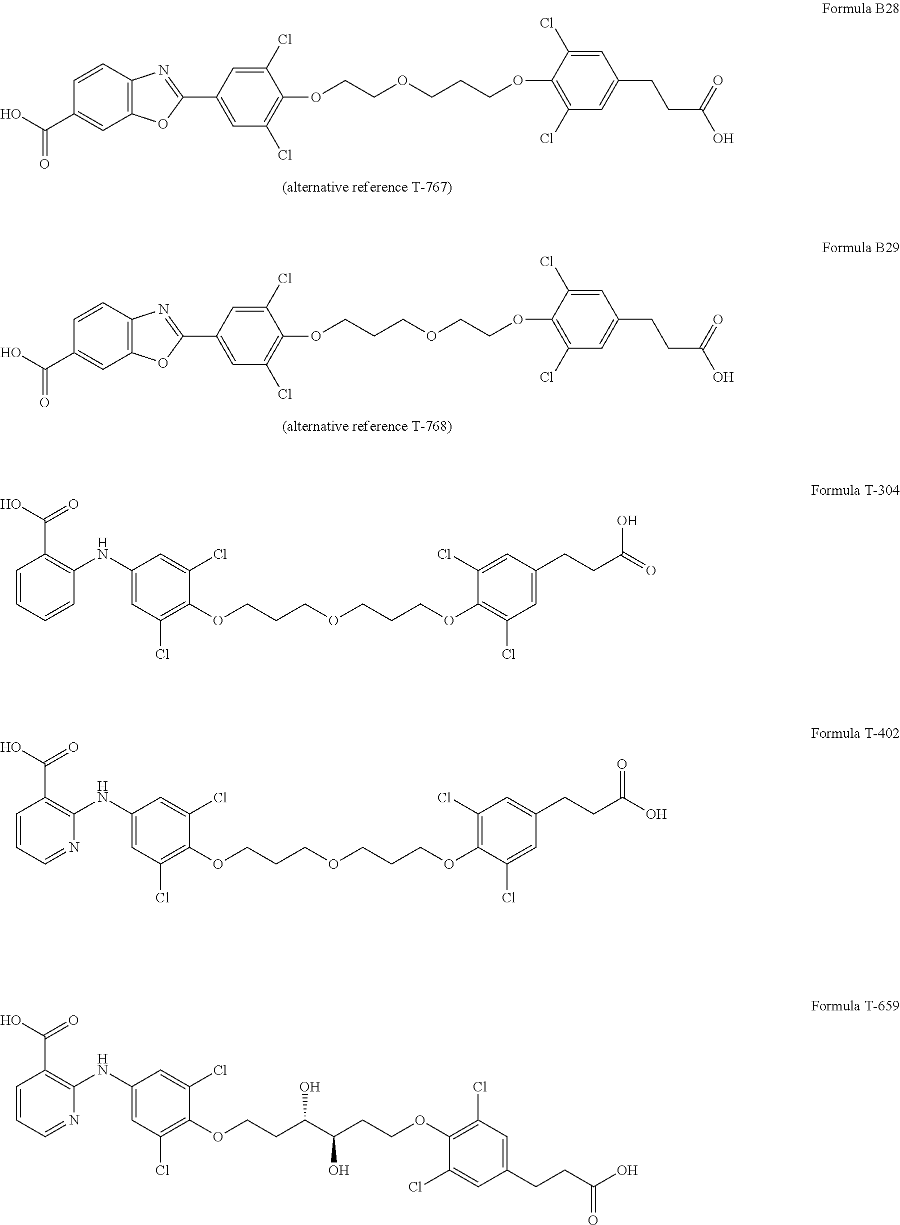 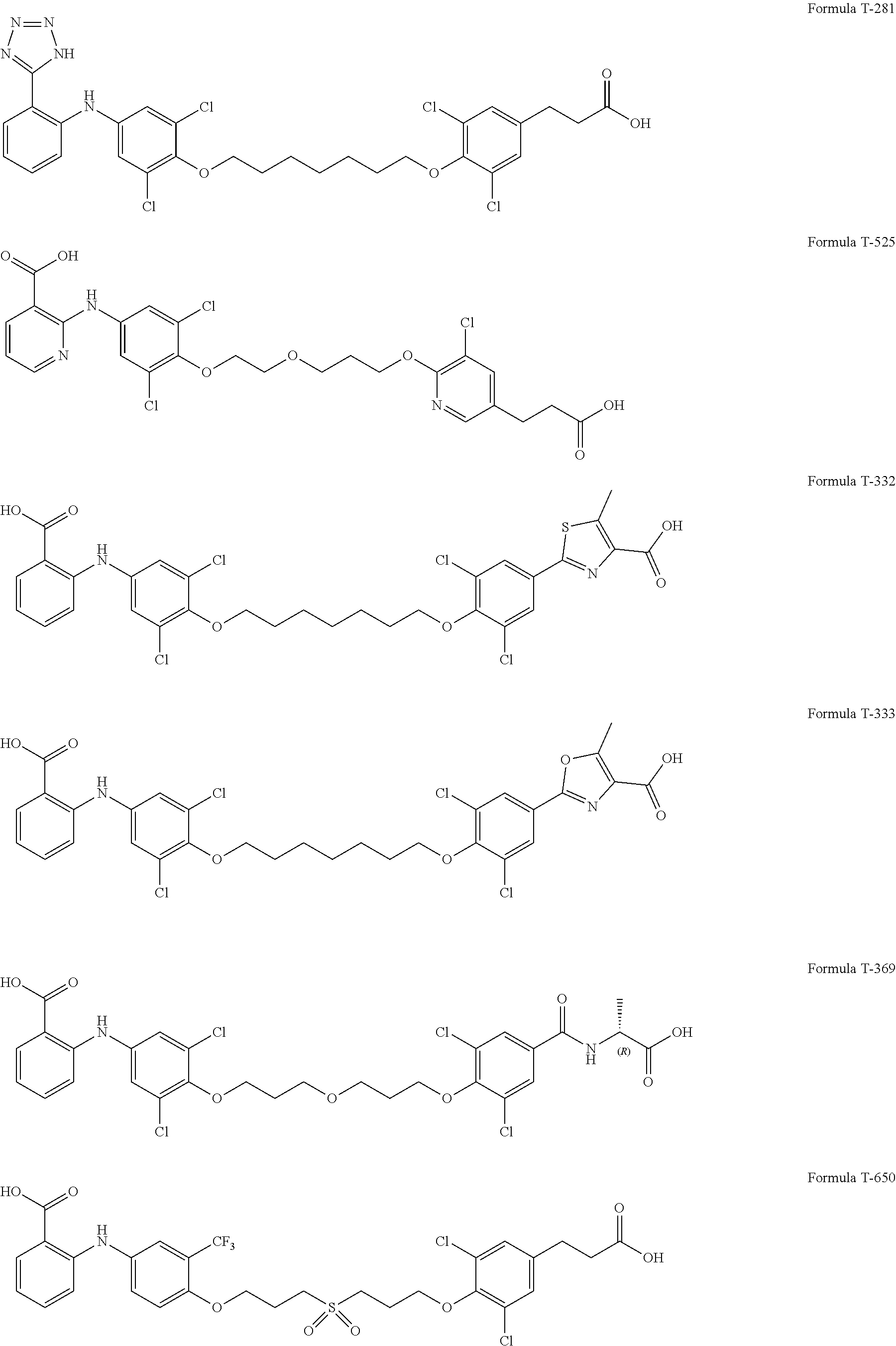 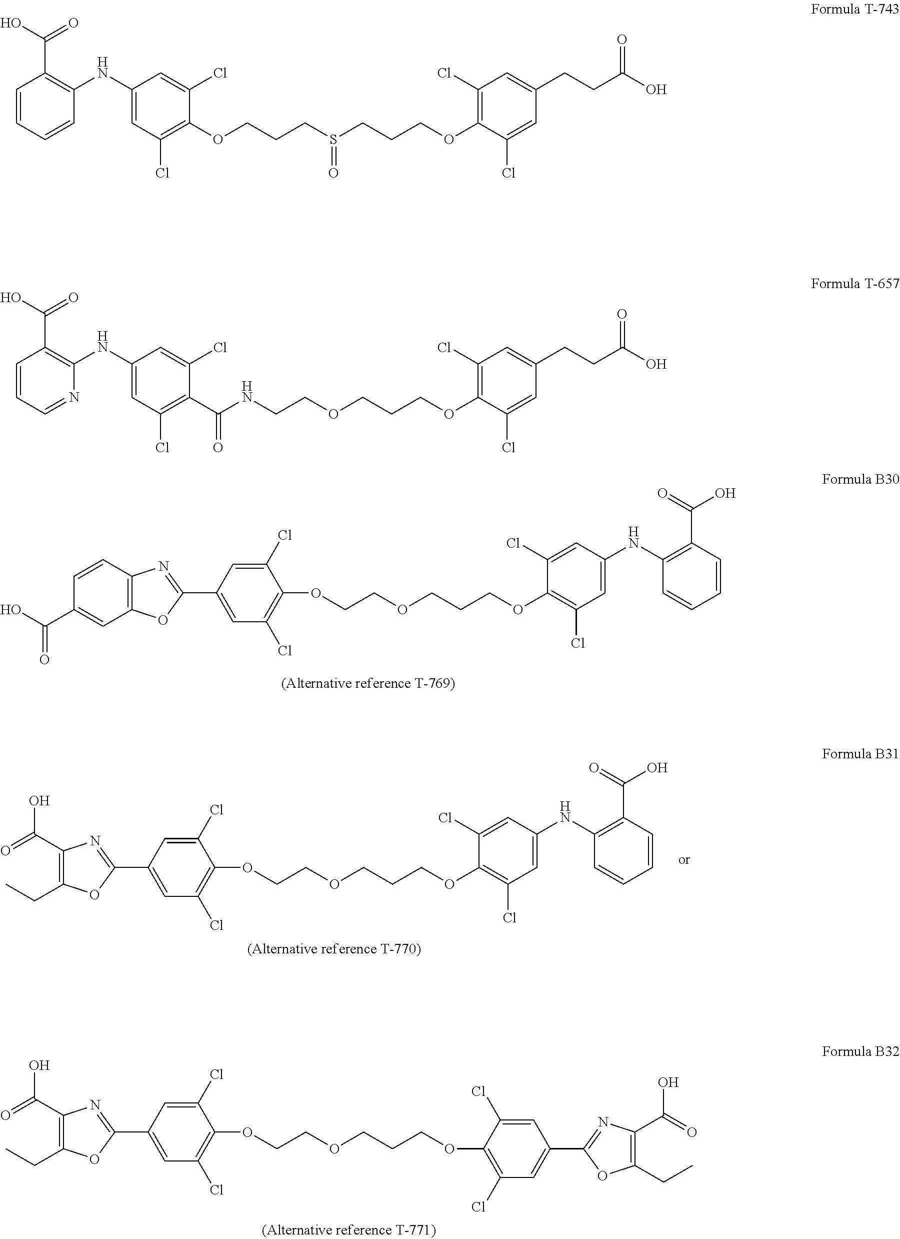
or a pharmaceutically acceptable salt or ester thereof.

21. Agent according to any preceding claim, wherein the compound of Formula (I) has a [D₅₀] for displacement of ¹²⁵I-T4 from isolated TTR as measured by the method disclosed herein of less than about 1 μM, preferably less than about 0.25 μM, more preferably less than about 0.15 μM.

22. Agent according to any preceding claim, wherein the compound of Formula (I) has a [D₅₀] for displacement of ¹²⁵-T4 from TTR in whole human plasma as measured by the method disclosed herein of less than about 15 μM, preferably less than about 10 μM, more preferably less than about 5 μM.

23. Agent according to any preceding claim, wherein the compound of Formula (I) inhibits mechano-enzymatic fibrillogenesis of Val122Ile TTR as determined by turbidity measurement and thioflavin T fluorescence measurement, according to the methods disclosed herein, such that the % aggregation at 96 h is less than about 25%, preferably less than about 20%.

24. Agent according to any preceding claim, wherein the compound of Formula (I) has a hydrophilic/lipophilic partition coefficient (log P) less than about 10, suitably less than about 8, more suitably less than about 6.

25. Agent according to any preceding claim, for use in the treatment or prevention of transthyretin amyloidosis.

26. Agent according to claim 25, wherein the transthyretin amyloidosis comprises systemic transthyretin amyloidosis.

27. Use of an agent according to any one of claims 1 to 24, for the manufacture of a medicament for treatment or prevention of transthyretin amyloidosis.

28. Use according to claim 27, wherein the transthyretin amyloidosis comprises systemic transthyretin amyloidosis.

29. A pharmaceutical composition comprising an agent according to any of claims 1 to 24 in admixture with one or more pharmaceutically acceptable excipients, diluents or carriers.

30. A method for stabilising the tetrameric form of transthyretin in a patient in need thereof, comprising administering to the patient a therapeutic amount of an agent according to any of claims 1 to 24 or a pharmaceutical composition according to claim 29.
